(19) United States
(12) Patent Application Publication
(10) Pub. No.: US 2022/0251241 A1
(43) Pub. Date: Aug. 11, 2022

(54) HTRA1-BINDING AGENTS AND METHODS OF USE THEREOF

(71) Applicant: NGM Biopharmaceuticals, Inc., South San Francisco, CA (US)

(72) Inventors: Chun Chu, Foster City, CA (US); Raj Haldankar, Redwood, CA (US); Betty Chan Li, Millbrae, CA (US); Zhonghao Liu, Redwood City, CA (US); Alexander V. Loklev, Palo Alto, CA (US); Jie Tang, Palo Alto, CA (US); Yan Wang, Foster City, CA (US)

(21) Appl. No.: 17/666,381

(22) Filed: Feb. 7, 2022

Related U.S. Application Data
(60) Provisional application No. 63/146,992, filed on Feb. 8, 2021.

(51) Int. Cl. C07K 16/40; A61K 39/395; A61K 31/7105; A61K 38/17

(52) U.S. Cl. CPC C07K 16/40; A61K 39/3955; A61K 31/7105; A61K 38/179; A61K 39/00; C07K 2317/565; C07K 2317/51; C07K 2317/31; C07K 2317/24; C07K 2317/56

(57) ABSTRACT

The present disclosure provides binding agents, such as antibodies, that specifically bind HTRA1, including human HTRA1, as well as compositions comprising the binding agents, and methods of their use. The disclosure also provides related polynucleotides and vectors encoding the binding agents and cells comprising and expressing the binding agents.

### CROSS REFERENCE

This application claims the benefit of U.S. Provisional Application No. 63/146,992 filed Feb. 8, 2021, the disclosure of which is incorporated by reference herein in its entirety.

### SEQUENCE LISTING

This application incorporates by reference in its entirety a Sequence Listing submitted with this application as a text filed entitled “13370-125-999_Sequence_Listing_ST25.txt”, created on Feb. 3, 2022, and is 134,818 bytes in size.

### FIELD OF THE INVENTION

The present disclosure generally relates to agents that bind high-temperature requirement A serine peptidase 1 (HTRA1), particularly antibodies that bind human HTRA1, as well as compositions comprising the HTRA1-binding agents and methods of using the agents and compositions.

### BACKGROUND

Aged-related macular degeneration (AMD) is the leading cause of irreversible blindness in adults over 50 years old. It is estimated that 170 million people have AMD globally, and this is expected to increase to 288 million by 2040 (Pennington et al., 2016, Eye and Vision, 3:34).

AMD is a neurodegenerative disease that preferentially affects the macular (central) region of the retina. Some of the common symptoms of AMD include visual distortions, such as straight lines seeming bent, reduced central vision in one or both eyes, decreased intensity or brightness of colors, a well-defined blurry spot or blind spot in your field of vision, and a general haziness in your overall vision. The disease is categorized into early stage, intermediate stage, or advanced (late) stage based on the severity of symptoms, and other characteristics such as the number and size of drusen and the presence or absence of choroidal neovascularization (CNV). There are two forms of AMD, (i) the neovascular/exudative form, referred to as wet AMD, and (ii) the atrophic form, referred to as dry AMD. The advanced form of dry AMD is referred to as geographic atrophy (GA). Wet AMD generally begins as the dry form and symptoms usually appear suddenly and worsen rapidly. GA causes a more gradual, but relentlessly progressive and irreversible, vision loss than wet AMD. It has been estimated that of patients diagnosed with AMD, approximately 80% have GA, while the rest have neovascular/wet AMD.

Given the poor outcomes associated with both forms of AMD, there is considerable interest in therapeutic interventions that can halt the progression of early or intermediate stage AMD to advanced AMD. Most current therapies are directed toward wet AMD, and target abnormal blood vessel growth through inhibition of vascular endothelial growth factor A (VEGF-A). However, the majority of patients require indefinite treatment and/or demonstrate progression of disease despite therapeutic intervention (Amoaku et al., 2015, Eye, 29:721-31). Although there has been some progress in the development of therapeutics for wet AMD, there are no approved treatments for dry AMD and/or GA. Thus, new therapeutic agents for the treatment of AMD are desperately needed.

### BRIEF SUMMARY

The present disclosure provides agents that bind high-temperature requirement A serine peptidase 1 (HTRA1). The agents include, but are not limited to, polypeptides such as antibodies that specifically bind HTRA1. The agents may be referred to herein as “HTRA1-binding agents”. The disclosure provides methods of making and of using a HTRA1-binding agent. In some embodiments, a HTRA1-binding agent inhibits HTRA1 activity. In some embodiments, a HTRA1-binding agent inhibits HTRA1 protease activity. In some embodiments, a HTRA1-binding agent is used in a combination therapy. In some embodiments, a HTRA1-binding agent is used in combination with at least one additional therapeutic agent.

The disclosure also provides compositions comprising the HTRA1-binding agents described herein. In some embodiments, the disclosure provides pharmaceutical compositions comprising the HTRA1-binding agents described herein. Polynucleotides and/or vectors encoding the HTRA1-binding agents are provided. Cells comprising the polynucleotides and/or the vectors described herein are also provided. Cells comprising or producing the HTRA1-binding agents described herein are provided. Methods of making the binding agents described herein are also provided.

In one aspect, the present disclosure provides agents that bind HTRA1. In some embodiments, an agent binds human HTRA1. In some embodiments, an agent binds SEQ ID NO:2. In some embodiments, an agent binds cynomolgus monkey (“cyno”) HTRA1. In some embodiments, an agent binds SEQ ID NO:8. In some embodiments, an agent binds human HTRA1 and cyno HTRA1. In some embodiments, an agent is an antibody. In some embodiments, an agent is an antibody that binds human HTRA1. In some embodiments, an agent is an antibody that binds cyno HTRA1. In some embodiments, an agent is an antibody that binds human HTRA1 and cyno HTRA1.

In some embodiments, an agent binds within the catalytic domain of HTRA1. In some embodiments, an agent does not bind within the N-terminal domain of HTRA1. In some embodiments, an agent does not binds within amino acids 1-100 of SEQ ID NO:1. In some embodiments, an agent does not binds within amino acids 23-100 of SEQ ID NO:1. In some embodiments, an agent binds within amino acids 101-480 of SEQ ID NO:1. In some embodiments, an agent binds within amino acids 156-480 of SEQ ID NO:1. In some embodiments, an agent binds within amino acids 156-364 of SEQ ID NO:1. In some embodiments, an agent binds a conformational epitope within the catalytic domain of HTRA1. In some embodiments, an agent binds a conformational epitope comprising at least one (e.g., 1, 2, 3, 4, 5, 6, 7, 8, 9) amino acid within amino acids 185-200 of SEQ ID NO:1. In some embodiments, an agent binds a conformational epitope comprising one or more amino acids R190, L192, and R197 of SEQ ID NO:1. In some embodiments, an agent binds a conformational epitope comprising amino acids R190, L192, and R197 of SEQ ID NO:1.

In one aspect, the present disclosure provides agents that bind human HTRA1 and have at least one or more of the following properties: (a) binds cyno HTRA1; (b) binds rabbit HTRA1; (c) inhibits HTRA1 protease activity; (d) inhibits HTRA1 protease activity in an allosteric manner; and (e) does not inhibit protease activity of other proteases in HTRA family. In some embodiments, the agent has a KD for human HTRA1 in the range of approximately 5×10⁻¹⁰ to 1×10⁻¹¹ M.

In one aspect, the present disclosure provides agents that specifically binds human HTRA1. In some embodiments, a HTRA1-binding agent comprises: (a) a heavy chain variable region comprising a heavy chain variable region CDR1 comprising the amino acid sequence GYTFTDYEMH (SEQ ID NO:9), a heavy chain variable region CDR2 comprising the amino acid sequence AIDPETGGTAYNQKFKG (SEQ ID NO:10), and a heavy chain variable region CDR3 comprising the amino acid sequence EGYSYDGGGYYFDY (SEQ ID NO:11) or the amino acid sequence EGYSYEGGGYYFDY (SEQ ID NO:25); and/or (b) a light chain variable region comprising a light chain variable region CDR1 comprising the amino acid sequence SVSSSVSYMY (SEQ ID NO:12), a light chain variable region CDR2 comprising the amino acid sequence DTSNLAS (SEQ ID NO:13), and a light chain variable region CDR3 comprising the amino acid sequence QQWSSYPT (SEQ ID NO:14). In some embodiments, a HTRA1-binding agent comprises: (a) a heavy chain variable region comprising a heavy chain variable region CDR1 comprising the amino acid sequence GYTFTDYEMH (SEQ ID NO:9), a heavy chain variable region CDR2 comprising the amino acid sequence AIDPETGGTAYNQKFKG (SEQ ID NO:10), a heavy chain variable region CDR3 comprising the amino acid sequence EGYSYDGGGYYFDY (SEQ ID NO:11); and (b) a light chain variable region comprising a light chain variable region CDR1 comprising the amino acid sequence SVSSSVSYMY (SEQ ID NO:12), a light chain variable region CDR2 comprising the amino acid sequence DTSNLAS (SEQ ID NO:13), and a light chain variable region CDR3 comprising the amino acid sequence QQWSSYPT (SEQ ID NO:14). In some embodiments, a HTRA1-binding agent comprises: (a) a heavy chain variable region comprising a heavy chain variable region CDR1 comprising the amino acid sequence GYTFTDYEMH (SEQ ID NO:9), a heavy chain variable region CDR2 comprising the amino acid sequence AIDPETGGTAYNQKFKG (SEQ ID NO:10), a heavy chain variable region CDR3 comprising the amino acid sequence EGYSYEGGGYYFDY (SEQ ID NO:25); and (b) a light chain variable region comprising a light chain variable region CDR1 comprising the amino acid sequence SVSSSVSYMY (SEQ ID NO:12), a light chain variable region CDR2 comprising the amino acid sequence DTSNLAS (SEQ ID NO:13), and a light chain variable region CDR3 comprising the amino acid sequence QQWSSYPT (SEQ ID NO:14).

In some embodiments, the HTRA1 binding agent comprises an amino acid substitution in the light chain variable region CDR3. In some embodiments, the substitution is at position 91. In some embodiments, the substitution is at position 92. In some embodiments, the substitution is selected from the group consisting of S91Y, S91D, S91A, or S91L. In some embodiments, the substitution is S91Y. In some embodiments, the substitution is S92T, S92Y or S92D. In some embodiments, the substitution is S92T.

In some embodiments, the HTRA1-binding agent comprises: (a) a heavy chain variable region comprising a heavy chain variable region CDR1 comprising the amino acid sequence GYTFTDYEMH (SEQ ID NO:9), a heavy chain variable region CDR2 comprising the amino acid sequence AIDPETGGTAYNQKFKG (SEQ ID NO:10), and a heavy chain variable region CDR3 comprising the amino acid sequence EGYSYDGGGYYFDY (SEQ ID NO:11); and (b) a light chain variable region comprising a light chain variable region CDR1 comprising the amino acid sequence SVSSSVSYMY (SEQ ID NO:12), a light chain variable region CDR2 comprising the amino acid sequence DTSNLAS (SEQ ID NO:13), and a light chain variable region CDR3 comprising the amino acid sequence QQWYSYPT (SEQ ID NO:94). In some embodiments, the HTRA1-binding agent comprises: (a) a heavy chain variable region comprising a heavy chain variable region CDR1 comprising the amino acid sequence GYTFTDYEMH (SEQ ID NO:9), a heavy chain variable region CDR2 comprising the amino acid sequence AIDPETGGTAYNQKFKG (SEQ ID NO:10), and a heavy chain variable region CDR3 comprising the amino acid sequence EGYSYEGGGYYFDY (SEQ ID NO:25); and (b) a light chain variable region comprising a light chain variable region CDR1 comprising the amino acid sequence SVSSSVSYMY (SEQ ID NO:12), a light chain variable region CDR2 comprising the amino acid sequence DTSNLAS (SEQ ID NO:13), and a light chain variable region CDR3 comprising the amino acid sequence QQWYSYPT (SEQ ID NO:94). In some embodiments, the HTRA1-binding agent comprises: (a) a heavy chain variable region comprising a heavy chain variable region CDR1 comprising the amino acid sequence GYTFTDYEMH (SEQ ID NO:9), a heavy chain variable region CDR2 comprising the amino acid sequence AIDPETGGTAYNQKFKG (SEQ ID NO:10), and a heavy chain variable region CDR3 comprising the amino acid sequence EGYSYDGGGYYFDY (SEQ ID NO:11); and (b) a light chain variable region comprising a light chain variable region CDR1 comprising the amino acid sequence SVSSSVSYMY (SEQ ID NO:12), a light chain variable region CDR2 comprising the amino acid sequence DTSNLAS (SEQ ID NO:13), and a light chain variable region CDR3 comprising the amino acid sequence QQWSTYPT (SEQ ID NO:99). In some embodiments, the HTRA1-binding agent comprises: (a) a heavy chain variable region comprising a heavy chain variable region CDR1 comprising the amino acid sequence GYTFTDYEMH (SEQ ID NO:9), a heavy chain variable region CDR2 comprising the amino acid sequence AIDPETGGTAYNQKFKG (SEQ ID NO:10), and a heavy chain variable region CDR3 comprising the amino acid sequence EGYSYEGGGYYFDY (SEQ ID NO:25); and (b) a light chain variable region comprising a light chain variable region CDR1 comprising the amino acid sequence SVSSSVSYMY (SEQ ID NO:12), a light chain variable region CDR2 comprising the amino acid sequence DTSNLAS (SEQ ID NO:13), and a light chain variable region CDR3 comprising the amino acid sequence QQWSTYPT (SEQ ID NO:99). In some embodiments, the HTRA1-binding agent comprises: a heavy chain variable region comprising a heavy chain variable region CDR1 comprising the amino acid sequence GYTFTDYEMH (SEQ ID NO:9), a heavy chain variable region CDR2 comprising the amino acid sequence AIDPETGGTAYNQKFKG (SEQ ID NO:10), and a heavy chain variable region CDR3 comprising the amino acid sequence EGYSYDGGGYYFDY (SEQ ID NO:11); and a light chain variable region comprising a light chain variable region CDR1 comprising the amino acid sequence SVSSSVSYMY (SEQ ID NO:12), a light chain variable region CDR2 comprising the amino acid sequence DTSNLAS (SEQ ID NO:13), and a light chain variable region CDR3 comprising the amino acid sequence QQWDSYPT (SEQ ID NO:104). In some embodiments, the HTRA1-binding agent comprises: a heavy chain variable region comprising a heavy chain variable region CDR1 comprising the amino acid sequence GYTFTDYEMH (SEQ ID NO:9), a heavy chain variable region CDR2 comprising the amino acid sequence AIDPETGGTAYNQKFKG (SEQ ID NO:10), and a heavy chain variable region CDR3 comprising the amino acid sequence EGYSYEGGGYYFDY (SEQ ID NO:25); and a light chain variable region comprising a light chain variable region CDR1 comprising the amino acid sequence SVSSSVSYMY (SEQ ID NO:12), a light chain variable region CDR2 comprising the amino acid sequence DTSNLAS (SEQ ID NO:13), and a light chain variable region CDR3 comprising the amino acid sequence QQWDSYPT (SEQ ID NO:104). In some embodiments, the HTRA1-binding agent comprises: a heavy chain variable region comprising a heavy chain variable region CDR1 comprising the amino acid sequence GYTFTDYEMH (SEQ ID NO:9), a heavy chain variable region CDR2 comprising the amino acid sequence AIDPETGGTAYNQKFKG (SEQ ID NO:10), and a heavy chain variable region CDR3 comprising the amino acid sequence EGYSYDGGGYYFDY (SEQ ID NO:11); and a light chain variable region comprising a light chain variable region CDR1 comprising the amino acid sequence SVSSSVSYMY (SEQ ID NO:12), a light chain variable region CDR2 comprising the amino acid sequence DTSNLAS (SEQ ID NO:13), and a light chain variable region CDR3 comprising the amino acid sequence QQWTSYPT (SEQ ID NO:107). In some embodiments, the HTRA1-binding agent comprises: a heavy chain variable region comprising a heavy chain variable region CDR1 comprising the amino acid sequence GYTFTDYEMH (SEQ ID NO:9), a heavy chain variable region CDR2 comprising the amino acid sequence AIDPETGGTAYNQKFKG (SEQ ID NO:10), and a heavy chain variable region CDR3 comprising the amino acid sequence EGYSYEGGGYYFDY (SEQ ID NO:25); and a light chain variable region comprising a light chain variable region CDR1 comprising the amino acid sequence SVSSSVSYMY (SEQ ID NO:12), a light chain variable region CDR2 comprising the amino acid sequence DTSNLAS (SEQ ID NO:13), and a light chain variable region CDR3 comprising the amino acid sequence QQWTSYPT (SEQ ID NO:107). In some embodiments, the HTRA1-binding agent comprises: a heavy chain variable region comprising a heavy chain variable region CDR1 comprising the amino acid sequence GYTFTDYEMH (SEQ ID NO:9), a heavy chain variable region CDR2 comprising the amino acid sequence AIDPETGGTAYNQKFKG (SEQ ID NO:10), and a heavy chain variable region CDR3 comprising the amino acid sequence EGYSYDGGGYYFDY (SEQ ID NO:11); and a light chain variable region comprising a light chain variable region CDR1 comprising the amino acid sequence SVSSSVSYMY (SEQ ID NO:12), a light chain variable region CDR2 comprising the amino acid sequence DTSNLAS (SEQ ID NO:13), and a light chain variable region CDR3 comprising the amino acid sequence QQWASYPT (SEQ ID NO:110). In some embodiments, the HTRA1-binding agent comprises: a heavy chain variable region comprising a heavy chain variable region CDR1 comprising the amino acid sequence GYTFTDYEMH (SEQ ID NO:9), a heavy chain variable region CDR2 comprising the amino acid sequence AIDPETGGTAYNQKFKG (SEQ ID NO:10), and a heavy chain variable region CDR3 comprising the amino acid sequence EGYSYEGGGYYFDY (SEQ ID NO:25); and a light chain variable region comprising a light chain variable region CDR1 comprising the amino acid sequence SVSSSVSYMY (SEQ ID NO:12), a light chain variable region CDR2 comprising the amino acid sequence DTSNLAS (SEQ ID NO:13), and a light chain variable region CDR3 comprising the amino acid sequence QQWASYPT (SEQ ID NO:110). In some embodiments, the HTRA1-binding agent comprises: a heavy chain variable region comprising a heavy chain variable region CDR1 comprising the amino acid sequence GYTFTDYEMH (SEQ ID NO:9), a heavy chain variable region CDR2 comprising the amino acid sequence AIDPETGGTAYNQKFKG (SEQ ID NO:10), and a heavy chain variable region CDR3 comprising the amino acid sequence EGYSYDGGGYYFDY (SEQ ID NO:11); and a light chain variable region comprising a light chain variable region CDR1 comprising the amino acid sequence SVSSSVSYMY (SEQ ID NO:12), a light chain variable region CDR2 comprising the amino acid sequence DTSNLAS (SEQ ID NO:13), and a light chain variable region CDR3 comprising the amino acid sequence QQWLSYPT (SEQ ID NO:113). In some embodiments, the HTRA1-binding agent comprises: a heavy chain variable region comprising a heavy chain variable region CDR1 comprising the amino acid sequence GYTFTDYEMH (SEQ ID NO:9), a heavy chain variable region CDR2 comprising the amino acid sequence AIDPETGGTAYNQKFKG (SEQ ID NO:10), and a heavy chain variable region CDR3 comprising the amino acid sequence EGYSYEGGGYYFDY (SEQ ID NO:25); and a light chain variable region comprising a light chain variable region CDR1 comprising the amino acid sequence SVSSSVSYMY (SEQ ID NO:12), a light chain variable region CDR2 comprising the amino acid sequence DTSNLAS (SEQ ID NO:13), and a light chain variable region CDR3 comprising the amino acid sequence QQWLSYPT (SEQ ID NO:113). In some embodiments, the HTRA1-binding agent comprises: a heavy chain variable region comprising a heavy chain variable region CDR1 comprising the amino acid sequence GYTFTDYEMH (SEQ ID NO:9), a heavy chain variable region CDR2 comprising the amino acid sequence AIDPETGGTAYNQKFKG (SEQ ID NO:10), and a heavy chain variable region CDR3 comprising the amino acid sequence EGYSYDGGGYYFDY (SEQ ID NO:11); and a light chain variable region comprising a light chain variable region CDR1 comprising the amino acid sequence SVSSSVSYMY (SEQ ID NO:12), a light chain variable region CDR2 comprising the amino acid sequence DTSNLAS (SEQ ID NO:13), and a light chain variable region CDR3 comprising the amino acid sequence QQWSYYPT (SEQ ID NO:116). In some embodiments, the HTRA1-binding agent comprises: a heavy chain variable region comprising a heavy chain variable region CDR1 comprising the amino acid sequence GYTFTDYEMH (SEQ ID NO:9), a heavy chain variable region CDR2 comprising the amino acid sequence AIDPETGGTAYNQKFKG (SEQ ID NO:10), and a heavy chain variable region CDR3 comprising the amino acid sequence EGYSYEGGGYYFDY (SEQ ID NO:25); and a light chain variable region comprising a light chain variable region CDR1 comprising the amino acid sequence SVSSSVSYMY (SEQ ID NO:12), a light chain variable region CDR2 comprising the amino acid sequence DTSNLAS (SEQ ID NO:13), and a light chain variable region CDR3 comprising the amino acid sequence QQWSYYPT (SEQ ID NO:116). In some embodiments, the HTRA1-binding agent comprises: a heavy chain variable region comprising a heavy chain variable region CDR1 comprising the amino acid sequence GYTFTDYEMH (SEQ ID NO:9), a heavy chain variable region CDR2 comprising the amino acid sequence AIDPETGGTAYNQKFKG (SEQ ID NO:10), and a heavy chain variable region CDR3 comprising the amino acid sequence EGYSYDGGGYYFDY (SEQ ID NO:11); and a light chain variable region comprising a light chain variable region CDR1 comprising the amino acid sequence SVSSSVSYMY (SEQ ID NO:12), a light chain variable region CDR2 comprising the amino acid sequence DTSNLAS (SEQ ID NO:13), and a light chain variable region CDR3 comprising the amino acid sequence QQWSDYPT (SEQ ID NO:119). In some embodiments, the HTRA1-binding agent comprises: a heavy chain variable region comprising a heavy chain variable region CDR1 comprising the amino acid sequence GYTFTDYEMH (SEQ ID NO:9), a heavy chain variable region CDR2 comprising the amino acid sequence AIDPETGGTAYNQKFKG (SEQ ID NO:10), and a heavy chain variable region CDR3 comprising the amino acid sequence EGYSYEGGGYYFDY (SEQ ID NO:25); and a light chain variable region comprising a light chain variable region CDR1 comprising the amino acid sequence SVSSSVSYMY (SEQ ID NO:12), a light chain variable region CDR2 comprising the amino acid sequence DTSNLAS (SEQ ID NO:13), and a light chain variable region CDR3 comprising the amino acid sequence QQWSDYPT (SEQ ID NO:119).

In some embodiments, a HTRA1-binding agent comprises: (a) a heavy chain variable region having at least 80% sequence identity to SEQ ID NO:68, SEQ ID NO:70, or SEQ ID NO:71; and/or (b) a light chain variable region having at least 80% sequence identity to SEQ ID NO:69 or SEQ ID NO:72. In some embodiments, a HTRA1-binding agent comprises a heavy chain variable region having least 80%, at least 85%, at least 90%, at least 95%, at least 96%, at least 97%, at least 98%, or at least 99% sequence identity to SEQ ID NO:68 and/or a light chain variable region having least 80%, at least 85%, at least 90%, at least 95%, at least 96%, at least 97%, at least 98%, or at least 99% sequence identity to SEQ ID NO:69. In some embodiments, a HTRA1-binding agent comprises a heavy chain variable region having least 80%, at least 85%, at least 90%, at least 95%, at least 96%, at least 97%, at least 98%, or at least 99% sequence identity to SEQ ID NO:70 and/or a light chain variable region having least 80%, at least 85%, at least 90%, at least 95%, at least 96%, at least 97%, at least 98%, or at least 99% sequence identity to SEQ ID NO:72. In some embodiments, a HTRA1-binding agent comprises a heavy chain variable region having least 80%, at least 85%, at least 90%, at least 95%, at least 96%, at least 97%, at least 98%, or at least 99% sequence identity to SEQ ID NO:71 and/or a light chain variable region having least 80%, at least 85%, at least 90%, at least 95%, at least 96%, at least 97%, at least 98%, or at least 99% sequence identity to SEQ ID NO:72. In some embodiments, a HTRA1-binding agent comprises a heavy chain variable region of SEQ ID NO:68. In some embodiments, a HTRA1-binding agent comprises a light chain variable region of SEQ ID NO:69. In some embodiments, a HTRA1-binding agent comprises a heavy chain variable region of SEQ ID NO:68 and a light chain variable region of SEQ ID NO:69. In some embodiments, a HTRA1-binding agent comprises a heavy chain variable region of SEQ ID NO:70. In some embodiments, a HTRA1-binding agent comprises a heavy chain variable region of SEQ ID NO:71. In some embodiments, a HTRA1-binding agent comprises a light chain variable region of SEQ ID NO:72. In some embodiments, a HTRA1-binding agent comprises a heavy chain variable region of SEQ ID NO:70 and a light chain variable region of SEQ ID NO:72. In some embodiments, a HTRA1-binding agent comprises a heavy chain variable region of SEQ ID NO:71 and a light chain variable region of SEQ ID NO:72. In some embodiments, a HTRA1-binding agent comprises a light chain variable region of SEQ ID NO:96. In some embodiments, a HTRA1-binding agent comprises a light chain variable region of SEQ ID NO:101. In some embodiments, a HTRA1-binding agent comprises a light chain variable region of SEQ ID NO:106. In some embodiments, a HTRA1-binding agent comprises a light chain variable region of SEQ ID NO:109. In some embodiments, a HTRA1-binding agent comprises a light chain variable region of SEQ ID NO:112. In some embodiments, a HTRA1-binding agent comprises a light chain variable region of SEQ ID NO:115. In some embodiments, a HTRA1-binding agent comprises a light chain variable region of SEQ ID NO:118. In some embodiments, a HTRA1-binding agent comprises a light chain variable region of SEQ ID NO:121. In some embodiments, a HTRA1-binding agent comprises a heavy chain variable region of SEQ ID NO:70 and a light chain variable region of SEQ ID NO:96. In some embodiments, a HTRA1-binding agent comprises a heavy chain variable region of SEQ ID NO:70 and a light chain variable region of SEQ ID NO:101. In some embodiments, a HTRA1-binding agent comprises a heavy chain variable region of SEQ ID NO:70 and a light chain variable region of SEQ ID NO:106. In some embodiments, a HTRA1-binding agent comprises a heavy chain variable region of SEQ ID NO:70 and a light chain variable region of SEQ ID NO:109. In some embodiments, a HTRA1-binding agent comprises a heavy chain variable region of SEQ ID NO:70 and a light chain variable region of SEQ ID NO:112. In some embodiments, a HTRA1-binding agent comprises a heavy chain variable region of SEQ ID NO:70 and a light chain variable region of SEQ ID NO:115. In some embodiments, a HTRA1-binding agent comprises a heavy chain variable region of SEQ ID NO:70 and a light chain variable region of SEQ ID NO:118. In some embodiments, a HTRA1-binding agent comprises a heavy chain variable region of SEQ ID NO:70 and a light chain variable region of SEQ ID NO:121. In some embodiments, a HTRA1-binding agent comprises a heavy chain variable region of SEQ ID NO:71 and a light chain variable region of SEQ ID NO:96. In some embodiments, a HTRA1-binding agent comprises a heavy chain variable region of SEQ ID NO:71 and a light chain variable region of SEQ ID NO:101. In some embodiments, a HTRA1-binding agent comprises a heavy chain variable region of SEQ ID NO:71 and a light chain variable region of SEQ ID NO:106. In some embodiments, a HTRA1-binding agent comprises a heavy chain variable region of SEQ ID NO:71 and a light chain variable region of SEQ ID NO:109. In some embodiments, a HTRA1-binding agent comprises a heavy chain variable region of SEQ ID NO:71 and a light chain variable region of SEQ ID NO:112. In some embodiments, a HTRA1-binding agent comprises a heavy chain variable region of SEQ ID NO:71 and a light chain variable region of SEQ ID NO:115. In some embodiments, a HTRA1-binding agent comprises a heavy chain variable region of SEQ ID NO:71 and a light chain variable region of SEQ ID NO:118. In some embodiments, a HTRA1-binding agent comprises a heavy chain variable region of SEQ ID NO:71 and a light chain variable region of SEQ ID NO:121.

In some embodiments, a HTRA1-binding agent comprises a heavy chain variable region CDR1, CDR2, and CDR3 from the amino acid sequence of SEQ ID NO:68 and a light chain variable region CDR1, CDR2, and CDR3 from the amino acid sequence of SEQ ID NO:69. In some embodiments, a HTRA1-binding agent comprises a heavy chain variable region comprising a heavy chain variable region CDR1, CDR2, and CDR3 from the amino acid sequence of SEQ ID NO:68 and a light chain variable region comprising a light chain variable region CDR1, CDR2, and CDR3 from the amino acid sequence of SEQ ID NO:69.

In some embodiments, a HTRA1-binding agent comprises a heavy chain variable region CDR1, CDR2, and CDR3 from the amino acid sequence of SEQ ID NO:70 and a light chain variable region CDR1, CDR2, and CDR3 from the amino acid sequence of SEQ ID NO:72. In some embodiments, a HTRA1-binding agent comprises a heavy chain variable region comprising a heavy chain variable region CDR1, CDR2, and CDR3 from the amino acid sequence of SEQ ID NO:70 and a light chain variable region comprising a light chain variable region CDR1, CDR2, and CDR3 from the amino acid sequence of SEQ ID NO:72. In some embodiments, a HTRA1-binding agent comprises a heavy chain variable region CDR1, CDR2, and CDR3 from the amino acid sequence of SEQ ID NO:71 and a light chain variable region CDR1, CDR2, and CDR3 from the amino acid sequence of SEQ ID NO:72. In some embodiments, a HTRA1-binding agent comprises a heavy chain variable region comprising a heavy chain variable region CDR1, CDR2, and CDR3 from the amino acid sequence of SEQ ID NO:71 and a light chain variable region comprising a light chain variable region CDR1, CDR2, and CDR3 from the amino acid sequence of SEQ ID NO:72.

In some embodiments, the HTRA1-binding agent comprises a heavy chain variable region comprising a CDR1, CDR2, and CDR3 from the amino acid sequence of SEQ ID NO:71 and a light chain variable region comprising a CDR1, CDR2, and CDR3 from the amino acid sequence of SEQ ID NO:96. In some embodiments, the HTRA1-binding agent comprises a heavy chain variable region comprising a CDR1, CDR2, and CDR3 from the amino acid sequence of SEQ ID NO:71 and a light chain variable region comprising a CDR1, CDR2, and CDR3 from the amino acid sequence of SEQ ID NO:101. In some embodiments, the HTRA1-binding agent comprises a heavy chain variable region comprising a CDR1, CDR2, and CDR3 from the amino acid sequence of SEQ ID NO:71 and a light chain variable region comprising a CDR1, CDR2, and CDR3 from the amino acid sequence of SEQ ID NO:106. In some embodiments, the HTRA1-binding agent comprises a heavy chain variable region comprising a CDR1, CDR2, and CDR3 from the amino acid sequence of SEQ ID NO:71 and a light chain variable region comprising a CDR1, CDR2, and CDR3 from the amino acid sequence of SEQ ID NO:109. In some embodiments, the HTRA1-binding agent comprises a heavy chain variable region comprising a CDR1, CDR2, and CDR3 from the amino acid sequence of SEQ ID NO:71 and a light chain variable region comprising a CDR1, CDR2, and CDR3 from the amino acid sequence of SEQ ID NO:112. In some embodiments, the HTRA1-binding agent comprises a heavy chain variable region comprising a CDR1, CDR2, and CDR3 from the amino acid sequence of SEQ ID NO:71 and a light chain variable region comprising a CDR1, CDR2, and CDR3 from the amino acid sequence of SEQ ID NO:115. In some embodiments, the HTRA1-binding agent comprises a heavy chain variable region comprising a CDR1, CDR2, and CDR3 from the amino acid sequence of SEQ ID NO:71 and a light chain variable region comprising a CDR1, CDR2, and CDR3 from the amino acid sequence of SEQ ID NO:118. In some embodiments, the HTRA1-binding agent comprises a heavy chain variable region comprising a CDR1, CDR2, and CDR3 from the amino acid sequence of SEQ ID NO:71 and a light chain variable region comprising a CDR1, CDR2, and CDR3 from the amino acid sequence of SEQ ID NO:121.

In some embodiments, a HTRA1-binding agent comprises the six CDRs of antibody 24F7 or a humanized version of antibody 24F7 and a heavy chain having least 80%, at least 85%, at least 90%, at least 95%, at least 96%, at least 97%, at least 98%, or at least 99% sequence identity to SEQ ID NO:88 and/or a light chain having least 80%, at least 85%, at least 90%, at least 95%, at least 96%, at least 97%, at least 98%, or at least 99% sequence identity to SEQ ID NO:90. In some embodiments, a HTRA1-binding agent is an antibody that comprises a heavy chain of SEQ ID NO:88 and/or a light chain of SEQ ID NO:90. In some embodiments, a HTRA1-binding agent is an antibody that comprises a heavy chain of SEQ ID NO:88. In some embodiments, a HTRA1-binding agent is an antibody that comprises a light chain of SEQ ID NO:90. In some embodiments, a HTRA1-binding agent is an antibody that comprises a heavy chain of SEQ ID NO:88 and a light chain of SEQ ID NO:90. In some embodiments, a HTRA1-binding agent is a monoclonal antibody that comprises a heavy chain comprising the amino acid sequence of SEQ ID NO:88 and a light chain comprising the amino acid sequence of SEQ ID NO:90. In some embodiments, the HTRA1-binding agent comprises the amino acid sequence of SEQ ID NO:88 and a light chain comprising the amino acid sequence of SEQ ID NO:98. In some embodiments, the HTRA1-binding agent comprises a heavy chain comprising the amino acid sequence of SEQ ID NO:88 and a light chain comprising the amino acid sequence of SEQ ID NO:103. In some embodiments, the HTRA1-binding agent comprises a heavy chain comprising the amino acid sequence of SEQ ID NO:88 and a light chain comprising the amino acid sequence of SEQ ID NO:123. In some embodiments, the HTRA1-binding agent comprises a heavy chain comprising the amino acid sequence of SEQ ID NO:88 and a light chain comprising the amino acid sequence of SEQ ID NO:125. In some embodiments, the HTRA1-binding agent comprises a heavy chain comprising the amino acid sequence of SEQ ID NO:88 and a light chain comprising the amino acid sequence of SEQ ID NO:127. In some embodiments, the HTRA1-binding agent comprises a heavy chain comprising the amino acid sequence of SEQ ID NO:88 and a light chain comprising the amino acid sequence of SEQ ID NO:129. In some embodiments, the HTRA1-binding agent comprises a heavy chain comprising the amino acid sequence of SEQ ID NO:88 and a light chain comprising the amino acid sequence of SEQ ID NO:131. In some embodiments, the HTRA1-binding agent comprises a heavy chain comprising the amino acid sequence of SEQ ID NO:88 and a light chain comprising the amino acid sequence of SEQ ID NO:133.

In some embodiments, a HTRA1-binding agent comprises: (a) a heavy chain variable region comprising a heavy chain variable region CDR1 comprising the amino acid sequence GYAFTTYWMH (SEQ ID NO:27), a heavy chain variable region CDR2 comprising the amino acid sequence NIDPSDSETHYNQKFRD (SEQ ID NO:28), and a heavy chain variable region CDR3 comprising the amino acid sequence DYGAFDV (SEQ ID NO:29); and/or (b) a light chain variable region comprising a light chain variable region CDR1 comprising the amino acid sequence RSSTGAVTTR (SEQ ID NO:30), a light chain variable region CDR2 comprising the amino acid sequence GTNNRAP (SEQ ID NO:31), and a light chain variable region CDR3 comprising the amino acid sequence ALWYSNLWV (SEQ ID NO:32). In some embodiments, a HTRA1-binding agent comprises a heavy chain variable region having at least 80% identity to SEQ ID NO:73 and/or a light chain variable region having at least 80% identity to SEQ ID NO:74. In some embodiments, a HTRA1-binding agent comprises a heavy chain variable region CDR1, CDR2, and CDR3 from the amino acid sequence of SEQ ID NO:73 and/or a light chain variable region CDR1, CDR2, and CDR3 from the amino acid sequence of SEQ ID NO:74. In some embodiments, a HTRA1-binding agent comprises a heavy chain variable region comprising a heavy chain variable region CDR1, CDR2, and CDR3 from the amino acid sequence of SEQ ID NO:73 and/or a light chain variable region comprising a light chain variable region CDR1, CDR2, and CDR3 from the amino acid sequence of SEQ ID NO:74.

In some embodiments, a HTRA1-binding agent comprises: (a) a heavy chain variable region comprising a heavy chain variable region CDR1 comprising the amino acid sequence GYTFTNYWMH (SEQ ID NO:43), a heavy chain variable region CDR2 comprising the amino acid sequence NIDPSDSETHYNQKFKD (SEQ ID NO:44), and a heavy chain variable region CDR3 comprising the amino acid sequence EDSSGYGAY (SEQ ID NO:45); and/or (b) a light chain variable region comprising a light chain variable region CDR1 comprising the amino acid sequence SASSSVNYMH (SEQ ID NO:46), a light chain variable region CDR2 comprising the amino acid sequence DTSKLAS (SEQ ID NO:47), and a light chain variable region CDR3 comprising the amino acid sequence QQWSSHPLT (SEQ ID NO:48). In some embodiments, a HTRA1-binding agent comprises a heavy chain variable region having at least 80% identity to SEQ ID NO:75 and/or a light chain variable region having at least 80% identity to SEQ ID NO:76. In some embodiments, a HTRA1-binding agent comprises a heavy chain variable region CDR1, CDR2, and CDR3 from the amino acid sequence of SEQ ID NO:75 and/or a light chain variable region CDR1, CDR2, and CDR3 from the amino acid sequence of SEQ ID NO:76. In some embodiments, a HTRA1-binding agent comprises a heavy chain variable region comprising a heavy chain variable region CDR1, CDR2, and CDR3 from the amino acid sequence of SEQ ID NO:75 and/or a light chain variable region comprising a light chain variable region CDR1, CDR2, and CDR3 from the amino acid sequence of SEQ ID NO:76.

In some embodiments, a HTRA1-binding agent comprises: (a) a heavy chain variable region comprising a heavy chain variable region CDR1 comprising the amino acid sequence GYSFTSYWMH (SEQ ID NO:56), a heavy chain variable region CDR2 comprising the amino acid sequence MIDPSDSETRLNQKFKD (SEQ ID NO:57), and a heavy chain variable region CDR3 comprising the amino acid sequence DYFDY (SEQ ID NO:58); and/or (b) a light chain variable region comprising a light chain variable region CDR1 comprising the amino acid sequence SASSSVSYMY (SEQ ID NO:59), a light chain variable region CDR2 comprising the amino acid sequence DTSNLAS (SEQ ID NO:13), and a light chain variable region CDR3 comprising the amino acid sequence QQWSSYPYT (SEQ ID NO:60). In some embodiments, a HTRA1-binding agent comprises a heavy chain variable region having at least 80% identity to SEQ ID NO:77 and/or a light chain variable region having at least 80% identity to SEQ ID NO:78. In some embodiments, a HTRA1-binding agent comprises a heavy chain variable region CDR1, CDR2, and CDR3 from the amino acid sequence of SEQ ID NO:77 and/or a light chain variable region CDR1, CDR2, and CDR3 from the amino acid sequence of SEQ ID NO:78. In some embodiments, a HTRA1-binding agent comprises a heavy chain variable region comprising a heavy chain variable region CDR1, CDR2, and CDR3 from the amino acid sequence of SEQ ID NO:77 and/or a light chain variable region comprising a light chain variable region CDR1, CDR2, and CDR3 from the amino acid sequence of SEQ ID NO:78.

In another aspect of the disclosure, provided herein is a binding agent that competes for binding to HTRA1 with any of the HTRA1-binding agents described herein. In some embodiments, provided herein is an agent that competes for binding to HTRA1 with a reference antibody, wherein the reference antibody comprises a heavy chain variable region comprising a heavy chain variable region CDR1 comprising the amino acid sequence GYTFTDYEMH (SEQ ID NO:9), a heavy chain variable region CDR2 comprising the amino acid sequence AIDPETGGTAYNQKFKG (SEQ ID NO:10), a heavy chain variable region CDR3 comprising the amino acid sequence EGYSYDGGGYYFDY (SEQ ID NO:11) or the amino acid sequence EGYSYEGGGYYFDY (SEQ ID NO:25), and a light chain variable region comprising a light chain variable region CDR1 comprising the amino acid sequence SVSSSVSYMY (SEQ ID NO:12), a light chain variable region CDR2 comprising the amino acid sequence DTSNLAS (SEQ ID NO:13), and a light chain variable region CDR3 comprising the amino acid sequence QQWSSYPT (SEQ ID NO:14). In some embodiments, provided herein is an agent that competes for binding to HTRA1 with a reference antibody, wherein the reference antibody comprises a heavy chain variable region comprising a heavy chain variable region CDR1 comprising the amino acid sequence GYTFTDYEMH (SEQ ID NO:9), a heavy chain variable region CDR2 comprising the amino acid sequence AIDPETGGTAYNQKFKG (SEQ ID NO:10), a heavy chain variable region CDR3 comprising the amino acid sequence EGYSYDGGGYYFDY (SEQ ID NO:11) or the amino acid sequence EGYSYEGGGYYFDY (SEQ ID NO:25), and a light chain variable region comprising a light chain variable region CDR1 comprising the amino acid sequence SVSSSVSYMY (SEQ ID NO:12), a light chain variable region CDR2 comprising the amino acid sequence DTSNLAS (SEQ ID NO:13), and a light chain variable region CDR3 comprising the amino acid sequence QQWSSYPT (SEQ ID NO:14) and wherein the agent that competes with the reference antibody comprises: (a) a heavy chain variable region comprising a heavy chain variable region CDR1 comprising the amino acid sequence GYAFTTYWMH (SEQ ID NO:27), a heavy chain variable region CDR2 comprising the amino acid sequence NIDPSDSETHYNQKFRD (SEQ ID NO:28), a heavy chain variable region CDR3 comprising the amino acid sequence DYGAFDV (SEQ ID NO:29), and a light chain variable region comprising a light chain variable region CDR1 comprising the amino acid sequence RSSTGAVTTR (SEQ ID NO:30), a light chain variable region CDR2 comprising the amino acid sequence GTNNRAP (SEQ ID NO:31), and a light chain variable region CDR3 comprising the amino acid sequence ALWYSNLWV (SEQ ID NO:32); (b) a heavy chain variable region comprising a heavy chain variable region CDR1 comprising the amino acid sequence GYTFTNYWMH (SEQ ID NO:43), a heavy chain variable region CDR2 comprising the amino acid sequence NIDPSDSETHYNQKFKD (SEQ ID NO:44), a heavy chain variable region CDR3 comprising the amino acid sequence EDSSGYGAY (SEQ ID NO:45), and a light chain variable region comprising a light chain variable region CDR1 comprising the amino acid sequence SASSSVNYMH (SEQ ID NO:46), a light chain variable region CDR2 comprising the amino acid sequence DTSKLAS (SEQ ID NO:47), and a light chain variable region CDR3 comprising the amino acid sequence QQWSSHPLT (SEQ ID NO:48); or (c) a heavy chain variable region comprising a heavy chain variable region CDR1 comprising the amino acid sequence GYSFTSYWMH (SEQ ID NO:56), a heavy chain variable region CDR2 comprising the amino acid sequence MIDPSDSETRLNQKFKD (SEQ ID NO:57), a heavy chain variable region CDR3 comprising the amino acid sequence DYFDY (SEQ ID NO:58), and a light chain variable region comprising a light chain variable region CDR1 comprising the amino acid sequence SASSSVSYMY (SEQ ID NO:59), a light chain variable region CDR2 comprising the amino acid sequence DTSNLAS (SEQ ID NO:13), and a light chain variable region CDR3 comprising the amino acid sequence QQWSSYPYT (SEQ ID NO: 60).

In some embodiments of each of the aforementioned aspects and embodiments, as well as other aspects and embodiments described herein, a HTRA1-binding agent is an antibody. In some embodiments, the antibody is a monoclonal antibody. In some embodiments, the antibody is a humanized antibody. In some embodiments, the antibody is a human antibody. In some embodiments, the antibody is a chimeric antibody. In some embodiments, the antibody is a whole or intact antibody. In some embodiments, the antibody is a bispecific antibody or a multispecific antibody. In some embodiments, the antibody is an antibody fragment comprising at least one antigen-binding site. In some embodiments, the antibody or antibody fragment is a Fab, Fab′, F(ab′)₂, Fv, scFv, (scFv)₂, single chain antibody, dual variable region antibody, single variable region antibody, linear antibody, diabody, nanobody, or a V region antibody. In some embodiments, the antibody is an IgG antibody. In some embodiments, the antibody is an IgG1 antibody, an IgG2 antibody, an IgG3 antibody, or an IgG4 antibody. In some embodiments, the antibody is a human IgG1 antibody, a human IgG2 antibody, a human IgG3 antibody, or a human IgG4 antibody. In some embodiments, the antibody comprises a kappa light chain. In some embodiments, the antibody comprises a human kappa light chain. In some embodiments, the antibody comprises a lambda light chain. In some embodiments, the antibody comprises a human lambda light chain.

In some embodiments of each of the aforementioned aspects and embodiments, as well as other aspects and embodiments described herein, a HTRA1-binding agent is attached (either directly or indirectly) to a half-life extending moiety.

In some embodiments of each of the aforementioned aspects and embodiments, as well as other aspects and embodiments described herein, a HTRA1-binding agent described herein is an antagonist of HTRA1. In some embodiments, a HTRA1-binding agent inhibits HTRA1 protease activity. In some embodiments, the HTRA1-binding agent is an antagonistic antibody. In some embodiments, the HTRA1-binding agent is an antagonistic antibody that inhibits HTRA1 protease activity.

In another aspect, the disclosure provides compositions comprising a HTRA1-binding agent described herein. In some embodiments, a composition comprises an anti-HTRA1 antibody described herein. In some embodiments, a composition comprises a monoclonal anti-HTRA1 antibody described herein. In some embodiments, a composition comprises an anti-HTRA1 antibody selected from the group consisting of: 24F7, hz24F7.v2, 9F8, 55B12, and 65G8. In some embodiments, a composition comprises a humanized version of an anti-HTRA1 antibody selected from the group consisting of: 24F7, 9F8, 55B12, and 65G8.

In another aspect, the disclosure provides pharmaceutical compositions comprising a HTRA1-binding agent described herein and a pharmaceutically acceptable carrier. In some embodiments, a pharmaceutical composition comprises an anti-HTRA1 antibody described herein and a pharmaceutically acceptable carrier. In some embodiments, a pharmaceutical composition comprises a monoclonal anti-HTRA1 antibody described herein and a pharmaceutically acceptable carrier. In some embodiments, a pharmaceutical composition comprises an anti-HTRA1 antibody selected from the group consisting of: 24F7, hz24F7.v2, 9F8, 55B12, and 65G8 and a pharmaceutically acceptable carrier. In some embodiments, a pharmaceutical composition comprises a humanized version of an anti-HTRA1 antibody selected from the group consisting of: 24F7, 9F8, 55B12, and 65G8 and a pharmaceutically acceptable carrier. In some embodiments, a pharmaceutical composition further comprises at least one additional therapeutic agent.

In some embodiments of each of the aforementioned aspects, as well as other aspects and/or embodiments described elsewhere herein, the HTRA1-binding agent is isolated. In some embodiments, the HTRA1-binding agent is substantially pure.

In another aspect, the disclosure provides polynucleotides comprising one or more polynucleotides that encode a HTRA1-binding agent described herein. In some embodiments, a polynucleotide comprises one or more polynucleotides that encode an anti-HTRA1 antibody described herein. In some embodiments, a first polynucleotide encodes a heavy chain variable region of a HTRA1-binding agent described herein, and a second polynucleotide encodes a light chain variable region of a HTRA1-binding agent described herein. In some embodiments, a polynucleotide encodes a heavy chain variable region and a light chain variable region of a HTRA1-binding agent described herein. In some embodiments, a first polynucleotide encodes a heavy chain of an anti-HTRA1 antibody described herein, and a second polynucleotide encodes a light chain of an anti-HTRA1 antibody described herein. In some embodiments, a polynucleotide encodes a heavy chain and a light chain of an HTRA1 antibody described herein. In some embodiments, the polynucleotide is isolated.

In some embodiments, a vector comprises one or more polynucleotides that encode a HTRA1-binding agent described herein. In some embodiments, a vector comprises a polynucleotide that encodes a HTRA1-binding agent described herein. In some embodiments, a vector comprises a first polynucleotide encoding a heavy chain variable region of a HTRA1-binding agent described herein, and a second polynucleotide encoding a light chain variable region of a HTRA1-binding agent described herein. In some embodiments, a vector comprises a first polynucleotide encoding a heavy chain variable region of a HTRA1-binding agent described herein, and a second vector comprises a second polynucleotide encoding a light chain variable region of a HTRA1-binding agent described herein. In some embodiments, a vector comprises a first polynucleotide encoding a heavy chain of an anti-HTRA1 antibody described herein, and a second polynucleotide encoding a light chain of an anti-HTRA1 antibody described herein. In some embodiments, a vector comprises a first polynucleotide encoding a heavy chain of an anti-HTRA1 antibody described herein, and a second vector comprises a second polynucleotide encoding a light chain of an anti-HTRA1 antibody described herein.

In another aspect, the disclosure provides cells comprising one or more polynucleotides that encode a HTRA1-binding agent described herein. In some embodiments, a cell comprises a polynucleotide that encodes a HTRA1-binding agent described herein. In some embodiments, a cell comprises a vector comprising a polynucleotide that encodes a HTRA1-binding agent described herein. In some embodiments, an isolated cell comprises a vector comprising a polynucleotide that encodes a HTRA1-binding agent described herein. In some embodiments, a cell comprises a HTRA1-binding agent described herein. In some embodiments, a cell produces a HTRA1-binding agent described herein. In some embodiments, a cell produces an anti-HTRA1 antibody described herein. In some embodiments, a cell is a monoclonal cell line. In some embodiments, a cell is a hybridoma.

In another aspect, the disclosure provides methods of making a HTRA1-binding agent described herein. In some embodiments, a method of making a HTRA1-binding agent comprises: (a) culturing a cell described herein, and (b) isolating the HTRA1-binding agent. In some embodiments, a method of making a HTRA1-binding agent comprises: (a) culturing a cell described herein under conditions that allow expression of the HTRA1-binding agent, and (b) isolating the HTRA1-binding agent. In some embodiments, the method further comprises purifying the HTRA1-binding agent. In some embodiments, the method further comprises formulating the HTRA1-binding agent as a pharmaceutical composition.

In another aspect, the disclosure provides methods of using the HTRA1-binding agents described herein. In some embodiments, a method comprises using a composition comprising a HTRA1-binding agent described herein. In some embodiments, a method comprises using a pharmaceutical composition comprising a HTRA1-binding agent described herein.

In some embodiments, a method of treating an eye disorder or eye disease in a subject comprises administering to the subject a therapeutically effective amount of a HTRA1-binding agent described herein. In some embodiments, the eye disorder is selected from the group consisting of: macular degeneration (maculopathy), age-related macular degeneration (AMD), wet AMD, dry AMD, geographic atrophy (GA), diabetic retinopathy, retinopathy of prematurity, macular dystrophy, retinal dystrophy, uveitis, keratitis, scleritis, retinitis pigmentosa, choroidal neovascularization (CNV), retinal neovascularization, ocular inflammation, polypoidal choroidal vasculopathy (PCV), idiopathic polypoidal choroidal vasculopathy (IPCV), Stargardt disease, and neuromyelitis optica. In some embodiments, the eye disorder is macular degeneration. In some embodiments, the eye disorder is AMD. In some embodiments, the eye disorder is wet AMD. In some embodiments, the eye disorder is AMD associated with neovascularization. In some embodiments, the eye disorder is AMD associated with CNV. In some embodiments, the eye disorder is dry AMD. In some embodiments, the eye disorder is geographic atrophy.

In some embodiments, a method of treating AMD in a subject comprises administering to the eye of the subject a therapeutically effective amount of a HTRA1-binding agent described herein. In some embodiments, the AMD is wet AMD. In some embodiments, the AMD is AMD associated with neovascularization. In some embodiments, the AMD is AMD associated with CNV. In some embodiments, the AMD is dry AMD. In some embodiments, the AMD is geographic atrophy.

In some embodiments, a method of treating geographic atrophy in a subject comprises administering to the eye of the subject a therapeutically effective amount of a HTRA1-binding agent described herein. In some embodiments, a method of inhibiting or suppressing drusen formation in an eye of a subject comprises administering to the eye of the subject a therapeutically effective amount of a HTRA1-binding agent described herein. In some embodiments, the number and/or size of the drusen are reduced.

In some embodiments, a method of inhibiting or suppressing retinal pigment epithelium atrophy in an eye of a subject comprises administering to the eye of the subject a therapeutically effective amount of a HTRA1-binding agent described herein.

In some embodiments, a method of treating AMD associated with neovascularization in an eye of a subject comprises administering to the eye of the subject a therapeutically effective amount of a HTRA1-binding agent described herein. In some embodiments, a method of treating CNV in an eye of a subject comprises administering to the eye of the subject a therapeutically effective amount of a HTRA1-binding agent described herein. In some embodiments, a method of inhibiting CNV in an eye of a subject comprises administering to the eye of the subject a therapeutically effective amount of a HTRA1-binding agent described herein.

In some embodiments, a method of inhibiting progression of early AMD to advanced AMD in an eye of a subject comprises administering to the eye of the subject a therapeutically effective amount of a HTRA1-binding agent described herein. In some embodiments, the method inhibits progression of early AMD to GA. In some embodiments, the method inhibits progression of early AMD to wet AMD. In some embodiments, the method inhibits progression of early AMD to CNV. In some embodiments, the method inhibits progression of dry AMD to wet AMD.

In some embodiments, a method of inhibiting progression of intermediate AMD to advanced AMD in an eye of a subject comprises administering to the eye of the subject a therapeutically effective amount of a HTRA1-binding agent described herein. In some embodiments, the method inhibits progression of intermediate AMD to GA. In some embodiments, the method inhibits progression of intermediate AMD to wet AMD. In some embodiments, the method inhibits progression of intermediate AMD to CNV. In some embodiments, the method inhibits progression of dry AMD to wet AMD.

In some embodiments, a method of inhibiting HTRA1 protease activity in an eye of a subject comprises administering to an eye of the subject a therapeutically effective amount of a HTRA1-binding agent described herein.

In some embodiments of the methods described herein, a HTRA1-binding agent is administrated to an eye of the subject by ocular injection, intraocular injection, or intravitreal injection. In some embodiments, a HTRA1-binding agent is administrated to an eye of the subject by intravitreal injection. In some embodiments of the methods described herein, a HTRA1-binding agent is administrated to an eye of the subject by topical delivery. In some embodiments, a HTRA1-binding agent is administered to an eye of the subject by eye drops.

In some embodiments of the methods described herein, a HTRA1-binding agent is administered to a subject as part of a combination therapy. In some embodiments, the combination therapy comprises photodynamic therapy. In some embodiments, the combination therapy comprises photodynamic therapy with verteporfin. In some embodiments, a HTRA1-binding agent is administered to a subject, wherein the subject is administered one or more additional therapeutic agents. In some embodiments, an additional therapeutic agent is a C3 inhibitor. In some embodiments, a C3 inhibitor is compstatin or an analog or derivative of compstatin (e.g., POT-4, APL-2, AMY-101). In some embodiments, an additional therapeutic agent is a C5 inhibitor. In some embodiments, a C5 inhibitor is selected from the group including, but not limited to, eculizumab, LFG316, or Zimura (anti-C5 aptamer). In some embodiments, an additional therapeutic agent is a properdin inhibitor (e.g., an anti-properdin antibody). In some embodiments, an additional therapeutic agent is a Factor D inhibitor. In some embodiments, a Factor D inhibitor is an anti-Factor D antibody (e.g., lampalizumab). In some embodiments, an additional therapeutic agent is a VEGF inhibitor. In some embodiments, a VEGF inhibitor is selected from the group including, but not limited to, pegaptanib (MACUGEN), ranibizumab (LUCENTIS), bevacizumab (AVASTIN), aflibercept (EYLEA), brolucizumab, and OPT-302. In some embodiments, an additional therapeutic agent is a PDGF inhibitor. In some embodiments, an additional therapeutic agent is a corticosteroid. In some embodiments, an additional therapeutic agent is a neuroprotective agent. In some embodiments, a neuroprotective agent is selected from the group including, but not limited to, ciliary neurotrophic factor (CNTF), tandospirone, and brimonidine.

Also disclosed is the use of a HTRA1-binding agent described herein in the manufacture of a medicament for treating an eye disorder. In some embodiments, the eye disorder is selected from the group consisting of: macular degeneration (maculopathy), age-related macular degeneration (AMD), wet AMD, dry AMD, geographic atrophy (GA), diabetic retinopathy, retinopathy of prematurity, macular dystrophy, retinal dystrophy, uveitis, keratitis, scleritis, retinitis pigmentosa, choroidal neovascularization (CNV), retinal neovascularization, ocular inflammation, polypoidal choroidal vasculopathy (PCV), idiopathic polypoidal choroidal vasculopathy (IPCV), Stargardt disease, and neuromyelitis optica.

In some embodiments of each of the aforementioned aspects and embodiments, as well as other aspects and embodiments described herein, the subject is human.

Where aspects or embodiments of the disclosure are described in terms of a Markush group or other grouping of alternatives, the present disclosure encompasses not only the entire group listed as a whole, but also each member of the group individually and all possible subgroups of the main group, and also the main group absent one or more of the group members. The present disclosure also envisages the explicit exclusion of one or more of any of the group members in the claimed disclosure.

### BRIEF DESCRIPTION OF THE FIGURES

FIG. 1. Inhibition of HTRA1 protease activity by antibody 24F7.

FIG. 2. Inhibition of HTRA1 protease activity by antibody 24F7 in the presence of increasing substrate.

FIG. 3. Inhibition of endogenous HIRA1 protease activity by antibody 24F7.

FIG. 4. Pharmacokinetic study of antibody hz24F7.v2 in cynomolgus monkeys.

FIG. 5. HTRA1 levels in serum from cynomolgus monkeys after IV or IVT dosing with hz24F7.v2 antibody.

FIGS. 6A-6C show the detection of a fragment when hz24F7.v2 is recombinantly expressed in CHO and HEK293 cell lines by non-reduced CE-SDS. FIG. 6A shows hz24F7.v2 expressed in a CHO cell line. FIG. 6B shows hz24F7.v2 expressed in the Expi293F cell line. FIG. 6C shows a negative control corresponding to a different antibody that is not fragmented. FIGS. 6A and 6B demonstrate that the fragmentation is cell-line independent. FIG. 6C shows that fragmentation is unique to hz24F7.v2.

FIGS. 7A-7D show identification/discovery of a hz24F7.v2 antibody fragment by RP-HPLC assay when a purified sample of hz24F7.v2 is incubated under stress at pH 7.5. The level of fragmented hz24F7.v2 significantly increased from 0 to 4 weeks at pH 7.5, 40° C. FIG. 7A shows hz24F7.v2 stored for 0 weeks at pH 7.5 (pH 7.5, T0). FIG. 7B shows an enlarged graph of the hz24F7.v2 fragment (clip) when stored for 0 weeks at pH 7.5 (pH 7.5, T0 Zoom). FIG. 7C shows hz24F7.v2 stored for 4 weeks at pH 7.5 and 40° C. (pH 7.5, 40° C., 4 Wks). FIG. 7D shows an enlarged graph of the hz24F7.v2 fragment (clip) when stored for 4 weeks at pH 7.5 and 40° C. (pH 7.5, 40° C., 4 Wks, Zoom).

FIGS. 8A-8H show identification/discovery of a hz24F7.v2 antibody fragment by CE-SDS when a purified sample of hz24F7.v2 is incubated under stress at pH 6.0 and pH 7.5 for up to 8 weeks at 40° C. The level of fragmented hz24F7.v2 significantly increased during the incubation of hz24F7.v2 from 0 to 8 weeks at 40° C. FIGS. 8A-8D show the increase in fragmentation of hz24F7.v2 in 2 week increments at pH 6.0 incubation and FIGS. 8E-8H show the increase in fragmentation of hz24F7.v2 in 2 week increments at pH 7.5 incubation, with the latter showing more pronounced fragmentation over the same time period. FIGS. 8A-8H show the pH-dependent fragmentation of hz24F7.v2.

FIGS. 9A and 9B show fragmentation of hz24F7.v2 by two orthogonal methods, by CE-SDS (FIG. 9A) or RP-HPLC (FIG. 9B). For FIG. 9A and FIG. 9B, each bar graph shows from left to right, pH 6.0, pH 6.5, pH 7.0 and pH 7.5, respectively).

FIG. 10 shows the pH-dependent and time-dependent fragmentation (clipped) of hz24F7.v2 analyzed by CE-SDS upon incubation at either 30° C. or 40° C. for up to 8 weeks in 2 week increments. For FIG. 10, each bar graph shows from left to right, pH 6.0, pH 6.5, pH 7.0 and pH 7.5, respectively).

FIG. 11 shows the percentage of intact hz24F7.v2 by CE-SDS analysis at pH 6.0, 6.5, 7.0 and 7.5 across 8 weeks at 30° C. or 40° C. Intact hz24F7.v2 represents the percentage of fragmentation that has been corrected for by molecular weight difference.

FIGS. 12A and 12B show confirmation of intact and fragmented species of hz24F7.v2 by Mass Spectrometry (MS). FIG. 12A shows the S91 fragmentation site at 0 weeks. FIG. 12B shows the S91 fragmentation site at 2 weeks and 40° C. In FIG. 12A and FIG. 12B, the 145237.8 Da peptide is intact hz24F7.v2, the 135362.1 Da peptide represents an hz24F7.v2 fragmented (clipped) at position S91 in only one light chain of the antibody, the 122049.7 Da peptide represents an hz24F7.v2 missing one of its light chains and the 23188.3 Da peptide represents an hz24F7.v2 missing its light chain.

FIGS. 13A-13D show that fragmented light chain CDR3 of hz24F7.v2 remains bound to the hz24F7.v2 antibody. FIG. 13A shows hz24F7.v2 stored for 0 weeks at pH 7.5 (pH 7.5, T0). FIG. 13B shows an enlarged graph of hz24F7.v2 fragment when stored for 0 weeks at pH 7.5 (pH 7.5, T0 Zoom).

FIG. 13C shows hz24F7.v2 stored for 4 weeks at pH 7.5 and 40° C. (pH 7.5, 40° C., 4 Wks). FIG. 13D shows hz24F7.v2 stored for 4 weeks at pH 7.5 and 40° C. (pH 7.5, 40° C., 4 Wks, Zoom).

FIG. 14 shows that fragmented hz24F7.v2 retains binding affinity to HTRA1 across 8 weeks. There was no observed difference in binding at T0 (0 weeks), 2 w (2 weeks), 4 w (4 weeks), or 8 w (8 weeks). A negative control (buffer) is indicated in the gray line at RU 0 on the y-axis.

FIGS. 15A and 15B show that fragmented hz24F7.v2 has minimal impact on its inhibitory activity. FIG. 15A shows the half maximal inhibitory concentration for hz24F7.v2 and FIG. 15B shows the percentage of active hz24F7.v2.

FIGS. 16A and 16B show that hz24F7.v2 does not undergo appreciable fragmentation upon thermal stress at pH 6.5 between 0 and 2 weeks. FIGS. 16C and 16D show that hz24F7.v2 does not undergo appreciable fragmentation upon thermal stress at pH 7.4 between 0 and 2 weeks.

FIG. 17 shows analysis of stressed hz24F7.v2 S91 and S92 mutants by CE-SDS analysis, at pH 6.0 and 7.4 at 40° C.

FIGS. 18A and 18B show evaluation of hz24F7.v2 S91Y and hz24F7.v2 S92T in an enzyme assay. FIG. 18A shows that IC₅₀ of hz24F7.v2 S91Y at 0, 2 and 4 weeks is not significantly different than hz24F7.v2. FIG. 18B shows that IC₅₀ of hz24F7.v2 S92T at 0, 2 and 4 weeks is not significantly different than hz24F7.v2.

### DETAILED DESCRIPTION

The present disclosure provides novel agents, including but not limited to polypeptides such as antibodies, that bind HTRA1. The HTRA1-binding agents include, but are not limited to, polypeptides, antibodies (including antigen-binding fragments thereof), scaffold proteins, and heterodimeric molecules. HTRA1-binding agents include, but are not limited to, antagonists of HTRA1 activity, inhibitors of HTRA1 activity, and/or agents that modulate HTRA1 activity. Related polypeptides, polynucleotides, vectors, compositions comprising the agents, cells comprising the related polynucleotides or vectors, and methods of making the agents are also provided. Methods of using the novel HTRA1-binding agents are also provided.

### I. Definitions

Unless otherwise defined herein, technical and scientific terms used in the present description have the meanings that are commonly understood by those of ordinary skill in the art. For purposes of interpreting this specification, the following description of terms will apply and whenever appropriate, terms used in the singular will also include the plural and vice versa.

The term “binding agent” as used herein refers to a molecule that binds a specific antigen or target (e.g., HTRA1). A binding agent may comprise a protein, peptide, nucleic acid, carbohydrate, lipid, or small molecular weight compound. In some embodiments, a binding agent comprises an antibody or an antigen-binding fragment thereof. In some embodiments, a binding agent is an antibody or an antigen-binding fragment thereof. In some embodiments, a binding agent comprises an alternative protein scaffold or artificial scaffold (e.g., a non-immunoglobulin backbone). In some embodiments, a binding agent is a fusion protein comprising an antigen-binding site. In some embodiments, a binding agent is a bispecific or multispecific molecule comprising at least one antigen-binding site.

The term “antibody” is used herein in the broadest sense and encompasses various antibody structures, including but not limited to, an immunoglobulin molecule that recognizes and binds a target through at least one antigen-binding site, polyclonal antibodies, recombinant antibodies, monoclonal antibodies, chimeric antibodies, humanized antibodies, human antibodies, bispecific antibodies, multispecific antibodies, diabodies, tribodies, tetrabodies, single chain Fv (scFv) antibodies, and antibody fragments as long as they exhibit the desired antigen-binding activity.

The term “intact antibody” or “full-length antibody” refers to an antibody having a structure substantially similar to a native antibody structure. This includes, for example, an antibody comprising two light chains each comprising a variable region and a light chain constant region (CL) and two heavy chains each comprising a variable region and at least heavy chain constant regions CH1, CH2, and CH3. Generally, an intact antibody includes a hinge region (or a portion thereof) between the CH1 and CH2 regions.

The term “antibody fragment” as used herein refers to a molecule other than an intact antibody that comprises a portion of an antibody and generally an antigen-binding site. Examples of antibody fragments include, but are not limited to, Fab, Fab′, F(ab′)2, Fv, single chain antibody molecules (e.g., scFv), sc(Fv)₂, disulfide-linked scFv (dsscFv), diabodies, tribodies, tetrabodies, minibodies, dual variable domain antibodies (DVD), single variable domain antibodies (e.g., camelid antibodies), and multispecific antibodies formed from antibody fragments.

The term “monoclonal antibody” as used herein refers to a substantially homogenous antibody population involved in the highly specific recognition and binding of a single antigenic determinant or epitope. The term “monoclonal antibody” encompasses intact and full-length monoclonal antibodies as well as antibody fragments, single chain antibodies, fusion proteins comprising an antibody fragment, and any other modified immunoglobulin molecule comprising at least one antigen-binding site. Furthermore, “monoclonal antibody” refers to such antibodies made by any number of techniques, including but not limited to, hybridoma production, phage library display, recombinant expression, and transgenic animals.

The term “chimeric antibody” refers to an antibody in which a portion of the heavy and/or light chain is derived from a first source or species, while the remainder of the heavy and/or light chain is derived from a different source or species.

The term “humanized antibody” as used herein refers to an antibody that comprises a human heavy chain variable region and a light chain variable region wherein the native CDR amino acid residues are replaced by residues from corresponding CDRs from a nonhuman antibody (e.g., mouse, rat, rabbit, or nonhuman primate), wherein the nonhuman antibody has the desired specificity, affinity, and/or activity. In some embodiments, one or more framework region amino acid residues of the human heavy chain or light chain variable regions are replaced by corresponding residues from nonhuman antibody. Furthermore, humanized antibodies can comprise amino acid residues that are not found in the human antibody or in the nonhuman antibody. In some embodiments, these modifications are made to further refine and/or optimize antibody characteristics. In some embodiments, the humanized antibody comprises at least a portion of an immunoglobulin constant region (e.g., CH1, CH2, CH3, Fc, and/or hinge region), typically that of a human immunoglobulin.

The term “human antibody” as used herein refers to an antibody that possesses an amino acid sequence that corresponds to an antibody produced by a human and/or an antibody that has been made using any of the techniques that are known to those of skill in the art for making human antibodies. These techniques include, but not limited to, phage display libraries, yeast display libraries, transgenic animals, recombinant protein production, and B-cell hybridoma technology.

The terms “epitope” and “antigenic determinant” are used interchangeably herein and refer to that portion of an antigen or target capable of being recognized and bound by a particular antibody. When the antigen or target is a polypeptide, epitopes can be formed both from contiguous amino acids and noncontiguous amino acids juxtaposed by tertiary folding of the protein. Epitopes formed from contiguous amino acids (also referred to as linear epitopes) are typically retained upon protein denaturing, whereas epitopes formed by tertiary folding (also referred to as conformational epitopes) are typically lost upon protein denaturing. An epitope typically includes at least 3, and more usually, at least 5, 6, 7, or 8-10 amino acids in a unique spatial conformation. Epitopes can be predicted using any one of a large number of software bioinformatic tools available on the internet. In some embodiments, X-ray crystallography is used to characterize an epitope on a target protein by analyzing the amino acid residue interactions of an antigen/antibody complex.

The term “specifically binds” as used herein refers to an agent that interacts more frequently, more rapidly, with greater duration, with greater affinity, or with some combination of the above to a particular antigen, epitope, protein, or target molecule than with alternative substances. A binding agent that specifically binds an antigen can be identified, for example, by immunoassays, ELISAs, surface plasmon resonance (SPR), or other techniques known to those of skill in the art. In some embodiments, an agent that specifically binds an antigen (e.g., human HTRA1) can bind related antigens (e.g., cyno HTRA1). A binding agent that specifically binds an antigen can bind the target antigen at a higher affinity than its affinity for a different antigen. The different antigen can be a related antigen. In some embodiments, a binding agent that specifically binds an antigen can bind the target antigen with an affinity that is at least 20 times greater, at least 30 times greater, at least 40 times greater, at least 50 times greater, at least 60 times greater, at least 70 times greater, at least 80 times greater, at least 90 times greater, or at least 100 times greater, than its affinity for a different antigen. In some embodiments, a binding agent that specifically binds a particular antigen binds a different antigen at such a low affinity that binding cannot be detected using an assay described herein or otherwise known in the art. In some embodiments, affinity is measured using SPR technology in a Biacore system as described herein or as known to those of skill in the art.

The terms “polypeptide” and “peptide” and “protein” are used interchangeably herein and refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification. Also included within the definition are, for example, polypeptides containing one or more analogs of an amino acid, including but not limited to, unnatural amino acids, as well as other modifications known in the art. It is understood that, because the polypeptides of this disclosure may be based upon antibodies, the term “polypeptide” encompasses polypeptides as a single chain and polypeptides of two or more associated chains.

The terms “polynucleotide” and “nucleic acid” and “nucleic acid molecule” are used interchangeably herein and refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase.

The terms “identical” or percent “identity” in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity may be measured using sequence comparison software or algorithms or by visual inspection. Various algorithms and software that may be used to obtain alignments of amino acid or nucleotide sequences are well-known in the art. These include, but are not limited to, BLAST, ALIGN, Megalign, BestFit, GCG Wisconsin Package, and variants thereof. In some embodiments, two nucleic acids or polypeptides of the disclosure are substantially identical, meaning they have at least 70%, at least 75%, at least 80%, at least 85%, at least 90%, and in some embodiments at least 95%, at least 96%, at least 97%, at least 98%, at least 99% nucleotide or amino acid residue identity, when compared and aligned for maximum correspondence, as measured using a sequence comparison algorithm or by visual inspection. In some embodiments, identity exists over a region of the sequences that is at least about 10, at least about 20, at least about 20-40, at least about 40-60 nucleotides or amino acid residues, at least about 60-80 nucleotides or amino acid residues in length or any integral value there between. In some embodiments, identity exists over a longer region than 60-80 nucleotides or amino acid residues, such as at least about 80-100 nucleotides or amino acid residues, and in some embodiments the sequences are substantially identical over the full length of the sequences being compared, for example, (i) the coding region of a nucleotide sequence or (ii) an amino acid sequence.

The phrase “conservative amino acid substitution” as used herein refers to a substitution in which one amino acid residue is replaced with another amino acid residue having a similar side chain. Families of amino acid residues having similar side chains have been generally defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is considered to be a conservative substitution. Generally, conservative substitutions in the sequences of polypeptides and/or antibodies do not abrogate the binding of the polypeptide or antibody to the target binding site. Methods of identifying nucleotide and amino acid conservative substitutions that do not eliminate binding are well-known in the art.

The term “vector” as used herein means a construct that is capable of delivering, and usually expressing, one or more gene(s) or sequence(s) of interest in a host cell. Examples of vectors include, but are not limited to, viral vectors, naked DNA or RNA expression vectors, plasmid, cosmid, or phage vectors, DNA or RNA expression vectors associated with cationic condensing agents, and DNA or RNA expression vectors encapsulated in liposomes.

The term “isolated” as used herein refers to a polypeptide, soluble protein, antibody, polynucleotide, vector, cell, or composition that is in a form not found in nature. An “isolated” antibody is substantially free of material from the cellular source from which it is derived. In some embodiments, isolated polypeptides, soluble proteins, antibodies, polynucleotides, vectors, cells, or compositions are those that have been purified to a degree that they are no longer in a form in which they are found in nature. In some embodiments, a polypeptide, soluble protein, antibody, polynucleotide, vector, cell, or composition that is isolated is substantially pure. A polypeptide, soluble protein, antibody, polynucleotide, vector, cell, or composition can be isolated from a natural source (e.g., tissue) or from a source such as an engineered cell line.

The term “substantially pure” as used herein refers to material that is at least 50% pure (i.e., free from contaminants), at least 90% pure, at least 95% pure, at least 98% pure, or at least 99% pure.

The term “subject” refers to any animal (e.g., a mammal), including, but not limited to, humans, non-human primates, canines, felines, rabbits, rodents, and the like.

The term “pharmaceutically acceptable” as used herein refers to a substance approved or approvable by a regulatory agency or listed in the U.S. Pharmacopeia, European Pharmacopeia, or other generally recognized pharmacopeia for use in animals, including humans.

The terms “pharmaceutically acceptable excipient, carrier, or adjuvant” or “acceptable pharmaceutical carrier” as used herein refer to an excipient, carrier, or adjuvant that can be administered to a subject, together with at least one therapeutic agent, and that is generally safe, non-toxic, and has no effect on the pharmacological activity of the therapeutic agent. In general, those of skill in the art and the U.S. FDA consider a pharmaceutically acceptable excipient, carrier, or adjuvant to be an inactive ingredient of any formulation.

The term “pharmaceutical formulation” or “pharmaceutical composition” as used herein refers to a preparation that is in such form as to permit the biological activity of the agent to be effective. A pharmaceutical formulation or composition generally comprises additional components, such as a pharmaceutically acceptable excipient, carrier, adjuvant, buffers, etc.

The term “effective amount” or “therapeutically effective amount” as used herein refers to the amount of an agent that is sufficient to reduce and/or ameliorate the severity and/or duration of (i) a disease, disorder or condition in a subject, and/or (ii) a symptom in a subject. The term also encompasses an amount of an agent necessary for the (i) reduction or amelioration of the advancement or progression of a given disease, disorder, or condition, (ii) reduction or amelioration of the recurrence, development, or onset of a given disease, disorder, or condition, and/or (iii) the improvement or enhancement of the prophylactic or therapeutic effect(s) of another agent or therapy (e.g., an agent other than the binding agents provided herein).

The term “therapeutic effect” as used herein refers to the effect and/or ability of an agent to reduce and/or ameliorate the severity and/or duration of (i) a disease, disorder, or condition in a subject, and/or (ii) a symptom in a subject. The term also encompasses the ability of an agent to (i) reduce or ameliorate the advancement or progression of a given disease, disorder, or condition, (ii) reduce or ameliorate the recurrence, development, or onset of a given disease, disorder, or condition, and/or (iii) to improve or enhance the prophylactic or therapeutic effect(s) of another agent or therapy (e.g., an agent other than the binding agents provided herein).

The term “treat” or “treatment” or “treating” or “to treat” or “alleviate” or alleviation” or “alleviating” or “to alleviate” as used herein refers to both (1) therapeutic measures that aim to cure, slow down, lessen symptoms of, and/or halt progression of a pathologic condition or disorder and (2) prophylactic or preventative measures that aim to prevent or slow the development of a targeted pathologic condition or disorder. Thus, those in need of treatment include those already with the disorder, those at risk of having/developing the disorder, and those in whom the disorder is to be prevented.

The term “prevent” or “prevention” or “preventing” as used herein refers to the partial or total inhibition of the development, recurrence, onset, or spread of a disease, disorder, or condition, or a symptom thereof in a subject.

As used herein, reference to “about” or “approximately” a value or parameter includes (and describes) embodiments that are directed to that value or parameter. For example, a description referring to “about X” includes description of “X”.

As used in the present disclosure and claims, the singular forms “a”, “an” and “the” include plural forms unless the context clearly dictates otherwise.

It is understood that wherever embodiments are described herein with the term “comprising” otherwise analogous embodiments described in terms of “consisting of” and/or “consisting essentially of” are also provided. It is also understood that wherever embodiments are described herein with the phrase “consisting essentially of” otherwise analogous embodiments described in terms of “consisting of” are also provided.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

### II. HTRA1-Binding Agents

The HTRA1 gene has been linked with development of AMD by several independent genome-wide analyses (GWAS) (Dewan et al., 2006, Science, 314:9989-992; Yang et al., 2006, Science, 314:992-993) and HTRA1 susceptibility loci have been associated with AMD progression (Yan et al., 2018, Human Mol. Genet., 27:929-940).

Additional evidence of the involvement of HTRA1 in the pathogenesis of AMD comes from transgenic mouse models expressing the human HTRA1 gene. Mice overexpressing wild-type human HTRA1 develop features reminiscent of advanced AMD, whereas mice overexpressing catalytically inactive versions of HTRA1 do not (Kumar et al., 2017, Am. J. Pathol., 187:2841-2857; lejima et al., 2015, J. Stem Cells, 10:193-203).

Amino acid (aa) sequences for human HTRA1 (UniProtKB No. Q92743), rabbit HTRA1 (NCBI Ref No. XP_008268840.1), and cynomolgus monkey (“cyno”) HTRA1 (NCBI Ref No. XP 015311437) are provided herein as SEQ ID NO:1, SEQ ID NO:5, and SEQ ID NO:7, respectively. As used herein, reference to amino acid positions of HTRA1 refer to the numbering of amino acid sequences including the signal sequence.

Human HTRA1 comprises an IGFBP domain, a Kazal-like domain, a catalytic domain, and a PDZ domain (see, e.g., Eigenbrot et al., 2012, Structure, 20:1040-1050). The boundaries of any domain are not definitively known and the amino acids used herein to define domains of human HTRA1 are based on information from UniProtKB. Therefore the boundaries of any domain and/or repeat may vary from those recited herein. The IGFBP domain can be referred to as the N-terminal domain. In some situations, the IGFBP domain and the Kazal-like domain can be referred to as the N-terminal domain. In some embodiments, the N-terminal domain of HTRA1 comprises amino acids 33-100 of SEQ ID NO:1. In some embodiments, the N-terminal domain of HTRA1 comprises amino acids 33-157 of SEQ ID NO:1. In some embodiments, the IGFBP domain comprises amino acids 33-100 of SEQ ID NO:1. In some embodiments, the Kazal-like domain comprises amino acids 98-157 of SEQ ID NO:1 In some embodiments, a HTRA1 fragment comprises the catalytic domain and the PDZ domain. In some embodiments, the catalytic domain and PDZ domain of HTRA1 comprise amino acids 158-467 of SEQ ID NO:1. In some embodiments, a HTRA1 fragment comprises the catalytic domain. In some embodiments, the catalytic domain of HTRA1 comprises amino acids 158-364 of SEQ ID NO:1. In some embodiments, the serine protease domain of HTRA1 comprises amino acids 204-364 of SEQ ID NO:1. In some embodiments, a HTRA1 fragment does not include the IGFBP domain. In some embodiments, a HTRA1 fragment does not include amino acids 33-100 of SEQ ID NO:1. In some embodiments, a HTRA1 fragment does not include amino acids 1-100 of SEQ ID NO:1. In some embodiments, a HTRA1 fragment does not include the IGFBP domain or the Kazal-like domain. In some embodiments, a HTRA1 fragment does not include amino acids 33-157 of SEQ ID NO:1. In some embodiments, a HTRA1 fragment does not include amino acids 1-157 of SEQ ID NO:1. In some embodiments, a HTRA1 fragment comprises amino acids 101-480 of SEQ ID NO:1. In some embodiments, a HTRA1 fragment comprises amino acids 158-480 of SEQ ID NO:1. In some embodiments, a HTRA1 fragment comprises amino acids 158-364 of SEQ ID NO:1. In some embodiments, a HTRA1 fragment comprises amino acids 161-379 of SEQ ID NO:1. In some embodiments, a HTRA1 fragment comprises amino acids 204-379 of SEQ ID NO:1. In some embodiments, a HTRA1 fragment comprises the amino acid sequence of SEQ ID NO:3. In some embodiments, a HTRA1 fragment comprises the amino acid sequence of SEQ ID NO:4. It is understood that the domains of HTRA1 may be defined differently by those of skill in the art, therefore the N-terminal amino acids and the C-terminal amino acids of any HTRA1 domain may vary by 1, 2, 3, 4, 5, or more amino acid residues.

The present disclosure provides agents that bind HTRA1. In some embodiments, a HTRA1-binding agent binds a fragment of HTRA1. In some embodiments, a HTRA1-binding agent binds within a specific region of HTRA1. In some embodiments, a HTRA1-binding agent binds within the catalytic domain of HTRA1. In some embodiments, a HTRA1-binding agent not does bind within the N-terminal domain. In some embodiments, a HTRA1-binding agent binds a HTRA1 fragment that does not contain the N-terminal domain. In some embodiments, a HTRA1-binding agent binds an epitope on HTRA1. In some embodiments, a HTRA1-binding agent binds a linear epitope on HTRA1. In some embodiments, a HTRA1-binding agent binds a conformational epitope on HTRA1. In some embodiments, a HTRA1-binding agent binds human HTRA1. In some embodiments, a HTRA1-binding agent binds rabbit HTRA1. In some embodiments, a HTRA1-binding agent binds cyno HTRA1. In some embodiments, a HTRA1-binding agent binds human HTRA1 and cyno HTRA1. In some embodiments, a HTRA1-binding agent binds SEQ ID NO:1. In some embodiments, a HTRA1-binding agent binds SEQ ID NO:2. In some embodiments, a HTRA1-binding agent binds within amino acids 158-480 of SEQ ID NO:1. In some embodiments, a HTRA1-binding agent binds within amino acids 158-364 of SEQ ID NO:1. In some embodiments, a HTRA1-binding agent binds within amino acids 204-364 of SEQ ID NO:1. In some embodiments, a HTRA1-binding agent binds SEQ ID NO:5. In some embodiments, a HTRA1-binding agent binds SEQ ID NO:6. In some embodiments, a HTRA1-binding agent binds within amino acids 199-523 of SEQ ID NO:5. In some embodiments, a HTRA1-binding agent binds SEQ ID NO:7. In some embodiments, a HTRA1-binding agent binds SEQ ID NO:8. In some embodiments, a HTRA1-binding agent binds within amino acids 138-462 of SEQ ID NO:7.

In some embodiments, a HTRA1-binding agent binds a polypeptide comprising the amino acid sequence of SEQ ID NO:2. In some embodiments, a HTRA1-binding agent binds a polypeptide comprising the amino acid sequence of SEQ ID NO:3. In some embodiments, a HTRA1-binding agent binds a polypeptide comprising the amino acid sequence of SEQ ID NO:4. In some embodiments, a HTRA1-binding agent binds a polypeptide comprising the amino acid sequence of SEQ ID NO:6. In some embodiments, a HTRA1-binding agent binds a polypeptide comprising the amino acid sequence of SEQ ID NO:8.

In some embodiments, the HTRA1-binding agent binds an epitope comprising amino acids within SEQ ID NO:2. In some embodiments, the HTRA1-binding agent binds an epitope comprising amino acids within SEQ ID NO:3. In some embodiments, the HTRA1-binding agent binds an epitope comprising at least one (e.g., 1, 2, 3, 4, 5, 6, 7, 8, 9) amino acid within amino acids 185-200 of SEQ ID NO:1. In some embodiments, the HTRA1-binding agent binds a conformational epitope comprising at least one (e.g., 1, 2, 3, 4, 5, 6, 7, 8, 9) amino acid within amino acids 185-200 of SEQ ID NO:1. In some embodiments, the HTRA1-binding agent binds a conformational epitope comprising at least amino acid R190, L192, and/or R197 of SEQ ID NO:1. In some embodiments, the HTRA1-binding agent binds an epitope comprising amino acids within SEQ ID NO:6. In some embodiments, the HTRA1-binding agent binds an epitope comprising amino acids within SEQ ID NO:8.

In some embodiments, the HTRA1-binding agent binds human HTRA1 and has at least one or more of the following properties: (a) binds cyno HTRA1, (b) binds rabbit HTRA1, (c) inhibits HTRA1 protease activity, (d) inhibits HTRA1 protease activity in an allosteric manner, and (e) does not inhibit protease activity of other proteases in HTRA family.

In some embodiments, a HTRA1-binding agent is an antibody. In some embodiments, the antibody is a recombinant antibody. In some embodiments, the antibody is a monoclonal antibody. In some embodiments, the antibody is a chimeric antibody. In some embodiments, the antibody is a humanized antibody. In some embodiments, the antibody is a human antibody. In some embodiments, the antibody is an IgA, IgD, IgE, IgG, or IgM antibody. In some embodiments, the antibody is a human IgA, IgD, IgE, IgG, or IgM antibody. In some embodiments, the antibody is an IgG1 antibody. In some embodiments, the antibody is a human IgG1 antibody. In some embodiments, the antibody is an IgG2 antibody. In some embodiments, the antibody is a human IgG2 antibody. In some embodiments, the antibody is an IgG3 antibody. In some embodiments, the antibody is a human IgG3 antibody. In some embodiments, the antibody is an IgG4 antibody. In some embodiments, the antibody is a human IgG4 antibody. In some embodiments, the antibody comprises an IgG heavy chain. In some embodiments, the antibody comprises an IgG1 heavy chain. In some embodiments, the antibody comprises an IgG2 heavy chain. In some embodiments, the antibody comprises an IgG4 heavy chain. In some embodiments, the antibody comprises a kappa light chain. In some embodiments, the antibody comprises a human kappa light chain. In some embodiments, the antibody comprises a kappa light chain constant region. In some embodiments, the antibody comprises a human kappa light chain constant region. In some embodiments, the antibody comprises a lambda light chain. In some embodiments, the antibody comprises a human lambda light chain. In some embodiments, the antibody comprises a lambda light chain constant region. In some embodiments, the antibody comprises a human lambda light chain constant region. In some embodiments, the antibody is an antibody fragment comprising an antigen-binding site. In some embodiments, the antibody is a scFv. In some embodiments, the antibody is a disulfide-linked scFv. In some embodiments, the antibody is a disulfide-linked sc(Fv)₂. In some embodiments, the antibody is a Fab, Fab′, or a F(ab′)₂ antibody. In some embodiments, the antibody is a diabody. In some embodiments, the antibody is a nanobody. In some embodiments, the antibody is a monospecific antibody. In some embodiments, the antibody is a bispecific antibody. In some embodiments, the antibody is a multispecific antibody. In some embodiments, the antibody is a monovalent antibody. In some embodiments, the antibody is a bivalent antibody. In some embodiments, the antibody is a tetravalent antibody.

In some embodiments, the antibody is isolated. In some embodiments, the antibody is substantially pure.

In some embodiments, a HTRA1-binding agent is a polyclonal antibody. Polyclonal antibodies can be prepared by any method known to those of skill in the art. In some embodiments, polyclonal antibodies are produced by immunizing an animal (e.g., a rabbit, rat, mouse, goat, donkey) with an antigen of interest (e.g., a purified peptide fragment, a recombinant protein, or a fusion protein) using multiple subcutaneous or intraperitoneal injections. In some embodiments, the antigen is conjugated to a carrier such as keyhole limpet hemocyanin (KLH), serum albumin, bovine thyroglobulin, or soybean trypsin inhibitor. The antigen (with or without a carrier protein) is diluted in sterile saline and usually combined with an adjuvant (e.g., Complete or Incomplete Freund's Adjuvant) to form a stable emulsion. After a period of time, polyclonal antibodies are recovered from the immunized animal (e.g., from blood or ascites). In some embodiments, the polyclonal antibodies are purified from serum or ascites according to standard methods in the art including, but not limited to, affinity chromatography, ion-exchange chromatography, gel electrophoresis, and/or dialysis.

In some embodiments, a HTRA1-binding agent is a monoclonal antibody. Monoclonal antibodies can be prepared by any method known to those of skill in the art. In some embodiments, monoclonal antibodies are prepared using hybridoma methods known to one of skill in the art. For example, using a hybridoma method, a mouse, rat, rabbit, hamster, or other appropriate host animal, is immunized as described above. In some embodiments, lymphocytes are immunized in vitro. In some embodiments, the immunizing antigen is a human protein or a fragment thereof. In some embodiments, the immunizing antigen is a mouse protein or a fragment thereof.

Following immunization, lymphocytes are isolated and fused with a suitable myeloma cell line using, for example, polyethylene glycol. The hybridoma cells are selected using specialized media as known in the art and unfused lymphocytes and myeloma cells do not survive the selection process. Hybridomas that produce monoclonal antibodies directed specifically against a chosen antigen can be identified by a variety of methods including, but not limited to, immunoprecipitation, immunoblotting, and in vitro binding assays (e.g., flow cytometry, FACS, ELISA, SPR (e.g., Biacore), and radioimmunoassay). Once hybridoma cells that produce antibodies of the desired specificity, affinity, and/or activity are identified, the clones may be subcloned by limiting dilution techniques. The hybridomas can be propagated either in in vitro culture using standard methods or in vivo as ascites tumors in an animal. The monoclonal antibodies can be purified from the culture medium or ascites fluid according to standard methods in the art including, but not limited to, affinity chromatography, ion-exchange chromatography, gel electrophoresis, and dialysis.

In some embodiments, monoclonal antibodies are made using recombinant DNA techniques as known to one skilled in the art. For example, the polynucleotides encoding an antibody are isolated from mature B-cells or hybridoma cells, such as by RT-PCR using oligonucleotide primers that specifically amplify the genes encoding the heavy and light chains of the antibody, and their sequence is determined using standard techniques. The isolated polynucleotides encoding the heavy and light chains are then cloned into suitable expression vectors which produce the monoclonal antibodies when transfected into host cells such as E. coli, simian COS cells, Chinese hamster ovary (CHO) cells, or myeloma cells that do not otherwise produce immunoglobulin proteins.

In some embodiments, recombinant monoclonal antibodies are isolated from phage display libraries expressing variable domains or CDRs of a desired species. Screening of phage libraries can be accomplished by various techniques known in the art.

In some embodiments, a monoclonal antibody is modified by using recombinant DNA technology to generate alternative antibodies. In some embodiments, the constant domains of the light chain and heavy chain of a mouse monoclonal antibody are substituted for constant regions of a human antibody to generate a chimeric antibody. In some embodiments, the constant regions are truncated or removed to generate a desired antibody fragment of a monoclonal antibody. In some embodiments, site-directed or high-density mutagenesis of the variable region(s) is used to optimize specificity and affinity of a monoclonal antibody.

In some embodiments, a HTRA1-binding agent is a humanized antibody. Various methods for generating humanized antibodies are known in the art. In some embodiments, a humanized antibody comprises one or more amino acid residues that have been introduced into it from a source that is non-human. In some embodiments, humanization is performed by substituting one or more non-human CDR sequences for the corresponding CDR sequences of a human antibody. In some embodiments, the humanized antibodies are constructed by substituting all six CDRs of a non-human antibody (e.g., a mouse antibody) for the corresponding CDRs of a human antibody.

The choice of which human heavy chain variable region and/or light chain variable region is used for generating humanized antibodies can be made based on a variety of factors and by a variety of methods known in the art. In some embodiments, the “best-fit” method is used where the sequence of the variable region of a non-human (e.g., rodent) antibody is screened against the entire library of known human variable region sequences. The human sequence that is most similar to that of the non-human (e.g., rodent) sequence is selected as the human variable region framework for the humanized antibody. In some embodiments, a particular variable region framework derived from a consensus sequence of all human antibodies of a particular subgroup of light or heavy chains is selected as the variable region framework. In some embodiments, the variable region framework sequence is derived from the consensus sequences of the most abundant human subclasses. In some embodiments, human germline genes are used as the source of the variable region framework sequences.

Other methods for humanization include, but are not limited to, a method called “superhumanization” which is described as the direct transfer of CDRs to a human germline framework, a method termed Human String Content (HSC) which is based on a metric of “antibody humanness”, methods based on generation of large libraries of humanized variants (including phage, ribosomal, and yeast display libraries), and methods based on framework region shuffling.

In some embodiments, a HTRA1-binding agent is a human antibody. Human antibodies can be prepared using various techniques known in the art. In some embodiments, human antibodies are generated from immortalized human B lymphocytes immunized in vitro. In some embodiments, human antibodies are generated from lymphocytes isolated from an immunized individual. In any case, cells that produce an antibody directed against a target antigen can be generated and isolated. In some embodiments, a human antibody is selected from a phage library, where that phage library expresses human antibodies. Alternatively, phage display technology may be used to produce human antibodies and antibody fragments in vitro, from immunoglobulin variable region gene repertoires from unimmunized donors. Techniques for the generation and use of antibody phage libraries are well known in the art. Once antibodies are identified, affinity maturation strategies known in the art, including but not limited to, chain shuffling and site-directed mutagenesis, may be employed to generate higher affinity human antibodies. In some embodiments, human antibodies are produced in transgenic mice that contain human immunoglobulin loci. Upon immunization these mice are capable of producing the full repertoire of human antibodies in the absence of endogenous immunoglobulin production.

In some embodiments, a HTRA1-binding agent is an antibody fragment. As used herein, the term “antibody fragment” refers to a molecule other than an intact antibody that comprises a portion of an antibody and generally an antigen-binding site. Examples of antibody fragments include, but are not limited to, Fab, Fab′, F(ab′)₂, Fv, single chain antibody molecules, scFv, disulfide-linked scFv (dsscFv), nanobodies, diabodies, tribodies, tetrabodies, minibodies, dual variable domain antibodies (DVD), single variable domain antibodies (e.g., camelid antibodies), and multispecific antibodies formed from antibody fragments.

In some embodiments, a HTRA1-binding agent is a scFv antibody. In some embodiments, the scFv is a disulfide-linked scFv, which is a scFv comprising an engineered disulfide bond between the light chain variable region and heavy chain variable region of the scFv. In some embodiments, the disulfide bond increases stability of the scFv molecule. In some embodiments, the disulfide bond increases thermostability of the scFv molecule.

In some embodiments, a HTRA1-binding agent is a Fv. In some embodiments, a HTRA1-binding agent is a Fab. In some embodiments, a HTRA1-binding agent is a F(ab′)₂. In some embodiments, a HTRA1-binding agent is a F(ab′).

Antibody fragments can be made by various techniques, including but not limited to proteolytic digestion of an intact antibody. The antibody fragments described herein can be produced using recombinant technologies known in the art (e.g., E. coli or phage expression).

In some embodiments, a HTRA1-binding agent is a bispecific antibody. Bispecific antibodies are capable of recognizing and binding at least two different antigens or epitopes. The different epitopes can either be within the same molecule (e.g., two epitopes on HTRA1) or on different molecules (e.g., one epitope on HTRA1 and one epitope on a different target). In some embodiments, a bispecific antibody has enhanced potency as compared to an individual antibody or to a combination of more than one antibody. In some embodiments, a bispecific antibody has reduced toxicity as compared to an individual antibody or to a combination of more than one antibody. It is known to those of skill in the art that any therapeutic agent may have unique pharmacokinetics (PK) (e.g., circulating half-life). In some embodiments, a bispecific antibody has the ability to synchronize the PK of two binding agents wherein the two individual binding agents have different PK profiles. In some embodiments, a bispecific antibody has the ability to target the actions of two agents in a common area (e.g., tissue) in a subject. In some embodiments, a bispecific antibody has the ability to target the actions of two agents to a common object (e.g., a specific cell type). In some embodiments, a bispecific antibody has the ability to target the actions of two agents to more than one biological pathway or function. In some embodiments, a bispecific antibody has the ability to target two different cells and bring them closer together.

In some embodiments, a bispecific antibody has decreased toxicity and/or side effects. In some embodiments, a bispecific antibody has decreased toxicity and/or side effects as compared to a mixture of the two individual antibodies or the antibodies as single agents. In some embodiments, a bispecific antibody has an increased therapeutic index. In some embodiments, a bispecific antibody has an increased therapeutic index as compared to a mixture of the two individual antibodies or the antibodies as single agents.

Many techniques for making bispecific antibodies are known to those skilled in the art. In some embodiments, a bispecific antibody comprises heavy chain constant regions with modifications in the amino acids that are part of the interface between the two heavy chains. These modifications are made to enhance heterodimer formation and generally reduce or eliminate homodimer formation. In some embodiments, the bispecific antibody is generated using a knobs-into-holes (KIH) strategy. In some embodiments, the bispecific antibody comprises variant hinge regions incapable of forming disulfide linkages between identical heavy chains. In some embodiments, the bispecific antibody comprises heavy chains with changes in amino acids that result in altered electrostatic interactions. In some embodiments, the bispecific antibodies comprise heavy chains with changes in amino acids that result in altered hydrophobic/hydrophilic interactions.

Bispecific antibodies can be intact antibodies or antibody fragments comprising antigen-binding sites.

In some embodiments, a HTRA1-binding agent is an antibody that binds HTRA1. In some embodiments, an anti-HTRA1 antibody binds human HTRA1. In some embodiments, an anti-HTRA1 antibody binds cyno HTRA1. In some embodiments, an anti-HTRA1 antibody binds human HTRA1 and cyno HTRA1. In some embodiments, an anti-HTRA1 antibody binds rabbit HTRA1. In some embodiments, an anti-HTRA1 antibody binds human HTRA1 and rabbit HTRA1. In some embodiments, an anti-HTRA1 antibody binds a HTRA1 epitope. In some embodiments, an anti-HTRA1 antibody binds a HTRA1 epitope within the catalytic domain of human HTRA1. In some embodiments, an anti-HTRA1 antibody binds a HTRA1 epitope within the catalytic domain of cyno HTRA1. In some embodiments, an anti-HTRA1 antibody binds an epitope comprising at least one amino acid within amino acids 185-200 of SEQ ID NO:1. In some embodiments, an anti-HTRA1 antibody binds an epitope comprising at least amino acid R190, L192, and/or R197 of SEQ ID NO:1. In some embodiments, an anti-HTRA1 antibody binds an epitope comprising amino acids within SEQ ID NO:6. In some embodiments, an anti-HTRA1 antibody binds an epitope comprising amino acids within SEQ ID NO:8. In some embodiments, the epitope is a conformational epitope. In some embodiments, the epitope is a linear epitope.

In some embodiments, a HTRA1-binding agent is an anti-HTRA1 antibody described herein. In some embodiments, the HTRA1-binding agent is a variant of an anti-HTRA1 antibody described herein. In some embodiments, a variant of an anti-HTRA1 antibody comprises one to thirty amino acid substitutions. In some embodiments, a variant of the anti-HTRA1 antibody comprises one to twenty-five amino acid substitutions. In some embodiments, a variant of the anti-HTRA1 antibody comprises one to twenty amino acid substitutions. In some embodiments, a variant of the anti-H IRA1 antibody comprises one to fifteen amino acid substitutions. In some embodiments, a variant of the anti-HTRA1 antibody comprises one to ten amino acid substitutions. In some embodiments, a variant of the anti-HTRA1 antibody comprises one to five amino acid substitutions. In some embodiments, the variant of the anti-HTRA1 antibody comprises one to three amino acid substitutions. In some embodiments, the amino acid substitution(s) is in a CDR of the antibody. In some embodiments, the amino acid substitution(s) is not in a CDR of the antibody. In some embodiments, the amino acid substitution(s) is in a framework region of the antibody. In some embodiments, the amino acid substitution(s) is in the heavy chain variable region of the antibody. In some embodiments, the amino acid substitution(s) is in the light chain variable region of the antibody. In some embodiments, the amino acid substitution(s) is a conservative amino acid substitution.

In some embodiments, a HTRA1-binding agent comprises one or more (e.g., 1, 2, 3, 4, etc.) amino acid substitutions in a CDR of an antibody described herein. In some embodiments, the amino acid substitutions are conservative substitutions. In some embodiments, a CDR comprises one amino acid substitution. In some embodiments, a CDR comprises two amino acid substitutions. In some embodiments, a CDR comprises three amino acid substitutions. In some embodiments, a CDR comprises four amino acid substitutions. In some embodiments, the CDR is a heavy chain variable region CDR1. In some embodiments, the CDR is a heavy chain variable region CDR2. In some embodiment, the CDR is a heavy chain variable region CDR3. In some embodiments, the CDR is a light chain variable region CDR1. In some embodiments, the CDR is a light chain variable region CDR2. In some embodiments, the CDR is a light chain variable region CDR3. In some embodiments, the substitutions are made as part of a humanization process. In some embodiments, the substitutions are made as part of a germline humanization process. In some embodiments, the substitutions are made as part of an affinity maturation process. In some embodiments, the substitutions are made as part of an optimization process.

CDRs of an antibody are defined using a variety of methods/systems by those skilled in the art. These systems and/or definitions have been developed and refined over a number of years and include Kabat, Chothia, IMGT, AbM, and Contact. The Kabat definition is based on sequence variability and is commonly used. The Chothia definition is based on the location of the structural loop regions. The IMGT system is based on sequence variability and location within the structure of the variable domain. The AbM definition is a compromise between Kabat and Chothia. The Contact definition is based on analyses of the available antibody crystal structures. An Exemplary system is a combination of Kabat and Chothia. Software programs (e.g., abYsis) are available and known to those of skill in the art for analysis of antibody sequences and determination of CDRs.

The specific CDR sequences defined herein are generally based on a combination of Kabat and Chothia definitions (Exemplary system). However, it will be understood that reference to a heavy chain variable region CDR or CDRs and/or a light chain variable region CDR or CDRs of a specific antibody will encompass all CDR definitions as known to those of skill in the art.

In some embodiments, an anti-HTRA1 antibody described herein comprises the six CDRs of antibody 24F7, hz24F7.v2, 9F8, 55B12, or 65G8 based on the Kabat definition. In some embodiments, an anti-HTRA1 antibody described herein comprises the six CDRs of antibody 24F7, hz24F7.v2, 9F8, 55B12, or 65G8 based on the Chothia definition. In some embodiments, an anti-HTRA1 antibody described herein comprises the six CDRs of antibody 24F7, hz24F7.v2, 9F8, 55B12, or 65G8 based on the AbM definition. In some embodiments, an anti-HTRA1 antibody described herein comprises the six CDRs of antibody 24F7, hz24F7.v2, 9F8, 55B12, or 65G8 based on the IMGT definition. In some embodiments, an anti-HTRA1 antibody described herein comprises the six CDRs of antibody 24F7, hz24F7.v2, 9F8, 55B12, or 65G8 based on the Contact definition. In some embodiments, an anti-HTRA1 antibody described herein comprises the six CDRs of antibody 24F7, hz24F7.v2, 9F8, 55B12, or 65G8 based on the Exemplary definition.

In some embodiments, a HTRA1-binding agent is an anti-HTRA1 antibody that comprises one, two, three, four, five, and/or six CDRs of any one of the antibodies described herein. In some embodiments, an anti-HTRA1 antibody comprises (i) a heavy chain variable region comprising one, two, and/or three heavy chain variable region CDRs from Table 1A, and/or (ii) a light chain variable region comprising one, two, and/or three light chain variable region CDRs from Table 1A. In some embodiments, an anti-HTRA1 antibody comprises (i) a heavy chain variable region comprising one, two, and/or three heavy chain variable region CDRs from Table 1B, and/or (ii) a light chain variable region comprising one, two, and/or three light chain variable region CDRs from Table 1B. In some embodiments, an anti-HTRA1 antibody comprises (i) a heavy chain variable region comprising one, two, and/or three heavy chain variable region CDRs from Table 1C, and/or (ii) a light chain variable region comprising one, two, and/or three light chain variable region CDRs from Table 1C. In some embodiments, an anti-HTRA1 antibody comprises (i) a heavy chain variable region comprising one, two, and/or three heavy chain variable region CDRs from Table 1D, and/or (ii) a light chain variable region comprising one, two, and/or three light chain variable region CDRs from Table 1D. In some embodiments, an anti-HTRA1 antibody comprises (i) a heavy chain variable region comprising one, two, and/or three heavy chain variable region CDRs from Table 1E, and/or (ii) a light chain variable region comprising one, two, and/or three light chain variable region CDRs from Table 1E. In some embodiments, an anti-HTRA1 antibody comprises (i) a heavy chain variable region comprising one, two, and/or three heavy chain variable region CDRs from Table 1F, and/or (ii) a light chain variable region comprising one, two, and/or three light chain variable region CDRs from Table 1F. In some embodiments, an anti-HTRA1 antibody comprises (i) a heavy chain variable region comprising one, two, and/or three heavy chain variable region CDRs from Table 1G, and/or (ii) a light chain variable region comprising one, two, and/or three light chain variable region CDRs from Table 1G. In some embodiments, an anti-HTRA1 antibody comprises (i) a heavy chain variable region comprising one, two, and/or three heavy chain variable region CDRs from Table 1H, and/or (ii) a light chain variable region comprising one, two, and/or three light chain variable region CDRs from Table 1H. In some embodiments, an anti-HTRA1 antibody comprises (i) a heavy chain variable region comprising one, two, and/or three heavy chain variable region CDRs from Table 1I, and/or (ii) a light chain variable region comprising one, two, and/or three light chain variable region CDRs from Table 1I. In some embodiments, an anti-HTRA1 antibody comprises (i) a heavy chain variable region comprising one, two, and/or three heavy chain variable region CDRs from Table 1J, and/or (ii) a light chain variable region comprising one, two, and/or three light chain variable region CDRs from Table 1J. In some embodiments, an anti-HTRA1 antibody comprises (i) a heavy chain variable region comprising one, two, and/or three heavy chain variable region CDRs from Table 2, and/or (ii) a light chain variable region comprising one, two, and/or three light chain variable region CDRs from Table 2. In some embodiments, an anti-HTRA1 antibody comprises (i) a heavy chain variable region comprising one, two, and/or three heavy chain variable region CDRs from Table 3, and/or (ii) a light chain variable region comprising one, two, and/or three light chain variable region CDRs from Table 3. In some embodiments, an anti-HTRA1 antibody comprises (i) a heavy chain variable region comprising one, two, and/or three heavy chain variable region CDRs from Table 4, and/or (ii) a light chain variable region comprising one, two, and/or three light chain variable region CDRs from Table 4. In some embodiments, an anti-HTRA1 antibody comprises (i) a heavy chain variable region comprising three heavy chain variable region CDRs from Table 1A, and (ii) a light chain variable region comprising three light chain variable region CDRs from Table 1A. In some embodiments, an anti-HTRA1 antibody comprises (i) a heavy chain variable region comprising three heavy chain variable region CDRs from Table 1B, and (ii) a light chain variable region comprising three light chain variable region CDRs from Table 1B. In some embodiments, an anti-HTRA1 antibody comprises (i) a heavy chain variable region comprising three heavy chain variable region CDRs from Table 1C, and (ii) a light chain variable region comprising three light chain variable region CDRs from Table 1C. In some embodiments, an anti-HTRA1 antibody comprises (i) a heavy chain variable region comprising three heavy chain variable region CDRs from Table 1D, and (ii) a light chain variable region comprising three light chain variable region CDRs from Table 1D. In some embodiments, an anti-HTRA1 antibody comprises (i) a heavy chain variable region comprising three heavy chain variable region CDRs from Table 1E, and (ii) a light chain variable region comprising three light chain variable region CDRs from Table 1E. In some embodiments, an anti-HTRA1 antibody comprises (i) a heavy chain variable region comprising three heavy chain variable region CDRs from Table 1F, and (ii) a light chain variable region comprising three light chain variable region CDRs from Table 1F. In some embodiments, an anti-HTRA1 antibody comprises (i) a heavy chain variable region comprising three heavy chain variable region CDRs from Table 1G, and (ii) a light chain variable region comprising three light chain variable region CDRs from Table 1G. In some embodiments, an anti-HTRA1 antibody comprises (i) a heavy chain variable region comprising three heavy chain variable region CDRs from Table 1H, and (ii) a light chain variable region comprising three light chain variable region CDRs from Table 1H. In some embodiments, an anti-HTRA1 antibody comprises (i) a heavy chain variable region comprising three heavy chain variable region CDRs from Table 1I, and (ii) a light chain variable region comprising three light chain variable region CDRs from Table 1I. In some embodiments, an anti-HTRA1 antibody comprises (i) a heavy chain variable region comprising three heavy chain variable region CDRs from Table 1J, and (ii) a light chain variable region comprising three light chain variable region CDRs from Table 1J. In some embodiments, an anti-HTRA1 antibody comprises (i) a heavy chain variable region comprising three heavy chain variable region CDRs from Table 2, and (ii) a light chain variable region comprising three light chain variable region CDRs from Table 2. In some embodiments, an anti-HTRA1 antibody comprises (i) a heavy chain variable region comprising three heavy chain variable region CDRs from Table 3, and (ii) a light chain variable region comprising three light chain variable region CDRs from Table 3. In some embodiments, an anti-HTRA1 antibody comprises (i) a heavy chain variable region comprising three heavy chain variable region CDRs from Table 4, and (ii) a light chain variable region comprising three light chain variable region CDRs from Table 4.

TABLE 1A
Antibody 24F7 Sequences

| | Exemplary | Chothia | AbM | Kabat | Contact |
|---|---|---|---|---|---|
| Heavy Chain | GYTFTDYEMH | GYTFTDY | GYTFTDYEMH | DYEMH | TDYEMH |
| variable | (SEQ ID NO: 9) | (SEQ ID NO: 15) | (SEQ ID NO: 9) | (SEQ ID NO: 18) | (SEQ ID NO: 19) |
| region CDR1 | | | | | |
| Heavy Chain | AIDPETGGTAYNQKFKG | DPETGG | AIDPETGGTA | AIDPETGGTAYNQKFKG | WIGAIDPETGGTA |
| variable | (SEQ ID NO: 10) | (SEQ ID NO: 16) | (SEQ ID NO: 17) | (SEQ ID NO: 10) | (SEQ ID NO: 20) |
| region CDR2 | | | | | |
| Heavy Chain | EGYSYDGGGYYFDY | EGYSYDGGGYYFDY | EGYSYDGGGYYFDY | EGYSYDGGGYYFDY | TREGYSYDGGGYYFD |
| variable | (SEQ ID NO: 11) | (SEQ ID NO: 11) | (SEQ ID NO: 11) | (SEQ ID NO: 11) | (SEQ ID NO: 21) |
| region CDR3 | | | | | |
| Light Chain | SVSSSVSYMY | SVSSSVSYMY | SVSSSVSYMY | SVSSSVSYMY | SYMYWY |
| variable | (SEQ ID NO: 12) | (SEQ ID NO: 12) | (SEQ ID NO: 12) | (SEQ ID NO: 12) | (SEQ ID NO: 22) |
| region CDR1 | | | | | |
| Light Chain | DTSNLAS | DTSNLAS | DTSNLAS | DTSNLAS | LLIYDTSNLA |
| variable | (SEQ ID NO: 13) | (SEQ ID NO: 13) | (SEQ ID NO: 13) | (SEQ ID NO: 13) | (SEQ ID NO: 23) |
| region CDR2 | | | | | |
| Light Chain | QQWSSYPT | QQWSSYPT | QQWSSYPT | QQWSSYPT | QQWSSYP |
| variable | (SEQ ID NO: 14) | (SEQ ID NO: 14) | (SEQ ID NO: 14) | (SEQ ID NO: 14) | (SEQ ID NO: 24) |
| region CDR3 | | | | | |

24F7 Heavy chain variable region (SEQ ID NO: 68)
QVQLQQSGAELVRPGASVKLSCKASGYTFTDYEMHWVKQTPVHGLEWIGAIDPETGGTAYNQKFKGKATL
TADKSSSTAYMELRSLTSEDSAVYYCTREGYSYDGGGYYFDYWGQGTTLTVSS
24F7 Light chain variable region (SEQ ID NO: 69)
QIVLTQSPAIMSASPGEKVTMTCSVSSSVSYMYWYQQKPGSSPRLLIYDTSNLASGVPVRFSGSGSGTSY
SLTISRMEAEDAATYYCQQWSSYPTFGGGTKLEIK

TABLE 1B
Antibody hz24F7.v2 Sequences

| | Exemplary | Chothia | AbM | Kabat | Contact |
|---|---|---|---|---|---|
| Heavy Chain | GYTFTDYEMH | GYTFTDY | GYTFTDYEMH | DYEMH | TDYEMH |
| variable | (SEQ ID NO: 9) | (SEQ ID NO: 15) | (SEQ ID NO: 9) | (SEQ ID NO: 18) | (SEQ ID NO: 19) |
| region CDR1 | | | | | |
| Heavy Chain | AIDPETGGTAYNQKFKG | DPETGG | AIDPETGGTA | AIDPETGGTAYNQKFKG | WIGAIDPETGGTA |
| variable | (SEQ ID NO: 10) | (SEQ ID NO: 16) | (SEQ ID NO: 17) | (SEQ ID NO: 10) | (SEQ ID NO: 20) |
| region CDR2 | | | | | |
| Heavy Chain | EGYSYEGGGYYFDY | EGYSYEGGGYYFDY | EGYSYEGGGYYFDY | EGYSYEGGGYYFDY | TREGYSYEGGGYYFD |
| variable | (SEQ ID NO: 25) | (SEQ ID NO: 25) | (SEQ ID NO: 25) | (SEQ ID NO: 25) | (SEQ ID NO: 26) |
| region CDR3 | | | | | |
| Light Chain | SVSSSVSYMY | SVSSSVSYMY | SVSSSVSYMY | SVSSSVSYMY | SYMYWY |
| variable | (SEQ ID NO: 12) | (SEQ ID NO: 12) | (SEQ ID NO: 12) | (SEQ ID NO: 12) | (SEQ ID NO: 22) |
| region CDR1 | | | | | |
| Light Chain | DTSNLAS | DTSNLAS | DTSNLAS | DTSNLAS | LLIYDTSNLA |
| variable | (SEQ ID NO: 13) | (SEQ ID NO: 13) | (SEQ ID NO: 13) | (SEQ ID NO: 13) | (SEQ ID NO: 23) |
| region CDR2 | | | | | |
| Light Chain | QQWSSYPT | QQWSSYPT | QQWSSYPT | QQWSSYPT | QQWSSYP |
| variable | (SEQ ID NO: 14) | (SEQ ID NO: 14) | (SEQ ID NO: 14) | (SEQ ID NO: 14) | (SEQ ID NO: 24) |
| region CDR3 | | | | | |

hz24F7.v2 Heavy chain variable region (SEQ ID NO: 71)
QVQLVQSGAEVKKPGASVKVSCKASGYTFTDYEMHWVRQAPGQRLEWMGAIDPETGGTAYNQKFKGRVTI
TADKSASTAYMELSSLRSEDTAVYYCTREGYSYEGGGYYFDYWGQGTLVTVSS
hz24F7.v2 Light chain variable region (SEQ ID NO: 72)
DIQMTQSPSSLSASVGDRVTITCSVSSSVSYMYWYQQKPGKAPKWYDTSNLASGVPSRFSGSGSGTDF
TFTISSLQPEDIATYYCQQWSSYPTFGQGTKLEIK

TABLE 1C
Antibody hz24F7.v2 S91Y Sequences

| | Exemplary | Chothia | AbM | Kabat | Contact |
|---|---|---|---|---|---|
| Heavy Chain | GYTFTDYEMH | GYTFTDY | GYTFTDYEMH | DYEMH | TDYEMH |
| variable | (SEQ ID NO: 9) | (SEQ ID NO: 15) | (SEQ ID NO: 9) | (SEQ ID NO: 18) | (SEQ ID NO: 19) |
| region CDR1 | | | | | |
| Heavy Chain | AIDPETGGTAYNQKFKG | DPETGG | AIDPETGGTA | AIDPETGGTAYNQKFKG | WIGAIDPETGGTA |
| variable | (SEQ ID NO: 10) | (SEQ ID NO: 16) | (SEQ ID NO: 17) | (SEQ ID NO: 10) | (SEQ ID NO: 20) |
| region CDR2 | | | | | |
| Heavy Chain | EGYSYEGGGYYFDY | EGYSYEGGGYYFDY | EGYSYEGGGYYFDY | EGYSYEGGGYYFDY | TREGYSYEGGGYYFD |
| variable | (SEQ ID NO: 25) | (SEQ ID NO: 25) | (SEQ ID NO: 25) | (SEQ ID NO: 25) | (SEQ ID NO: 26) |
| region CDR3 | | | | | |
| Light Chain | SVSSSVSYMY | SVSSSVSYMY | SVSSSVSYMY | SVSSSVSYMY | SYMYWY |
| variable | (SEQ ID NO: 12) | (SEQ ID NO: 12) | (SEQ ID NO: 12) | (SEQ ID NO: 12) | (SEQ ID NO: 22) |
| region CDR1 | | | | | |
| Light Chain | DTSNLAS | DTSNLAS | DTSNLAS | DTSNLAS | LLIYDTSNLA |
| variable | (SEQ ID NO: 13) | (SEQ ID NO: 13) | (SEQ ID NO: 13) | (SEQ ID NO: 13) | (SEQ ID NO: 23) |
| region CDR2 | | | | | |
| Light Chain | QQWYSYPT | QQWYSYPT | QQWYSYPT | QQWYSYPT | QQWYSYP |
| variable | (SEQ ID NO: 94) | (SEQ ID NO: 94) | (SEQ ID NO: 94) | (SEQ ID NO: 94) | (SEQ ID NO: 95) |
| region CDR3 | | | | | |

hz24F7.v2 Heavy chain variable region (SEQ ID NO: 71)
QVQLVQSGAEVKKPGASVKVSCKASGYTFTDYEMHWVRQAPGQRLEWMGAIDPETGGTAYNQKFKGRVTI
TADKSASTAYMELSSLRSEDTAVYYCTREGYSYEGGGYYFDYWGQGTLVTVSS
hz24F7.v2 Light chain variable region (SEQ ID NO: 96)
DIQMTQSPSSLSASVGDRVTITCSVSSSVSYMYWYQQKPGKAPKLLIYDTSNLASGVPSRFSGSGSGTDF
TFTISSLQPEDIATYYCQQWYSYPTFGQGTKLEIK

TABLE 1D
Antibody hz24F7.v2 S92T Sequences

| | Exemplary | Chothia | AbM | Kabat | Contact |
|---|---|---|---|---|---|
| Heavy Chain | GYTFTDYEMH | GYTFTDY | GYTFTDYEMH | DYEMH | TDYEMH |
| variable | (SEQ ID NO: 9) | (SEQ ID NO: 15) | (SEQ ID NO: 9) | (SEQ ID NO: 18) | (SEQ ID NO: 19) |
| region CDR1 | | | | | |
| Heavy Chain | AIDPETGGTAYNQKFKG | DPETGG | AIDPETGGTA | AIDPETGGTAYNQKFKG | WIGAIDPETGGTA |
| variable | (SEQ ID NO: 10) | (SEQ ID NO: 16) | (SEQ ID NO: 17) | (SEQ ID NO: 10) | (SEQ ID NO: 20) |
| region CDR2 | | | | | |
| Heavy Chain | EGYSYEGGGYYFDY | EGYSYEGGGYYFDY | EGYSYEGGGYYFDY | EGYSYEGGGYYFDY | TREGYSYEGGGYYFD |
| variable | (SEQ ID NO: 25) | (SEQ ID NO: 25) | (SEQ ID NO: 25) | (SEQ ID NO: 25) | (SEQ ID NO: 26) |
| region CDR3 | | | | | |
| Light Chain | SVSSSVSYMY | SVSSSVSYMY | SVSSSVSYMY | SVSSSVSYMY | SYMYWY |
| variable | (SEQ ID NO: 12) | (SEQ ID NO: 12) | (SEQ ID NO: 12) | (SEQ ID NO: 12) | (SEQ ID NO: 22) |
| region CDR1 | | | | | |
| Light Chain | DTSNLAS | DTSNLAS | DTSNLAS | DTSNLAS | LLIYDTSNLA |
| variable | (SEQ ID NO: 13) | (SEQ ID NO: 13) | (SEQ ID NO: 13) | (SEQ ID NO: 13) | (SEQ ID NO: 23) |
| region CDR2 | | | | | |
| Light Chain | QQWSTYPT | QQWSTYPT | QQWSTYPT | QQWSTYPT | QQWSTYP |
| variable | (SEQ ID NO: 99) | (SEQ ID NO: 99) | (SEQ ID NO: 99) | (SEQ ID NO: 99) | (SEQ ID NO: 100) |
| region CDR3 | | | | | |

hz24F7.v2 Heavy chain variable region (SEQ ID NO: 71)
QVQLVQSGAEVKKPGASVKVSCKASGYTFTDYEMHWVRQAPGQRLEWMGAIDPETGGTAYNQKFKGRVTI
TADKSASTAYMELSSLRSEDTAVYYCTREGYSYEGGGYYFDYWGQGTLVTVSS
hz24F7.v2 Light chain variable region (SEQ ID NO: 101)
DIQMTQSPSSLSASVGDRVTITCSVSSSVSYMYWYQQKPGKAPKWYDTSNLASGVPSRFSGSGSGTDF
TFTISSLQPEDIATYYCQQWSTYPTFGQGTKLEIK

TABLE 1E
Antibody hz24F7.v2 S91D Sequences

| | Exemplary | Chothia | AbM | Kabat | Contact |
|---|---|---|---|---|---|
| Heavy Chain | GYTFTDYEMH | GYTFTDY | GYTFTDYEMH | DYEMH | TDYEMH |
| variable | (SEQ ID NO: 9) | (SEQ ID NO: 15) | (SEQ ID NO: 9) | (SEQ ID NO: 18) | (SEQ ID NO: 19) |
| region CDR1 | | | | | |
| Heavy Chain | AIDPETGGTAYNQKFKG | DPETGG | AIDPETGGTA | AIDPETGGTAYNQKFKG | WIGAIDPETGGTA |
| variable | (SEQ ID NO: 10) | (SEQ ID NO: 16) | (SEQ ID NO: 17) | (SEQ ID NO: 10) | (SEQ ID NO: 20) |
| region CDR2 | | | | | |
| Heavy Chain | EGYSYEGGGYYFDY | EGYSYEGGGYYFDY | EGYSYEGGGYYFDY | EGYSYEGGGYYFDY | TREGYSYEGGGYYFD |
| variable | (SEQ ID NO: 25) | (SEQ ID NO: 25) | (SEQ ID NO: 25) | (SEQ ID NO: 25) | (SEQ ID NO: 26) |
| region CDR3 | | | | | |
| Light Chain | SVSSSVSYMY | SVSSSVSYMY | SVSSSVSYMY | SVSSSVSYMY | SYMYWY |
| variable | (SEQ ID NO: 12) | (SEQ ID NO: 12) | (SEQ ID NO: 12) | (SEQ ID NO: 12) | (SEQ ID NO: 22) |
| region CDR1 | | | | | |
| Light Chain | DTSNLAS | DTSNLAS | DTSNLAS | DTSNLAS | LLIYDTSNLA |
| variable | (SEQ ID NO: 13) | (SEQ ID NO: 13) | (SEQ ID NO: 13) | (SEQ ID NO: 13) | (SEQ ID NO: 23) |
| region CDR2 | | | | | |
| Light Chain | QQWDSYPT | QQWDSYPT | QQWDSYPT | QQWDSYPT | QQWDSYP |
| variable | (SEQ ID NO: 104) | (SEQ ID NO: 104) | (SEQ ID NO: 104) | (SEQ ID NO: 104) | (SEQ ID NO: 105) |
| region CDR3 | | | | | |

hz24F7.v2 Heavy chain variable region (SEQ ID NO: 71)
QVQLVQSGAEVKKPGASVKVSCKASGYTFTDYEMHWVRQAPGQRLEWMGAIDPETGGTAYNQKFKGRVTI
TADKSASTAYMELSSLRSEDTAVYYCTREGYSYEGGGYYFDYWGQGTLVTVSS
hz24F7.v2 Light chain variable region (SEQ ID NO: 106)
DIQMTQSPSSLSASVGDRVTITCSVSSSVSYMYWYQQKPGKAPKWYDTSNLASGVPSRFSGSGSGTDF
TFTISSLQPEDIATYYCQQWDSYPTFGQGTKLEIK

TABLE 1F
Antibody hz24F7.v2 S91T Sequences

| | Exemplary | Chothia | AbM | Kabat | Contact |
|---|---|---|---|---|---|
| Heavy Chain | GYTFTDYEMH | GYTFTDY | GYTFTDYEMH | DYEMH | TDYEMH |
| variable | (SEQ ID NO: 9) | (SEQ ID NO: 15) | (SEQ ID NO: 9) | (SEQ ID NO: 18) | (SEQ ID NO: 19) |
| region CDR1 | | | | | |
| Heavy Chain | AIDPETGGTAYNQKFKG | DPETGG | AIDPETGGTA | AIDPETGGTAYNQKFKG | WIGAIDPETGGTA |
| variable | (SEQ ID NO: 10) | (SEQ ID NO: 16) | (SEQ ID NO: 17) | (SEQ ID NO: 10) | (SEQ ID NO: 20) |
| region CDR2 | | | | | |
| Heavy Chain | EGYSYEGGGYYFDY | EGYSYEGGGYYFDY | EGYSYEGGGYYFDY | EGYSYEGGGYYFDY | TREGYSYEGGGYYFD |
| variable | (SEQ ID NO: 25) | (SEQ ID NO: 25) | (SEQ ID NO: 25) | (SEQ ID NO: 25) | (SEQ ID NO: 26) |
| region CDR3 | | | | | |
| Light Chain | SVSSSVSYMY | SVSSSVSYMY | SVSSSVSYMY | SVSSSVSYMY | SYMYWY |
| variable | (SEQ ID NO: 12) | (SEQ ID NO: 12) | (SEQ ID NO: 12) | (SEQ ID NO: 12) | (SEQ ID NO: 22) |
| region CDR1 | | | | | |
| Light Chain | DTSNLAS | DTSNLAS | DTSNLAS | DTSNLAS | LLIYDTSNLA |
| variable | (SEQ ID NO: 13) | (SEQ ID NO: 13) | (SEQ ID NO: 13) | (SEQ ID NO: 13) | (SEQ ID NO: 23) |
| region CDR2 | | | | | |
| Light Chain | QQWTSYPT | QQWTSYPT | QQWTSYPT | QQWTSYPT | QQWTSYP |
| variable | (SEQ ID NO: 107) | (SEQ ID NO: 107) | (SEQ ID NO: 107) | (SEQ ID NO: 107) | (SEQ ID NO: 108) |
| region CDR3 | | | | | |

hz24F7.v2 Heavy chain variable region (SEQ ID NO: 71)
QVQLVQSGAEVKKPGASVKVSCKASGYTFTDYEMHWVRQAPGQRLEWMGAIDPETGGTAYNQKFKGRVTI
TADKSASTAYMELSSLRSEDTAVYYCTREGYSYEGGGYYFDYWGQGTLVTVSS
hz24F7.v2 Light chain variable region (SEQ ID NO: 109)
DIQMTQSPSSLSASVGDRVTITCSVSSSVSYMYWYQQKPGKAPKWYDTSNLASGVPSRFSGSGSGTDF
TFTISSLQPEDIATYYCQQWTSYPTFGQGTKLEIK

TABLE 1G
Antibody hz24F7.v2 S91A Sequences

| | Exemplary | Chothia | AbM | Kabat | Contact |
|---|---|---|---|---|---|
| Heavy Chain | GYTFTDYEMH | GYTFTDY | GYTFTDYEMH | DYEMH | TDYEMH |
| variable | (SEQ ID NO: 9) | (SEQ ID NO: 15) | (SEQ ID NO: 9) | (SEQ ID NO: 18) | (SEQ ID NO: 19) |
| region CDR1 | | | | | |
| Heavy Chain | AIDPETGGTAYNQKFKG | DPETGG | AIDPETGGTA | AIDPETGGTAYNQKFKG | WIGAIDPETGGTA |
| variable | (SEQ ID NO: 10) | (SEQ ID NO: 16) | (SEQ ID NO: 17) | (SEQ ID NO: 10) | (SEQ ID NO: 20) |
| region CDR2 | | | | | |
| Heavy Chain | EGYSYEGGGYYFDY | EGYSYEGGGYYFDY | EGYSYEGGGYYFDY | EGYSYEGGGYYFDY | TREGYSYEGGGYYFD |
| variable | (SEQ ID NO: 25) | (SEQ ID NO: 25) | (SEQ ID NO: 25) | (SEQ ID NO: 25) | (SEQ ID NO: 26) |
| region CDR3 | | | | | |
| Light Chain | SVSSSVSYMY | SVSSSVSYMY | SVSSSVSYMY | SVSSSVSYMY | SYMYWY |
| variable | (SEQ ID NO: 12) | (SEQ ID NO: 12) | (SEQ ID NO: 12) | (SEQ ID NO: 12) | (SEQ ID NO: 22) |
| region CDR1 | | | | | |
| Light Chain | DTSNLAS | DTSNLAS | DTSNLAS | DTSNLAS | LLIYDTSNLA |
| variable | (SEQ ID NO: 13) | (SEQ ID NO: 13) | (SEQ ID NO: 13) | (SEQ ID NO: 13) | (SEQ ID NO: 23) |
| region CDR2 | | | | | |
| Light Chain | QQWASYPT | QQWASYPT | QQWASYPT | QQWASYPT | QQWASYP |
| variable | (SEQ ID NO: 110) | (SEQ ID NO: 110) | (SEQ ID NO: 110) | (SEQ ID NO: 110) | (SEQ ID NO: 111) |
| region CDR3 | | | | | |

hz24F7.v2 Heavy chain variable region (SEQ ID NO: 71)
QVQLVQSGAEVKKPGASVKVSCKASGYTFTDYEMHWVRQAPGQRLEWMGAIDPETGGTAYNQKFKGRVTI
TADKSASTAYMELSSLRSEDTAVYYCTREGYSYEGGGYYFDYWGQGTLVTVSS
hz24F7.v2 Light chain variable region (SEQ ID NO: 112)
DIQMTQSPSSLSASVGDRVTITCSVSSSVSYMYWYQQKPGKAPKWYDTSNLASGVPSRFSGSGSGTDF
TFTISSLQPEDIATYYCQQWASYPTFGQGTKLEIK

TABLE 1H
Antibody hz24F7.v2 S91L Sequences

| | Exemplary | Chothia | AbM | Kabat | Contact |
|---|---|---|---|---|---|
| Heavy Chain | GYTFTDYEMH | GYTFTDY | GYTFTDYEMH | DYEMH | TDYEMH |
| variable | (SEQ ID NO: 9) | (SEQ ID NO: 15) | (SEQ ID NO: 9) | (SEQ ID NO: 18) | (SEQ ID NO: 19) |
| region CDR1 | | | | | |
| Heavy Chain | AIDPETGGTAYNQKFKG | DPETGG | AIDPETGGTA | AIDPETGGTAYNQKFKG | WIGAIDPETGGTA |
| variable | (SEQ ID NO: 10) | (SEQ ID NO: 16) | (SEQ ID NO: 17) | (SEQ ID NO: 10) | (SEQ ID NO: 20) |
| region CDR2 | | | | | |
| Heavy Chain | EGYSYEGGGYYFDY | EGYSYEGGGYYFDY | EGYSYEGGGYYFDY | EGYSYEGGGYYFDY | TREGYSYEGGGYYFD |
| variable | (SEQ ID NO: 25) | (SEQ ID NO: 25) | (SEQ ID NO: 25) | (SEQ ID NO: 25) | (SEQ ID NO: 26) |
| region CDR3 | | | | | |
| Light Chain | SVSSSVSYMY | SVSSSVSYMY | SVSSSVSYMY | SVSSSVSYMY | SYMYWY |
| variable | (SEQ ID NO: 12) | (SEQ ID NO: 12) | (SEQ ID NO: 12) | (SEQ ID NO: 12) | (SEQ ID NO: 22) |
| region CDR1 | | | | | |
| Light Chain | DTSNLAS | DTSNLAS | DTSNLAS | DTSNLAS | LLIYDTSNLA |
| variable | (SEQ ID NO: 13) | (SEQ ID NO: 13) | (SEQ ID NO: 13) | (SEQ ID NO: 13) | (SEQ ID NO: 23) |
| region CDR2 | | | | | |
| Light Chain | QQWLSYPT | QQWLSYPT | QQWLSYPT | QQWLSYPT | QQWLSYP |
| variable | (SEQ ID NO: 113) | (SEQ ID NO: 113) | (SEQ ID NO: 113) | (SEQ ID NO: 113) | (SEQ ID NO: 114) |
| region CDR3 | | | | | |

hz24F7.v2 Heavy chain variable region (SEQ ID NO: 71)
QVQLVQSGAEVKKPGASVKVSCKASGYTFTDYEMHWVRQAPGQRLEWMGAIDPETGGTAYNQKFKGRVTI
TADKSASTAYMELSSLRSEDTAVYYCTREGYSYEGGGYYFDYWGQGTLVTVSS
hz24F7.v2 Light chain variable region (SEQ ID NO: 115)
DIQMTQSPSSLSASVGDRVTITCSVSSSVSYMYWYQQKPGKAPKWYDTSNLASGVPSRFSGSGSGTDF
TFTISSLQPEDIATYYCQQWSSYPTFGQGTKLEIK

TABLE 1I
Antibody hz24F7.v2 S92Y Sequences

| | Exemplary | Chothia | AbM | Kabat | Contact |
|---|---|---|---|---|---|
| Heavy Chain | GYTFTDYEMH | GYTFTDY | GYTFTDYEMH | DYEMH | TDYEMH |
| variable | (SEQ ID NO: 9) | (SEQ ID NO: 15) | (SEQ ID NO: 9) | (SEQ ID NO: 18) | (SEQ ID NO: 19) |
| region CDR1 | | | | | |
| Heavy Chain | AIDPETGGTAYNQKFKG | DPETGG | AIDPETGGTA | AIDPETGGTAYNQKFKG | WIGAIDPETGGTA |
| variable | (SEQ ID NO: 10) | (SEQ ID NO: 16) | (SEQ ID NO: 17) | (SEQ ID NO: 10) | (SEQ ID NO: 20) |
| region CDR2 | | | | | |
| Heavy Chain | EGYSYEGGGYYFDY | EGYSYEGGGYYFDY | EGYSYEGGGYYFDY | EGYSYEGGGYYFDY | TREGYSYEGGGYYFD |
| variable | (SEQ ID NO: 25) | (SEQ ID NO: 25) | (SEQ ID NO: 25) | (SEQ ID NO: 25) | (SEQ ID NO: 26) |
| region CDR3 | | | | | |
| Light Chain | SVSSSVSYMY | SVSSSVSYMY | SVSSSVSYMY | SVSSSVSYMY | SYMYWY |
| variable | (SEQ ID NO: 12) | (SEQ ID NO: 12) | (SEQ ID NO: 12) | (SEQ ID NO: 12) | (SEQ ID NO: 22) |
| region CDR1 | | | | | |
| Light Chain | DTSNLAS | DTSNLAS | DTSNLAS | DTSNLAS | LLIYDTSNLA |
| variable | (SEQ ID NO: 13) | (SEQ ID NO: 13) | (SEQ ID NO: 13) | (SEQ ID NO: 13) | (SEQ ID NO: 23) |
| region CDR2 | | | | | |
| Light Chain | QQWSYYPT | QQWSYYPT | QQWSYYPT | QQWSYYPT | QQWSYYP |
| variable | (SEQ ID NO: 116) | (SEQ ID NO: 116) | (SEQ ID NO: 116) | (SEQ ID NO: 116) | (SEQ ID NO: 117) |
| region CDR3 | | | | | |

hz24F7.v2 Heavy chain variable region (SEQ ID NO: 71)
QVQLVQSGAEVKKPGASVKVSCKASGYTFTDYEMHWVRQAPGQRLEWMGAIDPETGGTAYNQKFKGRVTI
TADKSASTAYMELSSLRSEDTAVYYCTREGYSYEGGGYYFDYWGQGTLVTVSS
hz24F7.v2 Light chain variable region (SEQ ID NO: 118)
DIQMTQSPSSLSASVGDRVTITCSVSSSVSYMYWYQQKPGKAPKWYDTSNLASGVPSRFSGSGSGTDF
TFTISSLQPEDIATYYCQQWSYYPTFGQGTKLEIK

TABLE 1J
Antibody hz24F7.v2 S92D Sequences

| | Exemplary | Chothia | AbM | Kabat | Contact |
|---|---|---|---|---|---|
| Heavy Chain | GYTFTDYEMH | GYTFTDY | GYTFTDYEMH | DYEMH | TDYEMH |
| variable | (SEQ ID NO: 9) | (SEQ ID NO: 15) | (SEQ ID NO: 9) | (SEQ ID NO: 18) | (SEQ ID NO: 19) |
| region CDR1 | | | | | |
| Heavy Chain | AIDPETGGTAYNQKFKG | DPETGG | AIDPETGGTA | AIDPETGGTAYNQKFKG | WIGAIDPETGGTA |
| variable | (SEQ ID NO: 10) | (SEQ ID NO: 16) | (SEQ ID NO: 17) | (SEQ ID NO: 10) | (SEQ ID NO: 20) |
| region CDR2 | | | | | |
| Heavy Chain | EGYSYEGGGYYFDY | EGYSYEGGGYYFDY | EGYSYEGGGYYFDY | EGYSYEGGGYYFDY | TREGYSYEGGGYYFD |
| variable | (SEQ ID NO: 25) | (SEQ ID NO: 25) | (SEQ ID NO: 25) | (SEQ ID NO: 25) | (SEQ ID NO: 26) |
| region CDR3 | | | | | |
| Light Chain | SVSSSVSYMY | SVSSSVSYMY | SVSSSVSYMY | SVSSSVSYMY | SYMYWY |
| variable | (SEQ ID NO: 12) | (SEQ ID NO: 12) | (SEQ ID NO: 12) | (SEQ ID NO: 12) | (SEQ ID NO: 22) |
| region CDR1 | | | | | |
| Light Chain | DTSNLAS | DTSNLAS | DTSNLAS | DTSNLAS | LLIYDTSNLA |
| variable | (SEQ ID NO: 13) | (SEQ ID NO: 13) | (SEQ ID NO: 13) | (SEQ ID NO: 13) | (SEQ ID NO: 23) |
| region CDR2 | | | | | |
| Light Chain | QQWSDYPT | QQWSDYPT | QQWSDYPT | QQWSDYPT | QQWSDYP |
| variable | (SEQ ID NO: 119) | (SEQ ID NO: 119) | (SEQ ID NO: 119) | (SEQ ID NO: 119) | (SEQ ID NO: 120) |
| region CDR3 | | | | | |

hz24F7.v2 Heavy chain variable region (SEQ ID NO: 71)
QVQLVQSGAEVKKPGASVKVSCKASGYTFTDYEMHWVRQAPGQRLEWMGAIDPETGGTAYNQKFKGRVTI
TADKSASTAYMELSSLRSEDTAVYYCTREGYSYEGGGYYFDYWGQGTLVTVSS
hz24F7.v2 Light chain variable region (SEQ ID NO: 121)
DIQMTQSPSSLSASVGDRVTITCSVSSSVSYMYWYQQKPGKAPKWYDTSNLASGVPSRFSGSGSGTDF
TFTISSLQPEDIATYYCQQWSDYPTFGQGTKLEIK

TABLE 2
Antibody 9F8 Sequences

| | Exemplary | Chothia | AbM | Kabat | Contact |
|---|---|---|---|---|---|
| Heavy Chain | GYAFTTYWMH | GYAFTTY | GYAFTTYWMH | TYWMH | TTYWMH |
| variable | (SEQ ID NO: 27) | (SEQ ID NO: 33) | (SEQ ID NO: 27) | (SEQ ID NO: 36) | (SEQ ID NO: 37) |
| region CDR1 | | | | | |
| Heavy Chain | NIDPSDSETHYNQKFRD | DPSDSE | NIDPSDSETH | NIDPSDSETHYNQKFRD | WIGNIDPSDSETH |
| variable | (SEQ ID NO: 28) | (SEQ ID NO: 34) | (SEQ ID NO: 35) | (SEQ ID NO: 28) | (SEQ ID NO: 38) |
| region CDR2 | | | | | |
| Heavy Chain | DYGAFDV | DYGAFDV | DYGAFDV | DYGAFDV | ARDYGAFD |
| variable | (SEQ ID NO: 29) | (SEQ ID NO: 29) | (SEQ ID NO: 29) | (SEQ ID NO: 29) | (SEQ ID NO: 39) |
| region CDR3 | | | | | |
| Light Chain | RSSTGAVTTRNFAS | RSSTGAVTTRNFAS | RSSTGAVTTRNFAS | RSSTGAVTTRNFAS | VTTRNFASWV |
| variable | (SEQ ID NO: 30) | (SEQ ID NO: 30) | (SEQ ID NO: 30) | (SEQ ID NO: 30) | (SEQ ID NO: 40) |
| region CDR1 | | | | | |
| Light Chain | GTNNRAP | GTNNRAP | GTNNRAP | GTNNRAP | GLIGGTNNRA |
| variable | (SEQ ID NO: 31) | (SEQ ID NO: 31) | (SEQ ID NO: 31) | (SEQ ID NO: 31) | (SEQ ID NO: 41) |
| region CDR2 | | | | | |
| Light Chain | ALWYSNLWV | ALWYSNLWV | ALWYSNLWV | ALWYSNLWV | ALWYSNLW |
| variable | (SEQ ID NO: 32) | (SEQ ID NO: 32) | (SEQ ID NO: 32) | (SEQ ID NO: 32) | (SEQ ID NO: 42) |
| region CDR3 | | | | | |

9F8 Heavy chain variable region (SEQ ID NO: 73)
QVQLQQPGAELVRPGSSVKLSCKASGYAFTTYWMHWVKQRPIQGLEWIGNIDPSDSETHYNQKFRDKATL
TVDKSSSTAYMQLSSLTSEDSAVYYCARDYGAFDVWGTGTTVTVSS
9F8 Light chain variable region (SEQ ID NO: 74)
QAVVTQESALTTSSGETVTLTCRSSTGAVTTRNFASWVQEKPDHLFTGLIGGTNNRAPGVPARFSGSLIG
DKAALTITGAQTEDEAIYFCALWYSNLWVFGGGTKLTVL

TABLE 3
Antibody 55B12 Sequences

| | Exemplary | Chothia | AbM | Kabat | Contact |
|---|---|---|---|---|---|
| Heavy Chain | GYTFTNYWMH | GYTFTNY | GYTFTNYWMH | NYWMH | TNYWMH |
| variable | (SEQ ID NO: 43) | (SEQ ID NO: 49) | (SEQ ID NO: 43) | (SEQ ID NO: 50) | (SEQ ID NO: 51) |
| region CDR1 | | | | | |
| Heavy Chain | NIDPSDSETHYNQKFKD | DPSDSE | NIDPSDSETH | NIDPSDSETHYNQKFKD | WIGNIDPSDSETH |
| variable | (SEQ ID NO: 44) | (SEQ ID NO: 34) | (SEQ ID NO: 35) | (SEQ ID NO: 44) | (SEQ ID NO: 38) |
| region CDR2 | | | | | |
| Heavy Chain | EDSSGYGAY | EDSSGYGAY | EDSSGYGAY | EDSSGYGAY | AREDSSGYGA |
| variable | (SEQ ID NO: 45) | (SEQ ID NO: 45) | (SEQ ID NO: 45) | (SEQ ID NO: 45) | (SEQ ID NO: 52) |
| region CDR3 | | | | | |
| Light Chain | SASSSVNYMH | SASSSVNYMH | SASSSVNYMH | SASSSVNYMH | NYMHWY |
| variable | (SEQ ID NO: 46) | (SEQ ID NO: 46) | (SEQ ID NO: 46) | (SEQ ID NO: 46) | (SEQ ID NO: 53) |
| region CDR1 | | | | | |
| Light Chain | DTSKLAS | DTSKLAS | DTSKLAS | DTSKLAS | RWIYDTSKLA |
| variable | (SEQ ID NO: 47) | (SEQ ID NO: 47) | (SEQ ID NO: 47) | (SEQ ID NO: 47) | (SEQ ID NO: 54) |
| region CDR2 | | | | | |
| Light Chain | QQWSSHPLT | QQWSSHPLT | QQWSSHPLT | QQWSSHPLT | QQWSSHPL |
| variable | (SEQ ID NO: 48) | (SEQ ID NO: 48) | (SEQ ID NO: 48) | (SEQ ID NO: 48) | (SEQ ID NO: 55) |
| region CDR3 | | | | | |

55B12 Heavy chain variable region (SEQ ID NO: 75)
QVQLQQPGAELVKPGASVKLSCKASGYTFTNYWMHWVKQRPGQGLEWIGNIDPSDSETHYNQKFKDKATL
AVDKSSSTAYMQLSSLTSEDSAVYYCAREDSSGYGAYWGQGTLVTVSA
55B12 Light chain variable region (SEQ ID NO: 76)
QIVLTQSPAIMSASPGEKVTMTCSASSSVNYMHWYQQKSGTSPKRWIYDTSKLASGVPDRFSGSGSGTSY
SLTISSMEAEDAATYYCQQWSSHPLTFGAGTKLELK

TABLE 4
Antibody 65G8 Sequences

| | Exemplary | Chothia | AbM | Kabat | Contact |
|---|---|---|---|---|---|
| Heavy Chain | GYSFTSYWMH | GYSFTSY | GYSFTSYWMH | SYWMH | TSYWMH |
| variable | (SEQ ID NO: 56) | (SEQ ID NO: 61) | (SEQ ID NO: 56) | (SEQ ID NO: 63) | (SEQ ID NO: 64) |
| region CDR1 | | | | | |
| Heavy Chain | MIDPSDSETRLNQKFKD | DPSDSE | MIDPSDSETR | MIDPSDSETRLNQKFKD | WIGMIDPSDSETR |
| variable | (SEQ ID NO: 57) | (SEQ ID NO: 34) | (SEQ ID NO: 62) | (SEQ ID NO: 57) | (SEQ ID NO: 65) |
| region CDR2 | | | | | |
| Heavy Chain | DYFDY | DYFDY | DYFDY | DYFDY | TRDYFD |
| variable | (SEQ ID NO: 58) | (SEQ ID NO: 58) | (SEQ ID NO: 58) | (SEQ ID NO: 58) | (SEQ ID NO: 66) |
| region CDR3 | | | | | |
| Light Chain | SASSSVSYMY | SASSSVSYMY | SASSSVSYMY | SASSSVSYMY | SYMYWY |
| variable | (SEQ ID NO: 59) | (SEQ ID NO: 59) | (SEQ ID NO: 59) | (SEQ ID NO: 59) | (SEQ ID NO: 22) |
| region CDR1 | | | | | |
| Light Chain | DTSNLAS | DTSNLAS | DTSNLAS | DTSNLAS | LLIYDTSNLA |
| variable | (SEQ ID NO: 13) | (SEQ ID NO: 13) | (SEQ ID NO: 13) | (SEQ ID NO: 13) | (SEQ ID NO: 23) |
| region CDR2 | | | | | |
| Light Chain | QQWSSYPYT | QQWSSYPYT | QQWSSYPYT | QQWSSYPYT | QQWSSYPY |
| variable | (SEQ ID NO: 60) | (SEQ ID NO: 60) | (SEQ ID NO: 60) | (SEQ ID NO: 60) | (SEQ ID NO: 67) |
| region CDR3 | | | | | |

65G8 Heavy chain variable region (SEQ ID NO: 77)
QVQLQQSGPQLVRPGASVKISCKASGYSFTSYWMHWVKQRPGQGLEWIGMIDPSDSETRLNQKFKDKATL
TIDKSSSTAYMQLSSPTSEDSAVYYCTRDYFDYWGQGTTLTVSS
65G8 Light chain variable region (SEQ ID NO: 78)
QIVLTQSPAIMSTSPGEKVTMTCSASSSVSYMYWYQQKPGSSPRLLIYDTSNLASGVPVRFSGSGSGTSY
SLTISRMEAEDAATYYCQQWSSYPYTFGGGTKLEIK

In some embodiments, a HTRA1-binding agent comprises a heavy chain variable region CDR1, CDR2, and CDR3 and/or a light chain variable region CDR1, CDR2, and CDR3 from an antibody described herein. In some embodiments, a HTRA1-binding agent comprises a heavy chain variable region CDR1, CDR2, and CDR3 from an antibody described herein. In some embodiments, a HTRA1-binding agent comprises a light chain variable region CDR1, CDR2, and CDR3 from an antibody described herein. In some embodiments, a HTRA1-binding agent comprises a heavy chain variable region CDR1, CDR2, and CDR3 and a light chain variable region CDR1, CDR2, and CDR3 from an antibody described herein. In some embodiments, a HTRA1-binding agent comprises a heavy chain variable region comprising a heavy chain variable region CDR1, CDR2, and CDR3 from an antibody described herein. In some embodiments, a HTRA1-binding agent comprises a light chain variable region comprising a light chain variable region CDR1, CDR2, and CDR3 from an antibody described herein. In some embodiments, a HTRA1-binding agent comprises: (a) a heavy chain variable region comprising a heavy chain variable region CDR1, CDR2, and CDR3 from an antibody described herein and (b) a light chain variable region comprising a light chain variable region CDR1, CDR2, and CDR3 from an antibody described herein. In some embodiments, a HTRA1-binding agent comprises a humanized version or humanized variant of an antibody described herein.

In some embodiments, a HTRA1-binding agent comprises a heavy chain variable region CDR1, CDR2, and CDR3 and/or a light chain variable region CDR1, CDR2, and CDR3 from antibody 24F7, a humanized version thereof, or a variant thereof. In some embodiments, a HTRA1-binding agent comprises a heavy chain variable region CDR1, a heavy chain variable region CDR2, and a heavy chain variable region CDR3 from antibody 24F7 or hz24F7.v2. In other embodiments, a HTRA1-binding agent comprises a light chain variable region CDR1, a light chain variable region CDR2, and a light chain variable region CDR3 from antibody 24F7 or hz24F7.v2. In certain embodiments, a HTRA1-binding agent comprises a heavy chain variable region CDR1, a heavy chain variable region CDR2, a heavy chain variable region CDR3, a light chain variable region CDR1, a light chain variable region CDR2, and a light chain variable region CDR3 from antibody 24F7 or hz24F7.v2. In certain embodiments, a HTRA1-binding agent comprises: (a) a heavy chain variable region comprising a heavy chain variable region CDR1, a heavy chain variable region CDR2, and a heavy chain variable region CDR3; and (b) a light chain variable region comprising a light chain variable region CDR1, a light chain variable region CDR2, and a light chain variable region CDR3 from antibody 24F7 or hz24F7.v2. In some embodiments, a HTRA1-binding agent is a humanized version of antibody 24F7. In some embodiments, a HTRA1-binding agent is a variant of antibody 24F7 or humanized 24F7. In some embodiments, a HTRA1-binding agent is antibody hz24F7.v2.

In some embodiments, a HTRA1-binding agent comprises a heavy chain variable region CDR1, CDR2, and CDR3 and/or a light chain variable region CDR1, CDR2, and CDR3 from antibody 9F8, a humanized version thereof, or a variant thereof. In some embodiments, a HTRA1-binding agent comprises a heavy chain variable region CDR1, a heavy chain variable region CDR2, and a heavy chain variable region CDR3 from antibody 9F8. In other embodiments, a HTRA1-binding agent comprises a light chain variable region CDR1, a light chain variable region CDR2, and a light chain variable region CDR3 from antibody 9F8. In certain embodiments, a HTRA1-binding agent comprises a heavy chain variable region CDR1, a heavy chain variable region CDR2, a heavy chain variable region CDR3, a light chain variable region CDR1, a light chain variable region CDR2, and a light chain variable region CDR3 from antibody 9F8. In certain embodiments, a HTRA1-binding agent comprises: (a) a heavy chain variable region comprising a heavy chain variable region CDR1, a heavy chain variable region CDR2, and a heavy chain variable region CDR3; and (b) a light chain variable region comprising a light chain variable region CDR1, a light chain variable region CDR2, and a light chain variable region CDR3 from antibody 9F8. In some embodiments, a HTRA1-binding agent is a humanized version of antibody 9F8. In some embodiments, a HTRA1-binding agent is a variant of antibody 9F8.

In some embodiments, a HTRA1-binding agent comprises a heavy chain variable region CDR1, CDR2, and CDR3 and/or a light chain variable region CDR1, CDR2, and CDR3 from antibody 55B12, a humanized version thereof, or a variant thereof. In some embodiments, a HTRA1-binding agent comprises a heavy chain variable region CDR1, a heavy chain variable region CDR2, and a heavy chain variable region CDR3 from antibody 55B12. In other embodiments, a HTRA1-binding agent comprises a light chain variable region CDR1, a light chain variable region CDR2, and a light chain variable region CDR3 from antibody 55B12. In certain embodiments, a HTRA1-binding agent comprises a heavy chain variable region CDR1, a heavy chain variable region CDR2, a heavy chain variable region CDR3, a light chain variable region CDR1, a light chain variable region CDR2, and a light chain variable region CDR3 from antibody 55B12. In certain embodiments, a HTRA1-binding agent comprises: (a) a heavy chain variable region comprising a heavy chain variable region CDR1, a heavy chain variable region CDR2, and a heavy chain variable region CDR3; and (b) a light chain variable region comprising a light chain variable region CDR1, a light chain variable region CDR2, and a light chain variable region CDR3 from antibody 55B12. In some embodiments, a HTRA1-binding agent is a humanized version of antibody 55B12. In some embodiments, a HTRA1-binding agent is a variant of antibody 55B12.

In some embodiments, a HTRA1-binding agent comprises a heavy chain variable region CDR1, CDR2, and CDR3 and/or a light chain variable region CDR1, CDR2, and CDR3 from antibody 65G8, a humanized version thereof, or a variant thereof. In some embodiments, a HTRA1-binding agent comprises a heavy chain variable region CDR1, a heavy chain variable region CDR2, and a heavy chain variable region CDR3 from antibody 65G8. In other embodiments, a HTRA1-binding agent comprises a light chain variable region CDR1, a light chain variable region CDR2, and a light chain variable region CDR3 from antibody 65G8. In certain embodiments, a HTRA1-binding agent comprises a heavy chain variable region CDR1, a heavy chain variable region CDR2, a heavy chain variable region CDR3, a light chain variable region CDR1, a light chain variable region CDR2, and a light chain variable region CDR3 from antibody 65G8. In certain embodiments, a HTRA1-binding agent comprises: (a) a heavy chain variable region comprising a heavy chain variable region CDR1, a heavy chain variable region CDR2, and a heavy chain variable region CDR3; and (b) a light chain variable region comprising a light chain variable region CDR1, a light chain variable region CDR2, and a light chain variable region CDR3 from antibody 65G8. In some embodiments, a HTRA1-binding agent is a humanized version of antibody 65G8. In some embodiments, a HTRA1-binding agent is a variant of antibody 65G8.

In some embodiments, a HTRA1-binding agent comprises: (a) a heavy chain variable region comprising a heavy chain variable region CDR1 comprising the amino acid sequence GYTFTDYEMH (SEQ ID NO:9), a heavy chain variable region CDR2 comprising the amino acid sequence AIDPETGGTAYNQKFKG (SEQ ID NO:10), and a heavy chain variable region CDR3 comprising the amino acid sequence EGYSYDGGGYYFDY (SEQ ID NO:11) or the amino acid sequence EGYSYEGGGYYFDY (SEQ ID NO:25), and a light chain variable region comprising a light chain variable region CDR1 comprising the amino acid sequence SVSSSVSYMY (SEQ ID NO:12), a light chain variable region CDR2 comprising the amino acid sequence DTSNLAS (SEQ ID NO:13), and a light chain variable region CDR3 comprising the amino acid sequence QQWSSYPT (SEQ ID NO:14); (b) a heavy chain variable region comprising a heavy chain variable region CDR1 comprising the amino acid sequence GYTFTDY (SEQ ID NO:15), a heavy chain variable region CDR2 comprising the amino acid sequence DPETGG (SEQ ID NO:16), and a heavy chain variable region CDR3 comprising the amino acid sequence EGYSYDGGGYYFDY (SEQ ID NO:11) or the amino acid sequence EGYSYEGGGYYFDY (SEQ ID NO:25), and a light chain variable region comprising a light chain variable region CDR1 comprising the amino acid sequence SVSSSVSYMY (SEQ ID NO:12), a light chain variable region CDR2 comprising the amino acid sequence DTSNLAS (SEQ ID NO:13), and a light chain variable region CDR3 comprising the amino acid sequence QQWSSYPT (SEQ ID NO:14); (c) a heavy chain variable region comprising a heavy chain variable region CDR1 comprising the amino acid sequence GYTFTDYEMH (SEQ ID NO:9), a heavy chain variable region CDR2 comprising the amino acid sequence AIDPETGGTA (SEQ ID NO:17), and a heavy chain variable region CDR3 comprising the amino acid sequence EGYSYDGGGYYFDY (SEQ ID NO:11) or the amino acid sequence EGYSYEGGGYYFDY (SEQ ID NO:25), and a light chain variable region comprising a light chain variable region CDR1 comprising the amino acid sequence SVSSSVSYMY (SEQ ID NO:12), a light chain variable region CDR2 comprising the amino acid sequence DTSNLAS (SEQ ID NO:13), and a light chain variable region CDR3 comprising the amino acid sequence QQWSSYPT (SEQ ID NO:14); (d) a heavy chain variable region comprising a heavy chain variable region CDR1 comprising the amino acid sequence DYEMH (SEQ ID NO:18), a heavy chain variable region CDR2 comprising the amino acid sequence AIDPETGGTAYNQKFKG (SEQ ID NO:10), and a heavy chain variable region CDR3 comprising the amino acid sequence EGYSYDGGGYYFDY (SEQ ID NO:11) or the amino acid sequence EGYSYEGGGYYFDY (SEQ ID NO:25), and a light chain variable region comprising a light chain variable region CDR1 comprising the amino acid sequence SVSSSVSYMY (SEQ ID NO:12), a light chain variable region CDR2 comprising the amino acid sequence DTSNLAS (SEQ ID NO:13), and a light chain variable region CDR3 comprising the amino acid sequence QQWSSYPT (SEQ ID NO:14); or (e) a heavy chain variable region comprising a heavy chain variable region CDR1 comprising the amino acid sequence TDYEMH (SEQ ID NO:19), a heavy chain variable region CDR2 comprising the amino acid sequence WIGAIDPETGGTA (SEQ ID NO:20), and a heavy chain variable region CDR3 comprising the amino acid sequence TREGYSYDGGGYYFD (SEQ ID NO:21) or the amino acid sequence TREGYSYEGGGYYFD (SEQ ID NO:26), and a light chain variable region comprising a light chain variable region CDR1 comprising the amino acid sequence SYMYWY (SEQ ID NO:22), a light chain variable region CDR2 comprising the amino acid sequence LLIYDTSNLA (SEQ ID NO:23), and a light chain variable region CDR3 comprising the amino acid sequence QQWSSYP (SEQ ID NO:24).

In some embodiments, a HTRA1-binding agent comprises: (a) a heavy chain variable region comprising a heavy chain variable region CDR1 comprising the amino acid sequence GYTFTDYEMH (SEQ ID NO:9), a heavy chain variable region CDR2 comprising the amino acid sequence AIDPETGGTAYNQKFKG (SEQ ID NO:10), a heavy chain variable region CDR3 comprising the amino acid sequence EGYSYDGGGYYFDY (SEQ ID NO:11) or the amino acid sequence EGYSYEGGGYYFDY (SEQ ID NO:25), and/or (b) a light chain variable region comprising a light chain variable region CDR1 comprising the amino acid sequence SVSSSVSYMY (SEQ ID NO:12), a light chain variable region CDR2 comprising the amino acid sequence DTSNLAS (SEQ ID NO:13), and a light chain variable region CDR3 comprising the amino acid sequence QQWSSYPT (SEQ ID NO:14). In certain embodiments, a HTRA1-binding agent comprises a heavy chain variable region comprising a heavy chain variable region CDR1 comprising the amino acid sequence GYTFTDYEMH (SEQ ID NO:9), a heavy chain variable region CDR2 comprising the amino acid sequence AIDPETGGTAYNQKFKG (SEQ ID NO:10), and a heavy chain variable region CDR3 comprising the amino acid sequence EGYSYDGGGYYFDY (SEQ ID NO:11). In certain embodiments, a HTRA1-binding agent comprises a heavy chain variable region comprising a heavy chain variable region CDR1 comprising the amino acid sequence GYTFTDYEMH (SEQ ID NO:9), a heavy chain variable region CDR2 comprising the amino acid sequence AIDPETGGTAYNQKFKG (SEQ ID NO:10), and a heavy chain variable region CDR3 comprising the amino acid sequence EGYSYEGGGYYFDY (SEQ ID NO:25). In some embodiments, a HTRA1-binding agent comprises a light chain variable region comprising a light chain variable region CDR1 comprising the amino acid sequence SVSSSVSYMY (SEQ ID NO:12), a light chain variable region CDR2 comprising the amino acid sequence DTSNLAS (SEQ ID NO:13), and a light chain variable region CDR3 comprising the amino acid sequence QQWSSYPT (SEQ ID NO:14). In certain embodiments, a HTRA1-binding agent comprises a heavy chain variable region comprising a heavy chain variable region CDR1 comprising the amino acid sequence GYTFTDYEMH (SEQ ID NO:9), a heavy chain variable region CDR2 comprising the amino acid sequence AIDPETGGTAYNQKFKG (SEQ ID NO:10), and a heavy chain variable region CDR3 comprising the amino acid sequence EGYSYDGGGYYFDY (SEQ ID NO:11), and a light chain variable region comprising a light chain variable region CDR1 comprising the amino acid sequence SVSSSVSYMY (SEQ ID NO:12), a light chain variable region CDR2 comprising the amino acid sequence DTSNLAS (SEQ ID NO:13), and a light chain variable region CDR3 comprising the amino acid sequence QQWSSYPT (SEQ ID NO:14). In certain embodiments, a HTRA1-binding agent comprises a heavy chain variable region comprising a heavy chain variable region CDR1 comprising the amino acid sequence GYTFTDYEMH (SEQ ID NO:9), a heavy chain variable region CDR2 comprising the amino acid sequence AIDPETGGTAYNQKFKG (SEQ ID NO:10), and a heavy chain variable region CDR3 comprising the amino acid sequence EGYSYEGGGYYFDY (SEQ ID NO:25), and a light chain variable region comprising a light chain variable region CDR1 comprising the amino acid sequence SVSSSVSYMY (SEQ ID NO:12), a light chain variable region CDR2 comprising the amino acid sequence DTSNLAS (SEQ ID NO:13), and a light chain variable region CDR3 comprising the amino acid sequence QQWSSYPT (SEQ ID NO:14).

In some embodiments, a HTRA1-binding agent comprises: (a) a heavy chain variable region comprising a heavy chain variable region CDR1 comprising the amino acid sequence GYTFTDYEMH (SEQ ID NO:9), a heavy chain variable region CDR2 comprising the amino acid sequence AIDPETGGTAYNQKFKG (SEQ ID NO:10), and a heavy chain variable region CDR3 comprising the amino acid sequence EGYSYDGGGYYFDY (SEQ ID NO:11); and (b) a light chain variable region comprising a light chain variable region CDR1 comprising the amino acid sequence SVSSSVSYMY (SEQ ID NO:12), a light chain variable region CDR2 comprising the amino acid sequence DTSNLAS (SEQ ID NO:13), and a light chain variable region CDR3 comprising the amino acid sequence QQWYSYPT (SEQ ID NO:94). In some embodiments, a HTRA1-binding agent comprises: (a) a heavy chain variable region comprising a heavy chain variable region CDR1 comprising the amino acid sequence GYTFTDYEMH (SEQ ID NO:9), a heavy chain variable region CDR2 comprising the amino acid sequence AIDPETGGTAYNQKFKG (SEQ ID NO:10), and a heavy chain variable region CDR3 comprising the amino acid sequence EGYSYEGGGYYFDY (SEQ ID NO:25); and (b) a light chain variable region comprising a light chain variable region CDR1 comprising the amino acid sequence SVSSSVSYMY (SEQ ID NO:12), a light chain variable region CDR2 comprising the amino acid sequence DTSNLAS (SEQ ID NO:13), and a light chain variable region CDR3 comprising the amino acid sequence QQWYSYPT (SEQ ID NO:94). In some embodiments, a HTRA1-binding agent comprises: (a) a heavy chain variable region comprising a heavy chain variable region CDR1 comprising the amino acid sequence GYTFTDYEMH (SEQ ID NO:9), a heavy chain variable region CDR2 comprising the amino acid sequence AIDPETGGTAYNQKFKG (SEQ ID NO:10), and a heavy chain variable region CDR3 comprising the amino acid sequence EGYSYDGGGYYFDY (SEQ ID NO:11); and (b) a light chain variable region comprising a light chain variable region CDR1 comprising the amino acid sequence SVSSSVSYMY (SEQ ID NO:12), a light chain variable region CDR2 comprising the amino acid sequence DTSNLAS (SEQ ID NO:13), and a light chain variable region CDR3 comprising the amino acid sequence QQWSTYPT (SEQ ID NO:99). In some embodiments, a HTRA1-binding agent comprises: (a) a heavy chain variable region comprising a heavy chain variable region CDR1 comprising the amino acid sequence GYTFTDYEMH (SEQ ID NO:9), a heavy chain variable region CDR2 comprising the amino acid sequence AIDPETGGTAYNQKFKG (SEQ ID NO:10), and a heavy chain variable region CDR3 comprising the amino acid sequence EGYSYEGGGYYFDY (SEQ ID NO:25); and (b) a light chain variable region comprising a light chain variable region CDR1 comprising the amino acid sequence SVSSSVSYMY (SEQ ID NO:12), a light chain variable region CDR2 comprising the amino acid sequence DTSNLAS (SEQ ID NO:13), and a light chain variable region CDR3 comprising the amino acid sequence QQWSTYPT (SEQ ID NO:99). In some embodiments, the HTRA1-binding agent comprises: a heavy chain variable region comprising a heavy chain variable region CDR1 comprising the amino acid sequence GYTFTDYEMH (SEQ ID NO:9), a heavy chain variable region CDR2 comprising the amino acid sequence AIDPETGGTAYNQKFKG (SEQ ID NO:10), and a heavy chain variable region CDR3 comprising the amino acid sequence EGYSYDGGGYYFDY (SEQ ID NO:11); and a light chain variable region comprising a light chain variable region CDR1 comprising the amino acid sequence SVSSSVSYMY (SEQ ID NO:12), a light chain variable region CDR2 comprising the amino acid sequence DTSNLAS (SEQ ID NO:13), and a light chain variable region CDR3 comprising the amino acid sequence QQWDSYPT (SEQ ID NO:104). In some embodiments, the HTRA1-binding agent comprises: a heavy chain variable region comprising a heavy chain variable region CDR1 comprising the amino acid sequence GYTFTDYEMH (SEQ ID NO:9), a heavy chain variable region CDR2 comprising the amino acid sequence AIDPETGGTAYNQKFKG (SEQ ID NO:10), and a heavy chain variable region CDR3 comprising the amino acid sequence EGYSYEGGGYYFDY (SEQ ID NO:25); and a light chain variable region comprising a light chain variable region CDR1 comprising the amino acid sequence SVSSSVSYMY (SEQ ID NO:12), a light chain variable region CDR2 comprising the amino acid sequence DTSNLAS (SEQ ID NO:13), and a light chain variable region CDR3 comprising the amino acid sequence QQWDSYPT (SEQ ID NO:104). In some embodiments, the HTRA1-binding agent comprises: a heavy chain variable region comprising a heavy chain variable region CDR1 comprising the amino acid sequence GYTFTDYEMH (SEQ ID NO:9), a heavy chain variable region CDR2 comprising the amino acid sequence AIDPETGGTAYNQKFKG (SEQ ID NO:10), and a heavy chain variable region CDR3 comprising the amino acid sequence EGYSYDGGGYYFDY (SEQ ID NO:11); and a light chain variable region comprising a light chain variable region CDR1 comprising the amino acid sequence SVSSSVSYMY (SEQ ID NO:12), a light chain variable region CDR2 comprising the amino acid sequence DTSNLAS (SEQ ID NO:13), and a light chain variable region CDR3 comprising the amino acid sequence QQWTSYPT (SEQ ID NO:107). In some embodiments, the HTRA1-binding agent comprises: a heavy chain variable region comprising a heavy chain variable region CDR1 comprising the amino acid sequence GYTFTDYEMH (SEQ ID NO:9), a heavy chain variable region CDR2 comprising the amino acid sequence AIDPETGGTAYNQKFKG (SEQ ID NO:10), and a heavy chain variable region CDR3 comprising the amino acid sequence EGYSYEGGGYYFDY (SEQ ID NO:25); and a light chain variable region comprising a light chain variable region CDR1 comprising the amino acid sequence SVSSSVSYMY (SEQ ID NO:12), a light chain variable region CDR2 comprising the amino acid sequence DTSNLAS (SEQ ID NO:13), and a light chain variable region CDR3 comprising the amino acid sequence QQWTSYPT (SEQ ID NO:107). In some embodiments, the HTRA1-binding agent comprises: a heavy chain variable region comprising a heavy chain variable region CDR1 comprising the amino acid sequence GYTFTDYEMH (SEQ ID NO:9), a heavy chain variable region CDR2 comprising the amino acid sequence AIDPETGGTAYNQKFKG (SEQ ID NO:10), and a heavy chain variable region CDR3 comprising the amino acid sequence EGYSYDGGGYYFDY (SEQ ID NO:11); and a light chain variable region comprising a light chain variable region CDR1 comprising the amino acid sequence SVSSSVSYMY (SEQ ID NO:12), a light chain variable region CDR2 comprising the amino acid sequence DTSNLAS (SEQ ID NO:13), and a light chain variable region CDR3 comprising the amino acid sequence QQWASYPT (SEQ ID NO:110). In some embodiments, the HTRA1-binding agent comprises: a heavy chain variable region comprising a heavy chain variable region CDR1 comprising the amino acid sequence GYTFTDYEMH (SEQ ID NO:9), a heavy chain variable region CDR2 comprising the amino acid sequence AIDPETGGTAYNQKFKG (SEQ ID NO:10), and a heavy chain variable region CDR3 comprising the amino acid sequence EGYSYEGGGYYFDY (SEQ ID NO:25); and a light chain variable region comprising a light chain variable region CDR1 comprising the amino acid sequence SVSSSVSYMY (SEQ ID NO:12), a light chain variable region CDR2 comprising the amino acid sequence DTSNLAS (SEQ ID NO:13), and a light chain variable region CDR3 comprising the amino acid sequence QQWASYPT (SEQ ID NO:110). In some embodiments, the HTRA1-binding agent comprises: a heavy chain variable region comprising a heavy chain variable region CDR1 comprising the amino acid sequence GYTFTDYEMH (SEQ ID NO:9), a heavy chain variable region CDR2 comprising the amino acid sequence AIDPETGGTAYNQKFKG (SEQ ID NO:10), and a heavy chain variable region CDR3 comprising the amino acid sequence EGYSYDGGGYYFDY (SEQ ID NO:11); and a light chain variable region comprising a light chain variable region CDR1 comprising the amino acid sequence SVSSSVSYMY (SEQ ID NO:12), a light chain variable region CDR2 comprising the amino acid sequence DTSNLAS (SEQ ID NO:13), and a light chain variable region CDR3 comprising the amino acid sequence QQWLSYPT (SEQ ID NO:113). In some embodiments, the HTRA1-binding agent comprises: a heavy chain variable region comprising a heavy chain variable region CDR1 comprising the amino acid sequence GYTFTDYEMH (SEQ ID NO:9), a heavy chain variable region CDR2 comprising the amino acid sequence AIDPETGGTAYNQKFKG (SEQ ID NO:10), and a heavy chain variable region CDR3 comprising the amino acid sequence EGYSYEGGGYYFDY (SEQ ID NO:25); and a light chain variable region comprising a light chain variable region CDR1 comprising the amino acid sequence SVSSSVSYMY (SEQ ID NO:12), a light chain variable region CDR2 comprising the amino acid sequence DTSNLAS (SEQ ID NO:13), and a light chain variable region CDR3 comprising the amino acid sequence QQWLSYPT (SEQ ID NO:113). In some embodiments, the HTRA1-binding agent comprises: a heavy chain variable region comprising a heavy chain variable region CDR1 comprising the amino acid sequence GYTFTDYEMH (SEQ ID NO:9), a heavy chain variable region CDR2 comprising the amino acid sequence AIDPETGGTAYNQKFKG (SEQ ID NO:10), and a heavy chain variable region CDR3 comprising the amino acid sequence EGYSYDGGGYYFDY (SEQ ID NO:11); and a light chain variable region comprising a light chain variable region CDR1 comprising the amino acid sequence SVSSSVSYMY (SEQ ID NO:12), a light chain variable region CDR2 comprising the amino acid sequence DTSNLAS (SEQ ID NO:13), and a light chain variable region CDR3 comprising the amino acid sequence QQWSYYPT (SEQ ID NO:116). In some embodiments, the HTRA1-binding agent comprises: a heavy chain variable region comprising a heavy chain variable region CDR1 comprising the amino acid sequence GYTFTDYEMH (SEQ ID NO:9), a heavy chain variable region CDR2 comprising the amino acid sequence AIDPETGGTAYNQKFKG (SEQ ID NO:10), and a heavy chain variable region CDR3 comprising the amino acid sequence EGYSYEGGGYYFDY (SEQ ID NO:25); and a light chain variable region comprising a light chain variable region CDR1 comprising the amino acid sequence SVSSSVSYMY (SEQ ID NO:12), a light chain variable region CDR2 comprising the amino acid sequence DTSNLAS (SEQ ID NO:13), and a light chain variable region CDR3 comprising the amino acid sequence QQWSYYPT (SEQ ID NO:116). In some embodiments, the HTRA1-binding agent comprises: a heavy chain variable region comprising a heavy chain variable region CDR1 comprising the amino acid sequence GYTFTDYEMH (SEQ ID NO:9), a heavy chain variable region CDR2 comprising the amino acid sequence AIDPETGGTAYNQKFKG (SEQ ID NO:10), and a heavy chain variable region CDR3 comprising the amino acid sequence EGYSYDGGGYYFDY (SEQ ID NO:11); and a light chain variable region comprising a light chain variable region CDR1 comprising the amino acid sequence SVSSSVSYMY (SEQ ID NO:12), a light chain variable region CDR2 comprising the amino acid sequence DTSNLAS (SEQ ID NO:13), and a light chain variable region CDR3 comprising the amino acid sequence QQWSDYPT (SEQ ID NO:119). In some embodiments, the HTRA1-binding agent comprises: a heavy chain variable region comprising a heavy chain variable region CDR1 comprising the amino acid sequence GYTFTDYEMH (SEQ ID NO:9), a heavy chain variable region CDR2 comprising the amino acid sequence AIDPETGGTAYNQKFKG (SEQ ID NO:10), and a heavy chain variable region CDR3 comprising the amino acid sequence EGYSYEGGGYYFDY (SEQ ID NO:25); and a light chain variable region comprising a light chain variable region CDR1 comprising the amino acid sequence SVSSSVSYMY (SEQ ID NO:12), a light chain variable region CDR2 comprising the amino acid sequence DTSNLAS (SEQ ID NO:13), and a light chain variable region CDR3 comprising the amino acid sequence QQWSDYPT (SEQ ID NO:119).

In certain embodiments, a HTRA1-binding agent comprises: (a) a heavy chain variable region comprising a heavy chain variable region CDR1 comprising the amino acid sequence GYTFTDYEMH (SEQ ID NO:9), or a variant thereof comprising 1, 2, 3, or 4 amino acid substitutions, a heavy chain variable region CDR2 comprising the amino acid sequence AIDPETGGTAYNQKFKG (SEQ ID NO:10), or a variant thereof comprising 1, 2, 3, or 4 amino acid substitutions, and a heavy chain variable region CDR3 comprising the amino acid sequence EGYSYDGGGYYFDY (SEQ ID NO:11), or a variant thereof comprising 1, 2, 3, or 4 amino acid substitutions; and (b) a light chain variable region comprising a light chain variable region CDR1 comprising the amino acid sequence SVSSSVSYMY (SEQ ID NO:12), or a variant thereof comprising 1, 2, 3, or 4 amino acid substitutions, a light chain variable region CDR2 comprising the amino acid sequence DTSNLAS (SEQ ID NO:13), or a variant thereof comprising 1, 2, 3, or 4 amino acid substitutions, and a light chain variable region CDR3 comprising the amino acid sequence QQWSSYPT (SEQ ID NO:14), or a variant thereof comprising 1, 2, 3, or 4 amino acid substitutions. In certain embodiments, a HTRA1-binding agent comprises: (a) a heavy chain variable region comprising a heavy chain variable region CDR1 comprising the amino acid sequence GYTFTDYEMH (SEQ ID NO:9), or a variant thereof comprising 1, 2, 3, or 4 amino acid substitutions, a heavy chain variable region CDR2 comprising the amino acid sequence AIDPETGGTAYNQKFKG (SEQ ID NO:10), or a variant thereof comprising 1, 2, 3, or 4 amino acid substitutions, and a heavy chain variable region CDR3 comprising the amino acid sequence EGYSYEGGGYYFDY (SEQ ID NO:25), or a variant thereof comprising 1, 2, 3, or 4 amino acid substitutions; and (b) a light chain variable region comprising a light chain variable region CDR1 comprising the amino acid sequence SVSSSVSYMY (SEQ ID NO:12), or a variant thereof comprising 1, 2, 3, or 4 amino acid substitutions, a light chain variable region CDR2 comprising the amino acid sequence DTSNLAS (SEQ ID NO:13), or a variant thereof comprising 1, 2, 3, or 4 amino acid substitutions, and a light chain variable region CDR3 comprising the amino acid sequence QQWSSYPT (SEQ ID NO:14), or a variant thereof comprising 1, 2, 3, or 4 amino acid substitutions.

In some embodiments, a HTRA1-binding agent comprises a heavy chain variable region and/or a light chain variable region that comprises a modification within the amino acid sequence wherein the modification reduces deamidation. In some embodiments, a HTRA1-binding agent comprises one or more heavy chain variable region CDRs or light chain variable region CDRs that have been modified to reduce deamidation within the CDR sequence. Deamidation is a chemical reaction in which an amide functional group in the side chain of the amino acids asparagine (Asn or N) or glutamine (Gln or Q) is removed or converted to another functional group. Generally, asparagine is converted to aspartic acid or isoaspartic acid and glutamine is converted to glutamic acid or polyglutamic acid. In some situations, deamidation may change the structure, function, and/or stability of a polypeptide, potentially resulting in decreased biological activity.

In some embodiments, a HTRA1-binding agent comprises a heavy chain variable region and/or a light chain variable region that comprises a modification within the amino acid sequence wherein the modification reduces isomerization. In some embodiments, a HTRA1-binding agent comprises one or more heavy chain variable region CDRs or light chain variable region CDRs that have been modified to reduce isomerization. Isomerization is a chemical process by which a compound is transformed into any of its isomeric forms, i.e., forms with the same chemical composition but with different structure or configuration and, potentially with different physical and chemical properties. Studies have shown that aspartate (Asp or D) isomerization within a CDR can impact antibody binding and/or stability.

In some embodiments, a HTRA1-binding agent comprises a heavy chain variable region comprising an amino acid sequence that has the three heavy chain variable region CDRs of antibody 24F7 and which has at least 75%, at least 80%, at least 85%, at least 90%, at least 95%, at least 96%, at least 97%, at least 98%, at least 99%, or 100% identity to the sequence of SEQ ID NO:68 and a light chain variable region comprising an amino acid sequence that has the three light chain variable region CDRs of antibody 24F7 and which has at least 75%, at least 80%, at least 85%, at least 90%, at least 95%, at least 96%, at least 97%, at least 98%, at least 99%, or 100% identity to the sequence of SEQ ID NO:69. In some embodiments, a HTRA1 binding agent comprises a heavy chain variable region comprising an amino acid sequence that has the three heavy chain variable region CDRs of antibody hz24F7.v2 and which has at least 75%, at least 80%, at least 85%, at least 90%, at least 95%, at least 96%, at least 97%, at least 98%, at least 99%, or 100% identity to the sequence of SEQ ID NO:71 and a light chain variable region comprising an amino acid sequence that has the three light chain variable region CDRs of antibody hz24F7.v2 and which has at least 75%, at least 80%, at least 85%, at least 90%, at least 95%, at least 96%, at least 97%, at least 98%, at least 99%, or 100% identity to the sequence of SEQ ID NO:72.

In some embodiments, a HTRA1-binding agent comprises a heavy chain variable region having at least 80% sequence identity to SEQ ID NO:68, SEQ ID NO:70, or SEQ ID NO:71. In some embodiments, a HTRA1-binding agent comprises a light chain variable region having at least 80% sequence identity to SEQ ID NO:69 or SEQ ID NO:72. In some embodiments, a HTRA1-binding agent comprises a heavy chain variable region having at least 85%, at least 90%, at least 95%, at least 97%, or at least 99% sequence identity to SEQ ID NO:68. In some embodiments, a HTRA1-binding agent comprises a heavy chain variable region having at least 85%, at least 90%, at least 95%, at least 97%, or at least 99% sequence identity to SEQ ID NO:70. In some embodiments, a HTRA1-binding agent comprises a heavy chain variable region having at least 85%, at least 90%, at least 95%, at least 97%, or at least 99% sequence identity to SEQ ID NO:71. In some embodiments, a HTRA1-binding agent comprises a light chain variable region having at least 85%, at least 90%, at least 95%, at least 97%, or at least 99% sequence identity to SEQ ID NO:69. In some embodiments, a HTRA1-binding agent comprises a light chain variable region having at least 85%, at least 90%, at least 95%, at least 97%, or at least 99% sequence identity to SEQ ID NO:72.

In some embodiments, a HTRA1-binding agent comprises a heavy chain variable region comprising an amino acid sequence of SEQ ID NO:68. In some embodiments, a HTRA1-binding agent comprises a heavy chain variable region comprising an amino acid sequence of SEQ ID NO:70. In some embodiments, a HTRA1-binding agent comprises a heavy chain variable region comprising an amino acid sequence of SEQ ID NO:71. In some embodiments, a HTRA1-binding agent comprises a light chain variable region comprising an amino acid sequence of SEQ ID NO:69. In some embodiments, a HTRA1-binding agent comprises a light chain variable region comprising an amino acid sequence of SEQ ID NO:72.

In some embodiments, a HTRA1-binding agent comprises a heavy chain variable region having at least 80% sequence identity to SEQ ID NO:68 and a light chain variable region having at least 80% sequence identity to SEQ ID NO:69. In some embodiments, a HTRA1-binding agent comprises a heavy chain variable region having at least 90% sequence identity to SEQ ID NO:68 and a light chain variable region having at least 90% sequence identity to SEQ ID NO:69. In some embodiments, a HTRA1-binding agent comprises a heavy chain variable region having at least 95% sequence identity to SEQ ID NO:68 and a light chain variable region having at least 95% sequence identity to SEQ ID NO:69. In some embodiments, a HTRA1-binding agent comprises a heavy chain variable region comprising an amino acid sequence of SEQ ID NO:68 and a light chain variable region comprising an amino acid sequence of SEQ ID NO:69.

In some embodiments, a HTRA1-binding agent comprises a heavy chain variable region having at least 80% sequence identity to SEQ ID NO:70 and a light chain variable region having at least 80% sequence identity to SEQ ID NO:72. In some embodiments, a HTRA1-binding agent comprises a heavy chain variable region having at least 90% sequence identity to SEQ ID NO:70 and a light chain variable region having at least 90% sequence identity to SEQ ID NO:72. In some embodiments, a HTRA1-binding agent comprises a heavy chain variable region having at least 95% sequence identity to SEQ ID NO:70 and a light chain variable region having at least 95% sequence identity to SEQ ID NO:72. In some embodiments, a HTRA1-binding agent comprises a heavy chain variable region comprising an amino acid sequence of SEQ ID NO:70 and a light chain variable region comprising an amino acid sequence of SEQ ID NO:72.

In some embodiments, a HTRA1-binding agent comprises a heavy chain variable region having at least 80% sequence identity to SEQ ID NO:71 and a light chain variable region having at least 80% sequence identity to SEQ ID NO:72. In some embodiments, a HTRA1-binding agent comprises a heavy chain variable region having at least 90% sequence identity to SEQ ID NO:71 and a light chain variable region having at least 90% sequence identity to SEQ ID NO:72. In some embodiments, a HTRA1-binding agent comprises a heavy chain variable region having at least 95% sequence identity to SEQ ID NO:71 and a light chain variable region having at least 95% sequence identity to SEQ ID NO:72. In some embodiments, a HTRA1-binding agent comprises a heavy chain variable region comprising an amino acid sequence of SEQ ID NO:71 and a light chain variable region comprising an amino acid sequence of SEQ ID NO:72.

In certain embodiments, a HTRA1-binding agent comprises: (a) a heavy chain variable region comprising a heavy chain variable region CDR1 comprising the amino acid sequence GYTFTDYEMH (SEQ ID NO:9), a heavy chain variable region CDR2 comprising the amino acid sequence AIDPETGGTAYNQKFKG (SEQ ID NO:10), and a heavy chain variable region CDR3 comprising the amino acid sequence EGYSYEGGGYYFDY (SEQ ID NO:25); and (b) a light chain variable region comprising a light chain variable region CDR1 comprising the amino acid sequence SVSSSVSYMY (SEQ ID NO:12), a light chain variable region CDR2 comprising the amino acid sequence DTSNLAS (SEQ ID NO:13), and a light chain variable region CDR3 comprising the amino acid sequence QQWSSYPT (SEQ ID NO:14), wherein the heavy chain comprises at least 80%, at least 85%, at least 90%, at least 95%, at least 97%, or 100% identity to the sequence of SEQ ID NO:88, and wherein the light chain comprises at least 80%, at least 85%, at least 90%, at least 95%, at least 97%, or 100% identity to the sequence of SEQ ID NO:90. In certain embodiments, a HTRA1-binding agent comprises: (a) a heavy chain variable region comprising a heavy chain variable region CDR1 comprising the amino acid sequence GYTFTDYEMH (SEQ ID NO:9), a heavy chain variable region CDR2 comprising the amino acid sequence AIDPETGGTAYNQKFKG (SEQ ID NO:10), and a heavy chain variable region CDR3 comprising the amino acid sequence EGYSYEGGGYYFDY (SEQ ID NO:25); and (b) a light chain variable region comprising a light chain variable region CDR1 comprising the amino acid sequence SVSSSVSYMY (SEQ ID NO:12), a light chain variable region CDR2 comprising the amino acid sequence DTSNLAS (SEQ ID NO:13), and a light chain variable region CDR3 comprising the amino acid sequence QQWSSYPT (SEQ ID NO:14), wherein the heavy chain comprises at least 95% identity to the sequence of SEQ ID NO:88, and wherein the light chain comprises at least 95% identity to the sequence of SEQ ID NO:90. In certain embodiments, a HTRA1-binding agent comprises (a) a heavy chain comprising the amino acid sequence of SEQ ID NO:88 and (b) a light chain comprising a light chain variable region CDR1 comprising the amino acid sequence SVSSSVSYMY (SEQ ID NO:12), a light chain variable region CDR2 comprising the amino acid sequence DTSNLAS (SEQ ID NO:13), and a light chain variable region CDR3 comprising the amino acid sequence QQWSSYPT (SEQ ID NO:14), wherein the light chain comprises at least 80%, at least 85%, at least 90%, at least 95%, at least 97%, or 100% identity to the sequence of SEQ ID NO:90. In certain embodiments, a HTRA1-binding agent comprises (a) a heavy chain comprising a heavy chain variable region CDR1 comprising the amino acid sequence GYTFTDYEMH (SEQ ID NO:9), a heavy chain variable region CDR2 comprising the amino acid sequence AIDPETGGTAYNQKFKG (SEQ ID NO:10), a heavy chain variable region CDR3 comprising the amino acid sequence EGYSYEGGGYYFDY (SEQ ID NO:25), wherein the heavy chain comprises at least 80%, at least 85%, at least 90%, at least 95%, at least 97%, or 100% identity to the sequence of SEQ ID NO:88, and (b) a light chain comprising the amino acid sequence of SEQ ID NO:90. In some embodiments, a HTRA1-binding agent is an antibody comprising a heavy chain comprising the amino acid sequence of SEQ ID NO:88 and a light chain comprising the amino acid sequence of SEQ ID NO:90.

In some embodiments, the HTRA1-binding agent is antibody 24F7. In some embodiments, the HTRA1-binding agent is antibody hz24F7. In some embodiments, the HTRA1-binding agent is antibody hz24F7.v2.

In certain embodiments, a HTRA1-binding agent comprises: (a) a heavy chain variable region comprising a heavy chain variable region CDR1 comprising the amino acid sequence GYAFTTYWMH (SEQ ID NO:27), a heavy chain variable region CDR2 comprising the amino acid sequence NIDPSDSETHYNQKFRD (SEQ ID NO:28), and a heavy chain variable region CDR3 comprising the amino acid sequence DYGAFDV (SEQ ID NO:29), and a light chain variable region comprising a light chain variable region CDR1 comprising the amino acid sequence RSSTGAVTTRNFAS (SEQ ID NO:30), a light chain variable region CDR2 comprising the amino acid sequence GTNNRAP (SEQ ID NO:31), and a light chain variable region CDR3 comprising the amino acid sequence ALWYSNLWV (SEQ ID NO:32); (b) a heavy chain variable region comprising a heavy chain variable region CDR1 comprising GYAFTTY (SEQ ID NO:33), a heavy chain variable region CDR2 comprising the amino acid sequence DPSDSE (SEQ ID NO:34), and a heavy chain variable region CDR3 comprising the amino acid sequence DYGAFDV (SEQ ID NO:29), and a light chain variable region comprising a light chain variable region CDR1 comprising the amino acid sequence RSSTGAVTTRNFAS (SEQ ID NO:30), a light chain variable region CDR2 comprising the amino acid sequence GTNNRAP (SEQ ID NO:31), and a light chain variable region CDR3 comprising the amino acid sequence ALWYSNLWV (SEQ ID NO:32); (c) a heavy chain variable region comprising a heavy chain variable region CDR1 comprising the amino acid sequence GYAFTTYWMH (SEQ ID NO:27), a heavy chain variable region CDR2 comprising the amino acid sequence NIDPSDSETH (SEQ ID NO:35), and a heavy chain variable region CDR3 comprising the amino acid sequence DYGAFDV (SEQ ID NO:29), and a light chain variable region comprising a light chain variable region CDR1 comprising the amino acid sequence RSSTGAVTTRNFAS (SEQ ID NO:30), a light chain variable region CDR2 comprising the amino acid sequence GTNNRAP (SEQ ID NO:31), and a light chain variable region CDR3 comprising the amino acid sequence ALWYSNLWV (SEQ ID NO:32); (d) a heavy chain variable region comprising a heavy chain variable region CDR1 comprising the amino acid sequence TYWMH (SEQ ID NO:36), a heavy chain variable region CDR2 comprising the amino acid sequence NIDPSDSETHYNQKFRD (SEQ ID NO:28), and a heavy chain variable region CDR3 comprising the amino acid sequence DYGAFDV (SEQ ID NO:29), and a light chain variable region comprising a light chain variable region CDR1 comprising the amino acid sequence RSSTGAVTTRNFAS (SEQ ID NO:30), a light chain variable region CDR2 comprising the amino acid sequence GTNNRAP (SEQ ID NO:31), and a light chain variable region CDR3 comprising the amino acid sequence ALWYSNLWV (SEQ ID NO:32); or (e) a heavy chain variable region comprising a heavy chain variable region CDR1 comprising TTYWMH (SEQ ID NO:37), a heavy chain variable region CDR2 comprising the amino acid sequence WIGNIDPSDSETH (SEQ ID NO:38), and a heavy chain variable region CDR3 comprising the amino acid sequence ARDYGAFD (SEQ ID NO:39), and a light chain variable region comprising a light chain variable region CDR1 comprising the amino acid sequence VTTRNFASWV (SEQ ID NO:40), a light chain variable region CDR2 comprising the amino acid sequence GLIGGTNNRA (SEQ ID NO:41), and a light chain variable region CDR3 comprising the amino acid sequence ALWYSNLW (SEQ ID NO:42).

In certain embodiments, a HTRA1-binding agent comprises a heavy chain variable region comprising a heavy chain variable region CDR1 comprising the amino acid sequence GYAFTTYWMH (SEQ ID NO:27), a heavy chain variable region CDR2 comprising the amino acid sequence NIDPSDSETHYNQKFRD (SEQ ID NO:28), and a heavy chain variable region CDR3 comprising the amino acid sequence DYGAFDV (SEQ ID NO:29). In some embodiments, a HTRA1-binding agent comprises a light chain variable region comprising a light chain variable region CDR1 comprising the amino acid sequence RSSTGAVTTRNFAS (SEQ ID NO:30), a light chain variable region CDR2 comprising the amino acid sequence GTNNRAP (SEQ ID NO:31), and a light chain variable region CDR3 comprising the amino acid sequence ALWYSNLWV (SEQ ID NO:32). In certain embodiments, a HTRA1-binding agent comprises: (a) a heavy chain variable region comprising a heavy chain variable region CDR1 comprising the amino acid sequence GYAFTTYWMH (SEQ ID NO:27), a heavy chain variable region CDR2 comprising the amino acid sequence NIDPSDSETHYNQKFRD (SEQ ID NO:28), and a heavy chain variable region CDR3 comprising the amino acid sequence DYGAFDV (SEQ ID NO:29); and (b) a light chain variable region comprising a light chain variable region CDR1 comprising the amino acid sequence RSSTGAVTTRNFAS (SEQ ID NO:30), a light chain variable region CDR2 comprising the amino acid sequence GTNNRAP (SEQ ID NO:31), and a light chain variable region CDR3 comprising the amino acid sequence ALWYSNLWV (SEQ ID NO:32).

In some embodiments, a HTRA1-binding agent comprises a heavy chain variable region having at least 80% sequence identity to SEQ ID NO:73. In some embodiments, a HTRA1-binding agent comprises a light chain variable region having at least 80% sequence identity to SEQ ID NO:74. In some embodiments, a HTRA1-binding agent comprises a heavy chain variable region having at least 85%, at least 90%, at least 95%, at least 97%, or at least 99% sequence identity to SEQ ID NO:73. In some embodiments, a HTRA1-binding agent comprises a light chain variable region having at least 85%, at least 90%, at least 95%, at least 97%, or at least 99% sequence identity to SEQ ID NO:74. In some embodiments, a HTRA1-binding agent comprises a heavy chain variable region comprising an amino acid sequence of SEQ ID NO:73. In some embodiments, a HTRA1-binding agent comprises a light chain variable region comprising an amino acid sequence of SEQ ID NO:74.

In some embodiments, a HTRA1-binding agent comprises a heavy chain variable region having at least 80% sequence identity to SEQ ID NO:73 and a light chain variable region having at least 80% sequence identity to SEQ ID NO:74. In some embodiments, a HTRA1-binding agent comprises a heavy chain variable region having at least 90% sequence identity to SEQ ID NO:73 and a light chain variable region having at least 90% sequence identity to SEQ ID NO:74. In some embodiments, a HTRA1-binding agent comprises a heavy chain variable region having at least 95% sequence identity to SEQ ID NO:73 and a light chain variable region having at least 95% sequence identity to SEQ ID NO:74. In some embodiments, a HTRA1-binding agent comprises a heavy chain variable region comprising an amino acid sequence of SEQ ID NO:73 and a light chain variable region comprising an amino acid sequence of SEQ ID NO:74.

In some embodiments, the HTRA1-binding agent is antibody 9F8. In some embodiments, the HTRA1-binding agent is a humanized version of antibody 9F8.

In certain embodiments, a HTRA1-binding agent comprises: (a) a heavy chain variable region comprising a heavy chain variable region CDR1 comprising the amino acid sequence GYTFTNYWMH (SEQ ID NO:43), a heavy chain variable region CDR2 comprising the amino acid sequence NIDPSDSETHYNQKFKD (SEQ ID NO:44), and a heavy chain variable region CDR3 comprising the amino acid sequence EDSSGYGAY (SEQ ID NO:45), and a light chain variable region comprising a light chain variable region CDR1 comprising the amino acid sequence SASSSVNYMH (SEQ ID NO:46), a light chain variable region CDR2 comprising the amino acid sequence DTSKLAS (SEQ ID NO:47), and a light chain variable region CDR3 comprising the amino acid sequence QQWSSHPLT (SEQ ID NO:48); (b) a heavy chain variable region comprising a heavy chain variable region CDR1 comprising the amino acid sequence GYTFTNY (SEQ ID NO:49), a heavy chain variable region CDR2 comprising DPSDSE (SEQ ID NO:34), and a heavy chain variable region CDR3 comprising the amino acid sequence EDSSGYGAY (SEQ ID NO:45), and a light chain variable region comprising a light chain variable region CDR1 comprising the amino acid sequence SASSSVNYMH (SEQ ID NO:46), a light chain variable region CDR2 comprising the amino acid sequence DTSKLAS (SEQ ID NO:47), and a light chain variable region CDR3 comprising the amino acid sequence QQWSSHPLT (SEQ ID NO:48); (c) a heavy chain variable region comprising a heavy chain variable region CDR1 comprising the amino acid sequence GYTFTNYWMH (SEQ ID NO:43), a heavy chain variable region CDR2 comprising the amino acid sequence NIDPSDSETH (SEQ ID NO:35), and a heavy chain variable region CDR3 comprising the amino acid sequence EDSSGYGAY (SEQ ID NO:45), and a light chain variable region comprising a light chain variable region CDR1 comprising the amino acid sequence SASSSVNYMH (SEQ ID NO:46), a light chain variable region CDR2 comprising the amino acid sequence DTSKLAS (SEQ ID NO:47), and a light chain variable region CDR3 comprising the amino acid sequence QQWSSHPLT (SEQ ID NO:48); (d) a heavy chain variable region comprising a heavy chain variable region CDR1 comprising the amino acid sequence NYWMH (SEQ ID NO:50), a heavy chain variable region CDR2 comprising the amino acid sequence NIDPSDSETHYNQKFKD (SEQ ID NO:44), and a heavy chain variable region CDR3 comprising the amino acid sequence EDSSGYGAY (SEQ ID NO:45), and a light chain variable region comprising a light chain variable region CDR1 comprising the amino acid sequence SASSSVNYMH (SEQ ID NO:46), a light chain variable region CDR2 comprising the amino acid sequence DTSKLAS (SEQ ID NO:47), and a light chain variable region CDR3 comprising the amino acid sequence QQWSSHPLT (SEQ ID NO:48); or (e) a heavy chain variable region comprising a heavy chain variable region CDR1 comprising the amino acid sequence TNYWMH (SEQ ID NO:51), a heavy chain variable region CDR2 comprising the amino acid sequence WIGNIDPSDSETH (SEQ ID NO:38), and a heavy chain variable region CDR3 comprising the amino acid sequence AREDSSGYGA (SEQ ID NO:52), and a light chain variable region comprising a light chain variable region CDR1 comprising the amino acid sequence NYMHWY (SEQ ID NO:53), a light chain variable region CDR2 comprising the amino acid sequence RWIYDTSKLA (SEQ ID NO:54), and a light chain variable region CDR3 comprising the amino acid sequence QQWSSHPL (SEQ ID NO:55).

In certain embodiments, a HTRA1-binding agent comprises a heavy chain variable region comprising a heavy chain variable region CDR1 comprising the amino acid sequence GYTFTNYWMH (SEQ ID NO:43), a heavy chain variable region CDR2 comprising the amino acid sequence NIDPSDSETHYNQKFKD (SEQ ID NO:44), and a heavy chain variable region CDR3 comprising the amino acid sequence EDSSGYGAY (SEQ ID NO:45). In some embodiments, a HTRA1-binding agent comprises a light chain variable region comprising a light chain variable region CDR1 comprising the amino acid sequence SASSSVNYMH (SEQ ID NO:46), a light chain variable region CDR2 comprising the amino acid sequence DTSKLAS (SEQ ID NO:47), and a light chain variable region CDR3 comprising the amino acid sequence QQWSSHPLT (SEQ ID NO:48). In certain embodiments, a HTRA1-binding agent comprises: (a) a heavy chain variable region comprising a heavy chain variable region CDR1 comprising the amino acid sequence GYTFTNYWMH (SEQ ID NO:43), a heavy chain variable region CDR2 comprising the amino acid sequence NIDPSDSETHYNQKFKD (SEQ ID NO:44), and a heavy chain variable region CDR3 comprising the amino acid sequence EDSSGYGAY (SEQ ID NO:45), and (b) a light chain variable region comprising a light chain variable region CDR1 comprising the amino acid sequence SASSSVNYMH (SEQ ID NO:46), a light chain variable region CDR2 comprising the amino acid sequence DTSKLAS (SEQ ID NO:47), and a light chain variable region CDR3 comprising the amino acid sequence QQWSSHPLT (SEQ ID NO:48).

In some embodiments, a HTRA1-binding agent comprises a heavy chain variable region having at least 80% sequence identity to SEQ ID NO:75. In some embodiments, a HTRA1-binding agent comprises a light chain variable region having at least 80% sequence identity to SEQ ID NO:76. In some embodiments, a HTRA1-binding agent comprises a heavy chain variable region having at least 85%, at least 90%, at least 95%, at least 97%, or at least 99% sequence identity to SEQ ID NO:75. In some embodiments, a HTRA1-binding agent comprises a light chain variable region having at least 85%, at least 90%, at least 95%, at least 97%, or at least 99% sequence identity to SEQ ID NO:76. In some embodiments, a HTRA1-binding agent comprises a heavy chain variable region comprising an amino acid sequence of SEQ ID NO:75. In some embodiments, a HTRA1-binding agent comprises a light chain variable region comprising an amino acid sequence of SEQ ID NO:76.

In some embodiments, a HTRA1-binding agent comprises a heavy chain variable region having at least 80% sequence identity to SEQ ID NO:75 and a light chain variable region having at least 80% sequence identity to SEQ ID NO:76. In some embodiments, a HTRA1-binding agent comprises a heavy chain variable region having at least 90% sequence identity to SEQ ID NO:75 and a light chain variable region having at least 90% sequence identity to SEQ ID NO:76. In some embodiments, a HTRA1-binding agent comprises a heavy chain variable region having at least 95% sequence identity to SEQ ID NO:75 and a light chain variable region having at least 95% sequence identity to SEQ ID NO:76. In some embodiments, a HTRA1-binding agent comprises a heavy chain variable region comprising an amino acid sequence of SEQ ID NO:75 and a light chain variable region comprising an amino acid sequence of SEQ ID NO:76.

In some embodiments, the HTRA1-binding agent is antibody 55B12. In some embodiments, the HTRA1-binding agent is a humanized version of antibody 55B12.

In certain embodiments, a HTRA1-binding agent comprises: (a) a heavy chain variable region comprising a heavy chain variable region CDR1 comprising the amino acid sequence GYSFTSYWMH (SEQ ID NO:56), a heavy chain variable region CDR2 comprising the amino acid sequence MIDPSDSETRLNQKFKD (SEQ ID NO:57), and a heavy chain variable region CDR3 comprising the amino acid sequence DYFDY (SEQ ID NO:58), and a light chain variable region comprising a light chain variable region CDR1 comprising the amino acid sequence SASSSVSYMY (SEQ ID NO:59), a light chain variable region CDR2 comprising the amino acid sequence DTSNLAS (SEQ ID NO:13), and a light chain variable region CDR3 comprising the amino acid sequence QQWSSYPYT (SEQ ID NO:60); (b) a heavy chain variable region comprising a heavy chain variable region CDR1 comprising the amino acid sequence GYSFTSY (SEQ ID NO:61), a heavy chain variable region CDR2 comprising the amino acid sequence DPSDSE (SEQ ID NO:34), and a heavy chain variable region CDR3 comprising the amino acid sequence DYFDY (SEQ ID NO:58), and a light chain variable region comprising a light chain variable region CDR1 comprising the amino acid sequence SASSSVSYMY (SEQ ID NO:59), a light chain variable region CDR2 comprising the amino acid sequence DTSNLAS (SEQ ID NO:13), and a light chain variable region CDR3 comprising the amino acid sequence QQWSSYPYT (SEQ ID NO:60); (c) a heavy chain variable region comprising a heavy chain variable region CDR1 comprising the amino acid sequence GYSFTSYWMH (SEQ ID NO:56), a heavy chain variable region CDR2 comprising the amino acid sequence MIDPSDSETR (SEQ ID NO:62), and a heavy chain variable region CDR3 comprising the amino acid sequence DYFDY (SEQ ID NO:58), and a light chain variable region comprising a light chain variable region CDR1 comprising the amino acid sequence SASSSVSYMY (SEQ ID NO:59), a light chain variable region CDR2 comprising the amino acid sequence DTSNLAS (SEQ ID NO:13), and a light chain variable region CDR3 comprising the amino acid sequence QQWSSYPYT (SEQ ID NO:60); (d) a heavy chain variable region comprising a heavy chain variable region CDR1 comprising the amino acid sequence SYWMH (SEQ ID NO:63), a heavy chain variable region CDR2 comprising the amino acid sequence MIDPSDSETRLNQKFKD (SEQ ID NO:57), and a heavy chain variable region CDR3 comprising the amino acid sequence DYFDY (SEQ ID NO:58), and a light chain variable region comprising a light chain variable region CDR1 comprising the amino acid sequence SASSSVSYMY (SEQ ID NO:59), a light chain variable region CDR2 comprising the amino acid sequence DTSNLAS (SEQ ID NO:13), and a light chain variable region CDR3 comprising the amino acid sequence QQWSSYPYT (SEQ ID NO:60); or (e) a heavy chain variable region comprising a heavy chain variable region CDR1 comprising the amino acid sequence TSYWMH (SEQ ID NO:64), a heavy chain variable region CDR2 comprising the amino acid sequence WIGMIDPSDSETR (SEQ ID NO:65), and a heavy chain variable region CDR3 comprising the amino acid sequence TRDYFD (SEQ ID NO:66), and a light chain variable region comprising a light chain variable region CDR1 comprising the amino acid sequence SYMYWY (SEQ ID NO:22), a light chain variable region CDR2 comprising the amino acid sequence LLIYDTSNLA (SEQ ID NO:23), and a light chain variable region CDR3 comprising the amino acid sequence QQWSSYPY (SEQ ID NO:67).

In certain embodiments, a HTRA1-binding agent comprises a heavy chain variable region comprising a heavy chain variable region CDR1 comprising the amino acid sequence GYSFTSYWMH (SEQ ID NO:56), a heavy chain variable region CDR2 comprising the amino acid sequence MIDPSDSETRLNQKFKD (SEQ ID NO:57), and a heavy chain variable region CDR3 comprising the amino acid sequence DYFDY (SEQ ID NO:58). In some embodiments, a HTRA1-binding agent comprises a light chain variable region comprising a light chain variable region CDR1 comprising the amino acid sequence SASSSVSYMY (SEQ ID NO:59), a light chain variable region CDR2 comprising the amino acid sequence DTSNLAS (SEQ ID NO:13), and a light chain variable region CDR3 comprising the amino acid sequence QQWSSYPYT (SEQ ID NO:60). In certain embodiments, a HTRA1-binding agent comprises: (a) a heavy chain variable region comprising a heavy chain variable region CDR1 comprising the amino acid sequence GYSFTSYWMH (SEQ ID NO:56), a heavy chain variable region CDR2 comprising the amino acid sequence MIDPSDSETRLNQKFKD (SEQ ID NO:57), and a heavy chain variable region CDR3 comprising the amino acid sequence DYFDY (SEQ ID NO:58); and (b) a light chain variable region comprising a light chain variable region CDR1 comprising the amino acid sequence SASSSVSYMY (SEQ ID NO:59), a light chain variable region CDR2 comprising the amino acid sequence DTSNLAS (SEQ ID NO:13), and a light chain variable region CDR3 comprising the amino acid sequence QQWSSYPYT (SEQ ID NO:60).

In some embodiments, a HTRA1-binding agent comprises a heavy chain variable region having at least 80% sequence identity to SEQ ID NO:77. In some embodiments, a HTRA1-binding agent comprises a light chain variable region having at least 80% sequence identity to SEQ ID NO:78. In some embodiments, a HTRA1-binding agent comprises a heavy chain variable region having at least 85%, at least 90%, at least 95%, at least 97%, or at least 99% sequence identity to SEQ ID NO:77. In some embodiments, a HTRA1-binding agent comprises a light chain variable region having at least 85%, at least 90%, at least 95%, at least 97%, or at least 99% sequence identity to SEQ ID NO:78. In some embodiments, a HTRA1-binding agent comprises a heavy chain variable region comprising an amino acid sequence of SEQ ID NO:77. In some embodiments, a HTRA1-binding agent comprises a light chain variable region comprising an amino acid sequence of SEQ ID NO:78.

In some embodiments, a HTRA1-binding agent comprises a heavy chain variable region having at least 80% sequence identity to SEQ ID NO:77 and a light chain variable region having at least 80% sequence identity to SEQ ID NO:78. In some embodiments, a HTRA1-binding agent comprises a heavy chain variable region having at least 90% sequence identity to SEQ ID NO:77 and a light chain variable region having at least 90% sequence identity to SEQ ID NO:78. In some embodiments, a HTRA1-binding agent comprises a heavy chain variable region having at least 95% sequence identity to SEQ ID NO:77 and a light chain variable region having at least 95% sequence identity to SEQ ID NO:78. In some embodiments, a HTRA1-binding agent comprises a heavy chain variable region comprising an amino acid sequence of SEQ ID NO:77 and a light chain variable region comprising an amino acid sequence of SEQ ID NO:78.

In some embodiments, the HTRA1-binding agent is antibody 65G8. In some embodiments, the HTRA1-binding agent is a humanized version of antibody 65G8.

Provided herein are agents that compete with one or more of the binding agents described herein for binding to HTRA1. In some embodiments, an agent competes with one or more of the antibodies described herein for binding to HTRA1. In some embodiments, an agent that competes with one or more of the antibodies described herein is an antibody. In some embodiments, an agent binds the same epitope as one of the antibodies described herein. In some embodiments, an agent binds an epitope overlapping with an epitope bound by one of the antibodies described herein. Antibodies and antigen-binding fragments that compete with, or bind the same epitope, as the antibodies described herein are expected to show similar functional properties.

In some embodiments, an agent competes for binding to human HIRA1 with a reference antibody, wherein the reference antibody comprises: (a) a heavy chain variable region comprising a heavy chain variable region CDR1 comprising the amino acid sequence GYTFTDYEMH (SEQ ID NO:9), a heavy chain variable region CDR2 comprising the amino acid sequence AIDPETGGTAYNQKFKG (SEQ ID NO:10), and a heavy chain variable region CDR3 comprising the amino acid sequence EGYSYDGGGYYFDY (SEQ ID NO:11) or the amino acid sequence EGYSYEGGGYYFDY (SEQ ID NO:25), and (b) a light chain variable region comprising a light chain variable region CDR1 comprising the amino acid sequence SVSSSVSYMY (SEQ ID NO:12), a light chain variable region CDR2 comprising the amino acid sequence DTSNLAS (SEQ ID NO:13), and a light chain variable region CDR3 comprising the amino acid sequence QQWSSYPT (SEQ ID NO:14). In some embodiments, an agent competes for binding to human HIRA1 with a reference antibody, wherein the reference antibody comprises: (a) a heavy chain variable region comprising a heavy chain variable region CDR1 comprising the amino acid sequence GYTFTDYEMH (SEQ ID NO:9), a heavy chain variable region CDR2 comprising the amino acid sequence AIDPETGGTAYNQKFKG (SEQ ID NO:10), and a heavy chain variable region CDR3 comprising the amino acid sequence EGYSYDGGGYYFDY (SEQ ID NO:11) or the amino acid sequence EGYSYEGGGYYFDY (SEQ ID NO:25), and (b) a light chain variable region comprising a light chain variable region CDR1 comprising the amino acid sequence SVSSSVSYMY (SEQ ID NO:12), a light chain variable region CDR2 comprising the amino acid sequence DTSNLAS (SEQ ID NO:13), and a light chain variable region CDR3 comprising the amino acid sequence QQWSSYPT (SEQ ID NO:14); and wherein the competing agent comprises a heavy chain variable region comprising a heavy chain variable region CDR1 comprising the amino acid sequence GYAFTTYWMH (SEQ ID NO:27), a heavy chain variable region CDR2 comprising the amino acid sequence NIDPSDSETHYNQKFRD (SEQ ID NO:28), and a heavy chain variable region CDR3 comprising the amino acid sequence DYGAFDV (SEQ ID NO:29), and a light chain variable region comprising a light chain variable region CDR1 comprising the amino acid sequence RSSTGAVTTRNFAS (SEQ ID NO:30), a light chain variable region CDR2 comprising the amino acid sequence GTNNRAP (SEQ ID NO:31), and a light chain variable region CDR3 comprising the amino acid sequence ALWYSNLWV (SEQ ID NO:32). In some embodiments, an agent competes for binding to human HTRA1 with a reference antibody, wherein the reference antibody comprises: (a) a heavy chain variable region comprising a heavy chain variable region CDR1 comprising the amino acid sequence GYTFTDYEMH (SEQ ID NO:9), a heavy chain variable region CDR2 comprising the amino acid sequence AIDPETGGTAYNQKFKG (SEQ ID NO:10), and a heavy chain variable region CDR3 comprising the amino acid sequence EGYSYDGGGYYFDY (SEQ ID NO:11) or the amino acid sequence EGYSYEGGGYYFDY (SEQ ID NO:25), and (b) a light chain variable region comprising a light chain variable region CDR1 comprising the amino acid sequence SVSSSVSYMY (SEQ ID NO:12), a light chain variable region CDR2 comprising the amino acid sequence DTSNLAS (SEQ ID NO:13), and a light chain variable region CDR3 comprising the amino acid sequence QQWSSYPT (SEQ ID NO:14); and wherein the competing agent comprises a heavy chain variable region comprising a heavy chain variable region CDR1 comprising the amino acid sequence GYTFTNYWMH (SEQ ID NO:43), a heavy chain variable region the amino acid sequence CDR2 comprising NIDPSDSETHYNQKFKD (SEQ ID NO:44), and a heavy chain variable region CDR3 comprising the amino acid sequence EDSSGYGAY (SEQ ID NO:45), and a light chain variable region comprising a light chain variable region CDR1 comprising the amino acid sequence SASSSVNYMH (SEQ ID NO:46), a light chain variable region CDR2 comprising the amino acid sequence DTSKLAS (SEQ ID NO:47), and a light chain variable region CDR3 comprising the amino acid sequence QQWSSHPLT (SEQ ID NO:48). In some embodiments, an agent competes for binding to human HTRA1 with a reference antibody, wherein the reference antibody comprises: (a) a heavy chain variable region comprising a heavy chain variable region CDR1 comprising the amino acid sequence GYTFTDYEMH (SEQ ID NO:9), a heavy chain variable region CDR2 comprising the amino acid sequence AIDPETGGTAYNQKFKG (SEQ ID NO:10), and a heavy chain variable region CDR3 comprising the amino acid sequence EGYSYDGGGYYFDY (SEQ ID NO:11) or the amino acid sequence EGYSYEGGGYYFDY (SEQ ID NO:25), and (b) a light chain variable region comprising a light chain variable region CDR1 comprising the amino acid sequence SVSSSVSYMY (SEQ ID NO:12), a light chain variable region CDR2 comprising the amino acid sequence DTSNLAS (SEQ ID NO:13), and a light chain variable region CDR3 comprising the amino acid sequence QQWSSYPT (SEQ ID NO:14); and wherein the competing agent comprises a heavy chain variable region comprising a heavy chain variable region CDR1 comprising the amino acid sequence GYSFTSYWMH (SEQ ID NO:56), a heavy chain variable region CDR2 comprising the amino acid sequence MIDPSDSETRLNQKFKD (SEQ ID NO:57), and a heavy chain variable region CDR3 comprising the amino acid sequence DYFDY (SEQ ID NO:58), and a light chain variable region comprising a light chain variable region CDR1 comprising the amino acid sequence SASSSVSYMY (SEQ ID NO:59), a light chain variable region CDR2 comprising the amino acid sequence DTSNLAS (SEQ ID NO:13), and a light chain variable region CDR3 comprising the amino acid sequence QQWSSYPYT (SEQ ID NO:60).

In some embodiments, a HTRA1-binding agent described herein comprises an antibody in which at least one or more of the constant regions of the antibody has been modified or deleted. In some embodiments, an antibody comprises one or more modifications to one or more of the three heavy chain constant regions (CH1, CH2 or CH3) and/or to the light chain constant region (CL). In some embodiments, an antibody comprises one or more modifications to the hinge region. In some embodiments, the heavy chain constant region of the modified antibody comprises at least one human constant region. In some embodiments, the heavy chain constant region of the modified antibody comprises more than one human constant region. In some embodiments, modifications to the constant region comprise additions, deletions, or substitutions of one or more amino acids in one or more regions. In some embodiments, one or more regions are partially or entirely deleted from the constant regions of a modified antibody. In some embodiments, one or more regions are partially or entirely deleted from the hinge region of a modified antibody. In some embodiments, the entire CH2 domain has been removed from an antibody (ΔCH2 constructs). In some embodiments, a deleted constant region is replaced by a short amino acid spacer that provides some of the molecular flexibility typically imparted by the absent constant region. In some embodiments, a modified antibody comprises a CH3 domain directly fused to the hinge region of the antibody. In some embodiments, a modified antibody comprises a peptide spacer inserted between the hinge region and modified CH2 and/or CH3 domains.

It is known in the art that the constant region(s) of an antibody mediates several effector functions and these effector functions can vary depending on the isotype of the antibody. For example, binding of the Cl component of complement to the Fc region of IgG or IgM antibodies (bound to antigen) activates the complement system. Activation of complement is important in the opsonization and lysis of cell pathogens. The activation of complement also stimulates the inflammatory response and can be involved in autoimmune hypersensitivity. In addition, the Fc region of an antibody can bind a cell expressing a Fc receptor (FcR). There are a number of Fc receptors that are specific for different classes of antibody, including IgG (gamma receptors), IgE (epsilon receptors), IgA (alpha receptors) and IgM (mu receptors). Binding of antibody to Fc receptors on cell surfaces triggers a number of important and diverse biological responses including engulfment and destruction of antibody-coated particles, clearance of immune complexes, lysis of antibody-coated target cells by killer cells (called antibody-dependent cell cytotoxicity or ADCC), release of inflammatory mediators, placental transfer, and control of immunoglobulin production.

In some embodiments, a HTRA1-binding agent comprises a variant Fc region. The amino acid sequences of the Fc region of human IgG1, IgG2, IgG3, and IgG4 are known to those of ordinary skill in the art. A representative human IgG1 Fc region is set forth in SEQ ID NO:79. In some cases, Fc regions with amino acid variations have been identified in native antibodies. In some embodiments, a variant Fc region is engineered with substitutions at specific amino acid positions as compared to a native Fc region (e.g., SEQ ID NOs:80-84).

In some embodiments, a modified antibody provides for altered effector functions that, in turn, affect the biological profile of the antibody. For example, in some embodiments, the deletion or inactivation (through point mutations or other means) of a constant region reduces Fc receptor binding of a modified antibody as it circulates. In some embodiments, constant region modifications increase the serum half-life of an antibody. In some embodiments, constant region modifications reduce the serum half-life of an antibody. In some embodiments, constant region modifications decrease or remove ADCC and/or complement dependent cytotoxicity (CDC) of an antibody. In some embodiments, specific amino acid substitutions in a human IgG1 Fc region with corresponding IgG2 or IgG4 residues reduce effector functions in a modified antibody. In some embodiments, a modified antibody does not have one or more effector functions. In some embodiments, a modified antibody has no ADCC activity and/or no CDC activity. In some embodiments, a modified antibody does not bind an Fc receptor and/or complement factors. In some embodiments, a modified antibody has no effector function(s) (e.g., an “effectorless” antibody). In some embodiments, constant region modifications increase or enhance ADCC and/or CDC of an antibody. In some embodiments, the constant region is modified to eliminate disulfide linkages or oligosaccharide moieties. In some embodiments, the constant region is modified to add/substitute one or more amino acids to provide one or more cytotoxin, oligosaccharide, or carbohydrate attachment sites.

In some embodiments, a HTRA1-binding agent comprises a heavy chain having at least 80%, at least 85%, at least 90%, at least 95%, at least 96%, at least 97%, at least 98%, or at least 99% sequence identity to the amino acid sequence of SEQ ID NO:88. In some embodiments, a HTRA1-binding agent comprises a light chain having at least 80%, at least 85%, at least 90%, at least 95%, at least 96%, at least 97%, at least 98%, or at least 99% sequence identity to the amino acid sequence of SEQ ID NO:90. In some embodiments, a HTRA1-binding agent comprises a heavy chain having at least 80%, at least 85%, at least 90%, at least 95%, at least 96%, at least 97%, at least 98%, or at least 99% sequence identity to the amino acid sequence of SEQ ID NO:88 and a light chain having at least 80%, at least 85%, at least 90%, at least 95%, at least 96%, at least 97%, at least 98%, or at least 99% sequence identity to the amino acid sequence of SEQ ID NO:90. In some embodiments, a HTRA1-binding agent comprises a heavy chain having at least 90% sequence identity to the amino acid sequence of SEQ ID NO:88. In some embodiments, a HTRA1-binding agent comprises a light chain having at least 90% sequence identity to the amino acid sequence of SEQ ID NO:90. In some embodiments, a HTRA1-binding agent comprises a heavy chain having at least 90% sequence identity to the amino acid sequence of SEQ ID NO:88 and a light chain having at least 90% sequence identity to the amino acid sequence of SEQ ID NO:90. In some embodiments, a HTRA1-binding agent comprises a heavy chain comprising the amino acid sequence of SEQ ID NO:88. In some embodiments, a HTRA1-binding agent comprises a light chain comprising the amino acid sequence of SEQ ID NO:90. In some embodiments, a HTRA1-binding agent comprises a heavy chain comprising the amino acid sequence of SEQ ID NO:88 and a light chain comprising the amino acid sequence of SEQ ID NO:90. In some embodiments, a HTRA1-binding agent is an antibody that comprises a heavy chain comprising the amino acid sequence of SEQ ID NO:88 and/or a light chain comprising the amino acid sequence of SEQ ID NO:90. In some embodiments, a HTRA1-binding agent is an antibody that comprises a heavy chain comprising the amino acid sequence of SEQ ID NO:88. In some embodiments, a HTRA1-binding agent is an antibody that comprises a light chain comprising the amino acid sequence of SEQ ID NO:90. In some embodiments, a HTRA1-binding agent is an antibody that comprises a heavy chain of amino acid sequence SEQ ID NO:88 and a light chain of amino acid sequence SEQ ID NO:90.

Modifications to the constant region of antibodies described herein may be made using well-known biochemical or molecular engineering techniques. In some embodiments, antibody variants are prepared by introducing appropriate nucleotide changes into the encoding DNA, and/or by synthesis of the desired antibody or polypeptide. Using these engineering techniques to modify an antibody it may be possible to disrupt the activity or effector function provided by a specific sequence or region while substantially maintaining the structure, binding activity, and other desired characteristics of the modified antibody.

The present disclosure further embraces additional variants and equivalents that are substantially homologous to the recombinant, monoclonal, chimeric, humanized, and human antibodies, or antibody fragments thereof, described herein. In some embodiments, it is desirable to improve the binding affinity of the antibody. In some embodiments, it is desirable to modulate biological properties of the antibody, including but not limited to, specificity, thermostability, expression level, effector function(s), glycosylation, immunogenicity, and/or solubility. Those skilled in the art will appreciate that amino acid changes may alter post-translational processes of an antibody, such as changing the number or position of glycosylation sites or altering membrane anchoring characteristics.

Variations may be a substitution, deletion, or insertion of one or more nucleotides encoding the antibody or polypeptide that results in a change in the amino acid sequence as compared with the native antibody or polypeptide sequence. In some embodiments, amino acid substitutions are the result of replacing one amino acid with another amino acid having similar structural and/or chemical properties, such as the replacement of a leucine with a serine (i.e., conservative amino acid replacements). Insertions or deletions may optionally be in the range of about 1 to 5 amino acids. In some embodiments, the substitution, deletion, or insertion includes less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, or less than 2 amino acid substitutions relative to the parent molecule. In some embodiments, variations in the amino acid sequence that are biologically useful and/or relevant are determined by systematically making insertions, deletions, or substitutions in the sequence and testing the resulting variant proteins for activity as compared to the parental antibody.

In some embodiments, variants may include addition of amino acid residues at the amino- and/or carboxyl-terminal end of the antibody or polypeptide. The length of additional amino acids residues may range from one residue to a hundred or more residues. In some embodiments, a variant comprises an N-terminal methionyl residue. In some embodiments, the variant comprises an additional polypeptide/protein to create a fusion protein. In some embodiments, a variant is engineered to be detectable and may comprise a detectable label and/or protein (e.g., a fluorescent tag, a fluorescent protein, or an enzyme).

In some embodiments, a cysteine residue not involved in maintaining the proper conformation of an antibody is substituted or deleted to modulate the antibody's characteristics, for example, to improve oxidative stability and/or prevent aberrant disulfide crosslinking. Conversely, in some embodiments, one or more cysteine residues are added to create disulfide bond(s) to improve stability.

In some embodiments, an antibody of the present disclosure is “deimmunized”. The deimmunization of antibodies generally consists of introducing specific amino acid mutations (e.g., substitutions, deletions, additions) that result in removal of T-cell epitopes (known or predicted) without significantly reducing the binding affinity or other desired activities of the antibody.

The variant antibodies or polypeptides described herein may be generated using methods known in the art, including but not limited to, site-directed mutagenesis, alanine scanning mutagenesis, and PCR mutagenesis.

In some embodiments, a HTRA1-binding agent described herein is chemically modified. In some embodiments, a HTRA1-binding agent is an anti-HTRA1 antibody that is chemically modified by glycosylation, acetylation, pegylation, phosphorylation, amidation, derivatization by known protecting/blocking groups, proteolytic cleavage, and/or linkage to a cellular ligand or other protein. Any of numerous chemical modifications may be carried out by known techniques. In some embodiments, a HTRA1-binding agent is an antibody fragment as described herein. In some embodiments, an antibody fragment (e.g., scFv, Fv, Fab, F(ab′)₂, or F(ab′)) is attached, either directly or indirectly, to a half-life extending moiety including, but not limited to, a Fc region, a CH3 domain of an immunoglobulin, polyethylene glycol (PEG), a PEG mimetic, XTEN®, serum albumin, polysialic acid, N-(2-hydroxypropyl)methacrylamide, or dextran.

The present disclosure encompasses HTRA1-binding agents built upon non-immunoglobulin backbones, wherein the agents bind the same epitope or essentially the same epitope as an anti-HTRA1 antibody disclosed herein. In some embodiments, a non-immunoglobulin-based binding agent is an agent that competes with an anti-HTRA1 antibody described herein in a competitive binding assay. In some embodiments, alternative HTRA1-binding agents comprise a scaffold protein. Generally, scaffold proteins can be assigned to one of three groups based on the architecture of their backbone (1) scaffolds consisting of α-helices; (2) small scaffolds with few secondary structures or an irregular architecture of α-helices and β-sheets; and (3) scaffolds consisting of predominantly β-sheets. Scaffold proteins include, but are not limited to, anticalins, which are based upon the lipocalin scaffold; adnectins, which are based on the 10^{th} domain of human fibronectin type 3; affibodies, which are based on the B-domain in the Ig-binding region of Staphylococcus aureus protein A; darpins, which are based on ankyrin repeat domain proteins; fynomers, which are based on the SH3 domain of the human Fyn protein kinase; affitins, which are based on Sac7d from Sulfolobus acidocaldarius; affilins, which are based on human γ-B-crystallin or human ubiquitin; avimers, which are based on the A-domains of membrane receptor proteins; knottins (cysteine knot miniproteins), which are based upon a stable 30-amino acid anti-parallel β-strand protein fold; and Kunitz domain inhibitor scaffolds, which are based upon a structure that contains three disulfide bonds and three loops.

In some embodiments, a HTRA1-binding agent comprises an engineered scaffold protein comprising a heavy chain variable region CDR1, CDR2, and CDR3 and a light chain variable region CDR1, CDR2, and CDR3 shown in Table 1A. In some embodiments, a HTRA1-binding agent comprises an engineered scaffold protein comprising a heavy chain variable region CDR1 comprising the amino acid sequence GYTFTDYEMH (SEQ ID NO:9), a heavy chain variable region CDR2 comprising the amino acid sequence AIDPETGGTAYNQKFKG (SEQ ID NO:10), a heavy chain variable region CDR3 comprising the amino acid sequence EGYSYDGGGYYFDY (SEQ ID NO:11), a light chain variable region CDR1 comprising the amino acid sequence SVSSSVSYMY (SEQ ID NO:12), a light chain variable region CDR2 comprising the amino acid sequence DTSNLAS (SEQ ID NO:13), and a light chain variable region CDR3 comprising the amino acid sequence QQWSSYPT (SEQ ID NO:14). In some embodiments, a HTRA1-binding agent comprises an engineered scaffold protein comprising a heavy chain variable region CDR1, CDR2, and CDR3 and a light chain variable region CDR1, CDR2, and CDR3 from antibody 24F7. In some embodiments, a HTRA1-binding agent comprises an engineered scaffold protein comprising a heavy chain variable region CDR1, CDR2, and CDR3 and a light chain variable region CDR1, CDR2, and CDR3 shown in Table 1B. In some embodiments, a HTRA1-binding agent comprises an engineered scaffold protein comprising a heavy chain variable region CDR1 comprising the amino acid sequence GYTFTDYEMH (SEQ ID NO:9), a heavy chain variable region CDR2 comprising the amino acid sequence AIDPETGGTAYNQKFKG (SEQ ID NO:10), a heavy chain variable region CDR3 comprising the amino acid sequence EGYSYEGGGYYFDY (SEQ ID NO:25), a light chain variable region CDR1 comprising the amino acid sequence SVSSSVSYMY (SEQ ID NO:12), a light chain variable region CDR2 comprising the amino acid sequence DTSNLAS (SEQ ID NO:13), and a light chain variable region CDR3 comprising the amino acid sequence QQWSSYPT (SEQ ID NO:14). In some embodiments, a HTRA1-binding agent comprises an engineered scaffold protein comprising a heavy chain variable region CDR1, CDR2, and CDR3 and a light chain variable region CDR1, CDR2, and CDR3 from antibody hz24F7.v2. In some embodiments, a HTRA1-binding agent comprises an engineered scaffold protein comprising a heavy chain variable region CDR1, CDR2, and CDR3 and a light chain variable region CDR1, CDR2, and CDR3 shown in Table 1C. In some embodiments, a HTRA1-binding agent comprises an engineered scaffold protein comprising a heavy chain variable region CDR1 comprising the amino acid sequence GYTFTDYEMH (SEQ ID NO:9), a heavy chain variable region CDR2 comprising the amino acid sequence AIDPETGGTAYNQKFKG (SEQ ID NO:10), a heavy chain variable region CDR3 comprising the amino acid sequence EGYSYEGGGYYFDY (SEQ ID NO:25), a light chain variable region CDR1 comprising the amino acid sequence SVSSSVSYMY (SEQ ID NO:12), a light chain variable region CDR2 comprising the amino acid sequence DTSNLAS (SEQ ID NO:13), and a light chain variable region CDR3 comprising the amino acid sequence QQWSSYPT (SEQ ID NO:94). In some embodiments, a HTRA1-binding agent comprises an engineered scaffold protein comprising a heavy chain variable region CDR1, CDR2, and CDR3 and a light chain variable region CDR1, CDR2, and CDR3 from antibody hz24F7.v2. In some embodiments, a HTRA1-binding agent comprises an engineered scaffold protein comprising a heavy chain variable region CDR1, CDR2, and CDR3 and a light chain variable region CDR1, CDR2, and CDR3 shown in Table 1D. In some embodiments, a HTRA1-binding agent comprises an engineered scaffold protein comprising a heavy chain variable region CDR1 comprising the amino acid sequence GYTFTDYEMH (SEQ ID NO:9), a heavy chain variable region CDR2 comprising the amino acid sequence AIDPETGGTAYNQKFKG (SEQ ID NO:10), a heavy chain variable region CDR3 comprising the amino acid sequence EGYSYEGGGYYFDY (SEQ ID NO:25), a light chain variable region CDR1 comprising the amino acid sequence SVSSSVSYMY (SEQ ID NO:12), a light chain variable region CDR2 comprising the amino acid sequence DTSNLAS (SEQ ID NO:13), and a light chain variable region CDR3 comprising the amino acid sequence QQWSSYPT (SEQ ID NO:99). In some embodiments, a HTRA1-binding agent comprises an engineered scaffold protein comprising a heavy chain variable region CDR1, CDR2, and CDR3 and a light chain variable region CDR1, CDR2, and CDR3 from antibody hz24F7.v2. In some embodiments, a HTRA1-binding agent comprises an engineered scaffold protein comprising a heavy chain variable region CDR1, CDR2, and CDR3 and a light chain variable region CDR1, CDR2, and CDR3 shown in Table 1E. In some embodiments, a HTRA1-binding agent comprises an engineered scaffold protein comprising a heavy chain variable region CDR1 comprising the amino acid sequence GYTFTDYEMH (SEQ ID NO:9), a heavy chain variable region CDR2 comprising the amino acid sequence AIDPETGGTAYNQKFKG (SEQ ID NO:10), a heavy chain variable region CDR3 comprising the amino acid sequence EGYSYEGGGYYFDY (SEQ ID NO:25), a light chain variable region CDR1 comprising the amino acid sequence SVSSSVSYMY (SEQ ID NO:12), a light chain variable region CDR2 comprising the amino acid sequence DTSNLAS (SEQ ID NO:13), and a light chain variable region CDR3 comprising the amino acid sequence QQWDSYPT (SEQ ID NO:104). In some embodiments, a HTRA1-binding agent comprises an engineered scaffold protein comprising a heavy chain variable region CDR1, CDR2, and CDR3 and a light chain variable region CDR1, CDR2, and CDR3 from antibody hz24F7.v2. In some embodiments, a HTRA1-binding agent comprises an engineered scaffold protein comprising a heavy chain variable region CDR1, CDR2, and CDR3 and a light chain variable region CDR1, CDR2, and CDR3 shown in Table 1F. In some embodiments, a HTRA1-binding agent comprises an engineered scaffold protein comprising a heavy chain variable region CDR1 comprising the amino acid sequence GYTFTDYEMH (SEQ ID NO:9), a heavy chain variable region CDR2 comprising the amino acid sequence AIDPETGGTAYNQKFKG (SEQ ID NO:10), a heavy chain variable region CDR3 comprising the amino acid sequence EGYSYEGGGYYFDY (SEQ ID NO:25), a light chain variable region CDR1 comprising the amino acid sequence SVSSSVSYMY (SEQ ID NO:12), a light chain variable region CDR2 comprising the amino acid sequence DTSNLAS (SEQ ID NO:13), and a light chain variable region CDR3 comprising the amino acid sequence QQWTSYPT (SEQ ID NO:107). In some embodiments, a HTRA1-binding agent comprises an engineered scaffold protein comprising a heavy chain variable region CDR1, CDR2, and CDR3 and a light chain variable region CDR1, CDR2, and CDR3 from antibody hz24F7.v2. In some embodiments, a HTRA1-binding agent comprises an engineered scaffold protein comprising a heavy chain variable region CDR1, CDR2, and CDR3 and a light chain variable region CDR1, CDR2, and CDR3 shown in Table 1G. In some embodiments, a HTRA1-binding agent comprises an engineered scaffold protein comprising a heavy chain variable region CDR1 comprising the amino acid sequence GYTFTDYEMH (SEQ ID NO:9), a heavy chain variable region CDR2 comprising the amino acid sequence AIDPETGGTAYNQKFKG (SEQ ID NO:10), a heavy chain variable region CDR3 comprising the amino acid sequence EGYSYEGGGYYFDY (SEQ ID NO:25), a light chain variable region CDR1 comprising the amino acid sequence SVSSSVSYMY (SEQ ID NO:12), a light chain variable region CDR2 comprising the amino acid sequence DTSNLAS (SEQ ID NO:13), and a light chain variable region CDR3 comprising the amino acid sequence QQWASYPT (SEQ ID NO:110). In some embodiments, a HTRA1-binding agent comprises an engineered scaffold protein comprising a heavy chain variable region CDR1, CDR2, and CDR3 and a light chain variable region CDR1, CDR2, and CDR3 from antibody hz24F7.v2. In some embodiments, a HTRA1-binding agent comprises an engineered scaffold protein comprising a heavy chain variable region CDR1, CDR2, and CDR3 and a light chain variable region CDR1, CDR2, and CDR3 shown in Table 1H. In some embodiments, a HTRA1-binding agent comprises an engineered scaffold protein comprising a heavy chain variable region CDR1 comprising the amino acid sequence GYTFTDYEMH (SEQ ID NO:9), a heavy chain variable region CDR2 comprising the amino acid sequence AIDPETGGTAYNQKFKG (SEQ ID NO:10), a heavy chain variable region CDR3 comprising the amino acid sequence EGYSYEGGGYYFDY (SEQ ID NO:25), a light chain variable region CDR1 comprising the amino acid sequence SVSSSVSYMY (SEQ ID NO:12), a light chain variable region CDR2 comprising the amino acid sequence DTSNLAS (SEQ ID NO:13), and a light chain variable region CDR3 comprising the amino acid sequence QQWLSYPT (SEQ ID NO:113). In some embodiments, a HTRA1-binding agent comprises an engineered scaffold protein comprising a heavy chain variable region CDR1, CDR2, and CDR3 and a light chain variable region CDR1, CDR2, and CDR3 from antibody hz24F7.v2. In some embodiments, a HTRA1-binding agent comprises an engineered scaffold protein comprising a heavy chain variable region CDR1, CDR2, and CDR3 and a light chain variable region CDR1, CDR2, and CDR3 shown in Table 1I. In some embodiments, a HTRA1-binding agent comprises an engineered scaffold protein comprising a heavy chain variable region CDR1 comprising the amino acid sequence GYTFTDYEMH (SEQ ID NO:9), a heavy chain variable region CDR2 comprising the amino acid sequence AIDPETGGTAYNQKFKG (SEQ ID NO:10), a heavy chain variable region CDR3 comprising the amino acid sequence EGYSYEGGGYYFDY (SEQ ID NO:25), a light chain variable region CDR1 comprising the amino acid sequence SVSSSVSYMY (SEQ ID NO:12), a light chain variable region CDR2 comprising the amino acid sequence DTSNLAS (SEQ ID NO:13), and a light chain variable region CDR3 comprising the amino acid sequence QQWSYYPT (SEQ ID NO:116). In some embodiments, a HTRA1-binding agent comprises an engineered scaffold protein comprising a heavy chain variable region CDR1, CDR2, and CDR3 and a light chain variable region CDR1, CDR2, and CDR3 from antibody hz24F7.v2. In some embodiments, a HTRA1-binding agent comprises an engineered scaffold protein comprising a heavy chain variable region CDR1, CDR2, and CDR3 and a light chain variable region CDR1, CDR2, and CDR3 shown in Table 1J. In some embodiments, a HTRA1-binding agent comprises an engineered scaffold protein comprising a heavy chain variable region CDR1 comprising the amino acid sequence GYTFTDYEMH (SEQ ID NO:9), a heavy chain variable region CDR2 comprising the amino acid sequence AIDPETGGTAYNQKFKG (SEQ ID NO:10), a heavy chain variable region CDR3 comprising the amino acid sequence EGYSYEGGGYYFDY (SEQ ID NO:25), a light chain variable region CDR1 comprising the amino acid sequence SVSSSVSYMY (SEQ ID NO:12), a light chain variable region CDR2 comprising the amino acid sequence DTSNLAS (SEQ ID NO:13), and a light chain variable region CDR3 comprising the amino acid sequence QQWSDYPT (SEQ ID NO:119). In some embodiments, a HTRA1-binding agent comprises an engineered scaffold protein comprising a heavy chain variable region CDR1, CDR2, and CDR3 and a light chain variable region CDR1, CDR2, and CDR3 from antibody hz24F7.v2.

In some embodiments, a HTRA1-binding agent comprises an engineered scaffold protein comprising a heavy chain variable region CDR1, CDR2, and CDR3 and a light chain variable region CDR1, CDR2, and CDR3 shown in Table 2. In some embodiments, a HTRA1-binding agent comprises an engineered scaffold protein comprising a heavy chain variable region CDR1 comprising the amino acid sequence GYAFTTYWMH (SEQ ID NO:27), a heavy chain variable region CDR2 comprising the amino acid sequence NIDPSDSETHYNQKFRD (SEQ ID NO:28), a heavy chain variable region CDR3 comprising the amino acid sequence DYGAFDV (SEQ ID NO:29), a light chain variable region CDR1 comprising the amino acid sequence RSSTGAVTTRNFAS (SEQ ID NO:30), a light chain variable region CDR2 comprising the amino acid sequence GTNNRAP (SEQ ID NO:31), and a light chain variable region CDR3 comprising the amino acid sequence ALWYSNLWV (SEQ ID NO:32). In some embodiments, a HTRA1-binding agent comprises an engineered scaffold protein comprising a heavy chain variable region CDR1, CDR2, and CDR3 and a light chain variable region CDR1, CDR2, and CDR3 from antibody 9F8.

In some embodiments, a HTRA1-binding agent comprises an engineered scaffold protein comprising a heavy chain variable region CDR1, CDR2, and CDR3 and a light chain variable region CDR1, CDR2, and CDR3 shown in Table 3. In some embodiments, a HTRA1-binding agent comprises an engineered scaffold protein comprising a heavy chain variable region CDR1 comprising the amino acid sequence GYTFTNYWMH (SEQ ID NO:43), a heavy chain variable region CDR2 comprising the amino acid sequence NIDPSDSETHYNQKFKD (SEQ ID NO:44), a heavy chain variable region CDR3 comprising the amino acid sequence EDSSGYGAY (SEQ ID NO:45), a light chain variable region CDR1 comprising the amino acid sequence SASSSVNYMH (SEQ ID NO:46), a light chain variable region CDR2 comprising the amino acid sequence DTSKLAS (SEQ ID NO:47), and a light chain variable region CDR3 comprising the amino acid sequence QQWSSHPLT (SEQ ID NO:48). In some embodiments, a HTRA1-binding agent comprises an engineered scaffold protein comprising a heavy chain variable region CDR1, CDR2, and CDR3 and a light chain variable region CDR1, CDR2, and CDR3 from antibody 55B12.

In some embodiments, a HTRA1-binding agent comprises an engineered scaffold protein comprising a heavy chain variable region CDR1, CDR2, and CDR3 and a light chain variable region CDR1, CDR2, and CDR3 shown in Table 4. In some embodiments, a HTRA1-binding agent comprises an engineered scaffold protein comprising a heavy chain variable region CDR1 comprising the amino acid sequence GYSFTSYWMH (SEQ ID NO:56), a heavy chain variable region CDR2 comprising the amino acid sequence MIDPSDSETRLNQKFKD (SEQ ID NO:57), a heavy chain variable region CDR3 comprising the amino acid sequence DYFDY (SEQ ID NO:58), a light chain variable region CDR1 comprising the amino acid sequence SASSSVSYMY (SEQ ID NO:59), a light chain variable region CDR2 comprising the amino acid sequence DTSNLAS (SEQ ID NO:13), and a light chain variable region CDR3 comprising the amino acid sequence QQWSSYPYT (SEQ ID NO:60). In some embodiments, a HTRA1-binding agent comprises an engineered scaffold protein comprising a heavy chain variable region CDR1, CDR2, and CDR3 and a light chain variable region CDR1, CDR2, and CDR3 from antibody 65G8.

In some embodiments, a composition comprises a HTRA1-binding agent described herein. In some embodiments, a composition comprises an anti-HTRA1 antibody described herein. In some embodiments, a composition comprises a monoclonal anti-HTRA1 antibody described herein. In some embodiments, a composition comprises an anti-HTRA1 antibody selected from the group consisting of: 24F7, hz24F7.v2, 9F8, 55B12, and 65G8.

In some embodiments, a pharmaceutical composition comprises a HTRA1-binding agent described herein and a pharmaceutically acceptable carrier. In some embodiments, a pharmaceutical composition comprises an anti-HTRA1 antibody described herein and a pharmaceutically acceptable carrier. In some embodiments, a pharmaceutical composition comprises a monoclonal anti-HTRA1 antibody described herein and a pharmaceutically acceptable carrier. In some embodiments, a pharmaceutical composition comprises an anti-HTRA1 antibody selected from the group consisting of: 24F7, hz24F7.v2, 9F8, 55B12, and 65G8 and a pharmaceutically acceptable carrier.

In some embodiments, a HTRA1-binding agent is isolated. In some embodiments, a HTRA1-binding agent is substantially pure.

Generally speaking, antigen-antibody interactions are non-covalent and reversible, formed by a combination of hydrogen bonds, hydrophobic interactions, electrostatic and van der Waals forces. When describing the strength of an antigen-antibody complex, the terms affinity and/or avidity are commonly used. The binding of an antibody to its antigen is a reversible process, and the affinity of the binding is typically reported as an equilibrium dissociation constant (K_{D}). K_{D} is the ratio of an antibody dissociation rate (k_{off}) (how quickly it dissociates from its antigen) to the antibody association rate (kₒₙ) (how quickly it binds to its antigen). In some embodiments, K_{D} values are determined by measuring the kₒₙ and k_{off} rates of a specific antibody/antigen interaction and then using a ratio of these values to calculate the K_{D} value. K_{D} values may be used to evaluate and rank order the strength of individual antibody/antigen interactions. The lower the K_{D} of an antibody, the higher the affinity of the antibody for its target. In some embodiments, affinity is measured using SPR technology in a Biacore system. Avidity gives a measure of the overall strength of an antibody-antigen complex. It is dependent on three major parameters: (i) affinity of the antibody for the target, (ii) valency of both the antibody and antigen, and (iii) structural arrangement of the parts that interact.

In some embodiments, a HTRA1-binding agent binds HTRA1 with a dissociation constant (K_{D}) of 1 μM or less, 100 nM or less, 40 nM or less, 20 nM or less, 10 nM or less, 1 nM or less, 0.1 nM or less, 50 pM or less, 10 pM or less, or 1 pM or less. In some embodiments, a HTRA1-binding agent binds HTRA1 with a K_{D} of 20 nM or less. In some embodiments, a HTRA1-binding agent binds HTRA1 with a K_{D} of 10 nM or less. In some embodiments, a HTRA1-binding agent binds HTRA1 with a K_{D} of 5 nM or less. In some embodiments, a HTRA1-binding agent binds HTRA1 with a K_{D} of 3 nM or less. In some embodiments, a HTRA1-binding agent binds HTRA1 with a K_{D} of 2 nM or less. In some embodiments, a HTRA1-binding agent binds HTRA1 with a K_{D} of 1 nM or less. In some embodiments, a HTRA1-binding agent binds HTRA1 with a K_{D} of 0.5 nM or less. In some embodiments, a HTRA1-binding agent binds HTRA1 with a K_{D} of 0.1 nM or less. In some embodiments, a HTRA1-binding agent binds HTRA1 with a K_{D} of 50 pM or less. In some embodiments, a HTRA1-binding agent binds HTRA1 with a K_{D} of 25 pM or less. In some embodiments, a HTRA1-binding agent binds HIRA1 with a K_{D} of 10 pM or less. In some embodiments, a HTRA1-binding agent binds HTRA1 with a K_{D} of 1 pM or less. In some embodiments, a HTRA1-binding agent binds HTRA1 with a K_{D} of 0.01 nM to 2.5 nM. In some embodiments, a HTRA1-binding agent binds HTRA1 with a K_{D} of 0.1 nM to 5 nM. In some embodiments, a HTRA1-binding agent binds HTRA1 with a K_{D} of 1 nM to 5 nM. In some embodiments, the dissociation constant of the binding agent for HTRA1 is the dissociation constant determined using a HTRA1 protein, or a fragment thereof, immobilized on a Biacore chip and the binding agent flowed over the chip. In some embodiments, the dissociation constant of the binding agent for HTRA1 is the dissociation constant determined using the binding agent captured by an anti-human IgG antibody on a Biacore chip and soluble HTRA1 flowed over the chip.

In some embodiments, a HTRA1-binding agent binds HTRA1 with a half maximal effective concentration (EC50) of 1 μM or less, 100 nM or less, 40 nM or less, 20 nM or less, 10 nM or less, 1 nM or less, or 0.1 nM or less. In some embodiments, a HTRA1-binding agent binds human HTRA1 with an EC50 of 1 μM or less, 100 nM or less, 40 nM or less, 20 nM or less, 10 nM or less, 1 nM or less, or 0.1 nM or less. In some embodiments, a HTRA1-binding agent binds cyno HTRA1 and/or human HTRA1 with an EC50 of 40 nM or less, 20 nM or less, 10 nM or less, 1 nM or less or 0.1 nM or less. In some embodiments, a HTRA1-binding agent binds HTRA1 with an EC50 of 0.1 nM to 3 nM, 0.1 nM to 2 nM, 0.1 nM to 1 nM, 0.5 nM to 3 nM, 0.5 nM to 2 nM, or 0.5 nM to 1 nM.

In some embodiments, a HTRA1-binding agent binds human HTRA1 and has at least one or more of the following properties: (a) binds cyno HTRA1, (b) binds rabbit HTRA1, (c) inhibits HTRA1 protease activity, (d) inhibits HTRA1 protease activity in an allosteric manner, and (e) does not inhibit protease activity of other proteases in HTRA family.

Assays for determining inhibition of protease activity are known in the art. In certain embodiments, the HTRA1-binding agent inhibits HTRA1 protease activity. In certain embodiments, a HTRA1-binding agent inhibits HTRA1 protease activity, wherein there is a reduction in the level of HTRA1 protease activity by at least 10%, at least 20%, at least 30%, at least 40%, at least 50%, at least 60%, at least 70%, at least 80%, at least 90%, or at least 95% as compared to HTRA1 protease activity in the absence of the HTRA1-binding agent.

The HTRA1-binding agents described herein can be produced by any suitable method known in the art. Such methods range from direct protein synthesis methods to constructing a DNA sequence encoding polypeptide sequences and expressing those sequences in a suitable host. In some embodiments, a DNA sequence is constructed using recombinant technology by isolating or synthesizing a DNA sequence encoding a wild-type protein of interest. Optionally, the sequence can be mutagenized by site-specific mutagenesis to provide functional variants thereof. In some embodiments, a DNA sequence encoding a polypeptide of interest is constructed by chemical synthesis using an oligonucleotide synthesizer. Oligonucleotides can be designed based on the amino acid sequence of the desired polypeptide and selecting those codons that are favored in the host cell in which the recombinant polypeptide of interest will be produced. Standard methods can be applied to synthesize a polynucleotide sequence encoding an isolated polypeptide of interest. For example, a complete amino acid sequence can be used to construct a back-translated gene. Further, a DNA oligomer containing a nucleotide sequence coding for the particular isolated polypeptide can be synthesized. For example, several small oligonucleotides coding for portions of the desired polypeptide can be synthesized and then ligated. The individual oligonucleotides typically contain 5′ or 3′ overhangs for complementary assembly.

Once assembled (by synthesis, site-directed mutagenesis, or another method), the polynucleotide sequences encoding a particular polypeptide of interest can be inserted into an expression vector and operatively linked to an expression control sequence appropriate for expression of the protein in a desired host. Proper assembly can be confirmed by nucleotide sequencing, restriction enzyme mapping, and/or expression of a biologically active polypeptide in a suitable host. As is well-known in the art, in order to obtain high expression levels of a transfected gene in a host, the gene must be operatively linked to transcriptional and translational expression control sequences that are functional in the chosen expression host.

In some embodiments, recombinant expression vectors are used to amplify and express DNA encoding antibodies, or fragments thereof, against human HTRA1. For example, recombinant expression vectors can be replicable DNA constructs which have synthetic or cDNA-derived DNA fragments encoding a polypeptide chain of a HTRA1-binding agent, such as an anti-HTRA1 antibody, or antigen-binding fragment thereof, operatively linked to suitable transcriptional and/or translational regulatory elements derived from mammalian, microbial, viral or insect genes. A transcriptional unit generally comprises an assembly of (1) a genetic element or elements having a regulatory role in gene expression, for example, transcriptional promoters or enhancers, (2) a structural or coding sequence that is transcribed into mRNA and translated into protein, and (3) appropriate transcription and translation initiation and termination sequences. Regulatory elements can include an operator sequence to control transcription. The ability to replicate in a host, usually conferred by an origin of replication, and a selection gene to facilitate recognition of transformants can additionally be incorporated. DNA regions are “operatively linked” when they are functionally related to each other. For example, DNA for a signal peptide (secretory leader) is operatively linked to DNA for a polypeptide if it is expressed as a precursor that participates in the secretion of the polypeptide; a promoter is operatively linked to a coding sequence if it controls the transcription of the sequence; or a ribosome binding site is operatively linked to a coding sequence if it is positioned so as to permit translation. In some embodiments, structural elements intended for use in yeast expression systems include a leader sequence enabling extracellular secretion of translated protein by a host cell. In some embodiments, in situations where recombinant protein is expressed without a leader or transport sequence, a polypeptide may include an N-terminal methionine residue. This residue can optionally be subsequently cleaved from the expressed recombinant protein to provide a final product.

The choice of an expression control sequence and an expression vector generally depends upon the choice of host. A wide variety of expression host/vector combinations can be employed. Useful expression vectors for eukaryotic hosts include, for example, vectors comprising expression control sequences from SV40, bovine papilloma virus, adenovirus, and cytomegalovirus. Useful expression vectors for bacterial hosts include known bacterial plasmids, such as plasmids from E. coli, including pCR1, pBR322, pMB9 and their derivatives, and wider host range plasmids, such as M13 and other filamentous single-stranded DNA phages. Mammalian expression vectors can comprise non-transcribed elements such as an origin of replication, a suitable promoter and enhancer linked to the gene to be expressed, and other 5′ or 3′ flanking non-transcribed sequences, and 5′ or 3′ non-translated sequences, such as necessary ribosome binding sites, a polyadenylation site, splice donor and acceptor sites, and transcriptional termination sequences.

In some embodiments, a HTRA1-binding agent of the present disclosure is expressed from one or more vectors. In some embodiments, a heavy chain variable region polypeptide is expressed by one vector and a light chain variable region polypeptide is expressed by a second vector. In some embodiments, a heavy chain variable region polypeptide and a light chain variable region polypeptide is expressed by one vector. In some embodiments, a heavy chain polypeptide is expressed by one vector and a light chain polypeptide is expressed by a second vector. In some embodiments, a heavy chain polypeptide and a light chain polypeptide are expressed by one vector. In some embodiments, a vector encodes a heavy chain variable region polypeptide of a HTRA1-binding agent described herein. In some embodiments, a vector encodes a light chain variable region polypeptide of a HTRA1-binding agent described herein. In some embodiments, a vector encodes a heavy chain variable region polypeptide and a light chain variable region polypeptide of a HTRA1-binding agent described herein. In some embodiments, a vector encodes a heavy chain polypeptide of a HTRA1-binding agent described herein. In some embodiments, a vector encodes a light chain polypeptide of a HTRA1-binding agent described herein. In some embodiments, a vector encodes a heavy chain polypeptide and a light chain polypeptide of a HTRA1-binding agent described herein.

Suitable host cells for expression of a HTRA1-binding agent or a HTRA1 protein or fragment thereof to use as an antigen or immunogen include prokaryotes, yeast cells, insect cells, or higher eukaryotic cells under the control of appropriate promoters. Prokaryotes include gram-negative or gram-positive organisms, for example E. coli or Bacillus. Higher eukaryotic cells include established cell lines of mammalian origin as described herein. Cell-free translation systems may also be employed. Appropriate cloning and expression vectors for use with bacterial, fungal, yeast, and mammalian cellular hosts, as well as methods of protein production, including antibody production are well known in the art.

Various mammalian culture systems may be used to express recombinant polypeptides. Expression of recombinant proteins in mammalian cells may be desirable because these proteins are generally correctly folded, appropriately modified, and biologically functional. Examples of suitable mammalian host cell lines include, but are not limited to, COS-7 (monkey kidney-derived), L-929 (murine fibroblast-derived), C127 (murine mammary tumor-derived), 3T3 (murine fibroblast-derived), CHO (Chinese hamster ovary-derived), HeLa (human cervical cancer-derived), BHK (hamster kidney fibroblast-derived), HEK-293 (human embryonic kidney-derived) cell lines and variants thereof.

Expression of recombinant proteins in insect cell culture systems (e.g., baculovirus) also offers a robust method for producing correctly folded and biologically functional proteins. Baculovirus systems for production of heterologous proteins in insect cells are well-known to those of skill in the art.

Thus, the present disclosure provides cells comprising the HTRA1-binding agents described herein. In some embodiments, a cell produces the HTRA1-binding agents described herein. In some embodiments, a cell produces an antibody. In some embodiments, a cell produces an antibody that binds human HTRA1. In some embodiments, a cell produces an antibody that binds cyno HTRA1. In some embodiments, a cell produces an antibody that binds human HTRA1 and cyno HTRA1. In some embodiments, a cell produces an antibody designated 24F7. In some embodiments, a cell produces a humanized version of antibody 24F7, referred to as hz24F7. In some embodiments, a cell produces a variant of hz24F7, for example, hz24F7.v2. In some embodiments, a cell produces an antibody designated 9F8. In some embodiments, a cell produces a humanized version of antibody 9F8. In some embodiments, a cell produces an antibody designated 55B12. In some embodiments, a cell produces a humanized version of antibody 55B12. In some embodiments, a cell produces an antibody designated 65G8. In some embodiments, a cell produces a humanized version of antibody 65G8. In some embodiments, the cell is a prokaryotic cell (e.g., E. coli). In some embodiments, the cell is an eukaryotic cell. In some embodiments, the cell is a mammalian cell. In some embodiments, the cell is a hybridoma cell.

Proteins produced by a host cell can be purified according to any suitable method. Standard methods include chromatography (e.g., ion exchange, affinity, and sizing column chromatography), centrifugation, differential solubility, or by any other standard technique for protein purification. Affinity tags such as hexa-histidine (SEQ ID NO:93), maltose binding domain, influenza coat sequence, and glutathione-S-transferase can be attached to the protein to allow easy purification by passage over an appropriate affinity column. Affinity chromatography methods used for purifying immunoglobulins can include, but are not limited to, Protein A, Protein G, and Protein L chromatography. Isolated proteins can be physically characterized using techniques that include, but are not limited to, proteolysis, size exclusion chromatography (SEC), mass spectrometry (MS), nuclear magnetic resonance (NMR), isoelectric focusing (IEF), high performance liquid chromatography (HPLC), and x-ray crystallography. The purity of isolated proteins can be determined using techniques known to those of skill in the art, including but not limited to, SDS-PAGE, SEC, capillary gel electrophoresis, IEF, and capillary isoelectric focusing (cIEF).

In some embodiments, supernatants from expression systems that secrete recombinant protein into culture media are first concentrated using a commercially available protein concentration filter, for example, an Amicon® or Millipore Pellicon® ultrafiltration unit. Following the concentration step, the concentrate can be applied to a suitable purification matrix. In some embodiments, an anion exchange resin is employed, for example, a matrix or substrate having pendant diethylaminoethyl (DEAE) groups. The matrices can be acrylamide, agarose, dextran, cellulose, or other types commonly employed in protein purification. In some embodiments, a cation exchange step is employed. Suitable cation exchangers include various insoluble matrices comprising sulfopropyl or carboxymethyl groups. In some embodiments, a hydroxyapatite media is employed, including but not limited to, ceramic hydroxyapatite (CHT). In some embodiments, one or more reverse-phase HPLC steps employing hydrophobic RP-HPLC media, e.g., silica gel having pendant methyl or other aliphatic groups, are employed to further purify a recombinant protein. In some embodiments, hydrophobic interaction chromatography (HIC) is used to separate recombinant proteins based on their hydrophobicity. HIC is a useful separation technique for purifying proteins while maintaining biological activity due to the use of conditions and matrices that operate under less denaturing conditions than some other techniques. In some embodiments, a viral inactivation step and/or a viral filtration step is employed. Some or all of the foregoing purification steps, in various combinations, can be employed to provide a homogeneous recombinant protein.

HTRA1-binding agents of the present disclosure may be analyzed for their physical/chemical properties and/or biological activities by various assays known in the art. In some embodiments, an anti-HTRA1 antibody is tested for its ability to bind HTRA1 (e.g., human HTRA1 and/or cyno HTRA1). Binding assays include, but are not limited to, SPR (e.g., Biacore), ELISA, and FACS. In some embodiments, an anti-HTRA1 antibody is tested for its ability to inhibit, reduce, or block HTRA1 protease activity. Assays include, but are not limited to, serine protease assays using protein and/or peptide substrates. In addition, antibodies may be evaluated for solubility, stability, thermostability, viscosity, expression levels, expression quality, and/or purification efficiency.

In some embodiments, monoclonal antibodies generated against HTRA1 are grouped based upon the epitope each individual antibody recognizes, a process known as “epitope binning” Generally, antibodies are tested in a pairwise combinatorial manner and antibodies that compete with each other are grouped together into bins. For example, in a premix binning assay, a first antibody is immobilized on a surface and a premixed solution of a second antibody and antigen is flowed over the immobilized first antibody. In tandem, the antigen is immobilized on a surface and the two antibodies are flowed over the immobilized antigen and compete to bind. Using these techniques, antibodies that block one another can be identified. A competitive blocking profile is created for each antibody relative to the other antibodies. The blocking results determine which bin each antibody is placed in. High-throughput methods of epitope binning are known in the art and allow for screening and characterization of large numbers of antibodies within a short period of time. Antibodies that bind similar epitopes often share similar functions and/or capabilities. Conversely, antibodies that bind different epitopes may have different functional activities.

In some embodiments, an epitope bin comprises at least one antibody from the group consisting of: 24F7, 9F8, 55B12, and 65G8. In some embodiments, an epitope bin comprises antibodies 24F7, 9F8, 55B12, and 65G8.

Epitope mapping is the process of identifying the binding site, or epitope on a target protein/antigen where an antibody (or other binding agent) binds. A variety of methods are known in the art for mapping epitopes on target proteins. These methods include (i) mutagenesis, including but not limited to, shotgun mutagenesis, site-directed mutagenesis, and alanine scanning; (ii) domain or fragment scanning; (iii) peptide scanning (e.g., Pepscan technology); (iv) display methods, including but not limited to, phage display, microbial display, and ribosome/mRNA display; (v) methods involving proteolysis and mass spectroscopy; (vi) methods involving amide hydrogen/deuterium exchange; and (vii) structural determination, including but not limited to, x-ray crystallography and NMR.

In some embodiments, purified anti-HTRA1 antibodies are characterized by assays including, but not limited to, N-terminal sequencing, amino acid analysis, HPLC, mass spectrometry, ion exchange chromatography, and papain digestion.

In some embodiments, assays are provided for identifying anti-HTRA1 antibodies that affect HTRA1 activity. “Affecting HTRA1 activity” may include, for example, inhibiting, reducing, blocking, antagonizing, suppressing, and/or interfering with the biological activity of HTRA1. In some embodiments, an anti-HTRA1 antibody inhibits the protease activity of HTRA1. In certain embodiments, the anti-HTRA1 antibody inhibits protease activity of HTRA1 by at least about 10%, at least about 20%, at least about 30%, at least about 50%, at least about 75%, at least about 90%, at least about 95%, at least about 97%, or about 100%. In some embodiments, the amount of inhibition is used to calculate an ICₛₒ (half maximal inhibitory concentration) for the anti-HTRA1 antibody. In some embodiments, the amount of inhibition is used to calculate a K, (inhibitory constant) for the anti-HTRA1 antibody. In some embodiments, an anti-HTRA1 antibody that inhibits protease activity of HTRA1 is antibody 24F7. In some embodiments, an anti-HTRA1 antibody that inhibits protease activity of HTRA1 is antibody hz24F7. In some embodiments, an anti-HTRA1 antibody that inhibits protease activity of HTRA1 is antibody hz24F7.v2. In some embodiments, an anti-HTRA1 antibody that inhibits protease activity of HTRA1 is antibody 9F8. In some embodiments, an anti-HTRA1 antibody that inhibits protease activity of HTRA1 is antibody 55B12. In some embodiments, an anti-HTRA1 antibody that inhibits protease activity of HTRA1 is antibody 65G8. In some embodiments, the terms “inhibiting”, “reducing”, “blocking”, “antagonizing”, “suppressing”, and “interfering” are relative to levels and/or activity in the absence of treatment with the HTRA1-binding agent. In some embodiments, the terms “inhibiting”, “reducing”, “blocking”, “antagonizing”, “suppressing”, and “interfering” are relative to levels and/or activity prior to treatment with the HTRA1-binding agent.

The present disclosure also provides conjugates comprising an anti-HTRA1 antibody described herein. In some embodiments, the antibody is attached to a second molecule. In some embodiments, the antibody is conjugated to a cytotoxic agent or moiety. In some embodiments, the antibody is conjugated to a cytotoxic agent to form an ADC (antibody-drug conjugate). In some embodiments, the cytotoxic agent is a chemotherapeutic agent including, but not limited to, methotrexate, adriamycin/doxorubicin, melphalan, mitomycin C, chlorambucil, duocarmycin, daunorubicin, pyrrolobenzodiazepines (PBDs), or other intercalating agents. In some embodiments, the cytotoxic agent is a microtubule inhibitor including, but not limited to, auristatins, maytansinoids (e.g., DM1 and DM4), and tubulysins. In some embodiments, the cytotoxic agent is an enzymatically active toxin of bacterial, fungal, plant, or animal origin, or fragments thereof, including, but not limited to, diphtheria A chain, non-binding active fragments of diphtheria toxin, exotoxin A chain, ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes. In some embodiments, an antibody is conjugated to one or more small molecule toxins, such as calicheamicins, maytansinoids, trichothenes, and CC1065. A derivative of any one of these toxins may be used as long as the derivative retains the cytotoxic activity of the parent molecule.

Conjugates comprising an anti-HTRA1 antibody described herein may be made using any suitable method known in the art. In some embodiments, conjugates are made using a variety of bifunctional protein-coupling agents such as N-succinimidyl-3-(2-pyridyidithiol) propionate (SPDP), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis(p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene).

In some embodiments, an anti-HTRA1 antibody described herein is conjugated to a detectable substance or molecule that allows the antibody to be used in diagnostic and/or detection methods. A detectable substance can include but is not limited to, enzymes, such as horseradish peroxidase, alkaline phosphatase, beta-galactosidase, and acetylcholinesterase; prosthetic groups, such as biotin and flavine(s); fluorescent materials, such as, umbelliferone, fluorescein, fluorescein isothiocyanate (FITC), rhodamine, tetramethylrhodamine isothiocyanate (TRITC), dichlorotriazinylamine fluorescein, dansyl chloride, cyanine (Cy3), and phycoerythrin; bioluminescent materials, such as luciferase; radioactive materials, such as ²¹²Bi, ¹⁴C, ⁵⁷Co, ⁵¹Cr, ⁶⁷Cu, ¹⁸F, ⁶⁸Ga, ⁶⁷Ga, ¹⁵³Gd, ¹⁵⁹Gd, ⁶⁸Ge, ³H, ¹⁶⁶Ho, ¹³¹I, ¹²⁵I, ¹²³I, ¹²¹I, ¹¹⁵In, ¹¹³In, ¹¹²In, ¹¹¹In, ¹⁴⁰La, ¹⁷⁷Lu, ⁵⁴Mn, ⁹⁹Mo, ³²P, ¹⁰³Pd, ¹⁴⁹Pm, ¹⁴²Pr, ¹⁸⁶Re, ¹⁸⁸Re, ⁹⁷Ru, ³⁵S, ⁴⁷Sc, ⁷⁵Se, ¹⁵³Sm, ¹¹³Sn, ¹¹⁷Sn, ⁸⁵Sr, ^{99m}Tc, ²⁰¹Ti, ¹³³Xe, ⁹⁰Y, ⁶⁹Yb, ¹⁷⁵Yb, ⁶⁵Zn; positron emitting metals; and magnetic metal ions.

An anti-HTRA1 antibody described herein can also be conjugated to a second antibody to form an antibody heteroconjugate.

An anti-HTRA1 antibody as described herein may be attached to a solid support. Such solid supports include, but are not limited to, glass, cellulose, polyacrylamide, nylon, polystyrene, polyvinyl chloride, or polypropylene. In some embodiments, immobilized anti-HTRA1 antibodies are used in immunoassays. In some embodiments, immobilized anti-HTRA1 antibodies are used in purification of the target antigen.

### III. Polynucleotides

In some embodiments, the disclosure encompasses polynucleotides comprising polynucleotides that encode a polypeptide described herein. The term “polynucleotides that encode a polypeptide” encompasses a polynucleotide that includes only coding sequences for the polypeptide as well as a polynucleotide that includes additional coding and/or non-coding sequences. The polynucleotides of the disclosure can be in the form of RNA or in the form of DNA. DNA includes cDNA, genomic DNA, and synthetic DNA, and can be double-stranded or single-stranded, and if single stranded can be the coding strand or non-coding (anti-sense) strand.

In some embodiments, a polynucleotide comprises a polynucleotide encoding a heavy chain variable region of a HTRA1-binding agent described herein. In some embodiments, a polynucleotide comprises a polynucleotide encoding a light chain variable region of a HTRA1-binding agent described herein. In some embodiments, a polynucleotide comprises a polynucleotide encoding a heavy chain variable region of a HTRA1-binding agent described herein and a polynucleotide encoding a light chain variable region of the HTRA1-binding agent. In some embodiments, a polynucleotide comprises a polynucleotide encoding a heavy chain of a HTRA1-binding agent described herein. In some embodiments, a polynucleotide comprises a polynucleotide encoding a light chain of a HTRA1-binding agent described herein. In some embodiments, a polynucleotide comprises a polynucleotide encoding a heavy chain of a HTRA1-binding agent described herein and a polynucleotide encoding a light chain of the HTRA1-binding agent.

In some embodiments, the polynucleotide comprises a polynucleotide encoding a polypeptide comprising an amino acid sequence selected from the group consisting of: SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:87, SEQ ID NO:88, SEQ ID NO:89, and SEQ ID NO:90. In some embodiments, the polynucleotide comprises a polynucleotide encoding a polypeptide comprising an amino acid sequence of SEQ ID NO:68. In some embodiments, the polynucleotide comprises a polynucleotide encoding a polypeptide comprising an amino acid sequence of SEQ ID NO:69. In some embodiments, the polynucleotide comprises a polynucleotide encoding a polypeptide comprising an amino acid sequence of SEQ ID NO:70. In some embodiments, the polynucleotide comprises a polynucleotide encoding a polypeptide comprising an amino acid sequence of SEQ ID NO:71. In some embodiments, the polynucleotide comprises a polynucleotide encoding a polypeptide comprising an amino acid sequence of SEQ ID NO:72. In some embodiments, the polynucleotide comprises a polynucleotide encoding a polypeptide comprising an amino acid sequence of SEQ ID NO:73. In some embodiments, the polynucleotide comprises a polynucleotide encoding a polypeptide comprising an amino acid sequence of SEQ ID NO:74. In some embodiments, the polynucleotide comprises a polynucleotide encoding a polypeptide comprising an amino acid sequence of SEQ ID NO:75. In some embodiments, the polynucleotide comprises a polynucleotide encoding a polypeptide comprising an amino acid sequence of SEQ ID NO:76. In some embodiments, the polynucleotide comprises a polynucleotide encoding a polypeptide comprising an amino acid sequence of SEQ ID NO:77. In some embodiments, the polynucleotide comprises a polynucleotide encoding a polypeptide comprising an amino acid sequence of SEQ ID NO:78. In some embodiments, the polynucleotide comprises a polynucleotide encoding a polypeptide comprising an amino acid sequence of SEQ ID NO:87. In some embodiments, the polynucleotide comprises a polynucleotide encoding a polypeptide comprising an amino acid sequence of SEQ ID NO:88. In some embodiments, the polynucleotide comprises a polynucleotide encoding a polypeptide comprising an amino acid sequence of SEQ ID NO:89. In some embodiments, the polynucleotide comprises a polynucleotide encoding a polypeptide comprising an amino acid sequence of SEQ ID NO:90.

In some embodiments, the polynucleotide comprises a polynucleotide encoding a polypeptide comprising more than one amino acid sequence selected from the group consisting of: SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:87, SEQ ID NO:88, SEQ ID NO:89, and SEQ ID NO:90. In some embodiments, the polynucleotide comprises a polynucleotide encoding (i) a polypeptide comprising an amino acid sequence of SEQ ID NO:68 and (ii) a polypeptide comprising an amino acid sequence of SEQ ID NO:69. In some embodiments, the polynucleotide comprises a polynucleotide encoding (i) a polypeptide comprising an amino acid sequence of SEQ ID NO:70 and (ii) a polypeptide comprising an amino acid sequence of SEQ ID NO:72. In some embodiments, the polynucleotide comprises a polynucleotide encoding (i) a polypeptide comprising an amino acid sequence of SEQ ID NO:71 and (ii) a polypeptide comprising an amino acid sequence of SEQ ID NO:72. In some embodiments, the polynucleotide comprises a polynucleotide encoding (i) a polypeptide comprising an amino acid sequence of SEQ ID NO:73 and (ii) a polypeptide comprising an amino acid sequence of SEQ ID NO:74. In some embodiments, the polynucleotide comprises a polynucleotide encoding (i) a polypeptide comprising an amino acid sequence of SEQ ID NO:75 and (ii) a polypeptide comprising an amino acid sequence of SEQ ID NO:76. In some embodiments, the polynucleotide comprises a polynucleotide encoding (i) a polypeptide comprising an amino acid sequence of SEQ ID NO:77 and (ii) a polypeptide comprising an amino acid sequence of SEQ ID NO:78. In some embodiments, the polynucleotide comprises a polynucleotide encoding (i) a polypeptide comprising an amino acid sequence of SEQ ID NO:87 and (ii) a polypeptide comprising an amino acid sequence of SEQ ID NO:89. In some embodiments, the polynucleotide comprises a polynucleotide encoding (i) a polypeptide comprising an amino acid sequence of SEQ ID NO:88 and (ii) a polypeptide comprising an amino acid sequence of SEQ ID NO:90.

The present disclosure also provides variants of the polynucleotides described herein, wherein the variant encodes, for example, fragments, analogs, and/or derivatives of a polypeptide. In some embodiments, the present disclosure provides a polynucleotide comprising a polynucleotide having a nucleotide sequence at least 80% identical, at least 85% identical, at least 90% identical, at least 95% identical, and in some embodiments, at least 96%, at least 97%, at least 98% or at least 99% identical to a polynucleotide encoding a polypeptide described herein.

In some embodiments, a polynucleotide comprises a polynucleotide having a nucleotide sequence at least 80% identical, at least 85% identical, at least 90% identical, at least 95% identical, and in some embodiments, at least 96%, at least 97%, at least 98%, or at least 99% identical to a polynucleotide encoding an amino acid sequence selected from the group consisting of: SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:87, SEQ ID NO:88, SEQ ID NO:89, and SEQ ID NO:90. Also provided is a polynucleotide that comprises a polynucleotide that hybridizes to a polynucleotide encoding an amino acid sequence selected from the group consisting of: SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:87, SEQ ID NO:88, SEQ ID NO:89, and SEQ ID NO:90. In some embodiments, the hybridization is under conditions of high stringency as is known to those skilled in the art.

As used herein, the phrase “a polynucleotide having a nucleotide sequence at least 95% identical to a polynucleotide sequence” is intended to mean that the nucleotide sequence of the polynucleotide is identical to a reference sequence except that the polynucleotide sequence can include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence can be deleted or substituted with another nucleotide, or a number of nucleotides up to 5% of the total nucleotides in the reference sequence can be inserted into the reference sequence. It is understood by those of skill in the art that an appropriate calculation would be made for other “% identical” statements, for example, 90% identical or 85% identical. The mutations of the reference sequence can occur at the 5′ or 3′ terminal positions of the reference nucleotide sequence or anywhere between those terminal positions, interspersed either individually among nucleotides in the reference sequence or in one or more contiguous groups within the reference sequence.

The polynucleotide variants can contain alterations in the coding regions, non-coding regions, or both. In some embodiments, a polynucleotide variant contains alterations that produce silent substitutions, additions, or deletions, but does not alter the properties or activities of the encoded polypeptide. In some embodiments, a polynucleotide variant comprises silent substitutions that results in no change to the amino acid sequence of the polypeptide (due to the degeneracy of the genetic code). In some embodiments, a polynucleotide variant comprises one or more mutated codons comprising one or more (e.g., 1, 2, or 3) substitutions to the codon that change the amino acid encoded by that codon. Methods for introducing one or more substitutions into a codon are known in the art, including by not limited to, PCR mutagenesis and site-directed mutagenesis. Polynucleotide variants can be produced for a variety of reasons, for example, to optimize codon expression for a particular host (e.g., change codons in the human mRNA to those preferred by a bacterial host such as E. coli). In some embodiments, a polynucleotide variant comprises at least one silent mutation in a non-coding or a coding region of the sequence.

In some embodiments, a polynucleotide variant is produced to modulate or alter expression (or expression levels) of the encoded polypeptide. In some embodiments, a polynucleotide variant is produced to increase expression of the encoded polypeptide. In some embodiments, a polynucleotide variant is produced to decrease expression of the encoded polypeptide. In some embodiments, a polynucleotide variant has increased expression of the encoded polypeptide as compared to a parental polynucleotide sequence. In some embodiments, a polynucleotide variant has decreased expression of the encoded polypeptide as compared to a parental polynucleotide sequence.

In some embodiments, a polynucleotide comprises the coding sequence for a polypeptide fused in the same reading frame to a polynucleotide that aids in expression and secretion of a polypeptide from a host cell. In some embodiments, the polynucleotide that aids in expression and secretion is a leader sequence that functions as a secretory sequence for controlling transport of a polypeptide. In some embodiments, the polypeptide has a leader sequence cleaved by the host cell to form a “mature” form of the polypeptide.

In some embodiments, a polynucleotide comprises the coding sequence for a polypeptide fused in the same reading frame to a marker or tag sequence. For example, in some embodiments, a marker sequence is a hexa-histidine tag (HIS-tag; SEQ ID NO:93) that allows for efficient purification of the polypeptide fused to the marker. In some embodiments, a marker sequence is a hemagglutinin (HA) tag derived from the influenza hemagglutinin protein when a mammalian host is used. In some embodiments, the marker sequence is a FLAG™ tag. In some embodiments, a marker may be used in conjunction with other markers or tags.

In some embodiments, a polynucleotide is isolated. In some embodiments, a polynucleotide is substantially pure.

Vectors and cells comprising each and every one of the polynucleotides described herein are also provided. In some embodiments, a vector comprises a polynucleotide molecule encoding a HTRA1-binding agent described herein. In some embodiments, a vector comprises a polynucleotide molecule encoding a polypeptide that is part of a HTRA1-binding agent described herein. In some embodiments, a cell comprises a vector comprising a polynucleotide molecule encoding a HTRA1-binding agent described herein. In some embodiments, a cell comprises a vector comprising a polynucleotide molecule encoding a polypeptide that is part of a HTRA1-binding agent described herein. In some embodiments, a cell comprises a polynucleotide molecule encoding a HTRA1-binding agent described herein. In some embodiments, a cell comprises one or more polynucleotides encoding a HTRA1-binding agent described herein. In some embodiments, a cell comprises a single polynucleotide encoding a HTRA1-binding agent described herein. In some embodiments, a cell comprises a first polynucleotide encoding a heavy chain variable region of a HTRA1-binding agent described herein and a second polynucleotide encoding a light chain variable region of a HTRA1-binding agent described herein. In some embodiments, a cell comprises a polynucleotide encoding a heavy chain variable region and a light chain variable region of a HTRA1-binding agent described herein. In some embodiments, a cell comprises a first polynucleotide encoding a heavy chain of a HTRA1-binding agent described herein and a second polynucleotide encoding a light chain of a HTRA1-binding agent described herein. In some embodiments, a cell comprises a polynucleotide encoding a heavy chain and a light chain of a HTRA1-binding agent described herein.

In some embodiments, a cell comprises one or more vectors encoding a HTRA1-binding agent described herein. In some embodiments, a cell comprises a vector encoding a HTRA1-binding agent described herein. In some embodiments, a cell comprises a first vector encoding a heavy chain variable region of a HTRA1-binding agent described herein and a second vector encoding a light chain variable region of a HTRA1-binding agent described herein. In some embodiments, a cell comprises a single vector encoding a heavy chain variable region and a light chain variable region of a HTRA1-binding agent described herein. In some embodiments, a cell comprises a first vector encoding a heavy chain of a HTRA1-binding agent described herein and a second vector encoding a light chain of a HTRA1-binding agent described herein. In some embodiments, a cell comprises a single vector encoding a heavy chain and a light chain of a HTRA1-binding agent described herein.

### IV. Methods of Making Binding Agents

The disclosure provides methods for making the HTRA1-binding agents described herein. In some embodiments, a method comprises providing a cell comprising a heavy chain variable region and/or light chain variable region of a HTRA1-binding agent described herein, culturing the cell under conditions that permit the expression of the binding agent, and isolating the binding agent. In some embodiments, a cell comprises one or more vectors encoding the heavy chain variable region and the light chain variable region of a HTRA1-binding agent described herein. In some embodiments, a method comprises providing a cell comprising a heavy chain and/or light chain of a HTRA1-binding agent described herein, incubating the cell under conditions that permit the expression of the binding agent, and isolating the binding agent. In some embodiments, a cell comprises one or more vectors encoding the heavy chain and the light chain of a HTRA1-binding agent described herein. In some embodiments, a cell comprises a first vector encoding the heavy chain of a HTRA1-binding agent and a second vector encoding the light chain of a HTRA1-binding agent described herein. In other embodiments, a cell comprises a vector encoding the heavy chain and the light chain of a HTRA1-binding agent described herein. In some embodiments, a cell comprises one or more polynucleotides encoding the heavy chain and the light chain of a HTRA1-binding agent described herein. In some embodiments, a cell comprises a first polynucleotide encoding the heavy chain of a HTRA1-binding agent and a second polynucleotide encoding the light chain of a HTRA1-binding agent described herein. In other embodiments, a cell comprises a polynucleotide encoding the heavy chain and the light chain of a HTRA1-binding agent described herein. In some embodiments, the method comprises purifying the antibody.

In some embodiments, the HTRA1-binding agent is an antibody fragment comprising at least one antigen-binding site and the method involves providing a cell comprising the fragment of an anti-HTRA1 antibody, incubating the cell under conditions that permit the expression of the antibody fragment, and isolating the antibody fragment. In certain embodiments, the cell comprises a vector encoding an antibody fragment described herein. In certain embodiments, the cell comprises a polynucleotide encoding an antibody fragment described herein. In some embodiments, the method comprises purifying the antibody fragment. In certain embodiments, the antibody fragment is a Fab, Fab′, F(ab′)₂, Fv, scFv, dsscFv, (scFv)₂, single chain antibody, dual variable region antibody, diabody, or nanobody.

In some embodiments, the HTRA1-binding agent is a scFv and the method involves providing a cell comprising the scFv, incubating the cell under conditions that permit the expression of the scFv, and isolating the scFv. In certain embodiments, the cell comprises a vector described herein encoding the scFv. In certain embodiments, the cell comprises a polynucleotide described herein encoding the scFv. In some embodiments, the method comprises purifying the scFv.

In some embodiments, a polynucleotide encoding a HTRA1-binding agent described herein is transiently transfected into a cell. In some embodiments, a polynucleotide encoding a HTRA1-binding agent described herein is stably transfected into a cell. In some embodiments, the cell used to make a HTRA1-binding agent is a bacterial cell (e.g., E. coli). In some embodiments, the cell used to make a HTRA1-binding agent is a yeast cell (e.g., Pichia pastoris). In some embodiments, the cell used to make a HTRA1-binding agent is a mammalian cell. In other embodiments, the cell used to make a HTRA1-binding agent is a HEK-293 cell. In other embodiments, the cell used to make a HTRA1-binding agent is a HEK-293 cell.

### V. Methods of Use and Pharmaceutical Compositions

The HTRA1-binding agents of the disclosure are useful in a variety of applications including, but not limited to, therapeutic treatment methods, such as treatment of a HTRA1-associated disorder. In some embodiments, a HTRA1-associated disorder is an eye disorder. In some embodiments, a HTRA1-binding agent described herein is useful in methods for inhibiting HTRA1 activity in a subject. In some embodiments, a HTRA1-binding agent described herein is useful in methods for inhibiting HTRA1 activity in an eye of a subject. In some embodiments, a HTRA1-binding agent described herein is useful in methods for treating an eye disorder. In some embodiments, a HTRA1-binding agent described herein is useful in methods for treating macular degeneration. In some embodiments, a HTRA1-binding agent described herein is useful in methods for treating a disorder associated with macular degeneration. In some embodiments, a HTRA1-binding agent described herein is useful in methods for treating age-related macular degeneration (AMD). In some embodiments, a HTRA1-binding agent described herein is useful in methods for treating wet AMD. In some embodiments, a HTRA1-binding agent described herein is useful in methods for treating dry AMD. In some embodiments, a HTRA1-binding agent described herein is useful in methods for treating early AMD. In some embodiments, a HTRA1-binding agent described herein is useful in methods for treating intermediate AMD. In some embodiments, a HTRA1-binding agent described herein is useful in methods for treating advanced AMD. In some embodiments, a HTRA1-binding agent described herein is useful in methods for treating advanced dry AMD/geographic atrophy (GA). The terms “advanced dry AMD” and “geographic atrophy” are used interchangeably herein.

In some embodiments, a HTRA1-binding agent is useful in methods for inhibiting HTRA1 activity (e.g., protease activity) in an eye of a subject, wherein the method comprises administering to the subject a therapeutically effective amount of a HTRA1-binding agent described herein. In some embodiments, a method of inhibiting HTRA1 activity in an eye of a subject comprises administering to the subject a therapeutically effective amount of an anti-HTRA1 antibody described herein. In some embodiments of the methods described herein, the HTRA1-binding agent is administered to an eye of the subject. In some embodiments of the methods described herein, the anti-HTRA1 antibody is administered to an eye of the subject.

In some embodiments, a method of treating an eye disorder in a subject comprises administering to the subject a therapeutically effective amount of a HTRA1-binding agent described herein. “Eye disorder or disease” and “ocular disorder or disease” are used interchangeably herein. In some embodiments, a method of treating an eye disorder in a subject comprises administering to the subject a therapeutically effective amount of an anti-HTRA1 antibody described herein. In some embodiments, the eye disorder is selected from the group consisting of: macular degeneration (maculopathy), age-related macular degeneration (AMD), diabetic retinopathy, retinopathy of prematurity, sickle cell retinopathy, macular dystrophy, retinal dystrophy, uveitis, keratitis, scleritis, retinitis pigmentosa, choroidal neovascularization (CNV), retinal neovascularization, ocular neovascularization, corneal neovascularization, ocular inflammation, polypoidal choroidal vasculopathy (PCV), idiopathic polypoidal choroidal vasculopathy (IPCV), Stargardt disease (also called Stargardt macular dystrophy, juvenile macular degeneration, or fundus flavimaculatus), ocular ischemia, macular edema, diabetic macular edema (DME), macular edema following retinal vein occlusion, diabetic blindness, retinopathy, primary diabetic retinopathy, rebeosis, and neuromyelitis optica. In some embodiments, the eye disorder is macular degeneration. In some embodiments, the eye disorder is AMD. In some embodiments, the eye disorder is wet AMD. In some embodiments, the eye disorder is dry AMD. In some embodiments, the eye disorder is early AMD. In some embodiments, the eye disorder is intermediate AMD. In some embodiments, the eye disorder is advanced AMD. In some embodiments, the eye disorder is geographic atrophy. In some embodiments, the eye disorder is neovascularization. In some embodiments, the eye disorder is CNV. In some embodiments, the eye disorder is PCV. In some embodiments, the eye disorder is IPCV. In some embodiments, the eye disorder is Stargardt disease.

In the early stages of AMD, the disease is characterized by the presence of drusen that can manifest with or without retinal pigment epithelium (RPE) irregularities. In the early stages when drusen are small and/or few, vision is generally not affected. As drusen enlarge and multiply in number, patients report that central vision is less sharp. Geographic atrophy (GA) is the advanced form of dry AMD. GA is characterized by the degeneration of RPE, wherein the degeneration of the RPE leads to the death of photoreceptor cells (the rods and cones). Thus, a patient has a spot or area (a “continent”) of atrophy surrounded by a “sea” of healthy or healthier-looking retina. Doctors can measure the area of atrophy (often referred to as “GA lesion area”) and estimate the loss of visual function. Changes in the GA lesion area can be used to follow progression of the disease over time.

Choroidal neovascularization (CNV) and/or wet AMD involves the growth of new blood vessels that originate from the choroid through a break in the Bruch membrane into the sub-retinal pigment epithelium (sub-RPE) or sub-retinal space. These new blood vessels may bleed and leak fluid, causing the macula to bulge or lift up from its normally flat position, thus distorting or destroying central vision. Under these circumstances, vision loss is often rapid and severe.

In some embodiments, a method of treating AMD in a subject comprises administering to the subject a therapeutically effective amount of a HTRA1-binding agent described herein. In some embodiments, a method of treating AMD in a subject comprises administering to the subject a therapeutically effective amount of an anti-HTRA1 antibody described herein. In some embodiments, the AMD is wet AMD. In some embodiments, the AMD is dry AMD. In some embodiments, the AMD is early AMD. In some embodiments, the AMD is intermediate AMD. In some embodiments, the AMD is advanced AMD. In some embodiments, the AMD is GA. In some embodiments, the AMD is associated with CNV.

In some embodiments, a method of treating geographic atrophy in a subject comprises administering to the subject a therapeutically effective amount of a HTRA1-binding agent described herein. In some embodiments, a method of treating geographic atrophy in a subject comprises administering to the subject a therapeutically effective amount of an anti-HTRA1 antibody described herein.

In some embodiments, a method of treating wet AMD in a subject comprises administering to the subject a therapeutically effective amount of a HTRA1-binding agent described herein. In some embodiments, a method of treating wet AMD in a subject comprises administering to the subject a therapeutically effective amount of an anti-HTRA1 antibody described herein.

In some embodiments, a method of treating CNV in a subject comprises administering to the subject a therapeutically effective amount of a HTRA1-binding agent described herein. In some embodiments, a method of treating CNV in a subject comprises administering to the subject a therapeutically effective amount of an anti-HTRA1 antibody described herein.

In some embodiments, a method of inhibiting progression of early AMD to advanced AMD in an eye of a subject comprises administering to the eye of the subject a therapeutically effective amount of a HTRA1-binding agent described herein. In some embodiments, a method of inhibiting progression of early AMD to advanced AMD in an eye of a subject comprises administering to the eye of the subject a therapeutically effective amount of an anti-HTRA1 antibody described herein. In some embodiments, the method inhibits progression of early AMD to GA. In some embodiments, the method inhibits progression of early AMD to wet AMD. In some embodiments, the method inhibits progression of early AMD to CNV. In some embodiments, the method inhibits progression of dry AMD to wet AMD.

In some embodiments, a method of inhibiting progression of intermediate AMD to advanced AMD in an eye of a subject comprises administering to the eye of the subject a therapeutically effective amount of a HTRA1-binding agent described herein. In some embodiments, a method of inhibiting progression of intermediate AMD to advanced AMD in an eye of a subject comprises administering to the eye of the subject a therapeutically effective amount of an anti-HTRA1 antibody described herein. In some embodiments, the method inhibits progression of intermediate AMD to GA. In some embodiments, the method inhibits progression of intermediate AMD to wet AMD. In some embodiments, the method inhibits progression of intermediate AMD to CNV. In some embodiments, the method inhibits progression of dry AMD to wet AMD.

In some embodiments, a method of inhibiting or suppressing drusen formation in an eye of a subject comprises administering to the subject a therapeutically effective amount of a HTRA1-binding agent described herein. In some embodiments, a method of inhibiting or suppressing drusen formation in an eye of a subject comprises administering to the subject a therapeutically effective amount of an anti-HTRA1 antibody described herein. In some embodiments, the method reduces the number and/or size of the drusen.

In some embodiments, a method of inhibiting or suppressing retinal pigment epithelium atrophy in an eye of a subject comprises administering to the subject a therapeutically effective amount of a HTRA1-binding agent described herein. In some embodiments, a method of inhibiting or suppressing retinal pigment epithelium atrophy in an eye of a subject comprises administering to the subject a therapeutically effective amount of an anti-HTRA1 antibody described herein.

In some embodiments, a method of treating a subject at risk of developing AMD comprises administering to the subject a therapeutically effective amount of a HTRA1-binding agent described herein. In some embodiments, a method of treating a subject at risk of developing AMD comprises administering to the subject a therapeutically effective amount of an anti-HTRA1 antibody described herein. In some embodiments, a method of treating a subject at risk of developing geographic atrophy comprises administering to the subject a therapeutically effective amount of a HTRA1-binding agent described herein. In some embodiments, a method of treating a subject at risk of developing geographic atrophy comprises administering to the subject a therapeutically effective amount of an anti-HTRA1 antibody described herein. In some embodiments, a method of treating a subject at risk of developing wet AMD and/or CNV comprises administering to the subject a therapeutically effective amount of a HTRA1-binding agent described herein. In some embodiments, a method of treating a subject at risk of developing wet AMD and/or CNV comprises administering to the subject a therapeutically effective amount of an anti-HTRA1 antibody described herein.

In some embodiments, a method of slowing down or reducing the progression of AMD in a subject comprises administering to the subject a therapeutically effective amount of a HTRA1-binding agent described herein. In some embodiments, a method of slowing down or reducing the progression of AMD in a subject comprises administering to the subject a therapeutically effective amount of an anti-HTRA1 antibody described herein. In some embodiments, the method slows down or reduces progression of early AMD to intermediate AMD. In some embodiments, the method slows down or reduces progression of intermediate AMD to advanced AMD. In some embodiments, the method slows down or reduces progression of intermediate AMD to geographic atrophy. In some embodiments, a method of slowing down or reducing the progression of geographic atrophy in a subject comprises administering to the subject a HTRA1-binding agent described herein. In some embodiments, a method of slowing down or reducing the progression of geographic atrophy in a subject comprises administering to the subject a therapeutically effective amount of an anti-HTRA1 antibody described herein. In some embodiments, the method slows down or reduces progression of intermediate AMD to wet AMD and/or CNV. In some embodiments, a method of slowing down or reducing the progression of wet AMD and/or CNV in a subject comprises administering to the subject a HTRA1-binding agent described herein. In some embodiments, a method of slowing down or reducing the progression of wet AMD and/or CNV in a subject comprises administering to the subject a therapeutically effective amount of an anti-HTRA1 antibody described herein.

In some embodiments, a method of treating AMD in an eye of a subject, wherein the subject has been previously treated with a VEGF inhibitor, wherein the method comprises administering to the subject a therapeutically effective amount of a HTRA1-binding agent described herein. In some embodiments, a method of treating AMD in an eye of a subject, wherein the subject has been previously treated with a VEGF inhibitor, wherein the method comprises administering to the subject a therapeutically effective amount of an anti-HTRA1 antibody described herein.

In some embodiments, a method of inhibiting or reducing a progression from dry AMD to wet AMD in an eye of a subject, wherein the method comprises administering to the subject a therapeutically effective amount of a HTRA1-binding agent described herein. In some embodiments, a method of inhibiting or reducing a progression from dry AMD to wet AMD in an eye of a subject, wherein the method comprises administering to the subject a therapeutically effective amount of an anti-HTRA1 antibody described herein.

In some embodiments of the methods described herein, a subject has AMD or GA. In some embodiments of the methods described herein, a subject has been diagnosed with AMD or GA. The criteria for diagnosis of AMD and GA differ across grading systems and may depend upon the imaging modalities that are used. In some embodiments, the criteria for AMD comprises: (1) No AMD—no or a few small (<63 μm in diameter) drusen; (2) Early AMD—intermediate-sized (63-124 μm in diameter) drusen; (3) Intermediate AMD—intermediate-sized drusen and pigmentary changes or at least 1 large (>125 μm) drusen; (4) Late AMD (dry)—geographic atrophy or Late AMD (wet)—CNV with signs including sub-retinal hemorrhage, serous retinal or retinal pigment epithelium detachments, lipid exudates, or fibrovascular scarring. In some embodiments, the criteria for GA comprises an area of pallor in the fundus with visibility of the underlying choroidal blood vessels and sharply defined borders, occupying (1) a diameter >175 μm; (2) a diameter >250 μm; or (3) a diameter of at least 433 μm. Currently, there is no consensus on the minimum diameter for the diagnosis of GA.

There are a number of imaging modalities used by medical practitioners skilled in the art, including but not limited to, color fundus photography (CFP), fundus autofluorescence (FAF), optical coherence tomography (OCT), OCT angiography, fluorescein angiography (FA), indocyanine green angiography (ICGA), and scanning laser ophthalmoscopy (SLO). Imaging modalities allow for the direct measurement and quantification of GA lesion areas and for visualization of blood vessel formation.

Thus, in some embodiments, a method of treating geographic atrophy in a subject comprises administering to the subject a therapeutically effective amount of an anti-HTRA1 antibody described herein, wherein the treatment reduces growth of the GA lesion area. In some embodiments, the GA lesion area is measured by color fundus photography, fluorescein angiography, and/or optical coherence tomography. In some embodiments, the GA lesion is measured prior to treatment with an anti-H IRA1 antibody and measured again after treatment. In some embodiments, growth of the GA lesion area is reduced by about 20%, about 30%, about 40%, about 50%, about 60%, about 70%, or about 80% or better. In some embodiments, growth of the GA lesion area is reduced by at least 25%. In some embodiments, growth of the GA lesion is reduced by at least 40%. In some embodiments, growth of the GA lesion is reduced by at least 50%. In some embodiments, the GA lesion area is measured about 6 months after treatment, about 1 year after treatment, about 18 months after treatment, and/or about 2 years after treatment. In some embodiments, the GA lesion area is measured every time the anti-HTRA1 antibody is administered. In some embodiments, the GA lesion area is measured at time points chosen by the medical practitioner.

In some embodiments of the methods described herein, the HTRA1-binding agent comprises a heavy chain variable region comprising a heavy chain variable region CDR1, CDR2, and CDR3 and a light chain variable region comprising a light chain variable region CDR1, CDR2, and CDR3 of antibody 24F7. In some embodiments of the methods described herein, the HTRA1-binding agent comprises a heavy chain variable region comprising a heavy chain variable region CDR1, CDR2, and CDR3 and a light chain variable region comprising a light chain variable region CDR1, CDR2, and CDR3 of antibody hz24F7. In some embodiments of the methods described herein, the HTRA1-binding agent comprises a heavy chain variable region comprising a heavy chain variable region CDR1, CDR2, and CDR3 and a light chain variable region comprising a light chain variable region CDR1, CDR2, and CDR3 of hz24F7.v2.

In some embodiments of the methods described herein, the HTRA1-binding agent (e.g. an antibody) comprises a heavy chain variable region comprising a heavy chain variable region CDR1, CDR2, and CDR3 and a light chain variable region comprising a light chain variable region CDR1, CDR2, and CDR3 of antibody 9F8 or a humanized version of 9F8. In some embodiments of the methods described herein, the HTRA1-binding agent comprises a heavy chain variable region comprising a heavy chain variable region CDR1, CDR2, and CDR3 and a light chain variable region comprising a light chain variable region CDR1, CDR2, and CDR3 of antibody 55B12 or a humanized version of 55B12. In some embodiments of the methods described herein, the HTRA1-binding agent comprises a heavy chain variable region comprising a heavy chain variable region CDR1, CDR2, and CDR3 and a light chain variable region comprising a light chain variable region CDR1, CDR2, and CDR3 of antibody 65G8 or a humanized version of 65G8.

In some embodiments of the methods described herein, the anti-HTRA1 antibody comprises: (a) a heavy chain variable region comprising a heavy chain variable region CDR1 comprising the amino acid sequence GYTFTDYEMH (SEQ ID NO:9), a heavy chain variable region CDR2 comprising the amino acid sequence AIDPETGGTAYNQKFKG (SEQ ID NO:10), and a heavy chain variable region CDR3 comprising the amino acid sequence EGYSYDGGGYYFDY (SEQ ID NO:11) or the amino acid sequence EGYSYEGGGYYFDY (SEQ ID NO:25), and (b) a light chain variable region comprising a light chain variable region CDR1 comprising the amino acid sequence SVSSSVSYMY (SEQ ID NO:12), a light chain variable region CDR2 comprising the amino acid sequence DTSNLAS (SEQ ID NO:13), and a light chain variable region CDR3 comprising the amino acid sequence QQWSSYPT (SEQ ID NO:14). In some embodiments of the methods described herein, the anti-HTRA1 antibody comprises: (a) a heavy chain variable region comprising a heavy chain variable region CDR1 comprising the amino acid sequence GYTFTDYEMH (SEQ ID NO:9), a heavy chain variable region CDR2 comprising the amino acid sequence AIDPETGGTAYNQKFKG (SEQ ID NO:10), and a heavy chain variable region CDR3 comprising the amino acid sequence EGYSYDGGGYYFDY (SEQ ID NO:11) and (b) a light chain variable region comprising a light chain variable region CDR1 comprising the amino acid sequence SVSSSVSYMY (SEQ ID NO:12), a light chain variable region CDR2 comprising the amino acid sequence DTSNLAS (SEQ ID NO:13), and a light chain variable region CDR3 comprising the amino acid sequence QQWSSYPT (SEQ ID NO:14). In some embodiments of the methods described herein, the anti-HTRA1 antibody comprises: (a) a heavy chain variable region comprising a heavy chain variable region CDR1 comprising the amino acid sequence GYTFTDYEMH (SEQ ID NO:9), a heavy chain variable region CDR2 comprising the amino acid sequence AIDPETGGTAYNQKFKG (SEQ ID NO:10), and a heavy chain variable region CDR3 comprising the amino acid sequence EGYSYEGGGYYFDY (SEQ ID NO:25), and (b) a light chain variable region comprising a light chain variable region CDR1 comprising the amino acid sequence SVSSSVSYMY (SEQ ID NO:12), a light chain variable region CDR2 comprising the amino acid sequence DTSNLAS (SEQ ID NO:13), and a light chain variable region CDR3 comprising the amino acid sequence QQWSSYPT (SEQ ID NO:14).

In some embodiments of the methods described herein, the anti-HTRA1 antibody comprises: (a) a heavy chain variable region comprising the amino acid sequence SEQ ID NO:68, SEQ ID NO:70, or SEQ ID NO:71; and (b) a light chain variable region comprising the amino acid sequence SEQ ID NO:69 or SEQ ID NO:72. In some embodiments of the methods described herein, the anti-HTRA1 antibody comprises: (a) a heavy chain variable region comprising the amino acid sequence SEQ ID NO:68 and (b) a light chain variable region comprising the amino acid sequence SEQ ID NO:69. In some embodiments of the methods described herein, the anti-HTRA1 antibody comprises: (a) a heavy chain variable region comprising the amino acid sequence SEQ ID NO:70 and (b) a light chain variable region comprising the amino acid sequence SEQ ID NO:72. In some embodiments of the methods described herein, the anti-HTRA1 antibody comprises: (a) a heavy chain variable region comprising the amino acid sequence SEQ ID NO:71 and (b) a light chain variable region comprising the amino acid sequence SEQ ID NO:72.

In some embodiments of the methods described herein, the anti-HTRA1 antibody comprises a polypeptide comprising the amino acid sequence SEQ ID NO:71. In some embodiments of the methods described herein, the anti-HTRA1 antibody comprises a polypeptide comprising the amino acid sequence SEQ ID NO:72. In some embodiments of the methods described herein, the anti-HTRA1 antibody comprises a polypeptide comprising the amino acid sequence SEQ ID NO:71 and a polypeptide comprising the amino acid sequence SEQ ID NO:72.

In some embodiments of the methods described herein, the anti-HTRA1 antibody is hz24F7. In some embodiments of the methods described herein, the anti-HTRA1 antibody is hz24F7.v2.

In some embodiments of the methods described herein, the anti-HTRA1 antibody comprises a heavy chain variable region and a light chain variable region, wherein the heavy chain variable region comprises heavy chain variable region CDRs 1, 2, and 3 of SEQ ID NO:73, and the light chain variable region comprises light chain variable region CDRs 1, 2, and 3 of SEQ ID NO:74. In certain embodiments, the heavy chain variable region comprises a heavy chain variable region CDR1 comprising the amino acid sequence GYAFTTYWMH (SEQ ID NO:27), a heavy chain variable region CDR2 comprising the amino acid sequence NIDPSDSETHYNQKFRD (SEQ ID NO:28), and a heavy chain variable region CDR3 comprising the amino acid sequence DYGAFDV (SEQ ID NO:29), and the light chain variable region comprises a light chain variable region CDR1 comprising the amino acid sequence RSSTGAVTTRNFAS (SEQ ID NO:30), a light chain variable region CDR2 comprising the amino acid sequence GTNNRAP (SEQ ID NO:31), and a light chain variable region the amino acid sequence CDR3 comprising ALWYSNLWV (SEQ ID NO:32).

In some embodiments of the methods described herein, the anti-HTRA1 antibody comprises a heavy chain variable region comprising the amino acid sequence SEQ ID NO:73, and a light chain variable region comprising the amino acid sequence SEQ ID NO:74. In some embodiments of the methods described herein, the anti-HTRA1 antibody comprises a polypeptide comprising the amino acid sequence SEQ ID NO:73. In some embodiments of the methods described herein, the anti-HTRA1 antibody comprises a polypeptide comprising the amino acid sequence SEQ ID NO:74. In some embodiments of the methods described herein, the anti-HTRA1 antibody comprises a polypeptide comprising the amino acid sequence SEQ ID NO:73 and a polypeptide comprising the amino acid sequence SEQ ID NO:74.

In some embodiments of the methods described herein, the anti-HTRA1 antibody is a humanized version of antibody 9F8. In some embodiments of the methods described herein, the anti-HTRA1 antibody is a variant of antibody 9F8.

In some embodiments of the methods described herein, the anti-HTRA1 antibody comprises a heavy chain variable region and a light chain variable region, wherein the heavy chain variable region comprises heavy chain variable region CDRs 1, 2, and 3 of SEQ ID NO:75, and the light chain variable region comprises light chain variable region CDRs 1, 2, and 3 of SEQ ID NO:76. In certain embodiments, the heavy chain variable region comprises a heavy chain variable region CDR1 comprising the amino acid sequence GYTFTNYWMH (SEQ ID NO:43), a heavy chain variable region CDR2 comprising the amino acid sequence NIDPSDSETHYNQKFKD (SEQ ID NO:44), and a heavy chain variable region CDR3 comprising the amino acid sequence EDSSGYGAY (SEQ ID NO:45), and the light chain variable region comprises a light chain variable region CDR1 comprising the amino acid sequence SASSSVNYMH (SEQ ID NO:46), a light chain variable region CDR2 comprising the amino acid sequence DTSKLAS (SEQ ID NO:47), and a light chain variable region CDR3 comprising the amino acid sequence QQWSSHPLT (SEQ ID NO:48).

In some embodiments of the methods described herein, the anti-HTRA1 antibody comprises a heavy chain variable region comprising the amino acid sequence SEQ ID NO:75, and a light chain variable region comprising the amino acid sequence SEQ ID NO:76. In some embodiments of the methods described herein, the anti-HTRA1 antibody comprises a polypeptide comprising the amino acid sequence SEQ ID NO:75. In some embodiments of the methods described herein, the anti-HTRA1 antibody comprises a polypeptide comprising the amino acid sequence SEQ ID NO:76. In some embodiments of the methods described herein, the anti-HTRA1 antibody comprises a polypeptide comprising the amino acid sequence SEQ ID NO:75 and a polypeptide comprising the amino acid sequence SEQ ID NO:76.

In some embodiments of the methods described herein, the anti-HTRA1 antibody is a humanized version of antibody 55B12. In some embodiments of the methods described herein, the anti-HTRA1 antibody is a variant of antibody 55B12.

In some embodiments of the methods described herein, the anti-HTRA1 antibody comprises a heavy chain variable region and a light chain variable region, wherein the heavy chain variable region comprises heavy chain variable region CDRs 1, 2, and 3 of SEQ ID NO:77, and the light chain variable region comprises light chain variable region CDRs 1, 2, and 3 of SEQ ID NO:78. In certain embodiments, the heavy chain variable region comprises a heavy chain variable region CDR1 comprising the amino acid sequence GYSFTSYWMH (SEQ ID NO:56), a heavy chain variable region CDR2 comprising the amino acid sequence MIDPSDSETRLNQKFKD (SEQ ID NO:57), and a heavy chain variable region CDR3 comprising the amino acid sequence DYFDY (SEQ ID NO:58), and the light chain variable region a light chain variable region CDR1 comprising the amino acid sequence SASSSVSYMY (SEQ ID NO:59), a light chain variable region CDR2 comprising the amino acid sequence DTSNLAS (SEQ ID NO:13), and a light chain variable region CDR3 comprising the amino acid sequence QQWSSYPYT (SEQ ID NO:60).

In some embodiments of the methods described herein, the anti-HTRA1 antibody comprises a heavy chain variable region comprising the amino acid sequence SEQ ID NO:77, and a light chain variable region comprising the amino acid sequence SEQ ID NO:78. In some embodiments of the methods described herein, the anti-HTRA1 antibody comprises a polypeptide comprising the amino acid sequence SEQ ID NO:77. In some embodiments of the methods described herein, the anti-HTRA1 antibody comprises a polypeptide comprising the amino acid sequence SEQ ID NO:78. In some embodiments of the methods described herein, the anti-HTRA1 antibody comprises a polypeptide comprising the amino acid sequence SEQ ID NO:77 and a polypeptide comprising the amino acid sequence SEQ ID NO:78.

In some embodiments of the methods described herein, the anti-HTRA1 antibody is a humanized version of antibody 65G8. In some embodiments of the methods described herein, the anti-HTRA1 antibody is a variant of antibody 65G8.

In some embodiments of the methods described herein, a method comprises administering a HTRA1-binding agent described herein in combination with at least one additional therapeutic agent or therapeutic therapy. Treatment with two or more therapeutic agents often uses agents that work by different mechanisms of action, although this is not required. Combination therapy using agents with different mechanisms of action may result in additive or synergetic effects. Combination therapy may allow for a lower dose of each agent than is used in monotherapy, thereby reducing toxic side effects and/or increasing the therapeutic index of the agent(s). Combination therapy may decrease the likelihood that resistance to an agent will develop.

In some embodiments, the combination of a HTRA1-binding agent described herein and at least one additional therapeutic agent results in additive or synergistic results. In some embodiments, the combination therapy results in an increase in the therapeutic index of the HTRA1-binding agent. In some embodiments, the combination therapy results in an increase in the therapeutic index of the additional therapeutic agent(s). In some embodiments, the combination therapy results in a decrease in the toxicity and/or side effects of the HTRA1-binding agent. In some embodiments, the combination therapy results in a decrease in the toxicity and/or side effects of the additional therapeutic agent(s).

In some embodiments, a combination treatment comprises one additional therapeutic agent or two or more additional therapeutic agents. In some embodiments, treatment with a HTRA1-binding agent can occur prior to, concurrently with, or subsequent to administration of the additional therapeutic agents. In some embodiments, combined administration includes co-administration, either in a single pharmaceutical formulation or using separate formulations, or consecutive administration in either order but generally within a time period such that all active agents can exert their biological activities. In some embodiments, preparation of agents and/or dosing schedules for additional therapeutic agents are according to manufacturers' instructions or as determined empirically by the skilled practitioner.

In some embodiments of the methods described herein, a HTRA1-binding agent is administered to a subject as part of a combination therapy. In some embodiments, the combination therapy comprises photodynamic therapy. In some embodiments, the combination therapy comprises photodynamic therapy with verteporfin. In some embodiments, a HTRA1-binding agent is administered to a subject, wherein the subject is administered one or more additional therapeutic agents. In some embodiments, an additional therapeutic agent is compstatin or an analog or derivative of compstatin (e.g., POT-4, APL-2, and AMY-101). In some embodiments, an additional therapeutic agent is a C5 inhibitor. In some embodiments, a C5 inhibitor is selected from the group including, but not limited to, eculizumab, LFG316, or Zimura (anti-05 aptamer). In some embodiments, an additional therapeutic agent is a properdin inhibitor (e.g., an anti-properdin antibody). In some embodiments, an additional therapeutic agent is a Factor D inhibitor. In some embodiments, a Factor D inhibitor is an anti-Factor D antibody (e.g., lampalizumab). In some embodiments, an additional therapeutic agent is a complement component C3 inhibitor. In some embodiments, a C3 inhibitor is an anti-C3 antibody. In some embodiments, an additional therapeutic agent is a VEGF inhibitor. In some embodiments, a VEGF inhibitor is selected from the group including, but not limited to, pegaptanib (MACUGEN), ranibizumab (LUCENTIS), bevacizumab (AVASTIN), aflibercept (EYLEA), brolucizumab, and OPT-302. In some embodiments, an additional therapeutic agent is a PDGF inhibitor. In some embodiments, an additional therapeutic agent is a corticosteroid. In some embodiments, an additional therapeutic agent is a neuroprotective agent. In some embodiments, a neuroprotective agent is selected from the group including, but not limited to, ciliary neurotrophic factor (CNTF), tandospirone, and brimonidine.

It will be appreciated that the combination of a HTRA1-binding agent described herein and at least one additional therapeutic agent may be administered in any order or concurrently. In some embodiments, a HTRA1-binding agent is administered to subjects that have previously undergone treatment with a therapeutic agent. In some embodiments, a HTRA1-binding agent and a second therapeutic agent are administered substantially simultaneously or concurrently. For example, a subject may be given a HTRA1-binding agent while undergoing a course of treatment with a second therapeutic agent (e.g., an angiogenesis inhibitor). In some embodiments, a HTRA1-binding agent is administered within 1 year of the treatment with a second therapeutic agent. In some embodiments, a HTRA1-binding agent is administered within 10, 8, 6, 4, or 2 months of any treatment with a second therapeutic agent. In some embodiments, a HTRA1-binding agent is administered within 4, 3, 2, or 1 weeks of any treatment with a second therapeutic agent. In some embodiments, a HTRA1-binding agent is administered within 5, 4, 3, 2, or 1 days of any treatment with a second therapeutic agent. It will further be appreciated that the two (or more) agents or treatments can be administered to the subject within a matter of hours or minutes (i.e., substantially simultaneously).

For the treatment of a disease, the appropriate dosage of a HTRA1-binding agent of the present disclosure depends on the disorder or disease to be treated, the severity and course of the disorder or disease, the responsiveness of the disorder or disease, whether the agent is administered for therapeutic or preventative purposes, previous therapy, the patient's clinical history, and so on. A HTRA1-binding agent can be administered one time or over a series of treatments lasting from several days to several months, or until a cure is effected or a diminution of the disease state is achieved.

In some embodiments, the HTRA1-binding agent is administered topically. In certain embodiments of topical administration, the HTRA1-binding agent is administered in combination with a cell penetrating peptide. In certain embodiments, the HTRA1-binding agent and the cell penetrating peptide are delivered as part of a single pharmaceutical composition. Cell penetrating peptides are known in the art (see, e.g., de Cogan et al., 2017, Investigative Ophthalmology & Visual Science, 58:2578-2590). Non-limiting examples of cell penetrating peptides are described in US Patent Publication Nos. 2019/0015521, 2017/0355730, 2018/0346531, and 2012/0065124.

The present disclosure provides compositions comprising a HTRA1-binding agent described herein. The present disclosure also provides pharmaceutical compositions comprising a HTRA1-binding agent described herein and a pharmaceutically acceptable vehicle.

Formulations are prepared for storage and use by combining a purified antibody or agent of the present disclosure with a pharmaceutically acceptable vehicle (e.g., a carrier or excipient). Those of skill in the art generally consider pharmaceutically acceptable carriers, excipients, and/or stabilizers to be inactive ingredients of a formulation or pharmaceutical composition.

Suitable pharmaceutically acceptable vehicles include, but are not limited to, nontoxic buffers such as phosphate, citrate, and other organic acids; salts such as sodium chloride; antioxidants including ascorbic acid and methionine; preservatives such as octadecyldimethylbenzyl ammonium chloride, hexamethonium chloride, benzalkonium chloride, benzethonium chloride, phenol, butyl or benzyl alcohol, alkyl parabens, such as methyl or propyl paraben, catechol, resorcinol, cyclohexanol, 3-pentanol, and m-cresol; low molecular weight polypeptides (e.g., less than about 10 amino acid residues); proteins such as serum albumin, gelatin, or immunoglobulins; hydrophilic polymers such as polyvinylpyrrolidone; amino acids such as glycine, glutamine, asparagine, histidine, arginine, or lysine; carbohydrates such as monosaccharides, disaccharides, glucose, mannose, or dextrins; chelating agents such as EDTA; sugars such as sucrose, mannitol, trehalose or sorbitol; salt-forming counter-ions such as sodium; metal complexes such as Zn-protein complexes; and non-ionic surfactants such as TWEEN or polyethylene glycol. (Remington: The Science and Practice of Pharmacy, 22^{nd} Edition, 2012, Pharmaceutical Press, London.). In some embodiments, the formulation is in the form of an aqueous solution. In some embodiments, the formulation is stored in a lyophilized or in an alternative dried form.

The binding agents of the present disclosure can be formulated in any suitable form for delivery to a target cell/tissue. In some embodiments, a HTRA1-binding agent can be formulated as a liposome, microparticle, microcapsule, albumin microsphere, microemulsion, nanoparticle, nanocapsule, or macroemulsion.

In some embodiments, a HTRA1-binding agent is formulated with liposomes. Methods to produce liposomes are known to those of skill in the art. For example, some liposomes can be generated by reverse phase evaporation with a lipid composition comprising phosphatidylcholine, cholesterol, and PEG-derivatized phosphatidylethanolamine (PEG-PE).

In some embodiments, a HTRA1-binding agent is formulated as a sustained-release preparation. Suitable examples of sustained-release preparations include semi-permeable matrices of solid hydrophobic polymers containing an agent, where the matrices are in the form of shaped articles (e.g., films or microcapsules). Sustained-release matrices include but are not limited to polyesters, hydrogels such as poly(2-hydroxyethyl-methacrylate) or poly(vinyl alcohol), polylactides, copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(−)-3-hydroxybutyric acid.

The pharmaceutical compositions or formulations of the present disclosure can be administered in any number of ways for either local or systemic treatment. Administration can be topical by epidermal or transdermal patches, ointments, lotions, creams, gels, drops, suppositories, sprays, liquids and powders; pulmonary by inhalation or insufflation of powders or aerosols, including by nebulizer, intratracheal, and intranasal; oral; or parenteral including intravenous, intra-arterial, intratumoral, subcutaneous, intraperitoneal, intramuscular (e.g., injection or infusion), intracranial (e.g., intrathecal or intraventricular), ocular, intraocular, or intravitreal.

In some embodiments of the methods described herein, a HTRA1-binding agent described herein is administered by ocular injection. In some embodiments of the methods described herein, a HTRA1-binding agent is administered by intraocular injection. In some embodiments of the methods described herein, a HTRA1-binding agent is administered by intravitreal injection. In some embodiments of the methods described herein, the HTRA1-binding agent is administered by eye drops. In some embodiments of the methods described herein, the HTRA1-binding agent is delivered topically.

### EXAMPLES

### Example 1

### Generation of Antibodies

Anti-HTRA1 antibodies were generated using human HTRA1 (delta N-HTRA1; amino acids 156-480 of SEQ ID NO:1) as the immunogen. Single cell suspensions of lymphocytes were obtained from the spleens and lymph nodes of immunized mice after the individual animals had been determined to have suitable antibody titers. Lymphocytes were fused with murine myeloma cells by standard methods. Hybridoma fusions were plated onto semi-solid media for HAT selection. After 5-7 days, single colonies were selected using a ClonePix™ system and plated into 96-well plates. Cell supernatants were screened by ELISA for binding to human HTRA1 and for inhibition of HTRA1 protease activity.

### Example 2

### Screening Assays

The binding affinities of anti-HTRA1 antibodies to human HTRA1 were measured using a Biacore system (GE Healthcare Life Sciences). Briefly, anti-Fc antibody (Sigma-Aldrich) was immobilized on all four flow cells of a CMS chip using amine coupling reagents (GE Healthcare LifeSciences). Antibodies were captured on flow cells 2, 3, and 4 using flow cell 1 as a reference. Concentrations ranging from 3.3-10 nM of human delta N-HTRA1 were injected at a flow rate of 50 μL/min at 37° C. Kinetic data were collected over time and fit using the simultaneous global fit equation to yield affinity constants (K_{D} values) for each antibody. Similar experiments were carried out to obtain Kd values at 25° C.

Two enzymatic assays were used to characterize HTRA1 protease activity and evaluate inhibitory activity of anti-HTRA1 antibodies. EnzChek® Protease Assay Kit (ThermoFisher Scientific) was used following the manufacturer's instructions. This kit contains casein protein that is heavily labeled with the pH-insensitive green-fluorescent BOPIPY® FL dye that results in almost total quenching of the conjugate's fluorescence. Protease-catalyzed hydrolysis of labeled casein releases highly fluorescent BODIPY® FL dye-labeled peptides. The concomitant increase in fluorescence is proportional to protease activity.

The second assay is a FRET-based method using a quenched substrate peptide. The peptide substrate is Mca-IRRVSYSF(K-Dnp)K (SEQ ID NO:91), referred to as H2-optimal substrate (Innovagen) or H2-Opt peptide. The H2-Opt peptide includes the donor fluorophore Mca (7-methoxycoumarin-4-yl acetyl) and the acceptor (quencher) Dnp (N-2,4-dinitrophenyl). In the intact H2-Opt substrate the Dnp quenches the fluorescence of the Mca donor. Protease-catalyzed hydrolysis of the H2-Opt peptide separates the donor fluorophore and the quencher, thereby resulting in increased Mca fluorescence. The concomitant increase in fluorescence is proportional to protease activity.

Antibodies identified in HTRA1 binding assays were tested for their ability to inhibit HTRA1 protease activity. The range of antibody concentrations was tested to establish a dose response curve and determine IC₅₀ values. Table 5 shows the binding affinity and protease inhibitory activity (e.g., IC50 values) of four representative antibodies.

TABLE 5

| | Protease | | Protease | | | | |
|---|---|---|---|---|---|---|---|
| | Assay— | | Assay— | | | | |
| | Casein | | H2-Opt | | | | |
| | substrate | | peptide | | Binding Assay—SPR | | |
| | IC₅₀ | Kᵢ^{app} | IC₅₀ | Kᵢ^{app} | Kₒₙ | K_{off} | K_{D} |
| Antibody | nM | pM | nM | pM | [1/Ms] | [s⁻¹] | M |
| 9F8 | 0.19 | 35 | 0.12 | 92 | 3.0 × 10⁷ | 4.4 × 10⁻³ | 1.4 × |
| | | | | | | | 10⁻¹⁰ |
| 24F7 | 0.15 | 54 | 0.12 | 118 | 7.4 × 10⁵ | 8.5 × 10⁻⁵ | 1.2 × |
| | | | | | | | 10⁻¹¹ |
| 55B12 | 0.13 | ND | 0.13 | ND | 3.8 × 10⁷ | 17 × 10⁻³ | 4.5 × |
| | | | | | | | 10⁻¹⁶ |
| 65G8 | 0.06 | ND | 0.09 | ND | 6.9 × 10⁶ | 3.0 × 10⁻³ | 4.2 × |
| | | | | | | | 10⁻¹⁰ |

### Example 3

### Sequence Analyses of Anti-HTRA1 Antibodies

Representative antibodies 9F8, 24F7, 55B12, and 65G8 were sequenced and the heavy chain variable region and light chain variable region amino acid sequences are disclosed herein and summarized in Table 6.

TABLE 6

| | Heavy Chain | Light Chain |
|---|---|---|
| Antibody | Variable Region | Variable Region |
| 9F8 | SEQ ID NO:73 | SEQ ID NO:74 |
| 24F7 | SEQ ID NO:68 | SEQ ID NO:69 |
| 55B12 | SEQ ID NO:75 | SEQ ID NO:76 |
| 65G8 | SEQ ID NO:77 | SEQ ID NO:78 |

The heavy chain and light chain variable region CDRs for the individual antibodies are disclosed in Tables 1-4 and as SEQ ID NOs:9-67.

### Example 4

### Characterization of Antibody 24F7

SPR assays were set up using a Biacore system as described herein. Binding data is shown in Table 7. These results demonstrated that antibody 24F7 bound to HTRA1 with high affinity.

TABLE 7

| | Kₒₙ | K_{off} | K_{D} |
|---|---|---|---|
| Temp | [1/Ms] | [s⁻¹] | M |
| 37° C. | 4.0 × 10⁶ | 8.2 × 10⁻⁵ | 2.1 × 10⁻¹¹ |
| 25° C. | 1.6 × 10⁶ | 5.0 × 10⁻⁵ | 3.0 × 10⁻¹¹ |

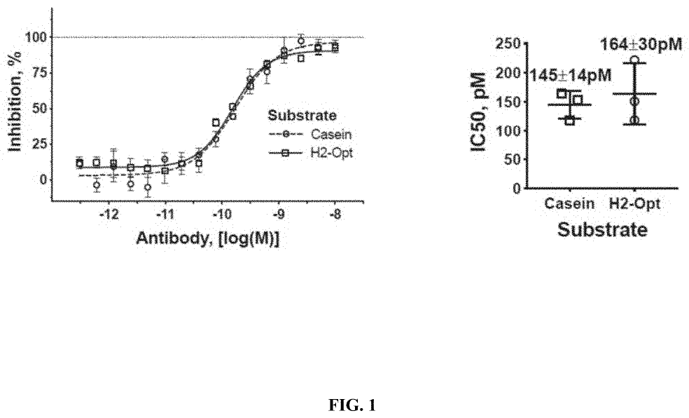

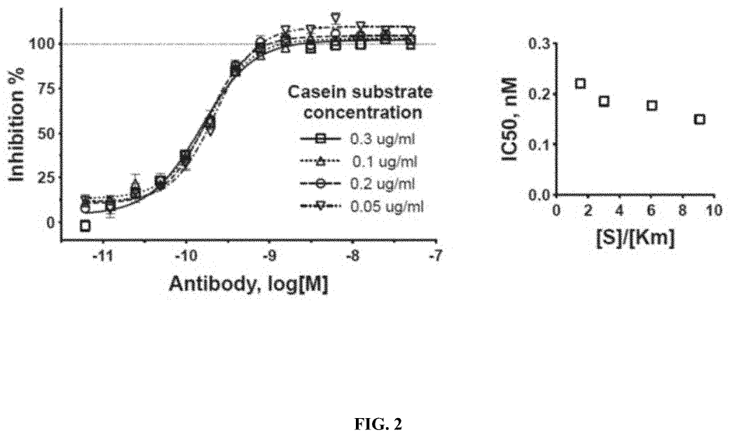

Inhibition of HTRA1 protease activity by antibody 24F7 was assessed as described herein. FIG. 1 shows that antibody 24F7 potently inhibits HTRA1 protease activity in vitro with an IC₅₀ of 145 pM with casein used as the substrate and an IC₅₀ of 164 pM with H2-Opt peptide used as the substrate. These results demonstrated that antibody 24F7 inhibited HTRA1-mediated proteolysis of both protein and peptide substrates. FIG. 2 shows that the IC₅₀ of antibody 24F7 did not change with increasing protein substrate concentration. These results suggest that 24F7 is a noncompetitive inhibitor of HTRA1 and that it is unlikely the antibody would be out-competed by an excess of protein substrate under in vivo conditions.

The human melanoma cell line C32 secretes endogenously expressed HTRA1 into its growth media. Concentrated serum-free conditioned media from C32 cells was used as the source of HTRA1 and used in enzymatic assays as described herein. The inhibitory activity of antibody 24F7 was tested using the conditioned C32 media and an EnzChek® protease assay.

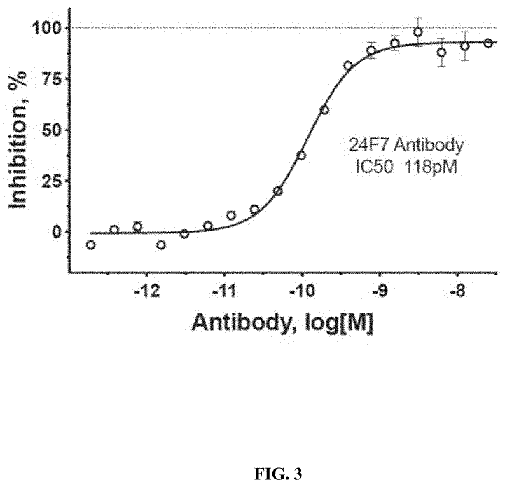

As shown in FIG. 3, antibody 24F7 inhibits protease activity of endogenously expressed HTRA1 with an IC₅₀ of 118 pM.

### Example 5

### Epitope Characterization

A series of mutated HTRA1-catalytic domain (HTRA1-CAT; amino acids 156-364 of SEQ ID NO:1) proteins containing single amino acid substitutions were generated by standard methods (e.g., site directed mutagenesis). Antibody 24F7 was tested by ELISA for binding to these mutant HTRA1-CAT proteins as well as a wild-type HTRA1 peptide consisting of amino acids RKLPFSKREVPV (SEQ ID NO:92; R190—V201 of SEQ ID NO:1).

Table 8 shows results from these binding studies.

TABLE 8

| HTRA1-CAT Mutant Proteins | 24F7 Antibody Binding |
|---|---|
| R190A mutant | − |
| L192A mutant | + |
| P193A mutant | ++ |
| F194A mutant | ++ |
| R197A mutant | − |
| Wild type peptide (R190 - V201) | − |

− no binding; + low binding; ++ normal binding

These results suggest that amino acids R190, L192, and/or R197 comprise at least part of the epitope that antibody 24F7 binds. The results also suggest that the epitope is a conformational epitope since there was no binding to the wild-type HTRA1 peptide consisting of amino acids RKLPFSKREVPV (amino acids 190-201 of SEQ ID NO:1).

### Example 6

### Generation of Humanized Antibody

Based on the antibody characterization data as well as additional studies, antibody 24F7 was selected for humanization. Antibody 24F7 was humanized by methods known to those skilled in the art and humanized 24F7 is referred to herein as hz24F7. The heavy chain variable region sequence of antibody hz24F7 is set forth in SEQ ID NO:70 and the light chain variable region sequence of antibody hz24F7 is set forth in SEQ ID NO:72. Antibody 24F7 was found to have a potential isomerization site in CDR3 of the heavy chain variable region, EGYSYDGGGYYFDY (SEQ ID NO:11). During the humanization process, the heavy chain variable region CDR3 was reengineered to remove the isomerization site resulting in a heavy chain variable region CDR3 comprising the amino acid sequence EGYSYEGGGYYFDY (SEQ ID NO:25). There was an additional amino acid change made in the FR3 region of the heavy chain variable region, resulting in a humanized variant of 24F7 referred to herein as hz24F7.v2. The heavy chain variable sequence of antibody hz24F7.v2 is SEQ ID NO:71 and the light chain variable sequence is SEQ ID NO:72; CDRs are disclosed in Table 1B.

The heavy chain sequence of antibody hz24F7.v2 is set forth in SEQ ID NO:87 and SEQ ID NO:88 (with and without signal sequence, respectively) and the light chain sequence of antibody hz24F7.v2 is set forth in SEQ ID NO:89 and SEQ ID NO:90 (with and without signal sequence, respectively).

### Example 7

Characterization of hz24F7.v2

The binding affinity of hz24F7.v2 to a HTRA1 full length protein (HTRA1-FL) or a HTRA1-CAT was determined using a Biacore system as described herein and compared with the binding affinity of the parental 24F7 antibody (see Table 9).

TABLE 9

| | HTRA1-FL | | | HTRA1-CAT | | |
|---|---|---|---|---|---|---|
| Antibody | Kₒₙ | K_{off} | K_{D} | Kₒₙ | K_{off} | K_{D} |
| Temp | [1/Ms] | [s−1] | M | [1/Ms] | [s−1] | M |
| hz24F7.v2 | 7.1 × 10⁵ | 4.2 × 10⁻⁵ | 5.8 × 10⁻¹¹ | 3.9 × 10⁶* | 5.6 × | <1 × |
| 25° C. | | | | | 10⁻⁵ | 10⁻¹⁰ |
| hz24F7.v2 | 1.5 × 10⁶ | 1.4 × 10⁻⁴ | 9.3 × 10⁻¹¹ | 1.1 × 10⁷* | 1.7 × | <1 × |
| 37° C. | | | | | 10⁻⁴ | 10⁻¹⁰ |
| 24F7 | 1.8 × 10⁶ | 8.6 × 10⁻⁵ | 4.7 × 10⁻¹¹ | ND | ND | ND |
| 37° C. | | | | | | |

*= Limit of Detection

Antibody hz24F7.v2 had a binding affinity to human HTRA1-FL of 9.3×10¹⁰ M at 37° C. as compared to the parental antibody's binding affinity of approximately 4.7×10⁻¹⁰ M. The binding affinity of hz24F7.v2 to a HTRA1 catalytic domain protein (HTRA1-CAT) was also determined. The binding affinity of hz24F7.v2 to HTRA1-CAT was less than 1×10⁻¹⁰ M. The Kₒₙ rate was at the limit of detection, so the K_{D} could not be quantitated to a more defined number.

These results demonstrated that the humanization process for antibody 24F7, as well as the removal of a potential isomerization site within CDR3 of the heavy chain, did not have a significant effect on the binding capabilities to human HTRA1.

To confirm that the humanized version of antibody 24F7 retained the ability to inhibit H IRA1 protease activity, hz24F7.v2 was assessed in casein and H2-Opt assays as described herein.

As shown in Table 10, the ability of antibody hz24F7.v2 to inhibit HTRA1 protease activity is equal to or better than the parental ability 24F7.

TABLE 10

| | Casein Assay | | H2-Opt Assay | |
|---|---|---|---|---|
| Antibody | IC₅₀, nM | Kᵢ^{app}, nM | IC₅₀, nM | Kᵢ^{app}, nM |
| hz24F7.v2 | 0.29 | 0.086 | 0.12 | 0.067 |
| 24F7 | 0.35 | 0.099 | 0.17 | 0.076 |

These results demonstrated that the humanization process for antibody 24F7, as well as the removal of a potential isomerization site within CDR3 of the heavy chain, did not have any effect on the ability of the antibody to inhibit protease activity of HTRA1. Importantly, the IC₅₀ and Kᵢ^{app} values show that hz24F7.v2 is an extremely potent inhibitor of HTRA1.

### Example 8

Pharmacokinetics of Antibody hz24F7.v2 in Cynomolgus Monkeys

The pharmacokinetics (PK) of anti-HTRA1 antibody hz24F7.v2 were assessed in cynomolgus monkey eyes after intravitreal injection (IVT) at a dose of 3.2 mg/eye or 12.8 mg/eye. PK was also assessed in monkeys after a single intravenous injection (IV) at a dose of 75 mg/kg. Antibody H24F7.v2 was administered by intravitreal injection (IVT) as a single dose of 75 μL/eye. Following IV or IVT injections serum samples were taken at 10 minutes, 30 minutes, 1 hour, 4 hours, 8 hours, and on days 2, 4, 8, 15, and 29. The concentration of hz24F7.v2 was evaluated in the vitreous humor and in serum.

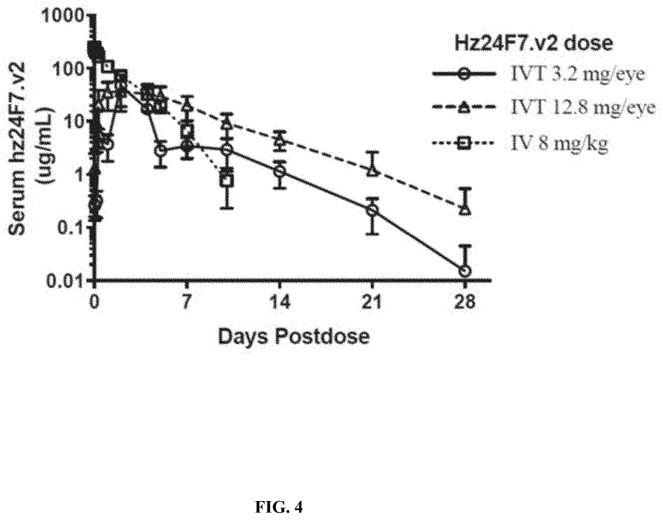

The results are shown in FIG. 4 and analyses of the data (summarized in Table 11) determined that the half-life of hz24F7.v2 in the vitreous humor was approximately 3 days. This is similar to the reported PKs of bevacizumab and aflibercept in vitreous humor of cynomolgus monkeys. The half-life of hz24F7.v2 in serum was approximately 1 day, which is consistent with a lack of FcRn binding and recycling (due to the mutated IgG1 heavy chain).

TABLE 11

| hz24F7.v2 | Cmax | AUC | T_{1/2} |
|---|---|---|---|
| Route/Dose | (μg/mL) | (day * μg/mL) | (day) |
| IV 8 mg/kg | 248 | 404 | 1.1 |
| IVT 3.2 mg/eye | 1460 | 6990 | 2.7 |
| IVT 12.8 mg/eye | 4010 | 30200 | 3.4 |

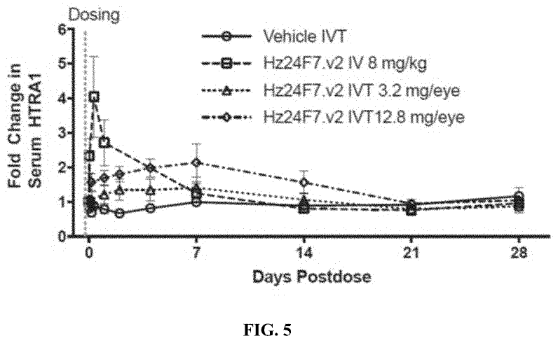

The amount of HTRA1 protein was measured in the PK serum samples. As shown in FIG. 5, HTRA1 protein transiently and quickly increased approximately 4-fold after IV dosing of hz24F7.v2. In contrast, HTRA1 protein increased approximately 2-fold over a longer period of time after IVT dosing of hz24F7.v2. No increase in HTRA1 protein was observed in the vehicle-treated group. The increase in HTRA1 protein in hz24F7.v2-injected animals suggests that the antibody binds HTRA1 and causes transient accumulation of HTRA1 in serum.

### Example 9

Structural Characterization of Fragmented hz24F7.v2

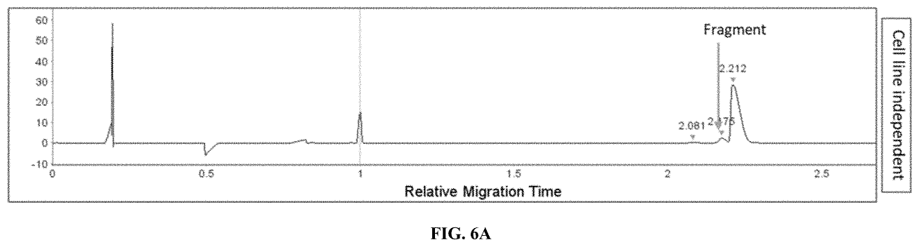

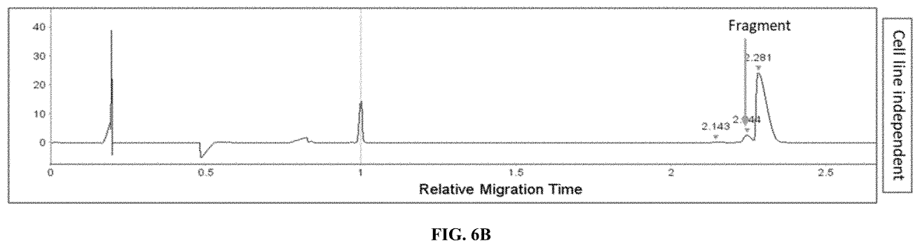

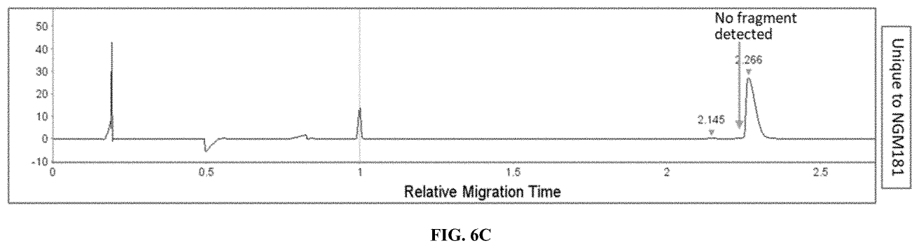

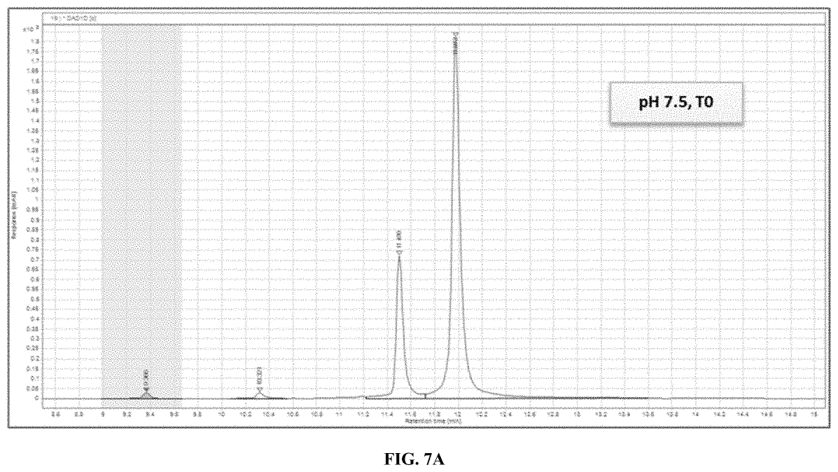

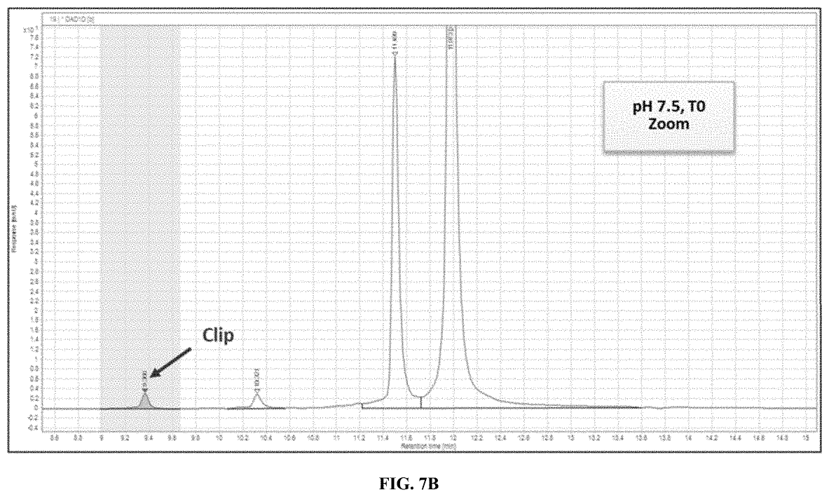

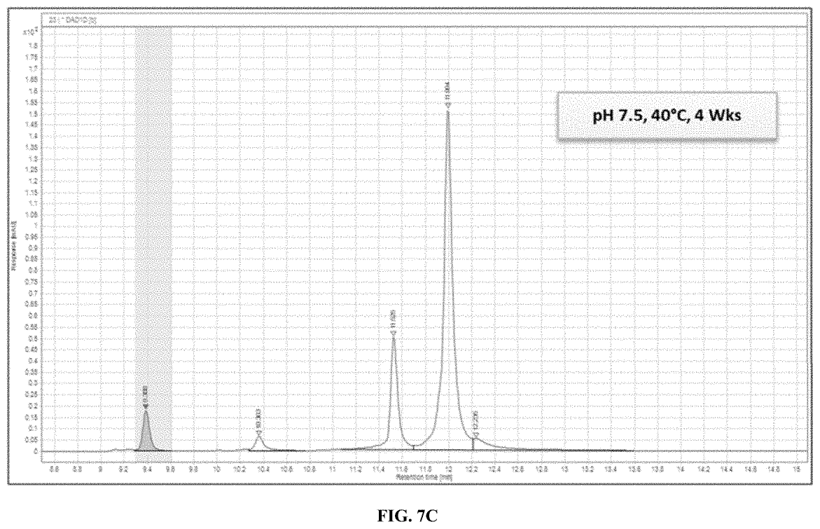

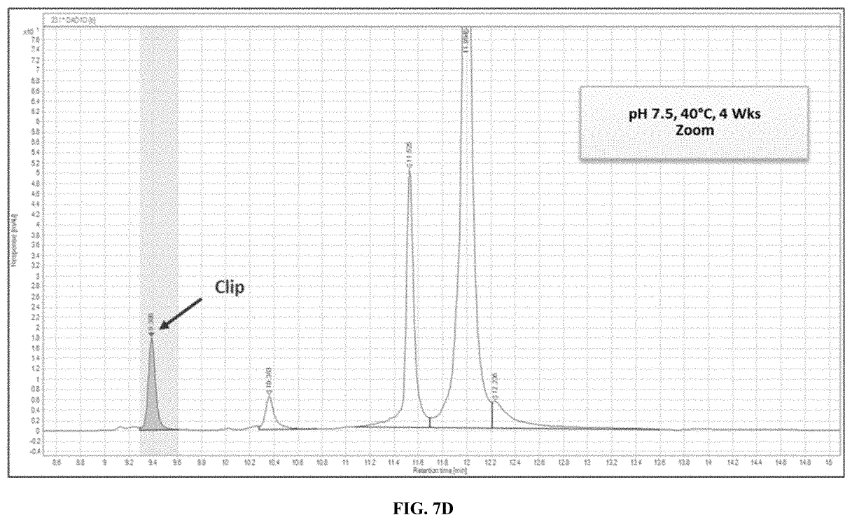

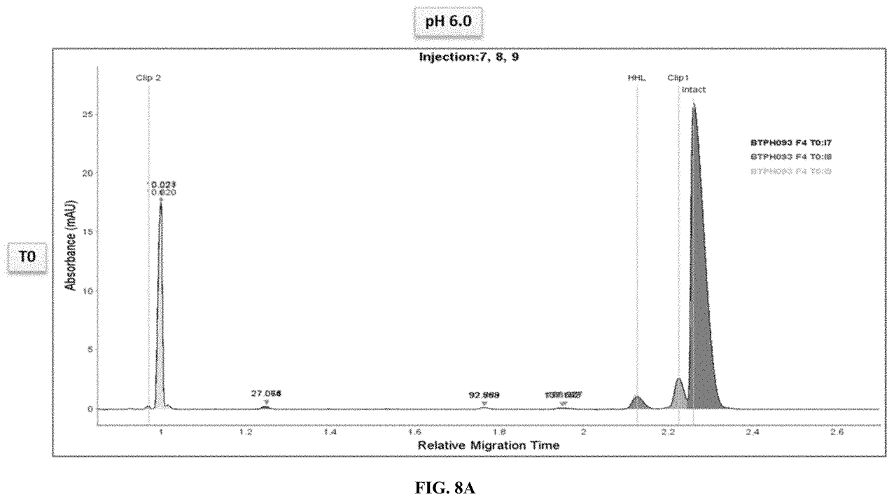

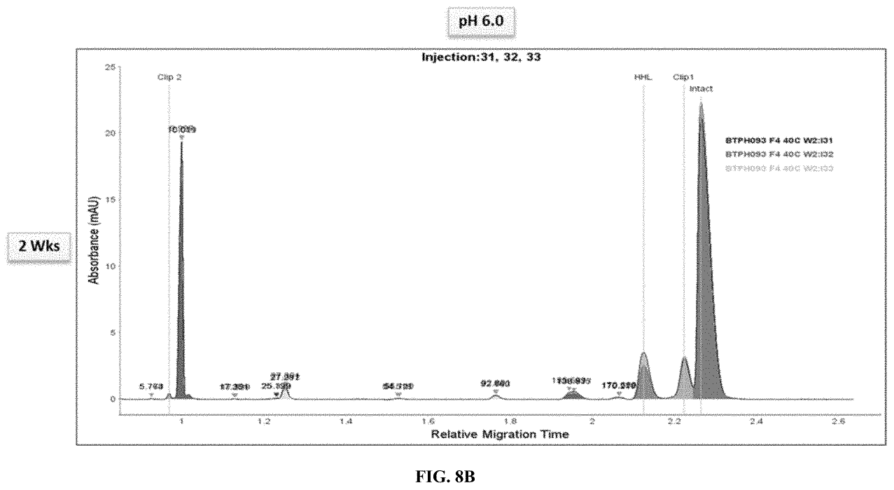

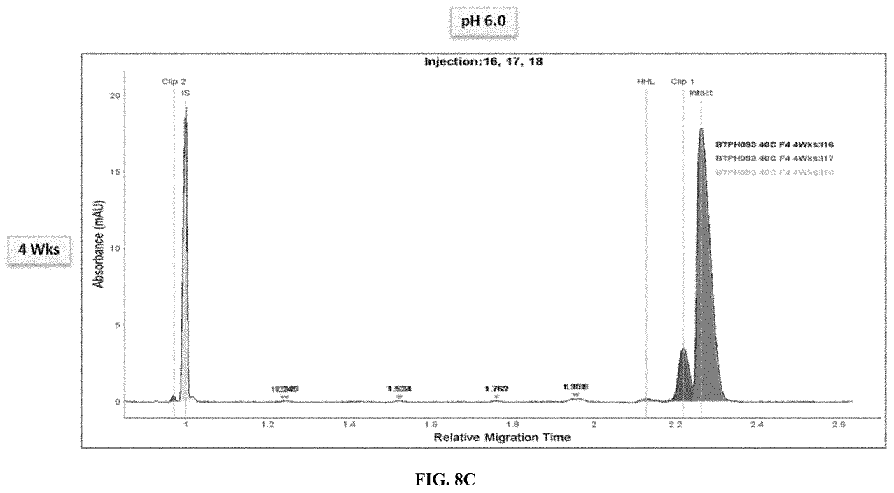

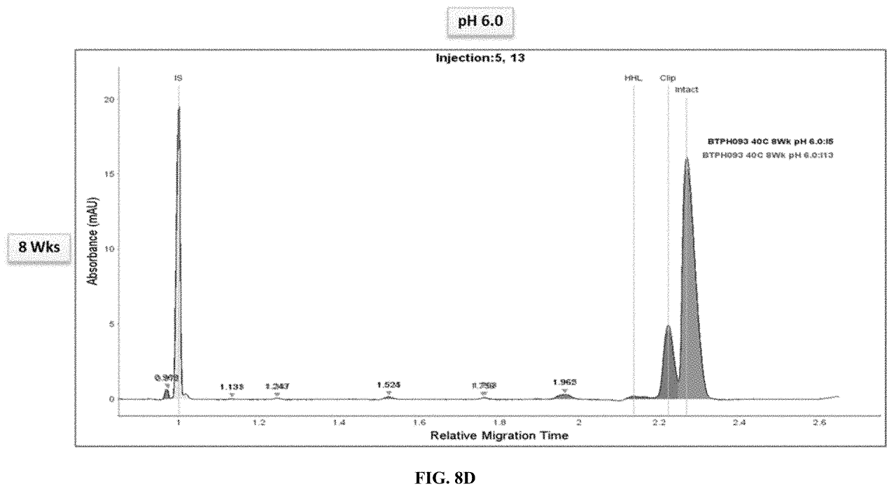

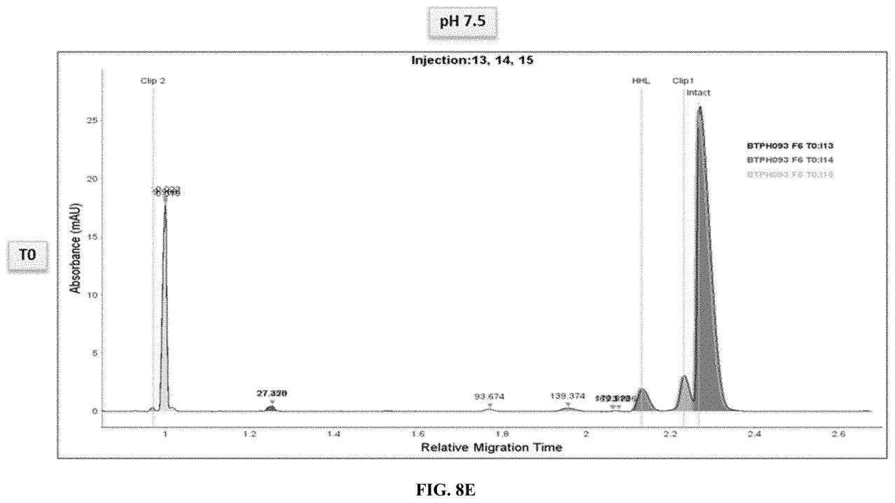

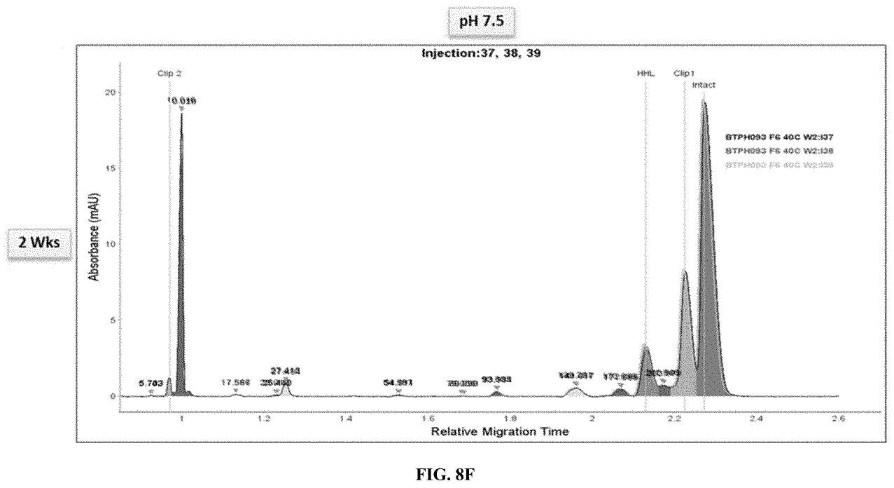

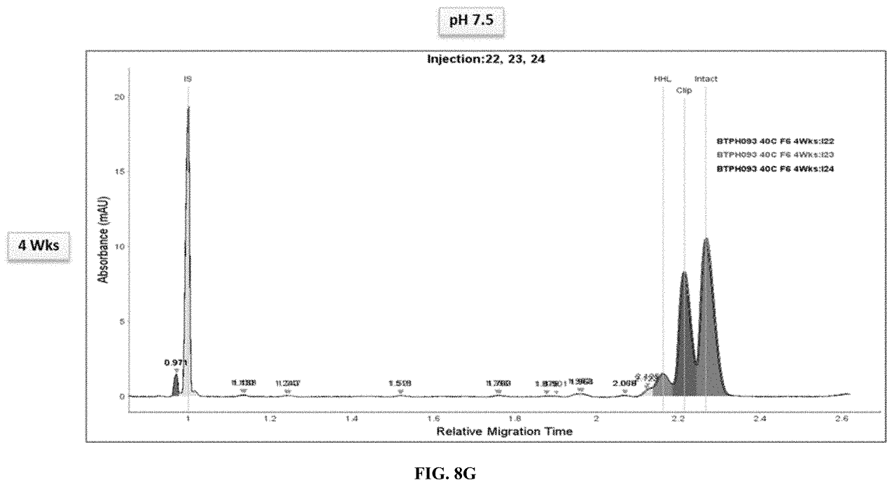

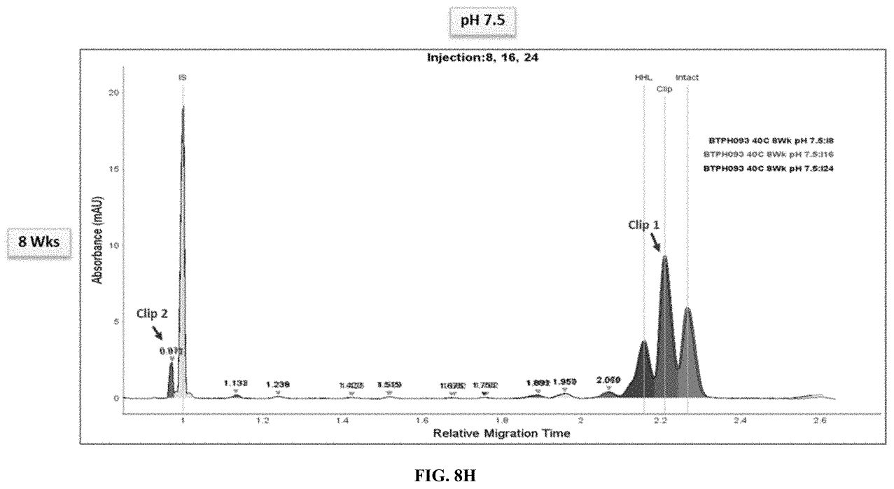

Recombinant expression of hz24F7.v2 antibodies in CHO cells (FIG. 6A) and HEK293 cells (FIG. 6B) produced a unique fragment relative to an unrelated negative control antibody (anti-GFRAL) (FIG. 6C). Together these data demonstrate that the fragmentation of hz24F7.v2 is cell-line independent and unique to hz24F7.v2. Further analysis by RP-HPLC (FIGS. 7A-7D) demonstrated that long-term storage (4 weeks) of hz24F7.v2, at a neutral pH (7.5) and temperature of 40° C., resulted in significant fragmentation of hz24F7.v2 (indicated by “clip” in FIGS. 7B and 7D). Additional analysis by CE-SDS (FIG. 8A-8H) further demonstrated that the level of fragmented hz24F7.v2 increased significantly from 0 to 8 weeks at a pH of 7.5 and temperature of 40° C. When stored at a more acidic pH of 6.0, fragmentation was less pronounced. These data indicate that hz24F7.v2 fragmentation is pH-dependent and time-dependent.

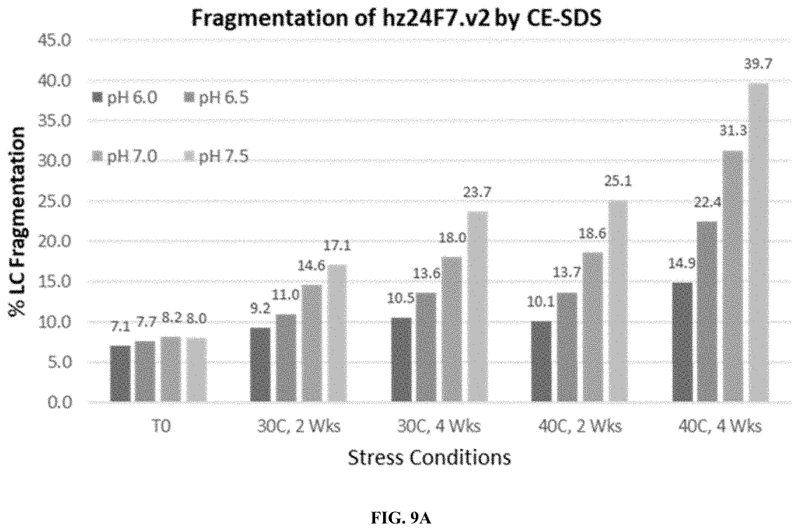

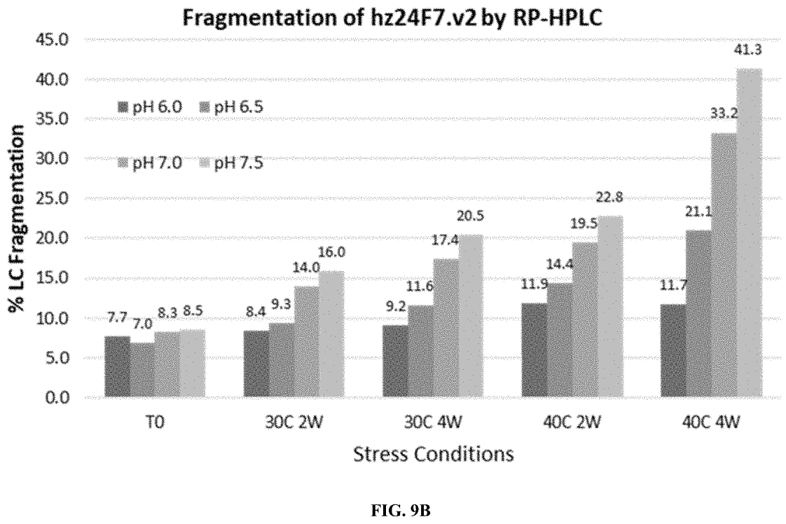

In order to determine whether the observed fragment was an artifact, fragmentation results were generated by two orthogonal methods, CE-SDS (FIG. 9A) and RP-HPLC (FIG. 9B) for comparison. FIGS. 9A and 9B demonstrate that the observed fragmentation results generated by CE-SDS and RP-HPLC were comparable, indicating that the observed hz24F7.v2 fragment was not an artifact. Note that while acidic conditions (pH 6.0 and 6.5) minimize hz24F7.v2 fragmentation, fragmentation still increased at higher temperatures and longer storage times. More significant fragmentation of hz24F7.v2 was observed at pH 7.5 particularly at higher temperatures and longer storage times.

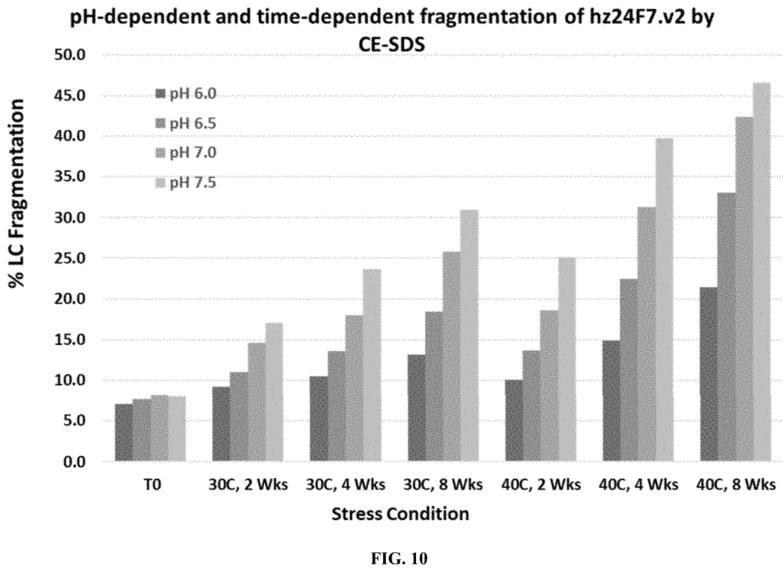

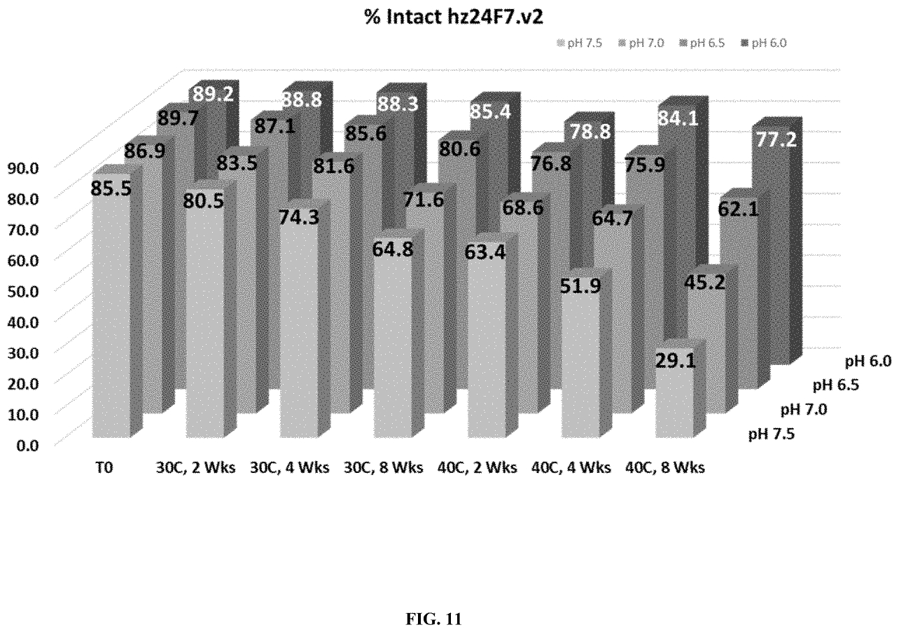

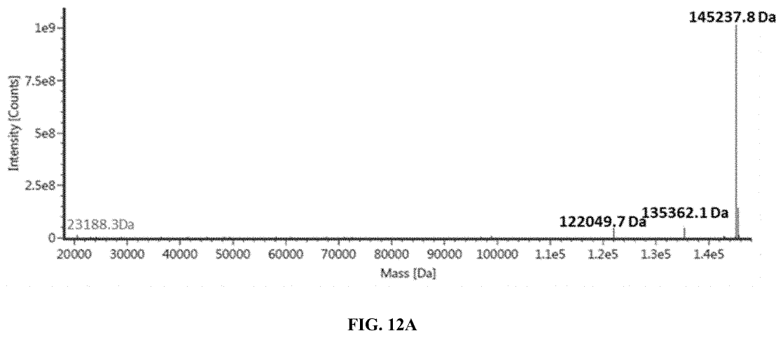

Similar results were obtained when hz24F7.v2 was stored at 30° C. and 40° C. FIG. 10 shows the pH-dependent and time-dependent fragmentation of hz24F7.v2 analyzed by CE-SDS upon storage at either 30° C. or 40° C. for up to 8 weeks in 2 week increments. Although fragmentation of hz24F7.v2 increases across all storage conditions tested, it is more pronounced at higher pH and over longer storage times. FIG. 11 also demonstrates that the percentage of intact hz24F7.v2 decreased over long-term storage (2 to 8 weeks) regardless of the pH of the storage conditions. Consistent with the previous results of more significant/pronounced fragmentation of hz24F7.v2 at higher temperature and longer storage times, the percentage of intact hz24F7.v2 decreased at higher temperature and longer storage times.

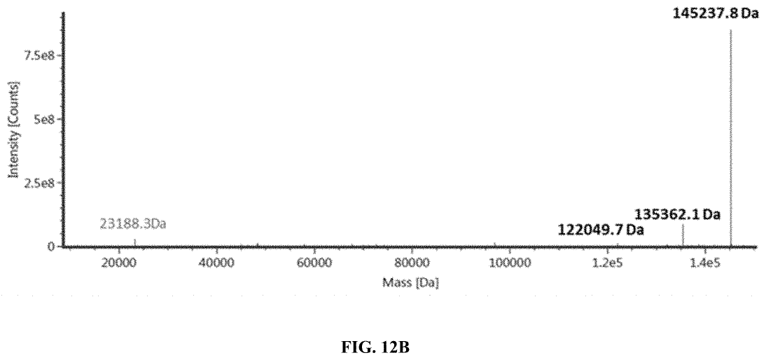

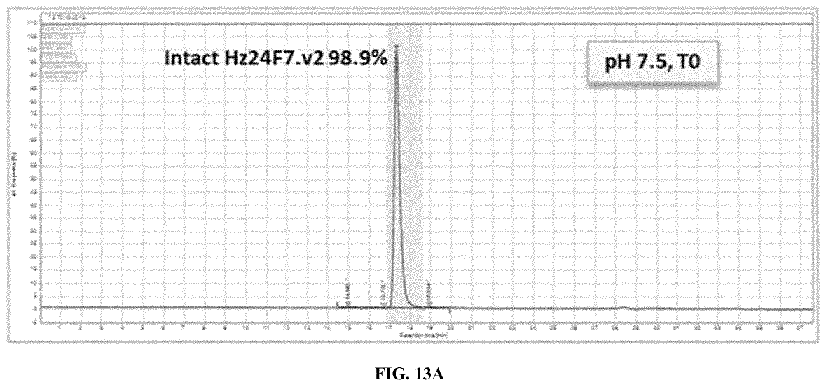

Finally, in order to determine the exact location of fragmentation of hz24F7.v2, intact mass spectrometry (MS) analysis was performed (FIGS. 12A and 12B and Tables 12 and 13). MS identified a fragmentation site between amino acid positions 91 and 92 on the light chain CDR3 of hz24F7.v2. FIG. 12A shows the S91 fragmentation site at 0 weeks. FIG. 12B shows the S91 fragmentation site at 2 weeks and 40° C. In FIG. 12A and FIG. 12B, the 145237.8 Da peptide mass corresponds to intact hz24F7.v2, the 135362.1 Da peptide mass corresponds to an hz24F7.v2 fragmented at position S91 in only one light chain of the antibody, the 122049.7 Da mass peptide corresponds to an hz24F7.v2 missing one of its light chains and the 23188.3 Da mass peptide corresponds to hz24F7.v2 free light chain. Fragmentation occurred via pH-dependent chemical hydrolysis of the peptide bond between amino acid positions 91 and 92. Fragmentation was initiated by a nucleophilic addition of the serine OH group to the neighboring N-terminal peptide bond to form an oxazolidine intermediate that further rearranges to an ester intermediate that is finally hydrolyzed.

TABLE 12

| | Observed | Theoretical |
|---|---|---|
| Assignment | Mass (Da) | Mass (Da) |
| Intact hz24F7.v2 | 145237.8 | 145235.2 |
| hz24F7.v2 one of LC | 135362.1 | 135360.2 |
| fragmented at S-91 | | |
| hz24F7.v2 | 122049.7 | 122050.4 |
| one of LC missing | | |
| Light Chain (LC) | 23188.3 | 23188.8 |

TABLE 13
Relative percent abundance of
one of the LC fragmented
at S-91

| | Tox DP Lot: |
|---|---|
| Tox DP Lot: | D25-BTPH-093- |
| D25-BTPH-093- | 1909M-010 |
| 1909M-010 | 2 weeks at 40 C. |
| 4.6% | 10.0% |

### Example 10

Functional Characterization of Fragmented hz24F7.v2

Functional assays were performed to determine whether hz24F7.v2 retained complete antigen-binding capability.

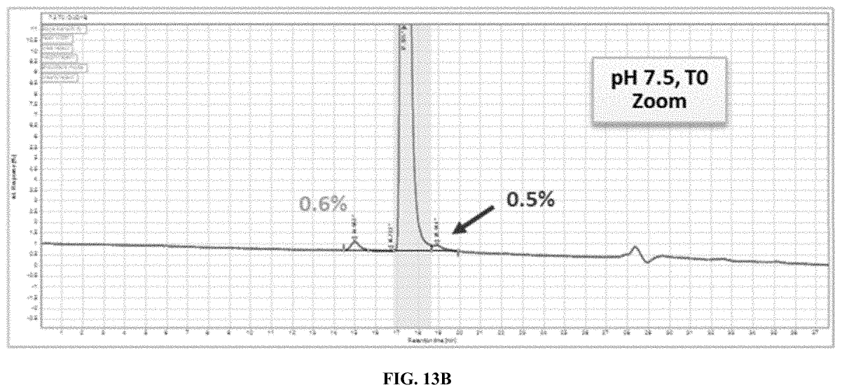

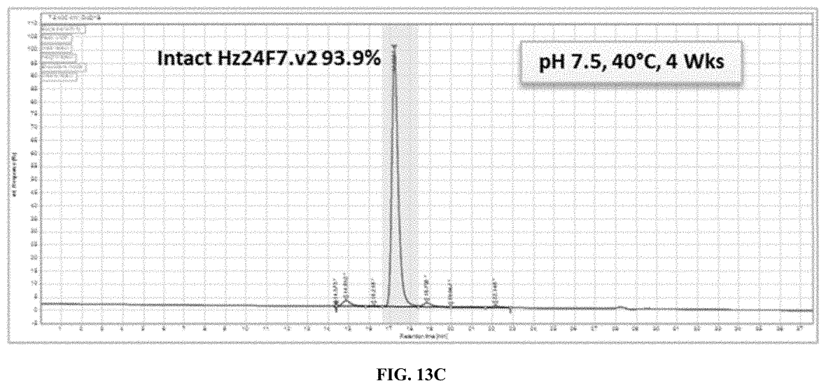

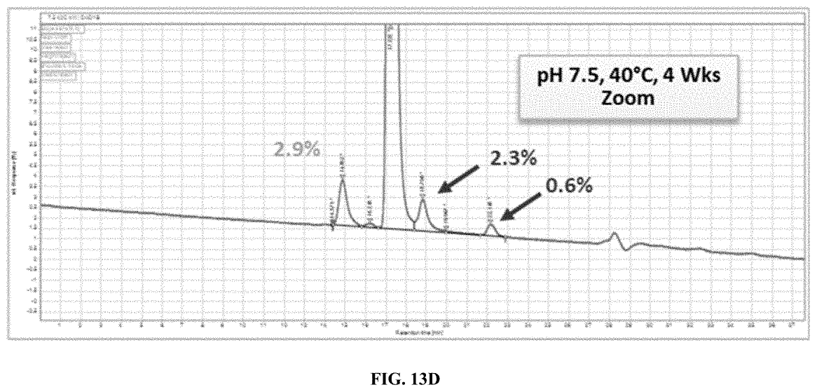

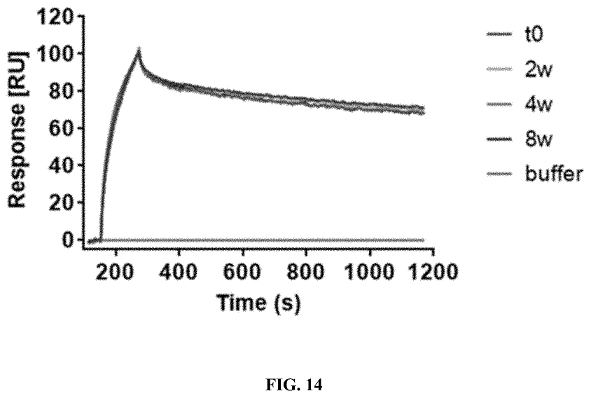

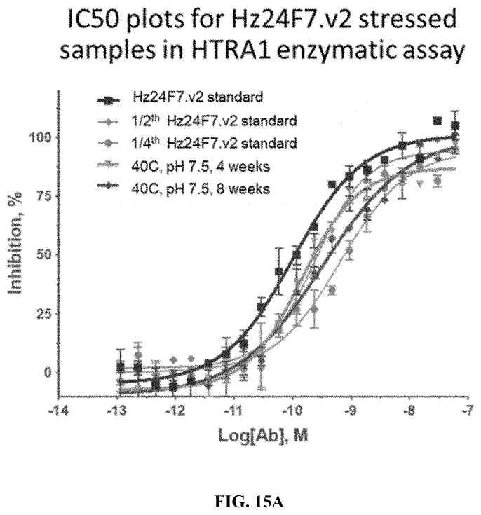

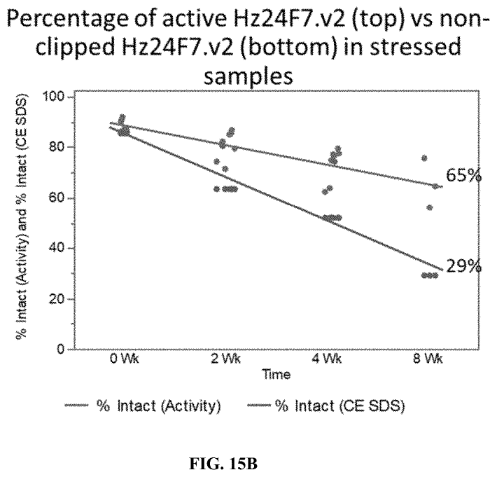

FIGS. 13A-13D show SEC-HPLC analysis performed under non-denaturing conditions of hz24F7.v2 upon storage at pH 7.5, 40° C. for 4 weeks. The results show that the main peak, which represents intact hz24F7.v2, was 98.9% at the start of the incubation period (T0=0 weeks) and that at 4 weeks was 93.9%, which represents a 5% decrease of intact hz24F7.v2. In contrast, the fragmented levels of hz24F.v2 as previously determined by RP-HPLC demonstrated a fragmentation level of 32.8% (FIG. 9A) at pH 7.5, 40° C. and 4 weeks storage time. Similarly contrasting results were obtained by CE-SDS analysis; the fragmented levels of hz24F7.v2 were 31.7% (FIG. 9B) at pH 7.5, 40° C. and 4 weeks storage times. These contrasting data put together (SEC-HPLC under native conditions compared to RP-HPLC and CE-SDS), demonstrated that the fragmented portion of hz24F7.v2 (light chain CDR3) remained bound to the hz24F7.v2 antibody. Next, SPR assays were set up using a Biacore system as described herein to determine whether fragmented hz24F7.v2 retains HTRA1 binding activity. Binding data, shown in Table 14 and FIG. 14, demonstrated that across 8 weeks, at pH 7.4, 40° C., hz24F7.v2 was still capable of binding to HTRA1, with no observable loss of binding which provides further evidence that the fragmented portion of hz24F7.v2 remained bound to the hz24F7.v2 antibody.

TABLE 14

| hz24F7.v2 | | Binding Kinetics | | |
|---|---|---|---|---|
| Samples, pH 7.4 | Capture | to HTRA1, 25° C. | | |
| @40° C. | [RU] | Kₒₙ | K_{off} | K_{D} |
| 0 weeks | 53 | 1.95E+06 | 4.16E−04 | 2.13E−10 |
| 2 weeks | 56 | 2.10E+06 | 3.74E−04 | 1.78E−10 |
| 4 weeks | 51.5 | 2.68E+06 | 3.88E−04 | 1.44E−10 |
| 8 weeks | 51 | 3.01E+06 | 5.02E−04 | 1.67E−10 |

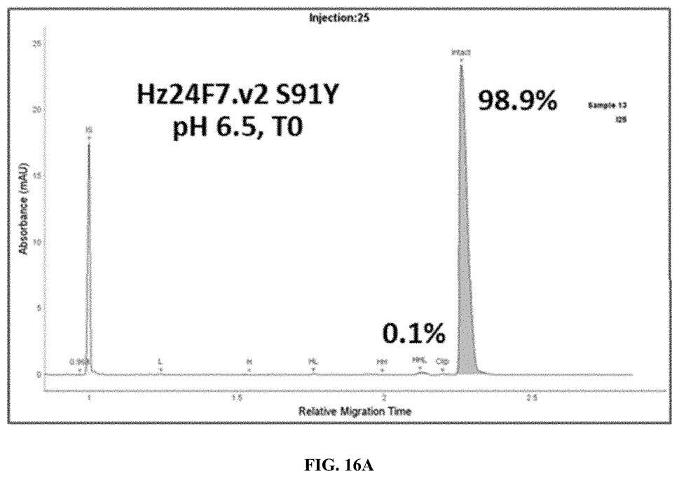

Further, an HTRA1 enzyme assay was performed to determine whether fragmented hz24F7.v2 retains its HTRA1 inhibitory capability. FIG. 15A demonstrates that the fragmented hz24F7.v2 as represented by the samples stored at pH 7.5, 40° C. for 4 weeks and 8 weeks had minimal impact on its maximal inhibitory enzymatic activity compared to hz24F7.v2 standard (T0=0 weeks). FIG. 15B demonstrates similar maximal inhibitory enzymatic activity to hz24F7.v2 (standard) T0 sample.

### Example 11

Introduction of Amino Acid Substitutions to hz24F7.v2

Based on the mechanism of hz24F7.v2 fragmentation, various amino acid substitutions were introduced at amino acid positions 91 and 92 in the light chain CDR3 of hz24F7.v2 to eliminate fragmentation. As Table 15 demonstrates, we identified several amino acid substitutions at positions S91 (S91D, S91Y) and S92 (S92Y, S92D, S92T) that reduced hz24F7.v2 fragmentation to background levels at 0 weeks, when stored at pH 7.4 and 40° C.

TABLE 15

| | | % Fragmentation |
|---|---|---|
| hz24F7.v2 LC | LC CDR3 | at 0 weeks, pH 7.4, |
| Mutation | Sequence | 40° C. |
| hz24F7.v2 | QQWSSYPT | 14.5 |
| S91D | QQWDSYPT | 0.0 |
| S91Y | QQWYSYPT | 0.0 |
| S91T | QQWTSYPT | 0.9 |
| S91A | QQWASYPT | 2.2 |
| S91L | QQWLSYPT | 1.0 |
| S92Y | QQWSYYPT | 0.4 |
| S92D | QQWSDYPT | 0.4 |
| S92T | QQWSTYPT | 0.3 |

TABLE 16

| Mutation | kₒₙ | k_{off} | K_{D} | IC₅₀, pM |
|---|---|---|---|---|
| S91Y | 4.54E+06 | 1.84E−04 | 4.05E−11 | 70 |
| S92T | 9.81E+06 | 1.97E−04 | 2.00E−11 | 98 |
| S91A | 3.63E+06 | 1.69E−04 | 4.65E−11 | ND |
| S91T | 7.10E+06 | 1.89E−04 | 2.66E−11 | ND |
| S91L | 3.67E+06 | 1.61E−04 | 4.39E−11 | 84 |
| S92D | 8.70E+06 | 2.17E−04 | 2.49E−11 | 67 |
| S92Y | 8.56E+06 | 2.75E−04 | 3.21E−11 | 97 |

In order to determine whether a hz24F7.v2 variant, comprising one of the amino acid substitutions disclosed in Table 15, retains its HTRA1 binding capability, functional characterization was performed via an SPR assay as described herein and an HTRA1 enzyme assay. The results are shown in Table 16. Table 16 demonstrates a variety of S91 and S92 substitutions that have comparable affinity and efficacy (IC₅₀) and any of these amino acids can be used to make substitutions at position 91 and 92 in order to overcome this observed fragmentation for hz24F7.v2.

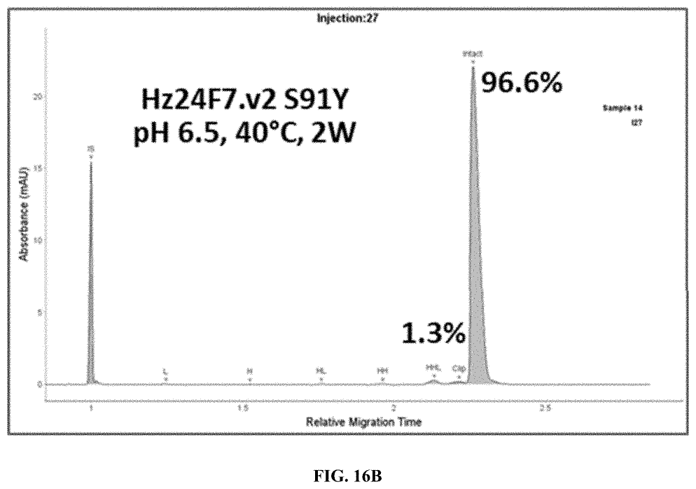

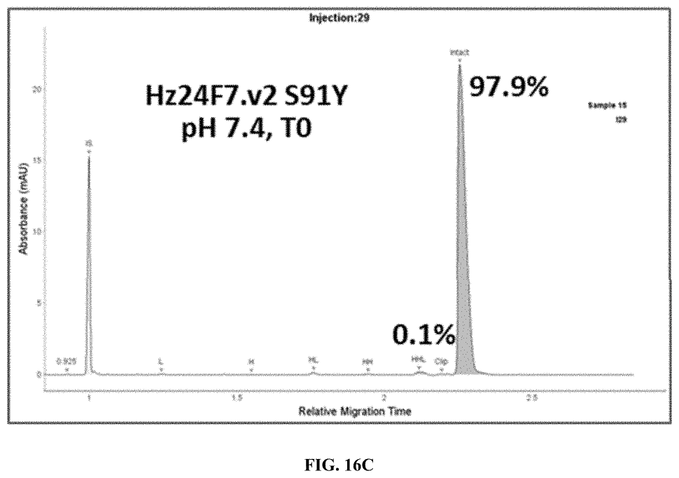

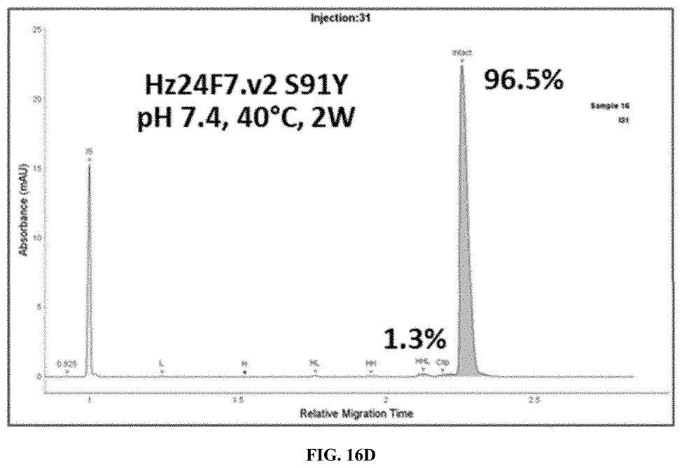

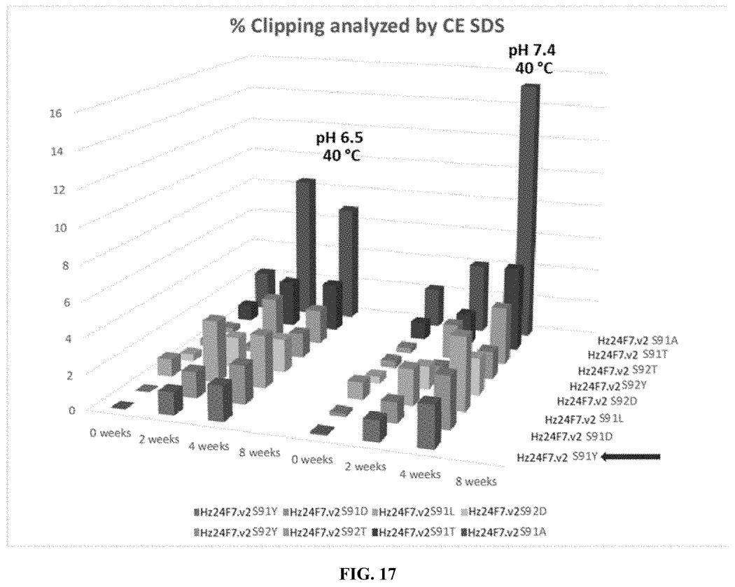

Fragmentation studies were further conducted with hz24F7.v2 S91Y and hz24F7.v2 S92T across 2 and 4 weeks. FIGS. 16A-16D demonstrate that hz24F7.v2 S91Y does not undergo appreciable fragmentation under 2 weeks storage at 40° C. at pH 6.5 and pH 7.4. FIG. 17 shows a trend across 4 weeks that S91Y, S91D, S92D, S92Y and S92T have reduced levels of fragmentation when stored at pH 7.4 and 40° C. compared to S91L, S91T and S91A. Table 17 shows that at 4 weeks incubation at pH 7.4 and 40° C., only 2.4% of the hz24F7.v2 S91Y was fragmented and only 2.1% of the hz24F7.v2 S92T was fragmented, compared to approximately 40% of hz24F7.v2 that was fragmented.

TABLE 17

| | | | % | | |
|---|---|---|---|---|---|
| | | % | Fragmentation | | |
| | | Fragmentation | at time | | |
| hz24F7.v2 LC | LC CDR3 | at time 0, | 4weeks, | Viscosity @ | Purification |
| Mutation | Sequence | pH 7.4, 40° C. | pH 7.4, 40° C. | (mg/ml) | yield (mg/L) |
| hz24F7.v2 | QQWSSYPT | 14.5 | 39.9 | 13.6 (175) | 300 |
| S91Y | QQWYSYPT | 0.0 | 2.4 | 18.6 (160) | 322 |
| S92T | QQWSTYPT | 0.3 | 2.1 | 12.9 (160) | 153 |

Table 18 further demonstrates by CE SDS analysis that, across 4 weeks, the percentage of fragmented hz24F7.v2 S91Y increased only minimally compared to hz24F7.v2, of which 48% was fragmented.

TABLE 18

| | % Fragmentation | | | | | | |
|---|---|---|---|---|---|---|---|
| | Determined by CE | | | | | | |
| | SDS pH 7.4, 40° C. | | | Affinity at Baseline | | | |
| | 0 | 2 | 4 | | | KD | IC₅₀ |
| Antibody | weeks | weeks | weeks | kₒₙ | k_{off} | pM | pM |
| hz24F7.v2 | 14.5 | 26.6 | 48.1 | 1.28E+07 | 2.25E−04 | 17.6 | 66 |
| hz24F7.v2 | 0.1 | 1.3 | 2.4 | 4.54E+06 | 1.84E−04 | 40.5 | 70 |
| S91Y | | | | | | | |

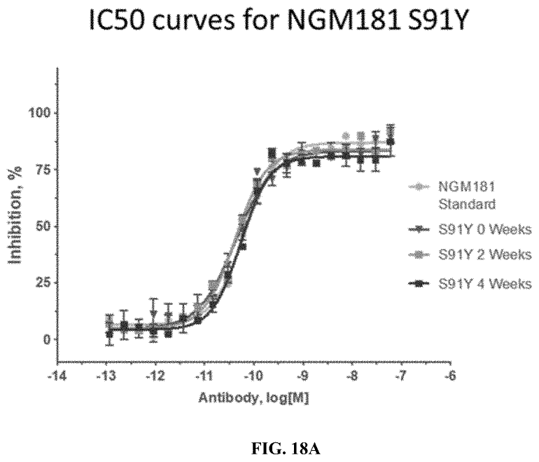

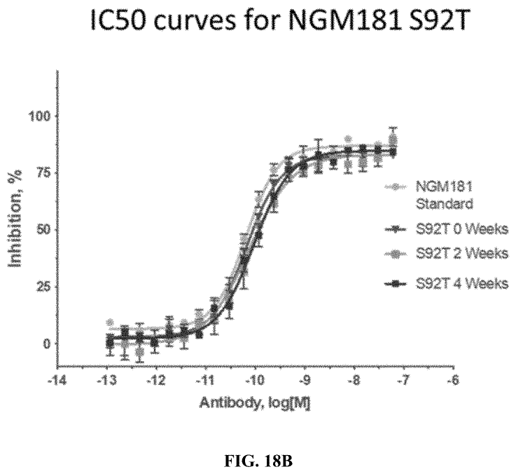

Table 19 depicts the data presented in FIG. 18, and further demonstrates that there was no significant difference for the IC₅₀ values of hz24F7.v2 S91Y or hz24F7.v2 S92T compared to hz24F7.v2. This data indicates that the inhibitory capability of hz24F7.v2 S91Y and hz24F7.v2 S92T is comparable to hz24F7.v2.

TABLE 19

| | hz24F7.v2 S91Y | hz24F7.v2 S92T |
|---|---|---|
| Storage conditions | IC₅₀ (pM) | IC₅₀ (pM) |
| pH 7.4, 40° C., 0 weeks | 43.4 | 67.5 |
| pH 7.4, 40° C., 2 weeks | 42.0 | 75.2 |
| pH 7.4, 40° C., 4 weeks | 54.3 | 93.7 |
| hz24F7.v2 | 64.5 | 64.5 |

Overall, this example demonstrates that hz24F7.v2 S91Y serves as a good alternative to hz24F7.v2 as introduction of the S91Y substitution reduces antibody fragmentation and preserves hz24F7.v2 binding affinity and inhibitory activity, with low viscosity and high expression.

Although the foregoing present disclosure has been described in some detail by way of illustration and example for purposes of clarity of understanding, the descriptions and examples should not be construed as limiting the scope of the present disclosure. The embodiments of the present disclosure described herein are intended to be merely exemplary, and those skilled in the art will recognize numerous equivalents to the specific procedures described herein. All such equivalents are considered to be within the scope of the present disclosure and are covered by the embodiments.

All publications, patents, patent applications, internet sites, and accession numbers/database sequences including both polynucleotide and polypeptide sequences cited herein are hereby incorporated by reference in their entirety for all purposes to the same extent as if each individual publication, patent, patent application, internet site, or accession number/database sequence were specifically and individually indicated to be so incorporated by reference.

Following are sequences disclosed in the application. CDR sequences are listed in Tables 1-4.

Human HTRA1 amino acid sequence with predicted signal sequence underlined (SEQ ID NO: 1)
MQIPRAALLPLLLLLLAAPASAQLSRAGRSAPLAAGCPDRCEPARCPPQPEHCEGGRARD
ACGCCEVCGAPEGAACGLQEGPCGEGLQCVVPFGVPASATVRRRAQAGLCVCASSEPVCG
SDANTYANLCQLRAASRRSERLHRPPVIVLQRGACGQGQEDPNSLRHKYNFIADVVEKIA
PAVVHIELFRKLPFSKREVPVASGSGFIVSEDGLIVINAHVVINKHRVKVELKNGATYEA
KIKDVDEKADIALIKIDHQGKLPVLLLGRSSELRPGEFVVAIGSPFSLQNTVTTGIVSTT
QRGGKELGLRNSDMDYIQTDAIINYGNSGGPLVNLDGEVIGINTLKVTAGISFAIPSDKI
KKFLTESHDRQAKGKAITKKKYIGIRMMSLTSSKAKELKDRHRDFPDVISGAYIIEVIPD
TPAEAGGLKENDVIISINGQSVVSANDVSDVIKRESTLNMVVRRGNEDIMITVIPEEIDP
Human HTRA1 amino acid sequence without predicted signal sequence (SEQ ID NO: 2)
QLSRAGRSAPLAAGCPDRCEPARCPPQPEHCEGGRARDACGCCEVCGAPEGAACGLQEGP
CGEGLQCVVPFGVPASATVRRRAQAGLCVCASSEPVCGSDANTYANLCQLRAASRRSERL
HRPPVIVLQRGACGQGQEDPNSLRHKYNFIADVVEKIAPAVVHIELFRKLPFSKREVPVA
SGSGFIVSEDGLIVINAHVVINKHRVKVELKNGATYEAKIKDVDEKADIALIKIDHQGKL
PVLLLGRSSELRPGEFVVAIGSPFSLQNTVITGIVSTTQRGGKELGLRNSDMDYIQTDAI
INYGNSGGPLVNLDGEVIGINTLKVTAGISFAIPSDKIKKFLTESHDRQAKGKAITKKKY
IGIRMMSLTSSKAKELKDRHRDFPDVISGAYIIEVIPDTPAEAGGLKENDVIISINGQSV
VSANDVSDVIKRESTLNMVVRRGNEDIMITVIPEEIDP
Human HTRA1 amino acid sequence without N-terminal domain (aa 101-480) (SEQ ID NO: 3)
VRRRAQAGLCVCASSEPVCGSDANTYANLCQLRAASRRSERLHRPPVIVLQRGACGQGQE
DPNSLRHKYNFIADVVEKIAPAVVHIELFRKLPFSKREVPVASGSGFIVSEDGLIVTNAH
VVTNKHRVKVELKNGATYEAKIKDVDEKADIALIKIDHQGKLPVLLLGRSSELRPGEFVV
AIGSPFSLQNTVITGIVSTTQRGGKELGLRNSDMDYIQTDAIINYGNSGGPLVNLDGEVI
GINTLKVTAGISFAIPSDKIKKFLTESHDRQAKGKAITKKKYIGIRMMSLTSSKAKELKD
RHRDFPDVISGAYIIEVIPDTPAEAGGLKENDVIISINGQSVVSANDVSDVIKRESTLNM
VVRRGNEDIMITVIPEEIDP
Human HTRA1 serine protease domain (SEQ ID NO: 4)
GSGFIVSEDGLIVINAHVVINKHRVKVELKNGATYEAKIKDVDEKADIALIKIDHQGKLP
VLLLGRSSELRPGEFVVAIGSPFSLQNTVTTGIVSTTQRGGKELGLRNSDMDYIQTDAII
NYGNSGGPLVNLDGEVIGINTLKVTAGISFAIPSDKIKKFL
Rabbit HTRA1 amino acid sequence with predicted signal sequence underlined (SEQ ID NO: 5)
MGWAARPRALAPAAPKALRSSPRRCCPARAQLAAVGAAMQSSRAARLLPLLLLLLAAPAW
AQPPRAGRSAPAATSPVAGCPERCEPARCAPPPTNCEGGRVRDACGCCEVCGAPEGAACG
LQEGPCGEGLQCVVSFGVPASATVRRRSQAGVCVCASNEPVCGSDANTYANLCQLRAASR
RSERLQQPPVIVLQRGACGQGQEDPNSLRHKYNFIADVVEKIAPAVVHIELFRKLPFSKR
EVPVASGSGFIVSEDGLIVINAHVVINKHRVKVELKNGATYEAKIKDVDEKADIALIKID
HQGKLPVLLLGRSSELRPGEFVVAIGSPFSLQNTVTTGIVSTTQRGGKELGLRNSDMDYI
QTDAIINYGNSGGPLVNLDGEVIGINTLKVTAGISFAIPSDKIKKFLTESHDRQAKGKAI
TKKKYIGIRMMSLTSSKAKELKDRHRDFPDVLSGAYIIEVIPDTPAEAGGLKENDVIISI
NGQSVVSANDVSDVIKKDSTLNMVVRRGNEDIMITVIPEEIDP
Rabbit HTRA1 amino acid sequence without predicted signal sequence (SEQ ID NO: 6)
QPPRAGRSAPAATSPVAGCPERCEPARCAPPPTNCEGGRVRDACGCCEVCGAPEGAACGL
QEGPCGEGLQCVVSFGVPASATVRRRSQAGVCVCASNEPVCGSDANTYANLCQLRAASRR
SERLQQPPVIVLQRGACGQGQEDPNSLRHKYNFIADVVEKIAPAVVHIELFRKLPFSKRE
VPVASGSGFIVSEDGLIVINAHVVINKHRVKVELKNGATYEAKIKDVDEKADIALIKIDH
QGKLPVLLLGRSSELRPGEFVVAIGSPFSLQNTVITGIVSTTQRGGKELGLRNSDMDYIQ
TDAIINYGNSGGPLVNLDGEVIGINTLKVTAGISFAIPSDKIKKFLTESHDRQAKGKAIT
KKKYIGIRMMSLTSSKAKELKDRHRDFPDVLSGAYIIEVIPDTPAEAGGLKENDVIISIN
GQSVVSANDVSDVIKKDSTLNMVVRRGNEDIMITVIPEEIDP
Cyno HTRA1 amino acid sequence with predicted signal sequence underlined (SEQ ID NO: 7)
MQIPRAALLPLLLLLLLAAPASAQLSRAGRSPEHCEGGRARDACGCCEVCGAPEGAACGL
QEGPCGEGLQCVVPFGVPASATVRRRAQAGLCVCASNEPVCGSDANTYANLCQLRAASRR
SERLHRPPVIVLQRGACGQGQEDPNSLRHKYNFIADVVEKIAPAVVHIELFRKLPFSKRE
VPVASGSGFIVSEDGLIVINAHVVINKHRVKVELKNGATYEAKIKDVDEKADIALIKIDH
QGKLPVLLLGRSSELRPGEFVVAIGSPFSLQNTVITGIVSTTQRGGKELGLRNSDMDYIQ
TDAIINYGNSGGPLVNLDGEVIGINTLKVTAGISFAIPSDKIKKFLTESHDRQAKGKAIT
KKKYIGIRMMSLTSSKAKELKDRHRDFPDVISGAYIIEVIPDTPAEAGGLKENDVIISIN
GQSVVSANDVSDVIKRESTLNMVVRRGNEDIMITVIPEEIDP
Cyno HTRA1 amino acid sequence without predicted signal sequence (SEQ ID NO: 8)
QLSRAGRSPEHCEGGRARDACGCCEVCGAPEGAACGLQEGPCGEGLQCVVPFGVPASATV
RRRAQAGLCVCASNEPVCGSDANTYANLCQLRAASRRSERLHRPPVIVLQRGACGQGQED
PNSLRHKYNFIADVVEKIAPAVVHIELFRKLPFSKREVPVASGSGFIVSEDGLIVTNAHV
VTNKHRVKVELKNGATYEAKIKDVDEKADIALIKIDHQGKLPVLLLGRSSELRPGEFVVA
IGSPFSLQNTVITGIVSTTQRGGKELGLRNSDMDYIQTDAIINYGNSGGPLVNLDGEVIG
INTLKVTAGISFAIPSDKIKKFLTESHDRQAKGKAITKKKYIGIRMMSLTSSKAKELKDR
HRDFPDVISGAYIIEVIPDTPAEAGGLKENDVIISINGQSVVSANDVSDVIKRESTLNMV
VRRGNEDIMITVIPEEIDP
24F7 Heavy chain variable region amino acid sequence (SEQ ID NO: 68)
QVQLQQSGAELVRPGASVKLSCKASGYTFTDYEMHWVKQTPVHGLEWIGAIDPETGGTAY
NQKFKGKATLTADKSSSTAYMELRSLTSEDSAVYYCTREGYSYDGGGYYFDYWGQGTTLT
VSS
24F7 Light chain variable region amino acid sequence (SEQ ID NO: 69)
QIVLIQSPAIMSASPGEKVTMTCSVSSSVSYMYWYQQKPGSSPRLLIYDTSNLASGVPVR
FSGSGSGTSYSLTISRMEAEDAATYYCQQWSSYPTFGGGTKLEIK
hz24F7 Heavy chain variable region amino acid sequence (SEQ ID NO: 70)
QVQLVQSGAEVKKPGASVKVSCKASGYTFTDYEMHWVRQAPGQRLEWMGAIDPETGGTAY
NQKFKGRVTITADKSASTAYMELSSLRSEDTAVYYCAREGYSYDGGGYYFDYWGQGTLVT
VSS
hz24F7.v2 variant Heavy chain variable region amino acid sequence (SEQ ID NO: 71)
QVQLVQSGAEVKKPGASVKVSCKASGYTFTDYEMHWVRQAPGQRLEWMGAIDPETGGTAY
NQKFKGRVTITADKSASTAYMELSSLRSEDTAVYYCTREGYSYEGGGYYFDYWGQGTLVT
VSS
hz24F7.v2 Light chain variable region amino acid sequence (SEQ ID NO: 72)
DIQMTQSPSSLSASVGDRVTITCSVSSSVSYMYWYQQKPGKAPKLLIYDTSNLASGVPSR
FSGSGSGTDFTFTISSLQPEDIATYYCQQWSSYPTFGQGTKLEIK
9F8 Heavy chain variable region amino acid sequence (SEQ ID NO: 73)
QVQLQQPGAELVRPGSSVKLSCKASGYAFTTYWMHWVKQRPIQGLEWIGNIDPSDSETHY
NQKFRDKATLTVDKSSSTAYMQLSSLTSEDSAVYYCARDYGAFDVWGIGTIVIVSS
9F8 Light chain variable region amino acid sequence (SEQ ID NO: 74)
QAVVTQESALTTSSGETVTLICRSSTGAVTTRNFASWVQEKPDHLFTGLIGGTNNRAPGV
PARFSGSLIGDKAALTITGAQTEDEAIYFCALWYSNLWVFGGGTKLTVL
55B12 Heavy chain variable region amino acid sequence (SEQ ID NO: 75)
QVQLQQPGAELVKPGASVKLSCKASGYTFTNYWMHWVKQRPGQGLEWIGNIDPSDSETHY
NQKFKDKATLAVDKSSSTAYMQLSSLTSEDSAVYYCAREDSSGYGAYWGQGTLVTVSA
55B12 Light chain variable region amino acid sequence (SEQ ID NO: 76)
QIVLTQSPAIMSASPGEKVTMTCSASSSVNYMHWYQQKSGTSPKRWIYDTSKLASGVPDR
FSGSGSGTSYSLTISSMEAEDAATYYCQQWSSHPLTFGAGTKLELK
65G8 Heavy chain variable region amino acid sequence (SEQ ID NO: 77)
QVQLQQSGPQLVRPGASVKISCKASGYSFTSYWMHWVKQRPGQGLEWIGMIDPSDSETRL
NQKFKDKATLTIDKSSSTAYMQLSSPTSEDSAVYYCTRDYFDYWGQGTTLTVSS
65G8 Light chain variable region amino acid sequence (SEQ ID NO: 78)
QIVLIQSPAIMSTSPGEKVTMTCSASSSVSYMYWYQQKPGSSPRLLIYDTSNLASGVPVR
FSGSGSGTSYSLTISRMEAEDAATYYCQQWSSYPYTFGGGTKLEIK
Human IgG1 constant region (SEQ ID NO: 79)
ASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTHTCPPCPAPELLGG
PSVFLFPPKPKDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQVYTLPPSREE
MTKNQVSLTCLVKGFYPSDIAVEWESNGQPENNYKTTPPVLDSDGSFFLYSKLTVDKSRW
QQGNVFSCSVMHEALHNHYTQKSLSLSPGK
Human IgG1 constant region E233A/L235A (SEQ ID NO: 80)
ASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTHTCPPCPAPALAGG
PSVFLFPPKPKDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQVYTLPPSREE
MTKNQVSLTCLVKGFYPSDIAVEWESNGQPENNYKTTPPVLDSDGSFFLYSKLTVDKSRW
QQGNVFSCSVMHEALHNHYTQKSLSLSPGK
Human IgG1 constant region L234A/L235A (SEQ ID NO: 81)
ASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTHTCPPCPAPEAAGG
PSVFLFPPKPKDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQVYTLPPSREE
MTKNQVSLTCLVKGFYPSDIAVEWESNGQPENNYKTTPPVLDSDGSFFLYSKLTVDKSRW
QQGNVFSCSVMHEALHNHYTQKSLSLSPGK
Human IgG1 constant region L234A/L235A/P329G (SEQ ID NO: 82)
ASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTHTCPPCPAPEAAGG
PSVFLFPPKPKDTLMISRTPEVTCVVVDVSHEDPEVKFNWYVDGVEVHNAKTKPREEQYN
STYRVVSVLTVLHQDWLNGKEYKCKVSNKALGAPIEKTISKAKGQPREPQVYTLPPSREE
MTKNQVSLTCLVKGFYPSDIAVEWESNGQPENNYKTTPPVLDSDGSFFLYSKLTVDKSRW
QQGNVFSCSVMHEALHNHYTQKSLSLSPGK
Human IgG1 constant region N297G (SEQ ID NO: 83)
ASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTHTCPPCPAPELLGG
PSVFLFPPKPKDTLMISRTPEVTCVVVDVSHEDPEVKFNWYVDGVEVHNAKTKPREEQYG
STYRVVSVLTVLHQDWLNGKEYKCKVSNKALPAPIEKTISKAKGQPREPQVYTLPPSREE
MTKNQVSLTCLVKGFYPSDIAVEWESNGQPENNYKTTPPVLDSDGSFFLYSKLTVDKSRW
QQGNVFSCSVMHEALHNHYTQKSLSLSPGK
Human IgG1 constant region N297G/H310A (SEQ ID NO: 84)
ASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTHTCPPCPAPELLGG
PSVFLFPPKPKDTLMISRTPEVTCVVVDVSHEDPEVKFNWYVDGVEVHNAKTKPREEQYG
STYRVVSVLTVLAQDWLNGKEYKCKVSNKALPAPIEKTISKAKGQPREPQVYTLPPSREE
MTKNQVSLTCLVKGFYPSDIAVEWESNGQPENNYKTTPPVLDSDGSFFLYSKLTVDKSRW
QQGNVFSCSVMHEALHNHYTQKSLSLSPGK
Human Kappa light chain constant region (SEQ ID NO: 85)
RTVAAPSVFIFPPSDEQLKSGTASVVCLLNNFYPREAKVQWKVDNALQSGNSQESVTEQD
SKDSTYSLSSTLTLSKADYEKHKVYACEVTHQGLSSPVTKSFNRGEC
Human Lambda light chain constant region (SEQ ID NO: 86)
GQPKAAPSVTLFPPSSEELQANKATLVCLISDFYPGAVTVAWKADSSPVKAGVETTTPSK
QSNNKYAASSYLSLTPEQWKSHRSYSCQVTHEGSTVEKTVAPTECS
hz24F7.v2 Heavy chain amino acid sequence with signal sequence underlined (SEQ ID NO: 87)
MDMRVPAQLLGLLLLWLRGARCQVQLVQSGAEVKKPGASVKVSCKASGYTFTDYEMHWVR
QAPGQRLEWMGAIDPETGGTAYNQKFKGRVTITADKSASTAYMELSSLRSEDTAVYYCTR
EGYSYEGGGYYFDYWGQGTLVTVSSASTKGPSVFPLAPSSKSTSGGTAALGCLVKDYFPE
PVTVSWNSGALTSGVHTFPAVLQSSGLYSLSSVVTVPSSSLGTQTYICNVNHKPSNTKVD
KKVEPKSCDKTHTCPPCPAPELLGGPSVFLFPPKPKDTLMISRIPEVTCVVVDVSHEDPE
VKFNWYVDGVEVHNAKTKPREEQYGSTYRVVSVLTVLAQDWLNGKEYKCKVSNKALPAPI
EKTISKAKGQPREPQVYTLPPSREEMTKNQVSLTCLVKGFYPSDIAVEWESNGQPENNYK
TTPPVLDSDGSFFLYSKLTVDKSRWQQGNVFSCSVMHEALHNHYTQKSLSLSPGK
hz24F7.v2 Heavy chain amino acid sequence without signal sequence (SEQ ID NO: 88)
QVQLVQSGAEVKKPGASVKVSCKASGYTFTDYEMHWVRQAPGQRLEWMGAIDPETGGTAY
NQKFKGRVTITADKSASTAYMELSSLRSEDTAVYYCTREGYSYEGGGYYFDYWGQGTLVT
VSSASTKGPSVFPLAPSSKSTSGGTAALGCLVKDYFPEPVTVSWNSGALTSGVHTFPAVL
QSSGLYSLSSVVTVPSSSLGTQTYICNVNHKPSNTKVDKKVEPKSCDKTHTCPPCPAPEL
LGGPSVFLFPPKPKDTLMISRIPEVTCVVVDVSHEDPEVKFNWYVDGVEVHNAKTKPREE
QYGSTYRVVSVLTVLAQDWLNGKEYKCKVSNKALPAPIEKTISKAKGQPREPQVYTLPPS
REEMTKNQVSLTCLVKGFYPSDIAVEWESNGQPENNYKTTPPVLDSDGSFFLYSKLTVDK
SRWQQGNVFSCSVMHEALHNHYTQKSLSLSPGK
hz24F7.v2 Light chain amino acid sequence with signal sequence underlined (SEQ ID NO: 89)
MDMRVPAQLLGLLLLWLRGARCDIQMTQSPSSLSASVGDRVTITCSVSSSVSYMYWYQQK
PGKAPKLLIYDTSNLASGVPSRFSGSGSGTDFTFTISSLQPEDIATYYCQQWSSYPTFGQ
GTKLEIKRTVAAPSVFIFPPSDEQLKSGTASVVCLLNNFYPREAKVQWKVDNALQSGNSQ
ESVTEQDSKDSTYSLSSTLTLSKADYEKHKVYACEVTHQGLSSPVTKSFNRGEC
hz24F7.v2 Light chain amino acid sequence without signal sequence (SEQ ID NO: 90)
DIQMTQSPSSLSASVGDRVTITCSVSSSVSYMYWYQQKPGKAPKLLIYDTSNLASGVPSR
FSGSGSGTDFTFTISSLQPEDIATYYCQQWSSYPTFGQGTKLEIKRTVAAPSVFIFPPSD
EQLKSGTASVVCLLNNFYPREAKVQWKVDNALQSGNSQESVTEQDSKDSTYSLSSTLTLS
KADYEKHKVYACEVTHQGLSSPVTKSFNRGEC
Substrate peptide (SEQ ID NO: 91)
IRRVSYSFK₁K
1 = wherein there is a dnp molecule attached to K
HTRA1 peptide (SEQ ID NO: 92)
RKLPFSKREVPV
Hexa-histidine tag (SEQ ID NO: 93)
HHHHHH
hz24F7.v2 S91Y Light chain variable region amino acid sequence (SEQ ID NO: 96)
DIQMTQSPSSLSASVGDRVTITCSVSSSVSYMYWYQQKPGKAPKLLIYDTSNLASGVPSR
FSGSGSGTDFTFTISSLQPEDIATYYCQQWYSYPTFGQGTKLEIK
hz24F7.v2 S91Y Light chain amino acid sequence with signal sequence underlined (SEQ ID
NO: 97)
MDMRVPAQLLGLLLLWLRGARCDIQMTQSPSSLSASVGDRVTITCSVSSSVSYMYWYQQK
PGKAPKLLIYDTSNLASGVPSRFSGSGSGTDFTFTISSLQPEDIATYYCQQWYSYPTFGQ
GTKLEIKRTVAAPSVFIFPPSDEQLKSGTASVVCLLNNFYPREAKVQWKVDNALQSGNSQ
ESVTEQDSKDSTYSLSSTLTLSKADYEKHKVYACEVTHQGLSSPVTKSFNRGEC
hz24F7.v2 S91Y Light chain amino acid sequence without signal sequence (SEQ ID NO: 98)
DIQMTQSPSSLSASVGDRVTITCSVSSSVSYMYWYQQK
PGKAPKLLIYDTSNLASGVPSRFSGSGSGTDFTFTISSLQPEDIATYYCQQWYSYPTFGQ
GTKLEIKRTVAAPSVFIFPPSDEQLKSGTASVVCLLNNFYPREAKVQWKVDNALQSGNSQ
ESVTEQDSKDSTYSLSSTLTLSKADYEKHKVYACEVTHQGLSSPVTKSFNRGEC
hz24F7.v2 S92T Light chain variable region amino acid sequence (SEQ ID NO: 101)
DIQMTQSPSSLSASVGDRVTITCSVSSSVSYMYWYQQKPGKAPKLLIYDTSNLASGVPSR
FSGSGSGTDFTFTISSLQPEDIATYYCQQWYSYPTFGQGTKLEIK
hz24F7.v2 S92T Light chain amino acid sequence with signal sequence underlined (SEQ ID
NO: 102)
MDMRVPAQLLGLLLLWLRGARCDIQMTQSPSSLSASVGDRVTITCSVSSSVSYMYWYQQK
PGKAPKLLIYDTSNLASGVPSRFSGSGSGTDFTFTISSLQPEDIATYYCQQWSTYPTFGQ
GTKLEIKRTVAAPSVFIFPPSDEQLKSGTASVVCLLNNFYPREAKVQWKVDNALQSGNSQ
ESVTEQDSKDSTYSLSSTLTLSKADYEKHKVYACEVTHQGLSSPVTKSFNRGEC
hz24F7.v2 S92T Light chain amino acid sequence without signal sequence (SEQ ID NO: 103)
DIQMTQSPSSLSASVGDRVTITCSVSSSVSYMYWYQQKPGKAPKLLIYDTSNLASGVPSR
FSGSGSGTDFTFTISSLQPEDIATYYCQQWSTYPTFGQGTKLEIKRTVAAPSVFIFPPSD
EQLKSGTASVVCLLNNFYPREAKVQWKVDNALQSGNSQESVTEQDSKDSTYSLSSTLTLS
KADYEKHKVYACEVTHQGLSSPVTKSFNRGEC
hz24F7.v2 S91D Light chain variable region amino acid sequence (SEQ ID NO: 106)
DIQMTQSPSSLSASVGDRVTITCSVSSSVSYMYWYQQKPGKAPKLLIYDTSNLASGVPSR
FSGSGSGTDFTFTISSLQPEDIATYYCQQWDSYPTFGQGTKLEIK
hz24F7.v2 S91T Light chain variable region amino acid sequence (SEQ ID NO: 109)
DIQMTQSPSSLSASVGDRVTITCSVSSSVSYMYWYQQKPGKAPKLLIYDTSNLASGVPSR
FSGSGSGTDFTFTISSLQPEDIATYYCQQWTSYPTFGQGTKLEIK
hz24F7.v2 S91A Light chain variable region amino acid sequence (SEQ ID NO: 112)
DIQMTQSPSSLSASVGDRVTITCSVSSSVSYMYWYQQKPGKAPKLLIYDTSNLASGVPSR
FSGSGSGTDFTFTISSLQPEDIATYYCQQWASYPTFGQGTKLEIK
hz24F7.v2 S91L Light chain variable region amino acid sequence (SEQ ID NO: 115)
DIQMTQSPSSLSASVGDRVTITCSVSSSVSYMYWYQQKPGKAPKLLIYDTSNLASGVPSR
FSGSGSGTDFTFTISSLQPEDIATYYCQQWSSYPTFGQGTKLEIK
hz24F7.v2 S92Y Light chain variable region amino acid sequence (SEQ ID NO: 118)
DIQMTQSPSSLSASVGDRVTITCSVSSSVSYMYWYQQKPGKAPKLLIYDTSNLASGVPSR
FSGSGSGTDFTFTISSLQPEDIATYYCQQWSYYPTFGQGTKLEIK
hz24F7.v2 S92D Light chain variable region amino acid sequence (SEQ ID NO: 121)
DIQMTQSPSSLSASVGDRVTITCSVSSSVSYMYWYQQKPGKAPKLLIYDTSNLASGVPSR
FSGSGSGTDFTFTISSLQPEDIATYYCQQWSDYPTFGQGTKLEIK
hz24F7.v2 S91D Light chain amino acid sequence with signal sequence underlined (SEQ ID
NO: 122)
MDMRVPAQLLGLLLLWLRGARCDIQMTQSPSSLSASVGDRVTITCSVSSSVSYMYWYQQK
PGKAPKLLIYDTSNLASGVPSRFSGSGSGTDFTFTISSLQPEDIATYYCQQWDSYPTFGQ
GTKLEIKRTVAAPSVFIFPPSDEQLKSGTASVVCLLNNFYPREAKVQWKVDNALQSGNSQ
ESVTEQDSKDSTYSLSSTLTLSKADYEKHKVYACEVTHQGLSSPVTKSFNRGEC
hz24F7.v2 S91D Light chain amino acid sequence without signal sequence (SEQ ID NO: 123)
DIQMTQSPSSLSASVGDRVTITCSVSSSVSYMYWYQQK
PGKAPKLLIYDTSNLASGVPSRFSGSGSGTDFTFTISSLQPEDIATYYCQQWDSYPTFGQ
GTKLEIKRTVAAPSVFIFPPSDEQLKSGTASVVCLLNNFYPREAKVQWKVDNALQSGNSQ
ESVTEQDSKDSTYSLSSTLTLSKADYEKHKVYACEVTHQGLSSPVTKSFNRGEC
hz24F7.v2 S91T Light chain amino acid sequence with signal sequence underlined (SEQ ID
NO: 124)
MDMRVPAQLLGLLLLWLRGARCDIQMTQSPSSLSASVGDRVTITCSVSSSVSYMYWYQQK
PGKAPKLLIYDTSNLASGVPSRFSGSGSGTDFTFTISSLQPEDIATYYCQQWTSYPTFGQ
GTKLEIKRTVAAPSVFIFPPSDEQLKSGTASVVCLLNNFYPREAKVQWKVDNALQSGNSQ
ESVTEQDSKDSTYSLSSTLTLSKADYEKHKVYACEVTHQGLSSPVTKSFNRGEC
hz24F7.v2 S91T Light chain amino acid sequence without signal sequence (SEQ ID NO: 125)
DIQMTQSPSSLSASVGDRVTITCSVSSSVSYMYWYQQK
PGKAPKLLIYDTSNLASGVPSRFSGSGSGTDFTFTISSLQPEDIATYYCQQWTSYPTFGQ
GTKLEIKRTVAAPSVFIFPPSDEQLKSGTASVVCLLNNFYPREAKVQWKVDNALQSGNSQ
ESVTEQDSKDSTYSLSSTLTLSKADYEKHKVYACEVTHQGLSSPVTKSFNRGEC
hz24F7.v2 S91A Light chain amino acid sequence with signal sequence underlined (SEQ ID
NO: 126)
MDMRVPAQLLGLLLLWLRGARCDIQMTQSPSSLSASVGDRVTITCSVSSSVSYMYWYQQK
PGKAPKLLIYDTSNLASGVPSRFSGSGSGTDFTFTISSLQPEDIATYYCQQWASYPTFGQ
GTKLEIKRTVAAPSVFIFPPSDEQLKSGTASVVCLLNNFYPREAKVQWKVDNALQSGNSQ
ESVTEQDSKDSTYSLSSTLTLSKADYEKHKVYACEVTHQGLSSPVTKSFNRGEC
hz24F7.v2 S91A Light chain amino acid sequence without signal sequence (SEQ ID NO: 127)
DIQMTQSPSSLSASVGDRVTITCSVSSSVSYMYWYQQK
PGKAPKLLIYDTSNLASGVPSRFSGSGSGTDFTFTISSLQPEDIATYYCQQWASYPTFGQ
GTKLEIKRTVAAPSVFIFPPSDEQLKSGTASVVCLLNNFYPREAKVQWKVDNALQSGNSQ
ESVTEQDSKDSTYSLSSTLTLSKADYEKHKVYACEVTHQGLSSPVTKSFNRGEC
hz24F7.v2 S91L Light chain amino acid sequence with signal sequence underlined (SEQ ID
NO: 128)
MDMRVPAQLLGLLLLWLRGARCDIQMTQSPSSLSASVGDRVTITCSVSSSVSYMYWYQQK
PGKAPKLLIYDTSNLASGVPSRFSGSGSGTDFTFTISSLQPEDIATYYCQQWSSYPTFGQ
GTKLEIKRTVAAPSVFIFPPSDEQLKSGTASVVCLLNNFYPREAKVQWKVDNALQSGNSQ
ESVTEQDSKDSTYSLSSTLTLSKADYEKHKVYACEVTHQGLSSPVTKSFNRGEC
hz24F7.v2 S91L Light chain amino acid sequence without signal sequence (SEQ ID NO: 129)
DIQMTQSPSSLSASVGDRVTITCSVSSSVSYMYWYQQK
PGKAPKLLIYDTSNLASGVPSRFSGSGSGTDFTFTISSLQPEDIATYYCQQWSSYPTFGQ
GTKLEIKRTVAAPSVFIFPPSDEQLKSGTASVVCLLNNFYPREAKVQWKVDNALQSGNSQ
ESVTEQDSKDSTYSLSSTLTLSKADYEKHKVYACEVTHQGLSSPVTKSFNRGEC
hz24F7.v2 S92Y Light chain amino acid sequence with signal sequence underlined (SEQ ID
NO: 130)
MDMRVPAQLLGLLLLWLRGARCDIQMTQSPSSLSASVGDRVTITCSVSSSVSYMYWYQQK
PGKAPKLLIYDTSNLASGVPSRFSGSGSGTDFTFTISSLQPEDIATYYCQQWSYYPTFGQ
GTKLEIKRTVAAPSVFIFPPSDEQLKSGTASVVCLLNNFYPREAKVQWKVDNALQSGNSQ
ESVTEQDSKDSTYSLSSTLTLSKADYEKHKVYACEVTHQGLSSPVTKSFNRGEC
hz24F7.v2 S92Y Light chain amino acid sequence without signal sequence (SEQ ID NO: 131)
DIQMTQSPSSLSASVGDRVTITCSVSSSVSYMYWYQQK
PGKAPKLLIYDTSNLASGVPSRFSGSGSGTDFTFTISSLQPEDIATYYCQQWSYYPTFGQ
GTKLEIKRTVAAPSVFIFPPSDEQLKSGTASVVCLLNNFYPREAKVQWKVDNALQSGNSQ
ESVTEQDSKDSTYSLSSTLTLSKADYEKHKVYACEVTHQGLSSPVTKSFNRGEC
hz24F7.v2 S92D Light chain amino acid sequence with signal sequence underlined (SEQ ID
NO: 132)
MDMRVPAQLLGLLLLWLRGARCDIQMTQSPSSLSASVGDRVTITCSVSSSVSYMYWYQQK
PGKAPKLLIYDTSNLASGVPSRFSGSGSGTDFTFTISSLQPEDIATYYCQQWSDYPTFGQ
GTKLEIKRTVAAPSVFIFPPSDEQLKSGTASVVCLLNNFYPREAKVQWKVDNALQSGNSQ
ESVTEQDSKDSTYSLSSTLTLSKADYEKHKVYACEVTHQGLSSPVTKSFNRGEC
hz24F7.v2 S92D Light chain amino acid sequence without signal sequence (SEQ ID NO: 133)
DIQMTQSPSSLSASVGDRVTITCSVSSSVSYMYWYQQK
PGKAPKLLIYDTSNLASGVPSRFSGSGSGTDFTFTISSLQPEDIATYYCQQWSDYPTFGQ
GTKLEIKRTVAAPSVFIFPPSDEQLKSGTASVVCLLNNFYPREAKVQWKVDNALQSGNSQ
ESVTEQDSKDSTYSLSSTLTLSKADYEKHKVYACEVTHQGLSSPVTKSFNRGEC

1. A binding agent that specifically binds human high-temperature requirement A serine peptidase 1 (HTRA1), which comprises:
(i) (a) a heavy chain variable region comprising a heavy chain variable region CDR1 comprising the amino acid sequence GYTFTDYEMH (SEQ ID NO:9), a heavy chain variable region CDR2 comprising the amino acid sequence AIDPETGGTAYNQKFKG (SEQ ID NO:10), and a heavy chain variable region CDR3 comprising the amino acid sequence EGYSYDGGGYYFDY (SEQ ID NO:11) or EGYSYEGGGYYFDY (SEQ ID NO:25); and
(b) a light chain variable region comprising a light chain variable region CDR1 comprising the amino acid sequence SVSSSVSYMY (SEQ ID NO:12), a light chain variable region CDR2 comprising the amino acid sequence DTSNLAS (SEQ ID NO:13), and a light chain variable region CDR3 comprising the amino acid sequence QQWSSYPT (SEQ ID NO:14);
(ii) (a) a heavy chain variable region comprising a heavy chain variable region CDR1 comprising the amino acid sequence GYTFTDYEMH (SEQ ID NO:9), a heavy chain variable region CDR2 comprising the amino acid sequence AIDPETGGTAYNQKFKG (SEQ ID NO:10), and a heavy chain variable region CDR3 comprising the amino acid sequence EGYSYDGGGYYFDY (SEQ ID NO:11) or EGYSYEGGGYYFDY (SEQ ID NO:25); and
(b) a light chain variable region comprising a light chain variable region CDR1 comprising the amino acid sequence SVSSSVSYMY (SEQ ID NO:12), a light chain variable region CDR2 comprising the amino acid sequence DTSNLAS (SEQ ID NO:13), and a light chain variable region CDR3 comprising the amino acid sequence QQWYSYPT (SEQ ID NO:94);
(iii) (a) a heavy chain variable region comprising a heavy chain variable region CDR1 comprising the amino acid sequence GYTFTDYEMH (SEQ ID NO:9), a heavy chain variable region CDR2 comprising the amino acid sequence AIDPETGGTAYNQKFKG (SEQ ID NO:10), and a heavy chain variable region CDR3 comprising the amino acid sequence EGYSYDGGGYYFDY (SEQ ID NO:11) or EGYSYEGGGYYFDY (SEQ ID NO:25); and
(b) a light chain variable region comprising a light chain variable region CDR1 comprising the amino acid sequence SVSSSVSYMY (SEQ ID NO:12), a light chain variable region CDR2 comprising the amino acid sequence DTSNLAS (SEQ ID NO:13), and a light chain variable region CDR3 comprising the amino acid sequence QQWSTYPT (SEQ ID NO:99);
(iv) (a) a heavy chain variable region comprising a heavy chain variable region CDR1 comprising the amino acid sequence GYTFTDYEMH (SEQ ID NO:9), a heavy chain variable region CDR2 comprising the amino acid sequence AIDPETGGTAYNQKFKG (SEQ ID NO:10), and a heavy chain variable region CDR3 comprising the amino acid sequence EGYSYDGGGYYFDY (SEQ ID NO:11) or EGYSYEGGGYYFDY (SEQ ID NO:25); and
(b) a light chain variable region comprising a light chain variable region CDR1 comprising the amino acid sequence SVSSSVSYMY (SEQ ID NO:12), a light chain variable region CDR2 comprising the amino acid sequence DTSNLAS (SEQ ID NO:13), and a light chain variable region CDR3 comprising the amino acid sequence QQWDSYPT (SEQ ID NO:104);
(v) (a) a heavy chain variable region comprising a heavy chain variable region CDR1 comprising the amino acid sequence GYTFTDYEMH (SEQ ID NO:9), a heavy chain variable region CDR2 comprising the amino acid sequence AIDPETGGTAYNQKFKG (SEQ ID NO:10), and a heavy chain variable region CDR3 comprising the amino acid sequence EGYSYDGGGYYFDY (SEQ ID NO:11) or EGYSYEGGGYYFDY (SEQ ID NO:25); and
(b) a light chain variable region comprising a light chain variable region CDR1 comprising the amino acid sequence SVSSSVSYMY (SEQ ID NO:12), a light chain variable region CDR2 comprising the amino acid sequence DTSNLAS (SEQ ID NO:13), and a light chain variable region CDR3 comprising the amino acid sequence QQWTSYPT (SEQ ID NO:107);
(vi) (a) a heavy chain variable region comprising a heavy chain variable region CDR1 comprising the amino acid sequence GYTFTDYEMH (SEQ ID NO:9), a heavy chain variable region CDR2 comprising the amino acid sequence AIDPETGGTAYNQKFKG (SEQ ID NO:10), and a heavy chain variable region CDR3 comprising the amino acid sequence EGYSYDGGGYYFDY (SEQ ID NO:11) or EGYSYEGGGYYFDY (SEQ ID NO:25); and
(b) a light chain variable region comprising a light chain variable region CDR1 comprising the amino acid sequence SVSSSVSYMY (SEQ ID NO:12), a light chain variable region CDR2 comprising the amino acid sequence DTSNLAS (SEQ ID NO:13), and a light chain variable region CDR3 comprising the amino acid sequence QQWASYPT (SEQ ID NO:110);
(vii) (a) a heavy chain variable region comprising a heavy chain variable region CDR1 comprising the amino acid sequence GYTFTDYEMH (SEQ ID NO:9), a heavy chain variable region CDR2 comprising the amino acid sequence AIDPETGGTAYNQKFKG (SEQ ID NO:10), and a heavy chain variable region CDR3 comprising the amino acid sequence EGYSYDGGGYYFDY (SEQ ID NO:11) or EGYSYEGGGYYFDY (SEQ ID NO:25); and
(b) a light chain variable region comprising a light chain variable region CDR1 comprising the amino acid sequence SVSSSVSYMY (SEQ ID NO:12), a light chain variable region CDR2 comprising the amino acid sequence DTSNLAS (SEQ ID NO:13), and a light chain variable region CDR3 comprising the amino acid sequence QQWLSYPT (SEQ ID NO:113);
(viii) (a) a heavy chain variable region comprising a heavy chain variable region CDR1 comprising the amino acid sequence GYTFTDYEMH (SEQ ID NO:9), a heavy chain variable region CDR2 comprising the amino acid sequence AIDPETGGTAYNQKFKG (SEQ ID NO:10), and a heavy chain variable region CDR3 comprising the amino acid sequence EGYSYDGGGYYFDY (SEQ ID NO:11) or EGYSYEGGGYYFDY (SEQ ID NO:25); and
(b) a light chain variable region comprising a light chain variable region CDR1 comprising the amino acid sequence SVSSSVSYMY (SEQ ID NO:12), a light chain variable region CDR2 comprising the amino acid sequence DTSNLAS (SEQ ID NO:13), and a light chain variable region CDR3 comprising the amino acid sequence QQWSYYPT (SEQ ID NO:116); or
(ix) (a) a heavy chain variable region comprising a heavy chain variable region CDR1 comprising the amino acid sequence GYTFTDYEMH (SEQ ID NO:9), a heavy chain variable region CDR2 comprising the amino acid sequence AIDPETGGTAYNQKFKG (SEQ ID NO:10), and a heavy chain variable region CDR3 comprising the amino acid sequence EGYSYDGGGYYFDY (SEQ ID NO:11) or EGYSYEGGGYYFDY (SEQ ID NO:25); and
(b) a light chain variable region comprising a light chain variable region CDR1 comprising the amino acid sequence SVSSSVSYMY (SEQ ID NO:12), a light chain variable region CDR2 comprising the amino acid sequence DTSNLAS (SEQ ID NO:13), and a light chain variable region CDR3 comprising the amino acid sequence QQWSDYPT (SEQ ID NO:119).

2. The binding agent of claim 1, which comprises:
(i) (a) a heavy chain variable region comprising a heavy chain variable region CDR1 comprising the amino acid sequence GYTFTDYEMH (SEQ ID NO:9), a heavy chain variable region CDR2 comprising the amino acid sequence AIDPETGGTAYNQKFKG (SEQ ID NO:10), and a heavy chain variable region CDR3 comprising the amino acid sequence EGYSYDGGGYYFDY (SEQ ID NO:11); and
(b) a light chain variable region comprising a light chain variable region CDR1 comprising the amino acid sequence SVSSSVSYMY (SEQ ID NO:12), a light chain variable region CDR2 comprising the amino acid sequence DTSNLAS (SEQ ID NO:13), and a light chain variable region CDR3 comprising the amino acid sequence QQWSSYPT (SEQ ID NO:14);
(ii) (a) a heavy chain variable region comprising a heavy chain variable region CDR1 comprising the amino acid sequence GYTFTDYEMH (SEQ ID NO:9), a heavy chain variable region CDR2 comprising the amino acid sequence AIDPETGGTAYNQKFKG (SEQ ID NO:10), and a heavy chain variable region CDR3 comprising the amino acid sequence EGYSYEGGGYYFDY (SEQ ID NO:25); and
(b) a light chain variable region comprising a light chain variable region CDR1 comprising the amino acid sequence SVSSSVSYMY (SEQ ID NO:12), a light chain variable region CDR2 comprising the amino acid sequence DTSNLAS (SEQ ID NO:13), and a light chain variable region CDR3 comprising the amino acid sequence QQWSSYPT (SEQ ID NO:14);
(iii) (a) a heavy chain variable region comprising a heavy chain variable region CDR1 comprising the amino acid sequence GYTFTDYEMH (SEQ ID NO:9), a heavy chain variable region CDR2 comprising the amino acid sequence AIDPETGGTAYNQKFKG (SEQ ID NO:10), and a heavy chain variable region CDR3 comprising the amino acid sequence EGYSYDGGGYYFDY (SEQ ID NO:11); and
(b) a light chain variable region comprising a light chain variable region CDR1 comprising the amino acid sequence SVSSSVSYMY (SEQ ID NO:12), a light chain variable region CDR2 comprising the amino acid sequence DTSNLAS (SEQ ID NO:13), and a light chain variable region CDR3 comprising the amino acid sequence QQWYSYPT (SEQ ID NO:94);
(iv) (a) a heavy chain variable region comprising a heavy chain variable region CDR1 comprising the amino acid sequence GYTFTDYEMH (SEQ ID NO:9), a heavy chain variable region CDR2 comprising the amino acid sequence AIDPETGGTAYNQKFKG (SEQ ID NO:10), and a heavy chain variable region CDR3 comprising the amino acid sequence EGYSYEGGGYYFDY (SEQ ID NO:25); and
(b) a light chain variable region comprising a light chain variable region CDR1 comprising the amino acid sequence SVSSSVSYMY (SEQ ID NO:12), a light chain variable region CDR2 comprising the amino acid sequence DTSNLAS (SEQ ID NO:13), and a light chain variable region CDR3 comprising the amino acid sequence QQWYSYPT (SEQ ID NO:94);
(v) (a) a heavy chain variable region comprising a heavy chain variable region CDR1 comprising the amino acid sequence GYTFTDYEMH (SEQ ID NO:9), a heavy chain variable region CDR2 comprising the amino acid sequence AIDPETGGTAYNQKFKG (SEQ ID NO:10), and a heavy chain variable region CDR3 comprising the amino acid sequence EGYSYDGGGYYFDY (SEQ ID NO:11); and
(b) a light chain variable region comprising a light chain variable region CDR1 comprising the amino acid sequence SVSSSVSYMY (SEQ ID NO:12), a light chain variable region CDR2 comprising the amino acid sequence DTSNLAS (SEQ ID NO:13), and a light chain variable region CDR3 comprising the amino acid sequence QQWSTYPT (SEQ ID NO:99);
(vi) (a) a heavy chain variable region comprising a heavy chain variable region CDR1 comprising the amino acid sequence GYTFTDYEMH (SEQ ID NO:9), a heavy chain variable region CDR2 comprising the amino acid sequence AIDPETGGTAYNQKFKG (SEQ ID NO:10), and a heavy chain variable region CDR3 comprising the amino acid sequence EGYSYEGGGYYFDY (SEQ ID NO:25); and
(b) a light chain variable region comprising a light chain variable region CDR1 comprising the amino acid sequence SVSSSVSYMY (SEQ ID NO:12), a light chain variable region CDR2 comprising the amino acid sequence DTSNLAS (SEQ ID NO:13), and a light chain variable region CDR3 comprising the amino acid sequence QQWSTYPT (SEQ ID NO:99);
(vii) (a) a heavy chain variable region comprising a heavy chain variable region CDR1 comprising the amino acid sequence GYTFTDYEMH (SEQ ID NO:9), a heavy chain variable region CDR2 comprising the amino acid sequence AIDPETGGTAYNQKFKG (SEQ ID NO:10), and a heavy chain variable region CDR3 comprising the amino acid sequence EGYSYDGGGYYFDY (SEQ ID NO:11); and
(b) a light chain variable region comprising a light chain variable region CDR1 comprising the amino acid sequence SVSSSVSYMY (SEQ ID NO:12), a light chain variable region CDR2 comprising the amino acid sequence DTSNLAS (SEQ ID NO:13), and a light chain variable region CDR3 comprising the amino acid sequence QQWDSYPT (SEQ ID NO:104);
(viii) (a) a heavy chain variable region comprising a heavy chain variable region CDR1 comprising the amino acid sequence GYTFTDYEMH (SEQ ID NO:9), a heavy chain variable region CDR2 comprising the amino acid sequence AIDPETGGTAYNQKFKG (SEQ ID NO:10), and a heavy chain variable region CDR3 comprising the amino acid sequence EGYSYEGGGYYFDY (SEQ ID NO:25); and
(b) a light chain variable region comprising a light chain variable region CDR1 comprising the amino acid sequence SVSSSVSYMY (SEQ ID NO:12), a light chain variable region CDR2 comprising the amino acid sequence DTSNLAS (SEQ ID NO:13), and a light chain variable region CDR3 comprising the amino acid sequence QQWDSYPT (SEQ ID NO:104);
(ix) (a) a heavy chain variable region comprising a heavy chain variable region CDR1 comprising the amino acid sequence GYTFTDYEMH (SEQ ID NO:9), a heavy chain variable region CDR2 comprising the amino acid sequence AIDPETGGTAYNQKFKG (SEQ ID NO:10), and a heavy chain variable region CDR3 comprising the amino acid sequence EGYSYDGGGYYFDY (SEQ ID NO:11); and
(b) a light chain variable region comprising a light chain variable region CDR1 comprising the amino acid sequence SVSSSVSYMY (SEQ ID NO:12), a light chain variable region CDR2 comprising the amino acid sequence DTSNLAS (SEQ ID NO:13), and a light chain variable region CDR3 comprising the amino acid sequence QQWTSYPT (SEQ ID NO:107);
(x) (a) a heavy chain variable region comprising a heavy chain variable region CDR1 comprising the amino acid sequence GYTFTDYEMH (SEQ ID NO:9), a heavy chain variable region CDR2 comprising the amino acid sequence AIDPETGGTAYNQKFKG (SEQ ID NO:10), and a heavy chain variable region CDR3 comprising the amino acid sequence EGYSYEGGGYYFDY (SEQ ID NO:25); and
(b) a light chain variable region comprising a light chain variable region CDR1 comprising the amino acid sequence SVSSSVSYMY (SEQ ID NO:12), a light chain variable region CDR2 comprising the amino acid sequence DTSNLAS (SEQ ID NO:13), and a light chain variable region CDR3 comprising the amino acid sequence QQWTSYPT (SEQ ID NO:107);
(xi) (a) a heavy chain variable region comprising a heavy chain variable region CDR1 comprising the amino acid sequence GYTFTDYEMH (SEQ ID NO:9), a heavy chain variable region CDR2 comprising the amino acid sequence AIDPETGGTAYNQKFKG (SEQ ID NO:10), and a heavy chain variable region CDR3 comprising the amino acid sequence EGYSYDGGGYYFDY (SEQ ID NO:11); and
(b) a light chain variable region comprising a light chain variable region CDR1 comprising the amino acid sequence SVSSSVSYMY (SEQ ID NO:12), a light chain variable region CDR2 comprising the amino acid sequence DTSNLAS (SEQ ID NO:13), and a light chain variable region CDR3 comprising the amino acid sequence QQWASYPT (SEQ ID NO:110);
(xii) (a) a heavy chain variable region comprising a heavy chain variable region CDR1 comprising the amino acid sequence GYTFTDYEMH (SEQ ID NO:9), a heavy chain variable region CDR2 comprising the amino acid sequence AIDPETGGTAYNQKFKG (SEQ ID NO:10), and a heavy chain variable region CDR3 comprising the amino acid sequence EGYSYEGGGYYFDY (SEQ ID NO:25); and
(b) a light chain variable region comprising a light chain variable region CDR1 comprising the amino acid sequence SVSSSVSYMY (SEQ ID NO:12), a light chain variable region CDR2 comprising the amino acid sequence DTSNLAS (SEQ ID NO:13), and a light chain variable region CDR3 comprising the amino acid sequence QQWASYPT (SEQ ID NO:110);
(xiii) (a) a heavy chain variable region comprising a heavy chain variable region CDR1 comprising the amino acid sequence GYTFTDYEMH (SEQ ID NO:9), a heavy chain variable region CDR2 comprising the amino acid sequence AIDPETGGTAYNQKFKG (SEQ ID NO:10), and a heavy chain variable region CDR3 comprising the amino acid sequence EGYSYDGGGYYFDY (SEQ ID NO:11); and
(b) a light chain variable region comprising a light chain variable region CDR1 comprising the amino acid sequence SVSSSVSYMY (SEQ ID NO:12), a light chain variable region CDR2 comprising the amino acid sequence DTSNLAS (SEQ ID NO:13), and a light chain variable region CDR3 comprising the amino acid sequence QQWLSYPT (SEQ ID NO:113);
(xiv) (a) a heavy chain variable region comprising a heavy chain variable region CDR1 comprising the amino acid sequence GYTFTDYEMH (SEQ ID NO:9), a heavy chain variable region CDR2 comprising the amino acid sequence AIDPETGGTAYNQKFKG (SEQ ID NO:10), and a heavy chain variable region CDR3 comprising the amino acid sequence EGYSYEGGGYYFDY (SEQ ID NO:25); and
(b) a light chain variable region comprising a light chain variable region CDR1 comprising the amino acid sequence SVSSSVSYMY (SEQ ID NO:12), a light chain variable region CDR2 comprising the amino acid sequence DTSNLAS (SEQ ID NO:13), and a light chain variable region CDR3 comprising the amino acid sequence QQWLSYPT (SEQ ID NO:113);
(xv) (a) a heavy chain variable region comprising a heavy chain variable region CDR1 comprising the amino acid sequence GYTFTDYEMH (SEQ ID NO:9), a heavy chain variable region CDR2 comprising the amino acid sequence AIDPETGGTAYNQKFKG (SEQ ID NO:10), and a heavy chain variable region CDR3 comprising the amino acid sequence EGYSYDGGGYYFDY (SEQ ID NO:11); and
(b) a light chain variable region comprising a light chain variable region CDR1 comprising the amino acid sequence SVSSSVSYMY (SEQ ID NO:12), a light chain variable region CDR2 comprising the amino acid sequence DTSNLAS (SEQ ID NO:13), and a light chain variable region CDR3 comprising the amino acid sequence QQWSYYPT (SEQ ID NO:116);
(xvi) (a) a heavy chain variable region comprising a heavy chain variable region CDR1 comprising the amino acid sequence GYTFTDYEMH (SEQ ID NO:9), a heavy chain variable region CDR2 comprising the amino acid sequence AIDPETGGTAYNQKFKG (SEQ ID NO:10), and a heavy chain variable region CDR3 comprising the amino acid sequence EGYSYEGGGYYFDY (SEQ ID NO:25); and
(b) a light chain variable region comprising a light chain variable region CDR1 comprising the amino acid sequence SVSSSVSYMY (SEQ ID NO:12), a light chain variable region CDR2 comprising the amino acid sequence DTSNLAS (SEQ ID NO:13), and a light chain variable region CDR3 comprising the amino acid sequence QQWSYYPT (SEQ ID NO:116);
(xvii) (a) a heavy chain variable region comprising a heavy chain variable region CDR1 comprising the amino acid sequence GYTFTDYEMH (SEQ ID NO:9), a heavy chain variable region CDR2 comprising the amino acid sequence AIDPETGGTAYNQKFKG (SEQ ID NO:10), and a heavy chain variable region CDR3 comprising the amino acid sequence EGYSYDGGGYYFDY (SEQ ID NO:11); and
(b) a light chain variable region comprising a light chain variable region CDR1 comprising the amino acid sequence SVSSSVSYMY (SEQ ID NO:12), a light chain variable region CDR2 comprising the amino acid sequence DTSNLAS (SEQ ID NO:13), and a light chain variable region CDR3 comprising the amino acid sequence QQWSDYPT (SEQ ID NO:119);
(xviii) (a) a heavy chain variable region comprising a heavy chain variable region CDR1 comprising the amino acid sequence GYTFTDYEMH (SEQ ID NO:9), a heavy chain variable region CDR2 comprising the amino acid sequence AIDPETGGTAYNQKFKG (SEQ ID NO:10), and a heavy chain variable region CDR3 comprising the amino acid sequence EGYSYEGGGYYFDY (SEQ ID NO:25); and
(b) a light chain variable region comprising a light chain variable region CDR1 comprising the amino acid sequence SVSSSVSYMY (SEQ ID NO:12), a light chain variable region CDR2 comprising the amino acid sequence DTSNLAS (SEQ ID NO:13), and a light chain variable region CDR3 comprising the amino acid sequence QQWSDYPT (SEQ ID NO:119);
(xix) (a) a heavy chain variable region comprising a heavy chain variable region CDR1 comprising the amino acid sequence GYAFTTYWMH (SEQ ID NO:27); a heavy chain variable region CDR2 comprising the amino acid sequence NIDPSDSETHYNQKFRD (SEQ ID NO:28), and a heavy chain variable region CDR3 comprising the amino acid sequence DYGAFDV (SEQ ID NO:29); and
(b) a light chain variable region comprising a light chain variable region CDR1 comprising the amino acid sequence RSSTGAVTTR (SEQ ID NO:30); a light chain variable region CDR2 comprising the amino acid sequence GTNNRAP (SEQ ID NO:31); and a light chain variable region CDR3 comprising the amino acid sequence ALWYSNLWV (SEQ ID NO:32);
(xx) (a) a heavy chain variable region comprising a heavy chain variable region CDR1 comprising the amino acid sequence GYTFTNYWMH (SEQ ID NO:43); a heavy chain variable region CDR2 comprising the amino acid sequence NIDPSDSETHYNQKFKD (SEQ ID NO:44), and a heavy chain variable region CDR3 comprising the amino acid sequence EDSSGYGAY (SEQ ID NO:45); and
(b) a light chain variable region comprising a light chain variable region CDR1 comprising the amino acid sequence SASSSVNYMH (SEQ ID NO:46); a light chain variable region CDR2 comprising the amino acid sequence DTSKLAS (SEQ ID NO:47); and a light chain variable region CDR3 comprising the amino acid sequence QQWSSHPLT (SEQ ID NO:48; or
(xxi) (a) a heavy chain variable region comprising a heavy chain variable region CDR1 comprising the amino acid sequence GYSFTSYWMH (SEQ ID NO:56); a heavy chain variable region CDR2 comprising the amino acid sequence MIDPSDSETRLNQKFKD (SEQ ID NO:57), and a heavy chain variable region CDR3 comprising the amino acid sequence DYFDY (SEQ ID NO:58); and
(b) a light chain variable region comprising a light chain variable region CDR1 comprising the amino acid sequence SASSSVSYMY (SEQ ID NO:59); a light chain variable region CDR2 comprising the amino acid sequence DTSNLAS (SEQ ID NO:13); and a light chain variable region CDR3 comprising the amino acid sequence QQWSSYPYT (SEQ ID NO:60).

3. (canceled)

4. The binding agent of claim 1, wherein the light chain variable region CDR3 comprises an amino acid substitution.

5. The binding agent of claim 4, wherein the substitution is at position 91 or 92.

6. The binding agent of claim 5, wherein the substitution is selected from the group consisting of S91Y, S91D, S91T, S91A and S91L, or S92T, s92Y or S92D.

7. The binding agent of claim 5 or 6, wherein the substitution is S91Y or S92T.

8.-34. (canceled)

35. The binding agent of claim 1, which comprises:
(i) a heavy chain variable region having at least 80% sequence identity to SEQ ID NO:68;
(ii) a light chain variable region having at least 80% sequence identity to SEQ ID NO:69;
(iii) a heavy chain variable region having at least 80% sequence identity to SEQ ID NO:68 and a light chain variable region having least 80% sequence identity to SEQ ID NO:69;
(iv) a heavy chain variable region having at least 85% sequence identity to SEQ ID NO:68;
(v) a light chain variable region having at least 85% sequence identity to SEQ ID NO:69;
(vi) a heavy chain variable region having at least 85% sequence identity to SEQ ID NO:68 and a light chain variable region having at least 85% sequence identity to SEQ ID NO:69;
(vii) a heavy chain variable region having at least 90% sequence identity to SEQ ID NO:68;
(viii) a light chain variable region having at least 90% sequence identity to SEQ ID NO:69;
(ix) a heavy chain variable region having at least 90% sequence identity to SEQ ID NO:68 and a light chain variable region having at least 90% sequence identity to SEQ ID NO:69;
(x) a heavy chain variable region having at least 95% sequence identity to SEQ ID NO:68;
(xi) a light chain variable region having at least 95% sequence identity to SEQ ID NO:69;
(xii) a heavy chain variable region having at least 95% sequence identity to SEQ ID NO:68 and a light chain variable region having at least 95% sequence identity to SEQ ID NO:69;
(xiii) a heavy chain variable region comprising the amino acid sequence of SEQ ID NO:68;
(xiv) a light chain variable region comprising the amino acid sequence of SEQ ID NO:69;
(xv) a heavy chain variable region comprising the amino acid sequence of SEQ ID NO:68 and a light chain variable region comprising the amino acid sequence of SEQ ID NO:69;
(xvi) a heavy chain variable region having at least 80% identity to SEQ ID NO:73 and a light chain variable region having at least 80% identity to SEQ ID NO:74;
(xvii) a heavy chain variable region having at least 80% identity to SEQ ID NO:75 and a light chain variable region having at least 80% identity to SEQ ID NO:76;
(xviii) comprises a heavy chain variable region having at least 80% identity to SEQ ID NO:77 and a light chain variable region having at least 80% identity to SEQ ID NO:78;
(ix) a heavy chain variable region having at least 80% sequence identity to SEQ ID NO:70;
(x) a heavy chain variable region having at least 80% sequence identity to SEQ ID NO:71;
(xi) a light chain variable region having least 80% sequence identity to SEQ ID NO:72;
(xii) a heavy chain variable region having least 80% sequence identity to SEQ ID NO:70 and a light chain variable region having least 80% sequence identity to SEQ ID NO:72;
(xiii) a heavy chain variable region having least 80% sequence identity to SEQ ID NO:71 and a light chain variable region having least 80% sequence identity to SEQ ID NO:72;
(xiv) a heavy chain variable region having least 85% sequence identity to SEQ ID NO:70;
(xv) a heavy chain variable region having least 85% sequence identity to SEQ ID NO:71;
(xvi) a light chain variable region having least 85% sequence identity to SEQ ID NO:72;
(xvii) a heavy chain variable region having least 85% sequence identity to SEQ ID NO:70 and a light chain variable region having least 85% sequence identity to SEQ ID NO:72;
(xviii) a heavy chain variable region having least 85% sequence identity to SEQ ID NO:71 and a light chain variable region having least 85% sequence identity to SEQ ID NO:72;
(xix) a heavy chain variable region having least 90% sequence identity to SEQ ID NO:70;
(xx) a heavy chain variable region having least 90% sequence identity to SEQ ID NO:71;
(xxi) a light chain variable region having least 90% sequence identity to SEQ ID NO:72;
(xxii) a heavy chain variable region having least 90% sequence identity to SEQ ID NO:70 and a light chain variable region having least 90% sequence identity to SEQ ID NO:72;
(xxiii) a heavy chain variable region having least 90% sequence identity to SEQ ID NO:71 and a light chain variable region having least 90% sequence identity to SEQ ID NO:72;
(xxiv) comprises a heavy chain variable region having least 95% sequence identity to SEQ ID NO:70;
(xxv) a heavy chain variable region having least 95% sequence identity to SEQ ID NO:71;
(xxvi) a light chain variable region having least 95% sequence identity to SEQ ID NO:72;
(xxvii) a heavy chain variable region having least 95% sequence identity to SEQ ID NO:70 and a light chain variable region having least 95% sequence identity to SEQ ID NO:72;
(xxviii) a heavy chain variable region having least 95% sequence identity to SEQ ID NO:71 and a light chain variable region having least 95% sequence identity to SEQ ID NO:72;
(xxix) a heavy chain variable region comprising the amino acid sequence of SEQ ID NO:70;
(xxx) a heavy chain variable region comprising the amino acid sequence of SEQ ID NO:71;
(xxxi) a light chain variable region comprising the amino acid sequence of SEQ ID NO:72;
(xxxii) a light chain variable region comprising the amino acid sequence of SEQ ID NO:96;
(xxxiii) a light chain variable region comprising the amino acid sequence of SEQ ID NO:101;
(xxxiv) a light chain variable region comprising the amino acid sequence of SEQ ID NO:106;
(xxxv) a light chain variable region comprising the amino acid sequence of SEQ ID NO:109;
(xxxvi) a light chain variable region comprising the amino acid sequence of SEQ ID NO:112;
(xxxvii) a light chain variable region comprising the amino acid sequence of SEQ ID NO:115;
(xxxviii) a light chain variable region comprising the amino acid sequence of SEQ ID NO:118;
(xxxix) a light chain variable region comprising the amino acid sequence of SEQ ID NO:121;
(xxxx) a heavy chain variable region comprising the amino acid sequence of SEQ ID NO:70 and a light chain variable region comprising the amino acid sequence of SEQ ID NO:72;
(xxxxi) a heavy chain variable region comprising the amino acid sequence of SEQ ID NO:70 and a light chain variable region comprising the amino acid sequence of SEQ ID NO:96;
(xxxxii) a heavy chain variable region comprising the amino acid sequence of SEQ ID NO:70 and a light chain variable region comprising the amino acid sequence of SEQ ID NO:101;
(xxxxiii) a heavy chain variable region comprising the amino acid sequence of SEQ ID NO:70 and a light chain variable region comprising the amino acid sequence of SEQ ID NO:106;
(xxxxiv) a heavy chain variable region comprising the amino acid sequence of SEQ ID NO:70 and a light chain variable region comprising the amino acid sequence of SEQ ID NO:109;
(xxxxv) a heavy chain variable region comprising the amino acid sequence of SEQ ID NO:70 and a light chain variable region comprising the amino acid sequence of SEQ ID NO:112;
(xxxxvi) a heavy chain variable region comprising the amino acid sequence of SEQ ID NO:70 and a light chain variable region comprising the amino acid sequence of SEQ ID NO:115;
(xxxxvii) a heavy chain variable region comprising the amino acid sequence of SEQ ID NO:70 and a light chain variable region comprising the amino acid sequence of SEQ ID NO:118;
(xxxxviii) a heavy chain variable region comprising the amino acid sequence of SEQ ID NO:70 and a light chain variable region comprising the amino acid sequence of SEQ ID NO:121;
(xxxxix) a heavy chain variable region comprising the amino acid sequence of SEQ ID NO:71 and a light chain variable region comprising the amino acid sequence of SEQ ID NO:72;
(xxxxx) a heavy chain variable region comprising the amino acid sequence of SEQ ID NO:71 and a light chain variable region comprising the amino acid sequence of SEQ ID NO:96;
(xxxxxi) a heavy chain variable region comprising the amino acid sequence of SEQ ID NO:71 and a light chain variable region comprising the amino acid sequence of SEQ ID NO:101;
(xxxxxii) a heavy chain variable region comprising the amino acid sequence of SEQ ID NO:71 and a light chain variable region comprising the amino acid sequence of SEQ ID NO:106;
(xxxxxiii) a heavy chain variable region comprising the amino acid sequence of SEQ ID NO:71 and a light chain variable region comprising the amino acid sequence of SEQ ID NO:109 (xxxxxiv) a heavy chain variable region comprising the amino acid sequence of SEQ ID NO:71 and a light chain variable region comprising the amino acid sequence of SEQ ID NO:112;
(xxxxxv) a heavy chain variable region comprising the amino acid sequence of SEQ ID NO:71 and a light chain variable region comprising the amino acid sequence of SEQ ID NO:115;
(xxxxxvi) a heavy chain variable region comprising the amino acid sequence of SEQ ID NO:71 and a light chain variable region comprising the amino acid sequence of SEQ ID NO:118; or
(xxxxxvii) a heavy chain variable region comprising the amino acid sequence of SEQ ID NO:71 and a light chain variable region comprising the amino acid sequence of SEQ ID NO:121.

36.-98. (canceled)

99. A binding agent that specifically binds human high-temperature requirement A serine peptidase 1 (HTRA1), which comprises:
(a) a heavy chain variable region comprising a CDR1, CDR2, and CDR3 from the amino acid sequence of SEQ ID NO:68 and a light chain variable region comprising a CDR1, CDR2, and CDR3 from the amino acid sequence of SEQ ID NO:69;
(b) a heavy chain variable region comprising a CDR1, CDR2, and CDR3 from the amino acid sequence of SEQ ID NO:71 and a light chain variable region comprising a CDR1, CDR2, and CDR3 from the amino acid sequence of SEQ ID NO:72;
(c) a heavy chain variable region comprising a CDR1, CDR2, and CDR3 from the amino acid sequence of SEQ ID NO:71 and a light chain variable region comprising a CDR1, CDR2, and CDR3 from the amino acid sequence of SEQ ID NO:96;
(d) a heavy chain variable region comprising a CDR1, CDR2, and CDR3 from the amino acid sequence of SEQ ID NO:71 and a light chain variable region comprising a CDR1, CDR2, and CDR3 from the amino acid sequence of SEQ ID NO:101;
(e) a heavy chain variable region comprising a CDR1, CDR2, and CDR3 from the amino acid sequence of SEQ ID NO:71 and a light chain variable region comprising a CDR1, CDR2, and CDR3 from the amino acid sequence of SEQ ID NO:106;
(f) a heavy chain variable region comprising a CDR1, CDR2, and CDR3 from the amino acid sequence of SEQ ID NO:71 and a light chain variable region comprising a CDR1, CDR2, and CDR3 from the amino acid sequence of SEQ ID NO:109;
(g) a heavy chain variable region comprising a CDR1, CDR2, and CDR3 from the amino acid sequence of SEQ ID NO:71 and a light chain variable region comprising a CDR1, CDR2, and CDR3 from the amino acid sequence of SEQ ID NO:112;
(h) a heavy chain variable region comprising a CDR1, CDR2, and CDR3 from the amino acid sequence of SEQ ID NO:71 and a light chain variable region comprising a CDR1, CDR2, and CDR3 from the amino acid sequence of SEQ ID NO:115;
(i) a heavy chain variable region comprising a CDR1, CDR2, and CDR3 from the amino acid sequence of SEQ ID NO:71 and a light chain variable region comprising a CDR1, CDR2, and CDR3 from the amino acid sequence of SEQ ID NO:118;
(j) a heavy chain variable region comprising a CDR1, CDR2, and CDR3 from the amino acid sequence of SEQ ID NO:71 and a light chain variable region comprising a CDR1, CDR2, and CDR3 from the amino acid sequence of SEQ ID NO:119;
(k) a heavy chain variable region comprising a CDR1, CDR2, and CDR3 from the amino acid sequence of SEQ ID NO:73 and a light chain variable region comprising a CDR1, CDR2, and CDR3 from the amino acid sequence of SEQ ID NO:74;
(l) a heavy chain variable region comprising a CDR1, CDR2, and CDR3 from the amino acid sequence of SEQ ID NO:75 and a light chain variable region comprising a CDR1, CDR2, and CDR3 from the amino acid sequence of SEQ ID NO:76; or
(m) a heavy chain variable region comprising a CDR1, CDR2, and CDR3 from the amino acid sequence of SEQ ID NO:77 and a light chain variable region comprising a CDR1, CDR2, and CDR3 from the amino acid sequence of SEQ ID NO:78.

100.-108. (canceled)

109. The binding agent of claim 1, which is:
(a) an antibody;
(b) a monoclonal antibody;
(c) a chimeric or humanized antibody;
(d) a bispecific antibody or a multispecific antibody; or
(e) an antibody fragment comprising at least one antigen-binding site.

110.-113. (canceled)

114. The binding agent of claim 109, wherein the antibody fragment is a Fab, Fab′, F(ab′)₂, Fv, scFv, (scFv)₂, single chain antibody, dual variable region antibody, diabody, or nanobody.

115. The binding agent of claim 109, which:
(a) is an IgG1 antibody;
(b) is an IgG2 antibody;
(c) is an IgG4 antibody;
(d) comprises a kappa light chain; or
(e) comprises a lambda light chain.

116.-119. (canceled)

120. An antibody that specifically binds HTRA1, which comprises:
(a) a heavy chain comprising the amino acid sequence of SEQ ID NO:88 and a light chain comprising the amino acid sequence of SEQ ID NO:90;
(b) a heavy chain comprising the amino acid sequence of SEQ ID NO:88 and a light chain comprising the amino acid sequence of SEQ ID NO:98;
(c) a heavy chain comprising the amino acid sequence of SEQ ID NO:88 and a light chain comprising the amino acid sequence of SEQ ID NO:103;
(d) a heavy chain comprising the amino acid sequence of SEQ ID NO:88 and a light chain comprising the amino acid sequence of SEQ ID NO:123;
(e) a heavy chain comprising the amino acid sequence of SEQ ID NO:88 and a light chain comprising the amino acid sequence of SEQ ID NO:125;
(f) a heavy chain comprising the amino acid sequence of SEQ ID NO:88 and a light chain comprising the amino acid sequence of SEQ ID NO:127;
(g) a heavy chain comprising the amino acid sequence of SEQ ID NO:88 and a light chain comprising the amino acid sequence of SEQ ID NO:129;
(h) a heavy chain comprising the amino acid sequence of SEQ ID NO:88 and a light chain comprising the amino acid sequence of SEQ ID NO:131; or
(i) a heavy chain comprising the amino acid sequence of SEQ ID NO:88 and a light chain comprising the amino acid sequence of SEQ ID NO:133.

121.-128. (canceled)

129. The antibody of claim 120, which:
(a) is an antagonist of HTRA1;
(b) inhibits HTRA1 activity; or
(c) inhibits HTRA1 protease activity.

130. (canceled)

131. (canceled)

132. The antibody of claim 120, which has one or more of the following properties:
(a) binds cyno HTRA1;
(b) binds rabbit HTRA1;
(c) inhibits HTRA1 protease activity;
(d) inhibits HTRA1 protease activity in an allosteric manner, and/or
(e) does not inhibit protease activity of other proteases in HTRA family.

133. The antibody of claim 120, which binds:
(a) the catalytic domain of human HTRA1;
(b) a conformational epitope comprising at least one amino acid within amino acids 185-200 of SEQ ID NO:1; or
(c) a conformational epitope comprising one or more amino acids R190, L192, or R197 of SEQ ID NO:1.

134. (canceled)

135. (canceled)

136. An antibody that competes with the antibody of claim 120 for binding to human HTRA1, which comprises:
(a) a heavy chain variable region comprising a heavy chain variable region CDR1 comprising the amino acid sequence GYAFTTYWMH (SEQ ID NO:27), a heavy chain variable region CDR2 comprising the amino acid sequence NIDPSDSETHYNQKFRD (SEQ ID NO:28), and a heavy chain variable region CDR3 comprising the amino acid sequence DYGAFDV (SEQ ID NO:29), and a light chain variable region comprising a light chain variable region CDR1 comprising the amino acid sequence RSSTGAVTTR (SEQ ID NO:30), a light chain variable region CDR2 comprising the amino acid sequence GTNNRAP (SEQ ID NO:31), and a light chain variable region CDR3 comprising the amino acid sequence ALWYSNLWV (SEQ ID NO:32);
(b) a heavy chain variable region comprising a heavy chain variable region CDR1 comprising the amino acid sequence GYTFTNYWMH (SEQ ID NO:43), a heavy chain variable region CDR2 comprising the amino acid sequence NIDPSDSETHYNQKFKD (SEQ ID NO:44), and a heavy chain variable region CDR3 comprising the amino acid sequence EDSSGYGAY (SEQ ID NO:45), and a light chain variable region comprising a light chain variable region CDR1 comprising the amino acid sequence SASSSVNYMH (SEQ ID NO:46), a light chain variable region CDR2 comprising the amino acid sequence DTSKLAS (SEQ ID NO:47), and a light chain variable region CDR3 comprising the amino acid sequence QQWSSHPLT (SEQ ID NO:48); or
(c) a heavy chain variable region comprising a heavy chain variable region CDR1 comprising the amino acid sequence GYSFTSYWMH (SEQ ID NO:56), a heavy chain variable region CDR2 comprising the amino acid sequence MIDPSDSETRLNQKFKD (SEQ ID NO:57), and a heavy chain variable region CDR3 comprising the amino acid sequence DYFDY (SEQ ID NO:58), and a light chain variable region comprising a light chain variable region CDR1 comprising the amino acid sequence SASSSVSYMY (SEQ ID NO:59), a light chain variable region CDR2 comprising the amino acid sequence DTSNLAS (SEQ ID NO:13), and a light chain variable region CDR3 comprising the amino acid sequence QQWSSYPYT (SEQ ID NO:60).

137.-157. (canceled)

158. The antibody of claim 120, wherein the antibody is attached to a half-life extending moiety.

159. A pharmaceutical composition that comprises the antibody of claim 120 and a pharmaceutically acceptable carrier.

160. An isolated polynucleotide encoding the antibody of claim 120.

161. A vector comprising the polynucleotide of claim 160.

162. An isolated cell comprising the vector of claim 161.

163. An isolated cell producing the antibody of claim 120.

164. A method of treating an eye disorder in a subject, the method comprising administering to the subject a therapeutically effective amount of the antibody of claim 120; wherein optionally, the eye disorder is selected from the group consisting of: macular degeneration (maculopathy), age-related macular degeneration (AMD), wet AMD, dry AMD, geographic atrophy (GA), diabetic retinopathy, retinopathy of prematurity, macular dystrophy, retinal dystrophy, uveitis, keratitis, scleritis, retinitis pigmentosa, choroidal neovascularization (CNV), retinal neovascularization, ocular inflammation, polypoidal choroidal vasculopathy (PCV), idiopathic polypoidal choroidal vasculopathy (IPCV), Stargardt disease, and neuromyelitis optica.

165. (canceled)

166. A method of treating age-related macular degeneration (AMD) in a subject, the method comprising administering to the subject a therapeutically effective amount of the antibody of claim 120; wherein optionally, the AMD is dry AMD, geographic atrophy, wet AMD, associated with neovascularization, or associated with CNV.

167.-171. (canceled)

172. A method of inhibiting or suppressing:
(a) drusen formation in an eye of a subject, the method comprising administering to an eye of the subject a therapeutically effective amount of the antibody of claim 120; wherein optionally, the number and/or size of drusen is reduced; or
(b) retinal pigment epithelium atrophy in an eye of a subject, the method comprising administering to an eye of the subject a therapeutically effective amount of the antibody of claim 120.

173. (canceled)

174. (canceled)

175. A method of inhibiting:
(a) HTRA1 protease activity in an eye of a subject, the method comprising administering to an eye of the subject a therapeutically effective amount of the antibody of claim 120;
(b) choroidal neovascularization in an eye of a subject, the method comprising administering to an eye of the subject a therapeutically effective amount of the antibody of claim 120; or
(c) progression of early or intermediate stage AMD to advanced stage AMD in an eye of a subject, the method comprising administering to an eye of the subject a therapeutically effective amount of the binding agent or antibody of the antibody of claim 120; wherein optionally, the advanced stage AMD is geographic atrophy or wet AMD.

176.-179. (canceled)

180. The method of claim 164, wherein the antibody is administered:
(a) to an eye of the subject by ocular injection, intraocular injection, or intravitreal injection;
(b) to an eye of the subject by intravitreal injection; or
(c) as part of a combination therapy.

181. (canceled)

182. (canceled)

183. The method of claim 180, wherein the combination therapy comprises:
(a) photodynamic therapy;
(b) at least one additional therapeutic agent wherein optionally, the additional therapeutic agent is:
(i) a VEGF inhibitor, a complement inhibitor, a PDGF inhibitor, a corticosteroid, or a neuroprotective agent; or
(b) a VEGF inhibitor; wherein optionally, the VEGF inhibitor is pegaptanib, ranibizumab, bevacizumab, aflibercept, or OPT-302.

184.-187. (canceled)

188. The method of claim 164, wherein the subject is a human.

189.-196. (canceled)

197. A method of making the antibody of claim 120, comprising: (a) culturing the cell of claim 163, and (b) isolating the antibody.

198. The method of claim 197, further comprising purifying the binding agent or antibody or formulating the binding agent or antibody as a pharmaceutical composition.

199. (canceled)


SEQUENCE LISTING (133 sequences)

SEQ ID NO: 1 - protein, 480 aa, Homo sapiens
  Human HTRA1 amino acid sequence with predicted signal sequence
MQIPRAALLPLLLLLLAAPASAQLSRAGRSAPLAAGCPDRCEPARCPPQPEHCEGGRARDACGCCEVCGAPEGAACGLQEGPCGEGLQCVVPFGVPASATVRRRAQAGLCVCASSEPVCGSDANTYANLCQLRAASRRSERLHRPPVIVLQRGACGQGQEDPNSLRHKYNFIADVVEKIAPAVVHIELFRKLPFSKREVPVASGSGFIVSEDGLIVTNAHVVTNKHRVKVELKNGATYEAKIKDVDEKADIALIKIDHQGKLPVLLLGRSSELRPGEFVVAIGSPFSLQNTVTTGIVSTTQRGGKELGLRNSDMDYIQTDAIINYGNSGGPLVNLDGEVIGINTLKVTAGISFAIPSDKIKKFLTESHDRQAKGKAITKKKYIGIRMMSLTSSKAKELKDRHRDFPDVISGAYIIEVIPDTPAEAGGLKENDVIISINGQSVVSANDVSDVIKRESTLNMVVRRGNEDIMITVIPEEIDP

SEQ ID NO: 2 - protein, 458 aa, Homo sapiens
  Human HTRA1 amino acid sequence without predicted signal sequence
QLSRAGRSAPLAAGCPDRCEPARCPPQPEHCEGGRARDACGCCEVCGAPEGAACGLQEGPCGEGLQCVVPFGVPASATVRRRAQAGLCVCASSEPVCGSDANTYANLCQLRAASRRSERLHRPPVIVLQRGACGQGQEDPNSLRHKYNFIADVVEKIAPAVVHIELFRKLPFSKREVPVASGSGFIVSEDGLIVTNAHVVTNKHRVKVELKNGATYEAKIKDVDEKADIALIKIDHQGKLPVLLLGRSSELRPGEFVVAIGSPFSLQNTVTTGIVSTTQRGGKELGLRNSDMDYIQTDAIINYGNSGGPLVNLDGEVIGINTLKVTAGISFAIPSDKIKKFLTESHDRQAKGKAITKKKYIGIRMMSLTSSKAKELKDRHRDFPDVISGAYIIEVIPDTPAEAGGLKENDVIISINGQSVVSANDVSDVIKRESTLNMVVRRGNEDIMITVIPEEIDP

SEQ ID NO: 3 - protein, 380 aa, Homo sapiens
  Human HTRA1 amino acid sequence without N-terminal domain, aa 101-480
VRRRAQAGLCVCASSEPVCGSDANTYANLCQLRAASRRSERLHRPPVIVLQRGACGQGQEDPNSLRHKYNFIADVVEKIAPAVVHIELFRKLPFSKREVPVASGSGFIVSEDGLIVTNAHVVTNKHRVKVELKNGATYEAKIKDVDEKADIALIKIDHQGKLPVLLLGRSSELRPGEFVVAIGSPFSLQNTVTTGIVSTTQRGGKELGLRNSDMDYIQTDAIINYGNSGGPLVNLDGEVIGINTLKVTAGISFAIPSDKIKKFLTESHDRQAKGKAITKKKYIGIRMMSLTSSKAKELKDRHRDFPDVISGAYIIEVIPDTPAEAGGLKENDVIISINGQSVVSANDVSDVIKRESTLNMVVRRGNEDIMITVIPEEIDP

SEQ ID NO: 4 - protein, 161 aa, Homo sapiens
  Human HTRA1 serine protease domain
GSGFIVSEDGLIVTNAHVVTNKHRVKVELKNGATYEAKIKDVDEKADIALIKIDHQGKLPVLLLGRSSELRPGEFVVAIGSPFSLQNTVTTGIVSTTQRGGKELGLRNSDMDYIQTDAIINYGNSGGPLVNLDGEVIGINTLKVTAGISFAIPSDKIKKFL

SEQ ID NO: 5 - protein, 523 aa, Rabbit
  Rabbit HTRA1 amino acid sequence with predicted signal sequence
MGWAARPRALAPAAPKALRSSPRRCCPARAQLAAVGAAMQSSRAARLLPLLLLLLAAPAWAQPPRAGRSAPAATSPVAGCPERCEPARCAPPPTNCEGGRVRDACGCCEVCGAPEGAACGLQEGPCGEGLQCVVSFGVPASATVRRRSQAGVCVCASNEPVCGSDANTYANLCQLRAASRRSERLQQPPVIVLQRGACGQGQEDPNSLRHKYNFIADVVEKIAPAVVHIELFRKLPFSKREVPVASGSGFIVSEDGLIVTNAHVVTNKHRVKVELKNGATYEAKIKDVDEKADIALIKIDHQGKLPVLLLGRSSELRPGEFVVAIGSPFSLQNTVTTGIVSTTQRGGKELGLRNSDMDYIQTDAIINYGNSGGPLVNLDGEVIGINTLKVTAGISFAIPSDKIKKFLTESHDRQAKGKAITKKKYIGIRMMSLTSSKAKELKDRHRDFPDVLSGAYIIEVIPDTPAEAGGLKENDVIISINGQSVVSANDVSDVIKKDSTLNMVVRRGNEDIMITVIPEEIDP

SEQ ID NO: 6 - protein, 462 aa, Rabbit
  Rabbit HTRA1 amino acid sequence without predicted signal sequence
QPPRAGRSAPAATSPVAGCPERCEPARCAPPPTNCEGGRVRDACGCCEVCGAPEGAACGLQEGPCGEGLQCVVSFGVPASATVRRRSQAGVCVCASNEPVCGSDANTYANLCQLRAASRRSERLQQPPVIVLQRGACGQGQEDPNSLRHKYNFIADVVEKIAPAVVHIELFRKLPFSKREVPVASGSGFIVSEDGLIVTNAHVVTNKHRVKVELKNGATYEAKIKDVDEKADIALIKIDHQGKLPVLLLGRSSELRPGEFVVAIGSPFSLQNTVTTGIVSTTQRGGKELGLRNSDMDYIQTDAIINYGNSGGPLVNLDGEVIGINTLKVTAGISFAIPSDKIKKFLTESHDRQAKGKAITKKKYIGIRMMSLTSSKAKELKDRHRDFPDVLSGAYIIEVIPDTPAEAGGLKENDVIISINGQSVVSANDVSDVIKKDSTLNMVVRRGNEDIMITVIPEEIDP

SEQ ID NO: 7 - protein, 462 aa, Cyno
  Cyno HTRA1 amino acid sequence with predicted signal sequence
MQIPRAALLPLLLLLLLAAPASAQLSRAGRSPEHCEGGRARDACGCCEVCGAPEGAACGLQEGPCGEGLQCVVPFGVPASATVRRRAQAGLCVCASNEPVCGSDANTYANLCQLRAASRRSERLHRPPVIVLQRGACGQGQEDPNSLRHKYNFIADVVEKIAPAVVHIELFRKLPFSKREVPVASGSGFIVSEDGLIVTNAHVVTNKHRVKVELKNGATYEAKIKDVDEKADIALIKIDHQGKLPVLLLGRSSELRPGEFVVAIGSPFSLQNTVTTGIVSTTQRGGKELGLRNSDMDYIQTDAIINYGNSGGPLVNLDGEVIGINTLKVTAGISFAIPSDKIKKFLTESHDRQAKGKAITKKKYIGIRMMSLTSSKAKELKDRHRDFPDVISGAYIIEVIPDTPAEAGGLKENDVIISINGQSVVSANDVSDVIKRESTLNMVVRRGNEDIMITVIPEEIDP

SEQ ID NO: 8 - protein, 439 aa, Cyno
  Cyno HTRA1 amino acid sequence without predicted signal sequence
QLSRAGRSPEHCEGGRARDACGCCEVCGAPEGAACGLQEGPCGEGLQCVVPFGVPASATVRRRAQAGLCVCASNEPVCGSDANTYANLCQLRAASRRSERLHRPPVIVLQRGACGQGQEDPNSLRHKYNFIADVVEKIAPAVVHIELFRKLPFSKREVPVASGSGFIVSEDGLIVTNAHVVTNKHRVKVELKNGATYEAKIKDVDEKADIALIKIDHQGKLPVLLLGRSSELRPGEFVVAIGSPFSLQNTVTTGIVSTTQRGGKELGLRNSDMDYIQTDAIINYGNSGGPLVNLDGEVIGINTLKVTAGISFAIPSDKIKKFLTESHDRQAKGKAITKKKYIGIRMMSLTSSKAKELKDRHRDFPDVISGAYIIEVIPDTPAEAGGLKENDVIISINGQSVVSANDVSDVIKRESTLNMVVRRGNEDIMITVIPEEIDP

SEQ ID NO: 9 - protein, 10 aa, Artificial Sequence
  Heavy Chain variable region CDR1 of Antibodies 24F7 & Hz24F7.v2 - Exemplary & AbM
GYTFTDYEMH

SEQ ID NO: 10 - protein, 17 aa, Artificial Sequence
  Heavy Chain variable region CDR2 of Antibodies 24F7 & Hz24F7.v2 - Exemplary & Kabat
AIDPETGGTAYNQKFKG

SEQ ID NO: 11 - protein, 14 aa, Artificial Sequence
  Heavy Chain variable region CDR3 - Exemplary, Chothia, AbM & Kabat
EGYSYDGGGYYFDY

SEQ ID NO: 12 - protein, 10 aa, Artificial Sequence
  Light Chain variable region CDR1 of Antibodies 24F7 & Hz24F7.v2 - Exemplary, Chothia, AbM & Kabat
SVSSSVSYMY

SEQ ID NO: 13 - protein, 7 aa, Artificial Sequence
  Light Chain variable region CDR2 of Antibodies 24F7, Hz24F7.v2 & 65G8 - Exemplary, Chothia, AbM & Kabat
DTSNLAS

SEQ ID NO: 14 - protein, 8 aa, Artificial Sequence
  Light Chain variable region CDR3 Antibodies 24F7 & Hz24F7.v2 - Exemplary, Chothia, AbM & Kabat
QQWSSYPT

SEQ ID NO: 15 - protein, 7 aa, Artificial Sequence
  Heavy Chain variable region CDR1 of Antibodies 24F7 & Hz24F7.v2 - Chothia
GYTFTDY

SEQ ID NO: 16 - protein, 6 aa, Artificial Sequence
  Heavy Chain variable region CDR2 of Antibodies 24F7 & Hz24F7.v2 - Chothia
DPETGG

SEQ ID NO: 17 - protein, 10 aa, Artificial Sequence
  Heavy Chain variable region CDR2 of Antibodies 24F7 & Hz24F7.v2 - AbM
AIDPETGGTA

SEQ ID NO: 18 - protein, 5 aa, Artificial Sequence
  Heavy Chain variable region CDR1 of Antibodies 24F7 & Hz24F7.v2 - Kabat
DYEMH

SEQ ID NO: 19 - protein, 6 aa, Artificial Sequence
  Heavy Chain variable region CDR1 of Antibodies 24F7 & Hz24F7.v2 - Contact
TDYEMH

SEQ ID NO: 20 - protein, 13 aa, Artificial Sequence
  Heavy Chain variable region CDR2 of Antibodies 24F7 & Hz24F7.v2 - Contact
WIGAIDPETGGTA

SEQ ID NO: 21 - protein, 15 aa, Artificial Sequence
  Heavy Chain variable region CDR3 - Contact
TREGYSYDGGGYYFD

SEQ ID NO: 22 - protein, 6 aa, Artificial Sequence
  Light Chain variable region CDR1 of Antibodies 24F7, Hz24F7.v2 & 65G8 - Contact
SYMYWY

SEQ ID NO: 23 - protein, 10 aa, Artificial Sequence
  Light Chain variable region CDR2 of Antibodies 24F7, Hz24F7.v2 & 65G8 - Contact
LLIYDTSNLA

SEQ ID NO: 24 - protein, 7 aa, Artificial Sequence
  Light Chain variable region CDR3 of Antibodies 24F7 & Hz24F7.v2 - Contact
QQWSSYP

SEQ ID NO: 25 - protein, 14 aa, Artificial Sequence
  Heavy Chain variable region CDR3 of Antibody Hz24F7.v2 - Exemplary, Chothia, AbM & Kabat
EGYSYEGGGYYFDY

SEQ ID NO: 26 - protein, 15 aa, Artificial Sequence
  Heavy Chain variable region CDR3 of Antibody Hz24F7.v2 - Contact
TREGYSYEGGGYYFD

SEQ ID NO: 27 - protein, 10 aa, Artificial Sequence
  Heavy Chain variable region CDR1 of Antibody 9F8 - Exemplary & AbM
GYAFTTYWMH

SEQ ID NO: 28 - protein, 17 aa, Artificial Sequence
  Heavy Chain variable region CDR2 of Antibody 9F8 - Exemplary & Kabat
NIDPSDSETHYNQKFRD

SEQ ID NO: 29 - protein, 7 aa, Artificial Sequence
  Heavy Chain variable region CDR3 of Antibody 9F8 - Exemplary, Chothia, AbM & Kabat
DYGAFDV

SEQ ID NO: 30 - protein, 14 aa, Artificial Sequence
  Light Chain variable region CDR1 of Antibody 9F8 - Exemplary, Chothia, AbM & Kabat
RSSTGAVTTRNFAS

SEQ ID NO: 31 - protein, 7 aa, Artificial Sequence
  Light Chain variable region CDR2 of Antibody 9F8 - Exemplary, Chothia, AbM & Kabat
GTNNRAP

SEQ ID NO: 32 - protein, 9 aa, Artificial Sequence
  Light Chain variable region CDR3 of Antibody 9F8 - Exemplary, Chothia, AbM & Kabat
ALWYSNLWV

SEQ ID NO: 33 - protein, 7 aa, Artificial Sequence
  Heavy Chain variable region CDR1 of Antibody 9F8 - Chothia
GYAFTTY

SEQ ID NO: 34 - protein, 6 aa, Artificial Sequence
  Heavy Chain variable region CDR2 of Antibodies 9F8, 65G8 & 55B12 - Chothia
DPSDSE

SEQ ID NO: 35 - protein, 10 aa, Artificial Sequence
  Heavy Chain variable region CDR2 of Antibodies 9F8 & 55B12 - AbM
NIDPSDSETH

SEQ ID NO: 36 - protein, 5 aa, Artificial Sequence
  Heavy Chain variable region CDR1 of Antibody 9F8 - Kabat
TYWMH

SEQ ID NO: 37 - protein, 6 aa, Artificial Sequence
  Heavy Chain variable region CDR1 of Antibody 9F8 - Contact
TTYWMH

SEQ ID NO: 38 - protein, 13 aa, Artificial Sequence
  Heavy Chain variable region CDR2 of Antibodies 9F8 & 55B12  - Contact
WIGNIDPSDSETH

SEQ ID NO: 39 - protein, 8 aa, Artificial Sequence
  Heavy Chain variable region CDR3 of Antibody 9F8 - Contact
ARDYGAFD

SEQ ID NO: 40 - protein, 10 aa, Artificial Sequence
  Light Chain variable region CDR1 of Antibody 9F8 - Contact
VTTRNFASWV

SEQ ID NO: 41 - protein, 10 aa, Artificial Sequence
  Light Chain variable region CDR2 of Antibody 9F8 - Contact
GLIGGTNNRA

SEQ ID NO: 42 - protein, 8 aa, Artificial Sequence
  Light Chain variable region CDR3 of Antibody 9F8 - Contact
ALWYSNLW

SEQ ID NO: 43 - protein, 10 aa, Artificial Sequence
  Heavy Chain variable region CDR1 of Antibody 55B12 - Exemplary & AbM
GYTFTNYWMH

SEQ ID NO: 44 - protein, 17 aa, Artificial Sequence
  Heavy Chain variable region CDR2 of Antibody 55B12 - Exemplary & Kabat
NIDPSDSETHYNQKFKD

SEQ ID NO: 45 - protein, 9 aa, Artificial Sequence
  Heavy Chain variable region CDR3 of Antibody 55B12 - Exemplary, Chothia, AbM & Kabat
EDSSGYGAY

SEQ ID NO: 46 - protein, 10 aa, Artificial Sequence
  Light Chain variable region CDR1 of Antibody 55B12 - Exemplary, Chothia, AbM & Kabat
SASSSVNYMH

SEQ ID NO: 47 - protein, 7 aa, Artificial Sequence
  Light Chain variable region CDR2 of Antibody 55B12 - Exemplary, Chothia, AbM & Kabat
DTSKLAS

SEQ ID NO: 48 - protein, 9 aa, Artificial Sequence
  Light Chain variable region CDR3 of Antibody 55B12 - Exemplary, Chothia, AbM & Kabat
QQWSSHPLT

SEQ ID NO: 49 - protein, 7 aa, Artificial Sequence
  Heavy Chain variable region CDR1 of Antibody 55B12 - Chothia
GYTFTNY

SEQ ID NO: 50 - protein, 5 aa, Artificial Sequence
  Heavy Chain variable region CDR1 of Antibody 55B12-Kabat
NYWMH

SEQ ID NO: 51 - protein, 6 aa, Artificial Sequence
  Heavy Chain variable region CDR1 of Antibody 55B12-Contact
TNYWMH

SEQ ID NO: 52 - protein, 10 aa, Artificial Sequence
  Heavy Chain variable region CDR3 of Antibody 55B12-Contact
AREDSSGYGA

SEQ ID NO: 53 - protein, 6 aa, Artificial Sequence
  Light Chain variable region CDR1 of Antibody 55B12-Contact
NYMHWY

SEQ ID NO: 54 - protein, 10 aa, Artificial Sequence
  Light Chain variable region CDR2 of Antibody 55B12 -Contact
RWIYDTSKLA

SEQ ID NO: 55 - protein, 8 aa, Artificial Sequence
  Light Chain variable region CDR3 of Antibody 55B12 -Contact
QQWSSHPL

SEQ ID NO: 56 - protein, 10 aa, Artificial Sequence
  Heavy Chain variable region CDR1 of Antibody 65G8 - Exemplary & AbM
GYSFTSYWMH

SEQ ID NO: 57 - protein, 17 aa, Artificial Sequence
  Heavy Chain variable region CDR2 of Antibody 65G8 - Exemplary & Kabat
MIDPSDSETRLNQKFKD

SEQ ID NO: 58 - protein, 5 aa, Artificial Sequence
  Heavy Chain variable region CDR3 of Antibody 65G8 - Exemplary, Chothia, AbM & Kabat
DYFDY

SEQ ID NO: 59 - protein, 10 aa, Artificial Sequence
  Light Chain variable region CDR1 of Antibody 65G8 - Exemplary, Chothia, AbM & Kabat
SASSSVSYMY

SEQ ID NO: 60 - protein, 9 aa, Artificial Sequence
  Light Chain variable region CDR3 of Antibody 65G8 - Exemplary, Chothia, AbM & Kabat
QQWSSYPYT

SEQ ID NO: 61 - protein, 7 aa, Artificial Sequence
  Heavy Chain variable region CDR1 of Antibody 65G8 -Chothia
GYSFTSY

SEQ ID NO: 62 - protein, 10 aa, Artificial Sequence
  Heavy Chain variable region CDR2 of Antibody 65G8 - AbM
MIDPSDSETR

SEQ ID NO: 63 - protein, 5 aa, Artificial Sequence
  Heavy Chain variable region CDR1 of Antibody 65G8 -Kabat
SYWMH

SEQ ID NO: 64 - protein, 6 aa, Artificial Sequence
  Heavy Chain variable region CDR1 of Antibody 65G8 -Contact
TSYWMH

SEQ ID NO: 65 - protein, 13 aa, Artificial Sequence
  Heavy Chain variable region CDR2 of Antibody 65G8 -Contact
WIGMIDPSDSETR

SEQ ID NO: 66 - protein, 6 aa, Artificial Sequence
  Heavy Chain variable region CDR3 of Antibody 65G8 -Contact
TRDYFD

SEQ ID NO: 67 - protein, 8 aa, Artificial Sequence
  Light Chain variable region CDR3 of Antibody 65G8 -Contact
QQWSSYPY

SEQ ID NO: 68 - protein, 123 aa, Artificial Sequence
  24F7 Heavy chain variable region amino acid sequence
QVQLQQSGAELVRPGASVKLSCKASGYTFTDYEMHWVKQTPVHGLEWIGAIDPETGGTAYNQKFKGKATLTADKSSSTAYMELRSLTSEDSAVYYCTREGYSYDGGGYYFDYWGQGTTLTVSS

SEQ ID NO: 69 - protein, 105 aa, Artificial Sequence
  24F7 Light chain variable region amino acid sequence
QIVLTQSPAIMSASPGEKVTMTCSVSSSVSYMYWYQQKPGSSPRLLIYDTSNLASGVPVRFSGSGSGTSYSLTISRMEAEDAATYYCQQWSSYPTFGGGTKLEIK

SEQ ID NO: 70 - protein, 123 aa, Artificial Sequence
  Hz24F7 Heavy chain variable region amino acid sequence
QVQLVQSGAEVKKPGASVKVSCKASGYTFTDYEMHWVRQAPGQRLEWMGAIDPETGGTAYNQKFKGRVTITADKSASTAYMELSSLRSEDTAVYYCAREGYSYDGGGYYFDYWGQGTLVTVSS

SEQ ID NO: 71 - protein, 123 aa, Artificial Sequence
  Hz24F7.v2 variant Heavy chain variable region amino acid sequence
QVQLVQSGAEVKKPGASVKVSCKASGYTFTDYEMHWVRQAPGQRLEWMGAIDPETGGTAYNQKFKGRVTITADKSASTAYMELSSLRSEDTAVYYCTREGYSYEGGGYYFDYWGQGTLVTVSS

SEQ ID NO: 72 - protein, 105 aa, Artificial Sequence
  Hz24F7.v2 Light chain variable region amino acid sequence
DIQMTQSPSSLSASVGDRVTITCSVSSSVSYMYWYQQKPGKAPKLLIYDTSNLASGVPSRFSGSGSGTDFTFTISSLQPEDIATYYCQQWSSYPTFGQGTKLEIK

SEQ ID NO: 73 - protein, 116 aa, Artificial Sequence
  9F8 Heavy chain variable region amino acid sequence
QVQLQQPGAELVRPGSSVKLSCKASGYAFTTYWMHWVKQRPIQGLEWIGNIDPSDSETHYNQKFRDKATLTVDKSSSTAYMQLSSLTSEDSAVYYCARDYGAFDVWGTGTTVTVSS

SEQ ID NO: 74 - protein, 109 aa, Artificial Sequence
  9F8 Light chain variable region amino acid sequence
QAVVTQESALTTSSGETVTLTCRSSTGAVTTRNFASWVQEKPDHLFTGLIGGTNNRAPGVPARFSGSLIGDKAALTITGAQTEDEAIYFCALWYSNLWVFGGGTKLTVL

SEQ ID NO: 75 - protein, 118 aa, Artificial Sequence
  55B12 Heavy chain variable region amino acid sequence
QVQLQQPGAELVKPGASVKLSCKASGYTFTNYWMHWVKQRPGQGLEWIGNIDPSDSETHYNQKFKDKATLAVDKSSSTAYMQLSSLTSEDSAVYYCAREDSSGYGAYWGQGTLVTVSA

SEQ ID NO: 76 - protein, 106 aa, Artificial Sequence
  55B12 Light chain variable region amino acid sequence
QIVLTQSPAIMSASPGEKVTMTCSASSSVNYMHWYQQKSGTSPKRWIYDTSKLASGVPDRFSGSGSGTSYSLTISSMEAEDAATYYCQQWSSHPLTFGAGTKLELK

SEQ ID NO: 77 - protein, 114 aa, Artificial Sequence
  65G8 Heavy chain variable region amino acid sequence
QVQLQQSGPQLVRPGASVKISCKASGYSFTSYWMHWVKQRPGQGLEWIGMIDPSDSETRLNQKFKDKATLTIDKSSSTAYMQLSSPTSEDSAVYYCTRDYFDYWGQGTTLTVSS

SEQ ID NO: 78 - protein, 106 aa, Artificial Sequence
  65G8 Light chain variable region amino acid sequence
QIVLTQSPAIMSTSPGEKVTMTCSASSSVSYMYWYQQKPGSSPRLLIYDTSNLASGVPVRFSGSGSGTSYSLTISRMEAEDAATYYCQQWSSYPYTFGGGTKLEIK

SEQ ID NO: 79 - protein, 330 aa, Homo sapiens
  Human IgG1 constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 80 - protein, 330 aa, Homo sapiens
  Human IgG1 constant region E233A/L235A
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 81 - protein, 330 aa, Homo sapiens
  Human IgG1 constant region L234A/L235A
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 82 - protein, 330 aa, Homo sapiens
  Human IgG1 constant region L234A/L235A/P329G
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 83 - protein, 330 aa, Homo sapiens
  Human IgG1 constant region N297G
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 84 - protein, 330 aa, Homo sapiens
  Human IgG1 constant region N297G/H310A
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLAQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 85 - protein, 107 aa, Homo sapiens [sequence omitted]

SEQ ID NO: 86 - protein, 106 aa, Homo sapiens
  Human Lambda light chain constant region
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 87 - protein, 475 aa, Artificial Sequence
  Hz24F7.v2 Heavy chain amino acid sequence with signal sequence
MDMRVPAQLLGLLLLWLRGARCQVQLVQSGAEVKKPGASVKVSCKASGYTFTDYEMHWVRQAPGQRLEWMGAIDPETGGTAYNQKFKGRVTITADKSASTAYMELSSLRSEDTAVYYCTREGYSYEGGGYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLAQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 88 - protein, 453 aa, Artificial Sequence
  Hz24F7.v2 Heavy chain amino acid sequence without signal sequence
QVQLVQSGAEVKKPGASVKVSCKASGYTFTDYEMHWVRQAPGQRLEWMGAIDPETGGTAYNQKFKGRVTITADKSASTAYMELSSLRSEDTAVYYCTREGYSYEGGGYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLAQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 89 - protein, 234 aa, Artificial Sequence
  Hz24F7.v2 Light chain amino acid sequence with signal sequence
MDMRVPAQLLGLLLLWLRGARCDIQMTQSPSSLSASVGDRVTITCSVSSSVSYMYWYQQKPGKAPKLLIYDTSNLASGVPSRFSGSGSGTDFTFTISSLQPEDIATYYCQQWSSYPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 90 - protein, 212 aa, Artificial Sequence
  Hz24F7.v2 Light chain amino acid sequence without signal sequence
DIQMTQSPSSLSASVGDRVTITCSVSSSVSYMYWYQQKPGKAPKLLIYDTSNLASGVPSRFSGSGSGTDFTFTISSLQPEDIATYYCQQWSSYPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 91 - protein, 10 aa, Artificial Sequence
  Substrate peptide
  MOD_RES (9)..(9): Xaa is Lys with a dnp molecule attached
IRRVSYSFXK

SEQ ID NO: 92 - protein, 12 aa, Artificial Sequence
  HTRA1 peptide
RKLPFSKREVPV

SEQ ID NO: 93 - protein, 6 aa, Artificial Sequence
  Hexa-histidine tag
HHHHHH

SEQ ID NO: 94 - protein, 8 aa, Artificial Sequence
  Hz24F7.v2 S91Y Light Chain CDR3 - Exemplary, Chothia, AbM & Kabat
QQWYSYPT

SEQ ID NO: 95 - protein, 7 aa, Artificial Sequence
  Hz24F7.v2 S91Y mutation Light Chain CDR3 - Contact
QQWYSYP

SEQ ID NO: 96 - protein, 105 aa, Artificial Sequence
  Hz24F7.v2 S91Y Light chain variable region amino acid sequence
DIQMTQSPSSLSASVGDRVTITCSVSSSVSYMYWYQQKPGKAPKLLIYDTSNLASGVPSRFSGSGSGTDFTFTISSLQPEDIATYYCQQWYSYPTFGQGTKLEIK

SEQ ID NO: 97 - protein, 234 aa, Artificial Sequence
  Hz24F7.v2 S91Y Light chain amino acid sequence with signal sequence
MDMRVPAQLLGLLLLWLRGARCDIQMTQSPSSLSASVGDRVTITCSVSSSVSYMYWYQQKPGKAPKLLIYDTSNLASGVPSRFSGSGSGTDFTFTISSLQPEDIATYYCQQWYSYPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 98 - protein, 212 aa, Artificial Sequence
  Hz24F7.v2 S91Y Light chain amino acid sequence without signal sequence
DIQMTQSPSSLSASVGDRVTITCSVSSSVSYMYWYQQKPGKAPKLLIYDTSNLASGVPSRFSGSGSGTDFTFTISSLQPEDIATYYCQQWYSYPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 99 - protein, 8 aa, Artificial Sequence
  Hz24F7.v2 S92T Light Chain CDR3 - Exemplary, Chothia, AbM & Kabat
QQWSTYPT

SEQ ID NO: 100 - protein, 7 aa, Artificial Sequence
  Hz24F7.v2 S92T Light Chain CDR3 - Contact
QQWSTYP

SEQ ID NO: 101 - protein, 105 aa, Artificial Sequence; identical to SEQ ID NO: 96
  Hz24F7.v2 S92T Light chain variable region amino acid sequence

SEQ ID NO: 102 - protein, 234 aa, Artificial Sequence
  Hz24F7.v2 S92T Light chain amino acid sequence with signal sequence
MDMRVPAQLLGLLLLWLRGARCDIQMTQSPSSLSASVGDRVTITCSVSSSVSYMYWYQQKPGKAPKLLIYDTSNLASGVPSRFSGSGSGTDFTFTISSLQPEDIATYYCQQWSTYPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 103 - protein, 212 aa, Artificial Sequence
  Hz24F7.v2 S92T Light chain amino acid sequence without signal sequence
DIQMTQSPSSLSASVGDRVTITCSVSSSVSYMYWYQQKPGKAPKLLIYDTSNLASGVPSRFSGSGSGTDFTFTISSLQPEDIATYYCQQWSTYPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 104 - protein, 8 aa, Artificial Sequence
  hz24F7.v2 S91D Light Chain CDR3 - Exemplary, Chothia, AbM and Kabat
QQWDSYPT

SEQ ID NO: 105 - protein, 7 aa, Artificial Sequence
  hz24F7.v2 S91D Light Chain CDR3 - Contact
QQWDSYP

SEQ ID NO: 106 - protein, 105 aa, Artificial Sequence
  hz24F7.v2 S91D Light chain variable region amino acid sequence
DIQMTQSPSSLSASVGDRVTITCSVSSSVSYMYWYQQKPGKAPKLLIYDTSNLASGVPSRFSGSGSGTDFTFTISSLQPEDIATYYCQQWDSYPTFGQGTKLEIK

SEQ ID NO: 107 - protein, 8 aa, Artificial Sequence
  hz24F7.v2 S91T Light Chain CDR3 - Exemplary, Chothia, AbM and Kabat
QQWTSYPT

SEQ ID NO: 108 - protein, 7 aa, Artificial Sequence
  hz24F7.v2 S91T Light Chain CDR3 - Contact
QQWTSYP

SEQ ID NO: 109 - protein, 105 aa, Artificial Sequence
  hz24F7.v2 S91T Light chain variable region amino acid sequence
DIQMTQSPSSLSASVGDRVTITCSVSSSVSYMYWYQQKPGKAPKLLIYDTSNLASGVPSRFSGSGSGTDFTFTISSLQPEDIATYYCQQWTSYPTFGQGTKLEIK

SEQ ID NO: 110 - protein, 8 aa, Artificial Sequence
  hz24F7.v2 S91A Light Chain CDR3 - Exemplary, Chothia, AbM and Kabat
QQWASYPT

SEQ ID NO: 111 - protein, 7 aa, Artificial Sequence
  hz24F7.v2 S91A Light Chain CDR3 - Contact
QQWASYP

SEQ ID NO: 112 - protein, 105 aa, Artificial Sequence
  hz24F7.v2 S91A Light chain variable region amino acid sequence
DIQMTQSPSSLSASVGDRVTITCSVSSSVSYMYWYQQKPGKAPKLLIYDTSNLASGVPSRFSGSGSGTDFTFTISSLQPEDIATYYCQQWASYPTFGQGTKLEIK

SEQ ID NO: 113 - protein, 8 aa, Artificial Sequence
  hz24F7.v2 S91L Light Chain CDR3 - Exemplary, Chothia, AbM and Kabat
QQWLSYPT

SEQ ID NO: 114 - protein, 7 aa, Artificial Sequence
  hz24F7.v2 S91L Light Chain CDR3 - Contact
QQWLSYP

SEQ ID NO: 115 - protein, 105 aa, Artificial Sequence; identical to SEQ ID NO: 72
  hz24F7.v2 S91L Light chain variable region amino acid sequence

SEQ ID NO: 116 - protein, 8 aa, Artificial Sequence
  hz24F7.v2 S92Y Light Chain CDR3 - Exemplary, Chothia, AbM and Kabat
QQWSYYPT

SEQ ID NO: 117 - protein, 7 aa, Artificial Sequence
  hz24F7.v2 S92Y Light Chain CDR3 - Contact
QQWSYYP

SEQ ID NO: 118 - protein, 105 aa, Artificial Sequence
  hz24F7.v2 S92Y Light chain variable region amino acid sequence
DIQMTQSPSSLSASVGDRVTITCSVSSSVSYMYWYQQKPGKAPKLLIYDTSNLASGVPSRFSGSGSGTDFTFTISSLQPEDIATYYCQQWSYYPTFGQGTKLEIK

SEQ ID NO: 119 - protein, 8 aa, Artificial Sequence
  hz24F7.v2 S92D Light Chain CDR3 - Exemplary, Chothia, AbM and Kabat
QQWSDYPT

SEQ ID NO: 120 - protein, 7 aa, Artificial Sequence
  hz24F7.v2 S92D Light Chain CDR3 - Contact
QQWSDYP

SEQ ID NO: 121 - protein, 105 aa, Artificial Sequence
  hz24F7.v2 S92D Light chain variable region amino acid sequence
DIQMTQSPSSLSASVGDRVTITCSVSSSVSYMYWYQQKPGKAPKLLIYDTSNLASGVPSRFSGSGSGTDFTFTISSLQPEDIATYYCQQWSDYPTFGQGTKLEIK

SEQ ID NO: 122 - protein, 234 aa, Artificial Sequence
  hz24F7.v2 S91D Light chain amino acid sequence with signal sequence
MDMRVPAQLLGLLLLWLRGARCDIQMTQSPSSLSASVGDRVTITCSVSSSVSYMYWYQQKPGKAPKLLIYDTSNLASGVPSRFSGSGSGTDFTFTISSLQPEDIATYYCQQWDSYPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 123 - protein, 212 aa, Artificial Sequence
  hz24F7.v2 S91D Light chain amino acid sequence without signal sequence
DIQMTQSPSSLSASVGDRVTITCSVSSSVSYMYWYQQKPGKAPKLLIYDTSNLASGVPSRFSGSGSGTDFTFTISSLQPEDIATYYCQQWDSYPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 124 - protein, 234 aa, Artificial Sequence
  hz24F7.v2 S91T Light chain amino acid sequence with signal sequence
MDMRVPAQLLGLLLLWLRGARCDIQMTQSPSSLSASVGDRVTITCSVSSSVSYMYWYQQKPGKAPKLLIYDTSNLASGVPSRFSGSGSGTDFTFTISSLQPEDIATYYCQQWTSYPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 125 - protein, 212 aa, Artificial Sequence
  hz24F7.v2 S91T Light chain amino acid sequence without signal sequence
DIQMTQSPSSLSASVGDRVTITCSVSSSVSYMYWYQQKPGKAPKLLIYDTSNLASGVPSRFSGSGSGTDFTFTISSLQPEDIATYYCQQWTSYPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 126 - protein, 234 aa, Artificial Sequence
  hz24F7.v2 S91A Light chain amino acid sequence with signal sequence
MDMRVPAQLLGLLLLWLRGARCDIQMTQSPSSLSASVGDRVTITCSVSSSVSYMYWYQQKPGKAPKLLIYDTSNLASGVPSRFSGSGSGTDFTFTISSLQPEDIATYYCQQWASYPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 127 - protein, 212 aa, Artificial Sequence
  hz24F7.v2 S91A Light chain amino acid sequence without signal sequence
DIQMTQSPSSLSASVGDRVTITCSVSSSVSYMYWYQQKPGKAPKLLIYDTSNLASGVPSRFSGSGSGTDFTFTISSLQPEDIATYYCQQWASYPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 128 - protein, 234 aa, Artificial Sequence; identical to SEQ ID NO: 89
  hz24F7.v2 S91L Light chain amino acid sequence with signal sequence

SEQ ID NO: 129 - protein, 212 aa, Artificial Sequence; identical to SEQ ID NO: 90
  hz24F7.v2 S91L Light chain amino acid sequence without signal sequence

SEQ ID NO: 130 - protein, 234 aa, Artificial Sequence
  hz24F7.v2 S92Y Light chain amino acid sequence with signal sequence
MDMRVPAQLLGLLLLWLRGARCDIQMTQSPSSLSASVGDRVTITCSVSSSVSYMYWYQQKPGKAPKLLIYDTSNLASGVPSRFSGSGSGTDFTFTISSLQPEDIATYYCQQWSYYPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 131 - protein, 212 aa, Artificial Sequence
  hz24F7.v2 S92Y Light chain amino acid sequence without signal sequence
DIQMTQSPSSLSASVGDRVTITCSVSSSVSYMYWYQQKPGKAPKLLIYDTSNLASGVPSRFSGSGSGTDFTFTISSLQPEDIATYYCQQWSYYPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 132 - protein, 234 aa, Artificial Sequence
  hz24F7.v2 S92D Light chain amino acid sequence with signal sequence
MDMRVPAQLLGLLLLWLRGARCDIQMTQSPSSLSASVGDRVTITCSVSSSVSYMYWYQQKPGKAPKLLIYDTSNLASGVPSRFSGSGSGTDFTFTISSLQPEDIATYYCQQWSDYPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 133 - protein, 212 aa, Artificial Sequence
  hz24F7.v2 S92D Light chain amino acid sequence without signal sequence
DIQMTQSPSSLSASVGDRVTITCSVSSSVSYMYWYQQKPGKAPKLLIYDTSNLASGVPSRFSGSGSGTDFTFTISSLQPEDIATYYCQQWSDYPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC